(19) United States
(12) Patent Application Publication
(10) Pub. No.: US 2018/0369268 A1
(43) Pub. Date: Dec. 27, 2018

(54) METHODS FOR IDENTIFYING INHIBITORS OF "STIMULATOR OF INTERFERON GENE"- DEPENDENT INTERFERON PRODUCTION

(71) Applicant: ADURO BIOTECH, INC., Berkeley (CA)

(72) Inventors: George Edwin KATIBAH, Fremont, CA (US); David KANNE, Corte Madera, CA (US); Leonard SUNG, San Mateo, CA (US); Justin LEONG, Union City, CA (US); Sarah M. McWHIRTER, Albany, CA (US); Thomas W. DUBENSKY, JR., Berkeley, CA (US)

(73) Assignee: ADURO BIOTECH, INC., BERKELEY, CA (US)

(21) Appl. No.: 16/062,601

(22) PCT Filed: Dec. 16, 2016
(86) PCT No.: PCT/US16/67315

§ 371 Date: Jun. 14, 2018

Related U.S. Application Data
(60) Provisional application No. 62/268,480, filed on Dec. 16, 2015.
(60) Provisional application No. 62/268,477, filed on Dec. 16, 2015.

(51) Int. Cl. A61K 31/7084; G01N 33/50; A61K 45/06; A61K 39/39; A61K 35/17; C07D 307/04; C07D 273/02

(52) U.S. Cl. CPC A61K 31/7084; G01N 33/5008; A61K 45/06; A61P 37/06; A61K 35/17; C07D 307/04; C07D 273/02; A61K 39/39

(57) ABSTRACT

The present invention relates to the use cyclic-di-nucleotide and related scaffold molecules that measurably inhibit STING signaling, and methods for their use in identifying more potent inhibitors of STING signaling. In particular, the methods provided can be used to identify potent inhibitors of STING signaling, which are useful in the treatment of autoimmune and inflammatory diseases. Also provided are compounds having STING inhibitory activity useful in the treatment of autoimmune and inflammatory diseases.

The present application claims priority to U.S. Provisional Patent Application 62/268,477, filed Dec. 16, 2015, and U.S. Provisional Patent Application 62/268,480, filed Dec. 16, 2015, each of which is hereby incorporated in its entirety including all tables, figures, and claims.

### BACKGROUND OF THE INVENTION

The following discussion of the background of the invention is merely provided to aid the reader in understanding the invention and is not admitted to describe or constitute prior art to the present invention.

The human immune system may generally be divided into two arms, referred to as “innate immunity” and “adaptive immunity.” The innate arm of the immune system is predominantly responsible for an initial inflammatory response via a number of soluble factors, including the complement system and the chemokine/cytokine system; and a number of specialized cell types including mast cells, macrophages, dendritic cells (DCs), and natural killer cells. In contrast, the adaptive immune arm involves a delayed and a longer lasting antibody response together with CD8+ and CD4+ T cell responses that play a critical role in immunological memory against an antigen. A third arm of the immune system may be identified as involving γδ T cells and T cells with limited T cell receptor repertoires such as NKT cells and MAIT cells.

For an effective immune response to an antigen, antigen presenting cells (APCs) must process and display the antigen in a proper MHC context to a T cell, which then will result in either T cell stimulation of cytotoxic and helper T cells. Following antigen presentation, successful interaction of co-stimulatory molecules on both APCs and T cells must occur or activation will be aborted. GM-CSF and IL-12 serve as effective pro-inflammatory molecules in many tumor models. For example, GM-CSF induces myeloid precursor cells to proliferate and differentiate into dendritic cells (DCs) although additional signals are necessary to activate their maturation to effective antigen-presenting cells necessary for activation of T cells. Barriers to effective immune therapies include tolerance to the targeted antigen that can limit induction of cytotoxic CD8 T cells of appropriate magnitude and function, poor trafficking of the generated T cells to sites of malignant cells, and poor persistence of the induced T cell response.

DCs that phagocytose tumor-cell debris process the material for major histocompatibility complex (MHC) presentation, upregulate expression of costimulatory molecules, and migrate to regional lymph nodes to stimulate tumor-specific lymphocytes. This pathway results in the proliferation and activation of CD4+ and CD8+ T cells that react to tumor-associated antigens. Indeed, such cells can be detected frequently in the blood, lymphoid tissues, and malignant lesions of patients.

New insights into the mechanisms underlying immune-evasion, together with combination treatment regimens that potentiate the potency of therapeutic vaccination-either directly or indirectly-through combination with immune checkpoint inhibitors or other therapies, have served as a basis for the development of vaccines that induce effective antitumor immunity. The CDNs cyclic-di-AMP (produced by Listeria monocytogenes) and its analog cyclic-di-GMP (produced by Legionella pneumophila) are recognized by the host cell as a PAMP (Pathogen Associated Molecular Pattern), which bind to the PRR (Pathogen Recognition Receptor) known as STING. STING is an adaptor protein in the cytoplasm of host mammalian cells which activates the TANK binding kinase (TBK1)-IRF3 signaling axis, resulting in the induction of IFN-β and other IRF-3 dependent gene products that strongly activate innate immunity. It is now recognized that STING is a component of the host cytosolic surveillance pathway that senses infection with intracellular pathogens and in response induces the production of IFN-β, leading to the development of an adaptive protective pathogen-specific immune response consisting of both antigen-specific CD4 and CD8 T cells as well as pathogen-specific antibodies. Examples of cyclic purine dinucleotides are described in some detail in, e.g., U.S. Pat. Nos. 7,709,458 and 7,592,326; WO2007/054279, WO2014/093936, and WO2014/189805; and Yan et al., Bioorg. Med. Chem Lett. 18: 5631 (2008), each of which is hereby incorporated by reference.

The STING-dependent type I interferon response is also associated with autoimmune disease (Gall et al., Immunity 36(1): 120-131 (2012); Liu et al., N Engl J Med. 371(6): 507-518 (2014); Jeremiah et al., J Clin Invest. 124(12): 5516-20 (2014)). The inhibition of STING-dependent activation of interferon can be beneficial in the therapeutic treatment in autoimmune diseases.

There remains a need for improved compositions and methods for immunologic strategies to treating diseases such as autoimmune diseases that may benefit from inhibition of the STING-dependent activation of type I interferon.

### SUMMARY OF THE INVENTION

It is an object of the present invention to provide scaffold molecules that inhibit STimulator of INTerferon Gene (“STING”)-dependent type I interferon production (STING inhibitory activity) for use in identifying more potent STING inhibitors.

In a first aspect, the present invention provides a scaffold molecule having the structure of Formula I, Formula II, Formula III, Formula IV or Formula V:

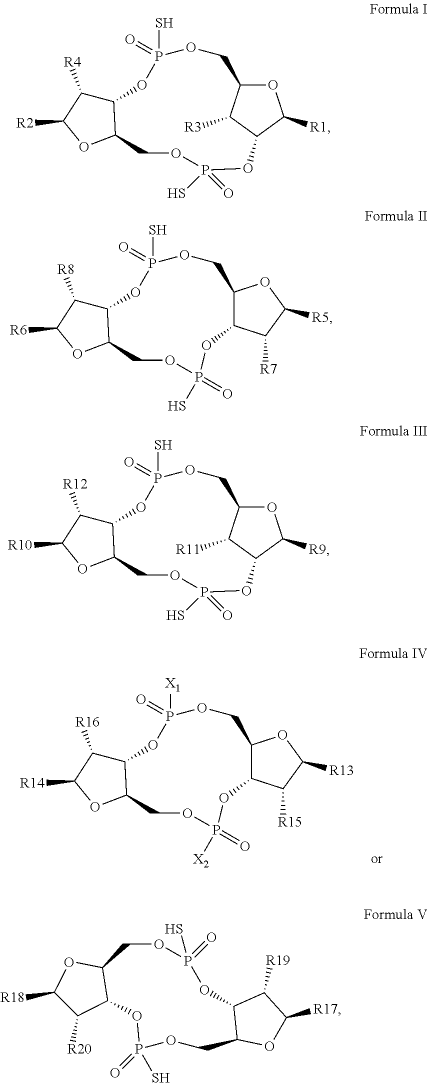

wherein:
R1 is adenine or adenine-6-benzamide linked to the structure via the N9 position;
R2 is guanine or guanine-2-isobutyramide linked to the structure via the N9 position;
R3 is —OH;
R4 is —OH;
R5 and R6 are both guanine-2-isobutyramide linked to the structure via the N9 position; or
one of R5 and R6 is adenine linked to the structure via the N9 position, and the other of R5 and R6 is cytosine linked to the structure via the N1 position;
R7 is —F;
R8 is —F;
R9 is adenine linked to the structure via the N9 position;
R10 is adenine, guanine or 2,6-diamino-purine linked to the structure via the N9 position;
R11 is —OH or —OTBS;
R12 is —F, —OH or —OTBS;
R13 is adenine, 2,6-diamino-purine, guanine or guanine-6-propargyl ether linked to the structure via the N9 position;
R14 is adenine, 2,6-diamino-purine, guanine or guanine-6-propargyl ether linked to the structure via the N9 position;
R15 is —F, —OH or —OTBS;
R16 is —F, —OH or —OTBS;
R17 and R18 are both adenine linked to the structure via the N9 position;
R19 is —OH;
R20 is —OH; and
X₁ and X₂ are independently —OH or —SH;
wherein the scaffold molecule (i) exhibits measurable STING inhibitory activity and/or (ii) exhibits measurable STING binding but is not a STING agonist.

In a first embodiment of the first aspect, the scaffold molecule measurably binds to at least one human STING (hSTING) allelic protein product (including any one of WT, REF, HAQ, AQ, and Q alleles). Preferably, the binding is measured using the isolated protein encoded by the hSTING (WT), hSTING (HAQ) or hSTING (REF) allele (Ishikawa, H., and Barber, G. N. (2008). Nature 455, 674-678; Yi et al., 2013, PLos One 2013 Oct. 21, 8(10):e77846; the protein sequence of the REF allele is NCBI Reference Sequence NP_938023). In some embodiments, the scaffold molecule measurably binds to one or more of hSTING (WT), hSTING (HAQ), or hSTING (REF). In some embodiments, the scaffold molecule measurably binds to two or more of hSTING (WT), hSTING (HAQ), or hSTING (REF). In some embodiments, the scaffold molecule measurably binds to each of hSTING (WT), hSTING (HAQ), or hSTING (REF). In some embodiments, the binding to the hSTING protein is measured by Tₘ shift in a differential scanning fluorometry assay, for example the assay according to Example 11. In some embodiments, the Tₘ shift measured for the scaffold molecule is in the range of about 2 to about 15° C. for hSTING(WT) or hSTING (REF) and in the range of about 2 to about 25° C. for hSTING (HAQ).

In a second embodiment of the first aspect and first embodiment thereof, the scaffold molecule has measurable STING inhibitory activity in a competition assay with a STING agonist. In some embodiments, the measurable STING inhibitory activity is the inhibition of STING dependent IFN-β production in a competition assay. In some embodiments, the competition assay is a cellular assay that measures the induction of STING dependent IFN-β production. In a preferred embodiment, the competition assay is a cellular assay that measures the induction of STING dependent IFN-β production, wherein the assay is performed without the addition of digitonin or other agent that increases permeabilization of the cell to the compounds being assayed. In some embodiments, the STING agonist is 2′3′-RR-(A)(A). In some embodiments, the competition assay measures the hSTING inhibitory activity of the scaffold molecule. In some embodiments, the scaffold molecule has measurable hSTING inhibitory activity in a competition assay with one or more of hSTLNG (WT), hSTING (HAQ), or hSTING (REF). In some embodiments, the scaffold molecule has measurable hSTING inhibitory activity in a competition assay with two or more of hSTING (WT), hSTING (HAQ), or hSTING (REF). In some embodiments, the scaffold molecule has measurable hSTING inhibitory activity in a competition assay with each of hSTING (WT), hSTING (HAQ), or hSTING (REF). In some embodiments, the scaffold molecule has an IC50 of less than 10 mM, less than 5 mM, or less than 1 mM, or in the range of 100 μM to 10 mM, 100 μM to 5 mM, or 100 μM to 1 mM for one or more of hSTING (WT), hSTING (HAQ), or hSTING (REF) in the competition assay with the STING agonist is 2′3′-RR-(A)(A). In some embodiments, the scaffold molecule has an IC50 of less than 10 mM, less than 5 mM, or less than 1 mM, or in the range of 100 μM to 10 mM, 100 μM to 5 mM, or 100 μM to 1 mM for two or more of hSTING (WT), hSTING (HAQ), or hSTING (REF) in the competition assay with the STING agonist is 2′3′-RR-(A)(A). In some embodiments, the scaffold molecule has an IC50 of less than 10 mM, less than 5 mM, or less than 1 mM, or in the range of 100 μM to 10 mM, 100 μM to 5 mM, or 100 μM to 1 mM for each of hSTING (WT), hSTING (HAQ), and hSTING (REF) in the competition assay with the STING agonist is 2′3′-RR-(A)(A). In some embodiments, the competition assay is the assay according to Example 13.

In a third embodiment of the first aspect and first and second embodiments thereof, the scaffold molecule has the structure of Formula Ia, Ib, Ic, Id, IIa, IIb, IIc, IId, IIIa, IIIb, IIIc, IIId, IVa, IVb, IVc, IVd, IVe, Va, Vb, Vc or Vd:

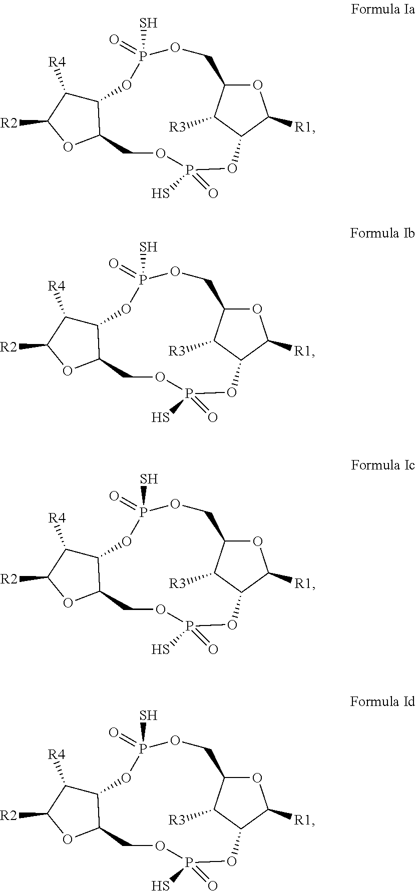

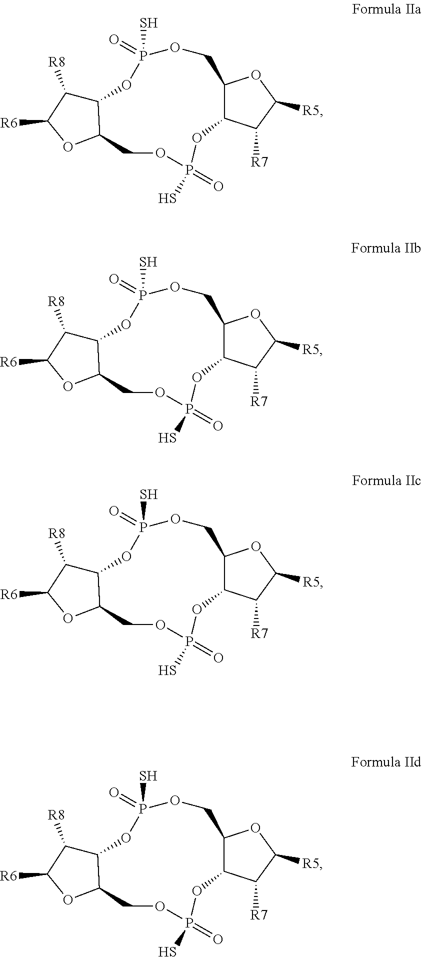

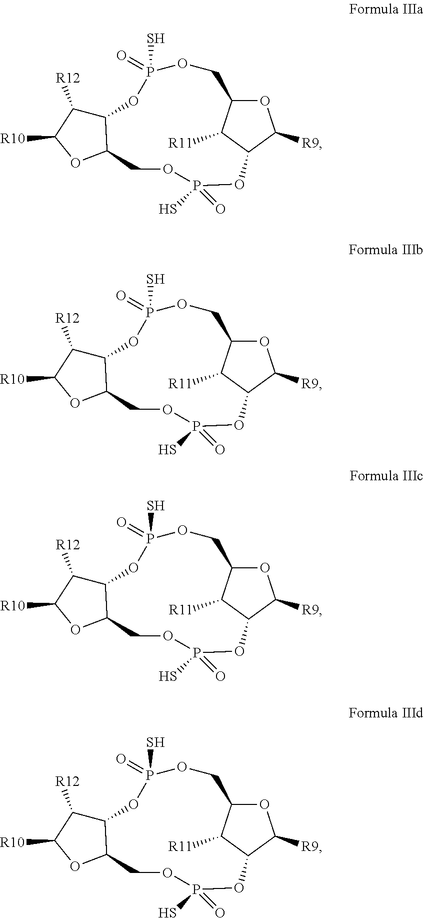

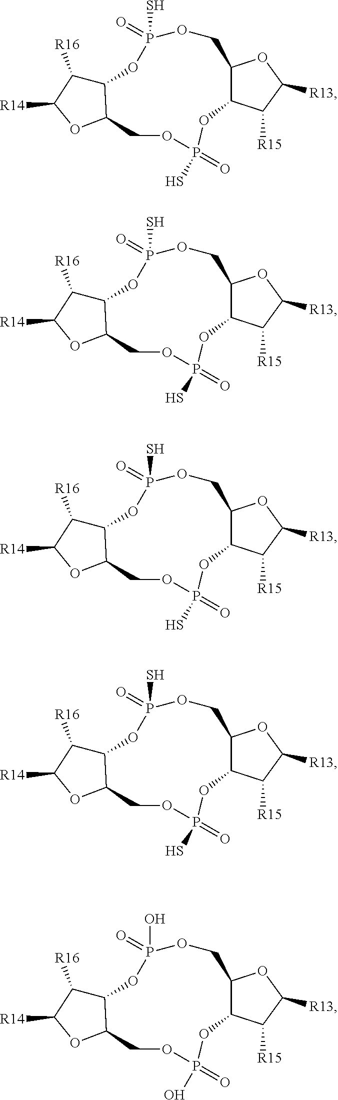

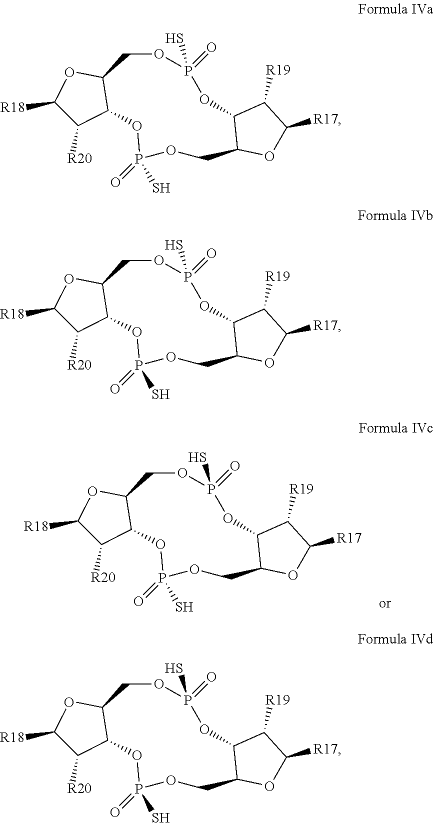

wherein R1, R2, R3, R4, R5, R6, R7, R8, R9, R10, R11, R12, R13, R14, R15, R16, R17, R18, R19, R20, X₁ and X₂ are as defined for the first aspect of the invention.

In some embodiments of the first aspect, the scaffold molecule is selected from the group consisting of:

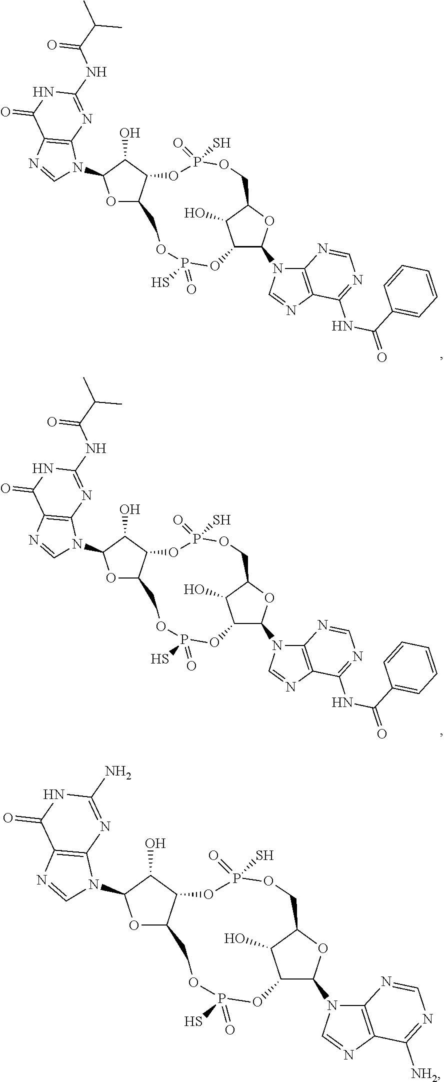 [SMILES: CC(C)C(=O)Nc1nc2c(ncn2[C@@H]2O[C@@H]3CO[P@@](=O)(S)O[C@@H]4[C@H](O)[C@@H](CO[P@@](=O)(S)O[C@H]3[C@H]2O)O[C@H]4n2cnc3c(NC(=O)c4ccccc4)ncnc32)c(=O)[nH]1 ; CC(C)C(=O)Nc1nc2c(ncn2[C@@H]2O[C@@H]3CO[P@](=O)(S)O[C@@H]4[C@H](O)[C@@H](CO[P@@](=O)(S)O[C@H]3[C@H]2O)O[C@H]4n2cnc3c(NC(=O)c4ccccc4)ncnc32)c(=O)[nH]1 ; Nc1nc2c(ncn2[C@@H]2O[C@@H]3CO[P@](=O)(S)O[C@@H]4[C@H](O)[C@@H](CO[P@@](=O)(S)O[C@H]3[C@H]2O)O[C@H]4n2cnc3c(N)ncnc32)c(=O)[nH]1]

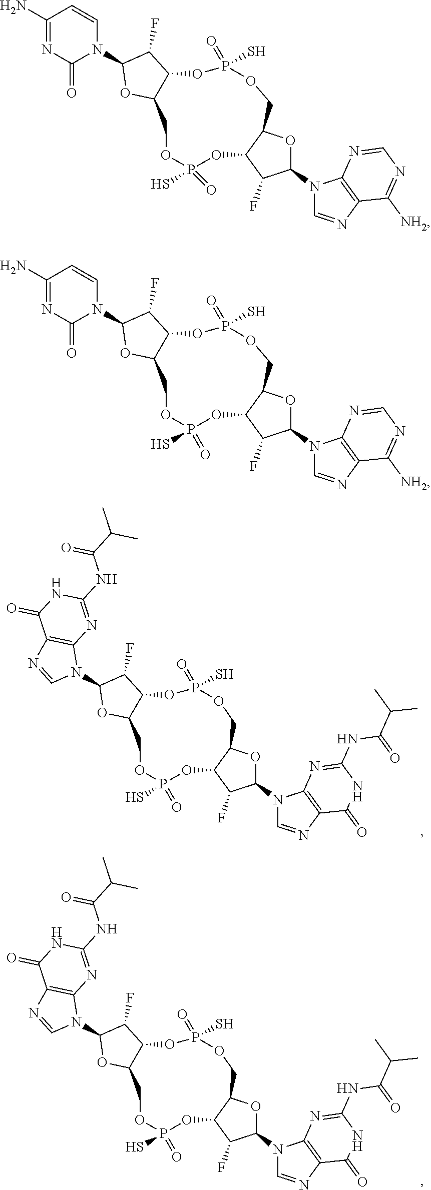

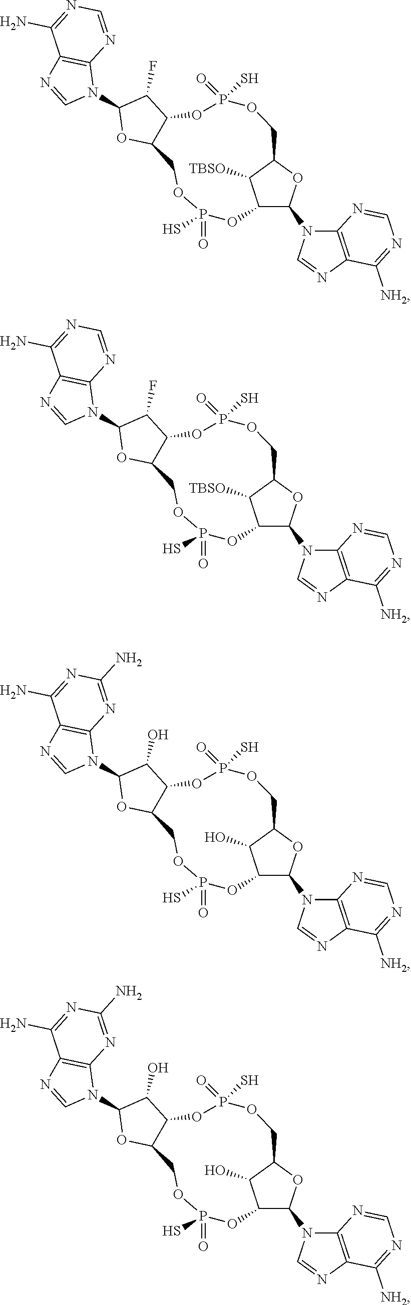

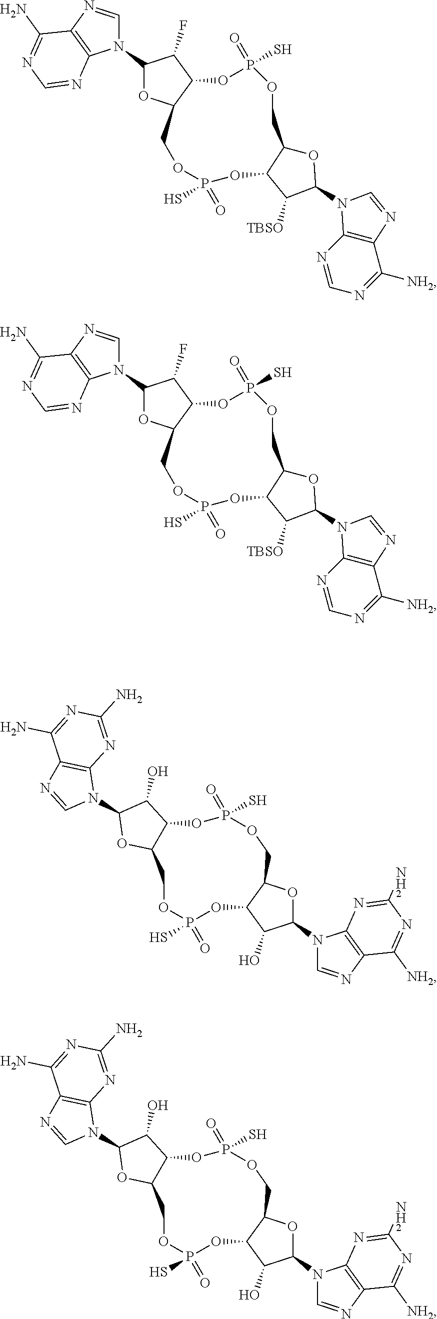

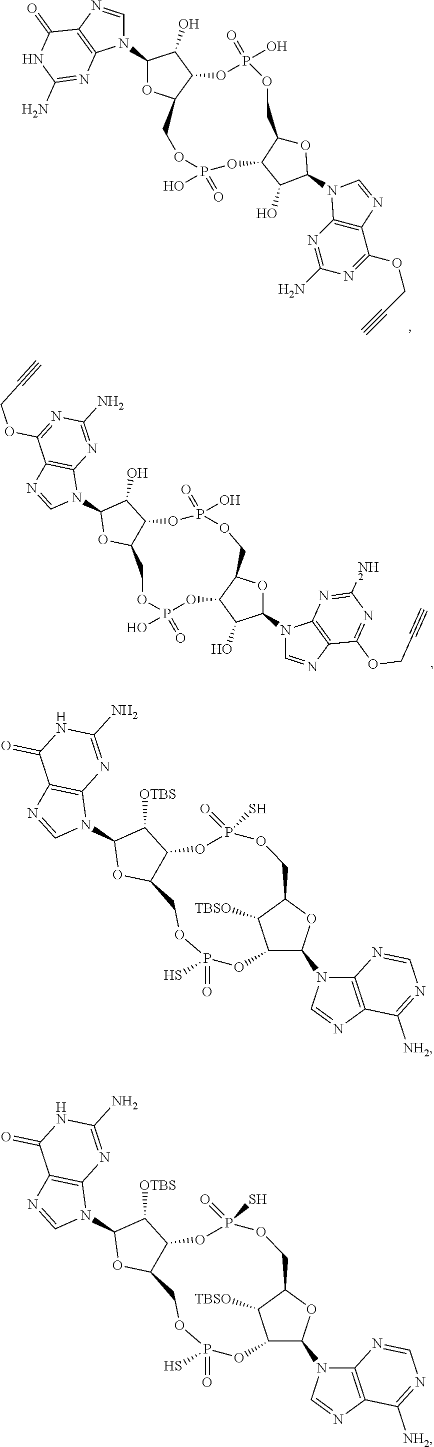

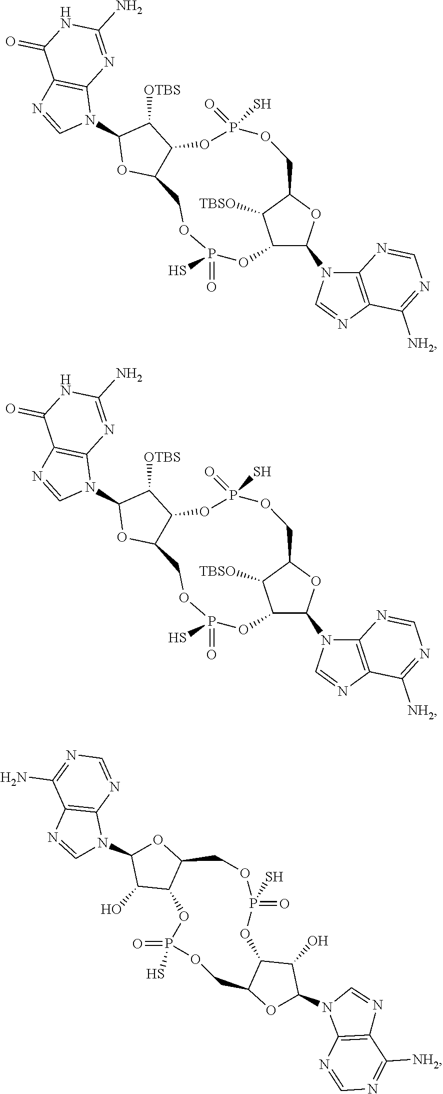 [SMILES: CC(C)(C)[Si](C)(C)O[C@@H]1[C@@H]2O[P@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C ; Nc1ncnc2c1ncn2[C@H]1O[C@H]2CO[P@](=O)(S)O[C@@H]3[C@H](O)[C@@H](n4cnc5c(N)ncnc54)O[C@H]3CO[P@](=O)(S)O[C@@H]2[C@@H]1O]

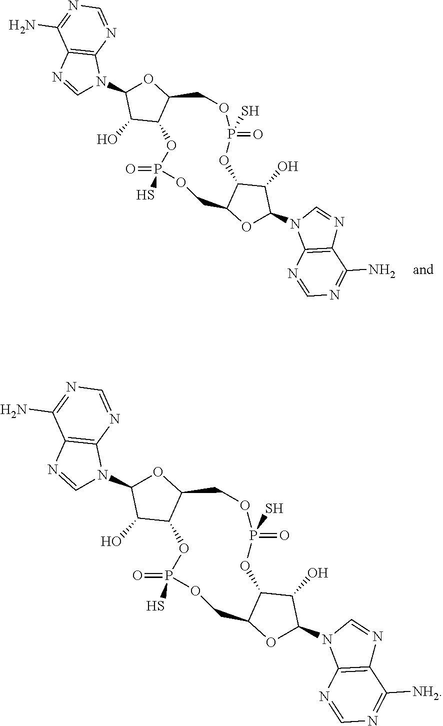 [SMILES: Nc1ncnc2c1ncn2[C@H]1O[C@H]2CO[P@@](=O)(S)O[C@@H]3[C@H](O)[C@@H](n4cnc5c(N)ncnc54)O[C@H]3CO[P@](=O)(S)O[C@@H]2[C@@H]1O ; Nc1ncnc2c1ncn2[C@H]1O[C@H]2CO[P@@](=O)(S)O[C@@H]3[C@H](O)[C@@H](n4cnc5c(N)ncnc54)O[C@H]3CO[P@@](=O)(S)O[C@@H]2[C@@H]1O]

In some embodiments of the first aspect, the scaffold molecule is selected from the group consisting of:

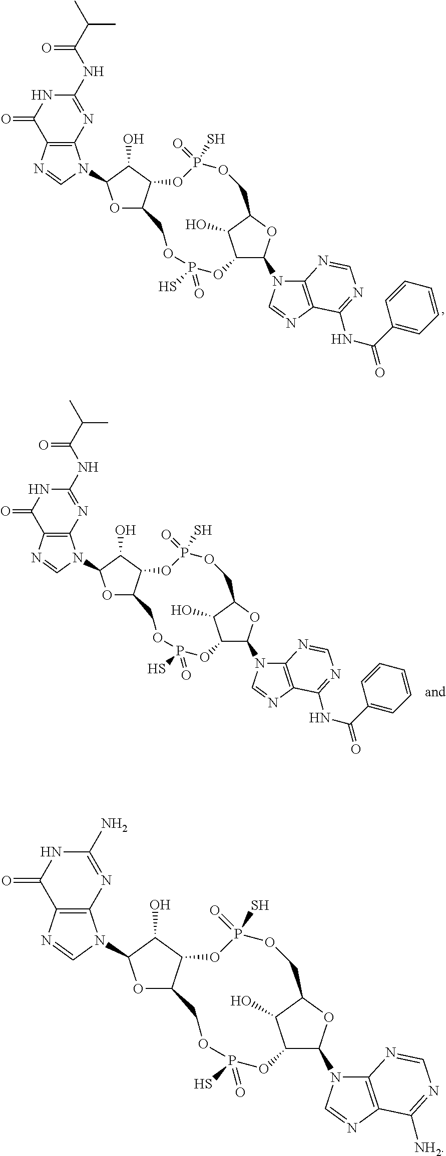 [SMILES: CC(C)C(=O)Nc1nc2c(ncn2[C@@H]2O[C@@H]3CO[P@@](=O)(S)O[C@@H]4[C@H](O)[C@@H](CO[P@@](=O)(S)O[C@H]3[C@H]2O)O[C@H]4n2cnc3c(NC(=O)c4ccccc4)ncnc32)c(=O)[nH]1 ; CC(C)C(=O)Nc1nc2c(ncn2[C@@H]2O[C@@H]3CO[P@](=O)(S)O[C@@H]4[C@H](O)[C@@H](CO[P@@](=O)(S)O[C@H]3[C@H]2O)O[C@H]4n2cnc3c(NC(=O)c4ccccc4)ncnc32)c(=O)[nH]1 ; Nc1nc2c(ncn2[C@@H]2O[C@@H]3CO[P@](=O)(S)O[C@@H]4[C@H](O)[C@@H](CO[P@](=O)(S)O[C@H]3[C@H]2O)O[C@H]4n2cnc3c(N)ncnc32)c(=O)[nH]1]

In some embodiments of the first aspect, the scaffold molecule is selected from the group consisting of:

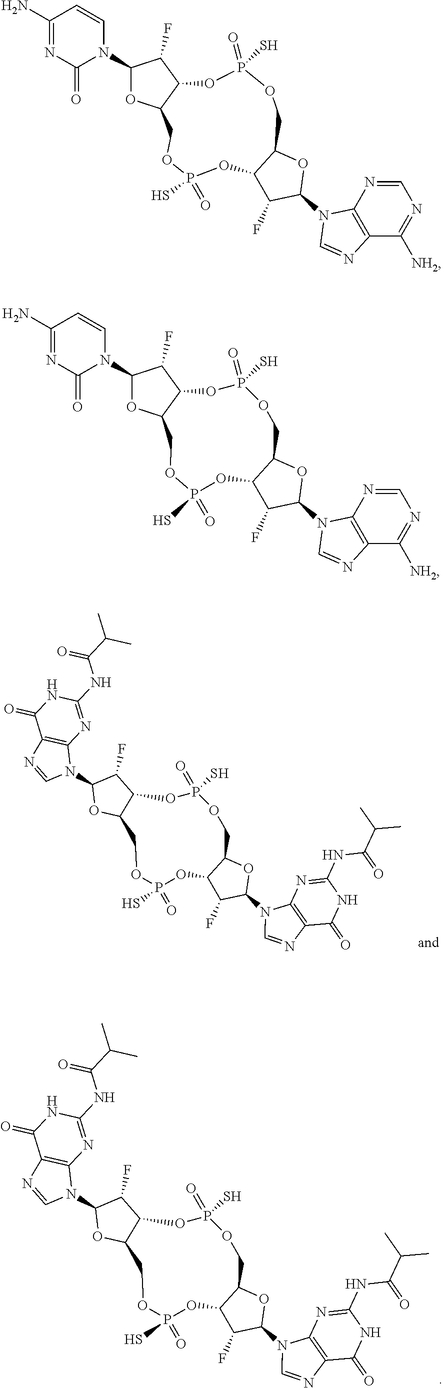

In some embodiments of the first aspect, the scaffold molecule is selected from the group consisting of:

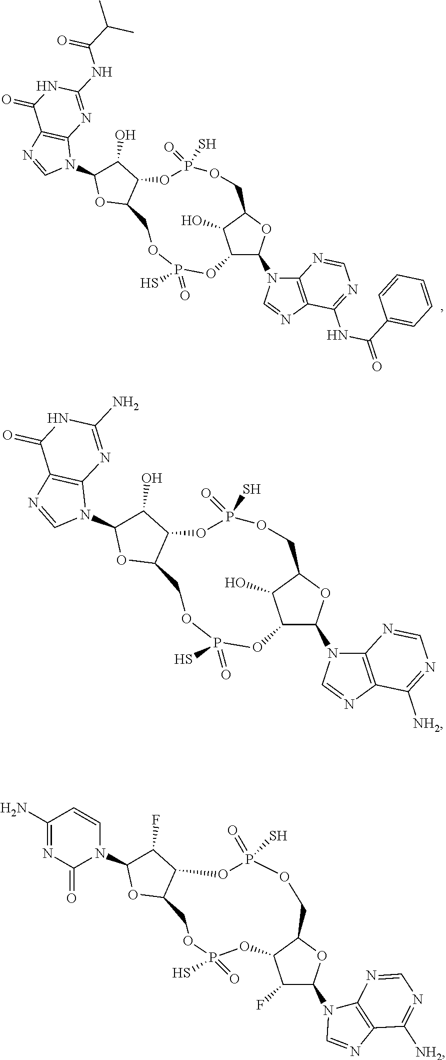 [SMILES: CC(C)C(=O)Nc1nc2c(ncn2[C@@H]2O[C@@H]3CO[P@@](=O)(S)O[C@@H]4[C@H](O)[C@@H](CO[P@@](=O)(S)O[C@H]3[C@H]2O)O[C@H]4n2cnc3c(NC(=O)c4ccccc4)ncnc32)c(=O)[nH]1 ; Nc1nc2c(ncn2[C@@H]2O[C@@H]3CO[P@](=O)(S)O[C@@H]4[C@H](O)[C@@H](CO[P@](=O)(S)O[C@H]3[C@H]2O)O[C@H]4n2cnc3c(N)ncnc32)c(=O)[nH]1 ; Nc1ccn([C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)c(=O)n1]

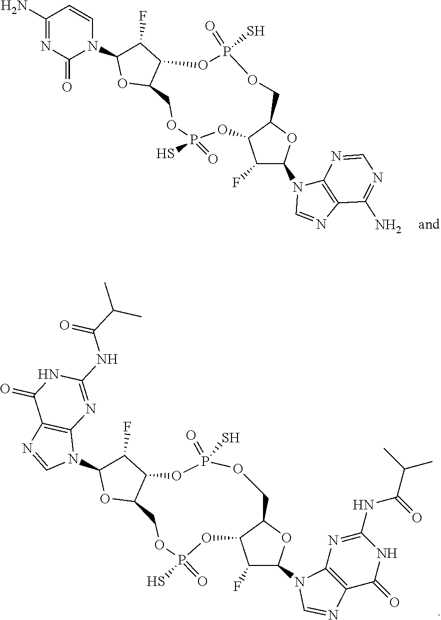 [SMILES: Nc1ccn([C@@H]2O[C@@H]3CO[P@](=O)(S)O[C@H]4[C@@H](F)[C@H](n5cnc6c(N)ncnc65)O[C@@H]4CO[P@@](=O)(S)O[C@H]3[C@H]2F)c(=O)n1 ; CC(C)C(=O)Nc1nc2c(ncn2[C@@H]2O[C@@H]3CO[P@@](=O)(S)O[C@H]4[C@@H](F)[C@H](n5cnc6c(=O)[nH]c(NC(=O)C(C)C)nc65)O[C@@H]4CO[P@@](=O)(S)O[C@H]3[C@H]2F)c(=O)[nH]1]

In some embodiments of the first aspect, the scaffold molecule is selected from the group consisting of:

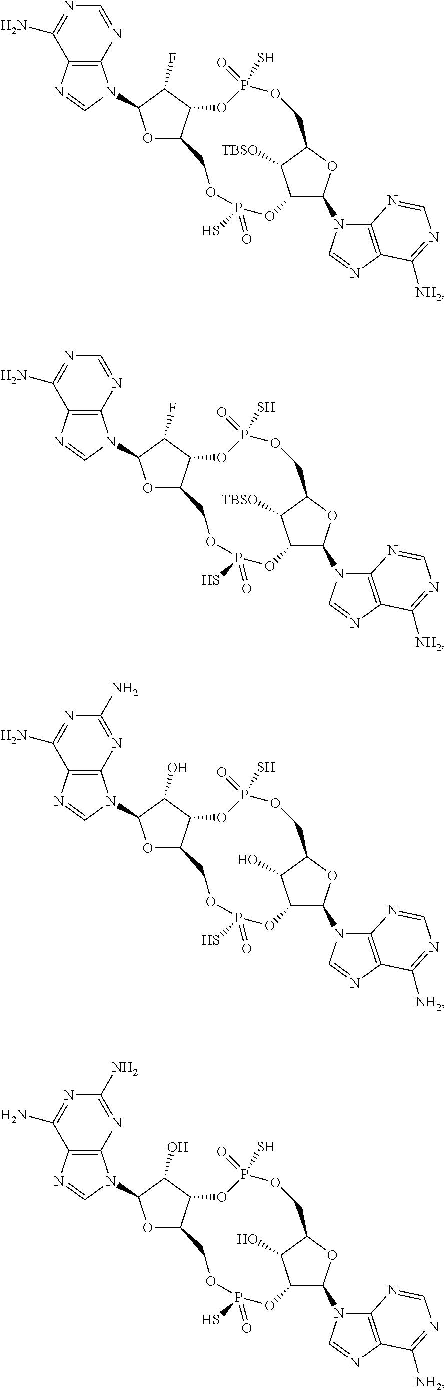

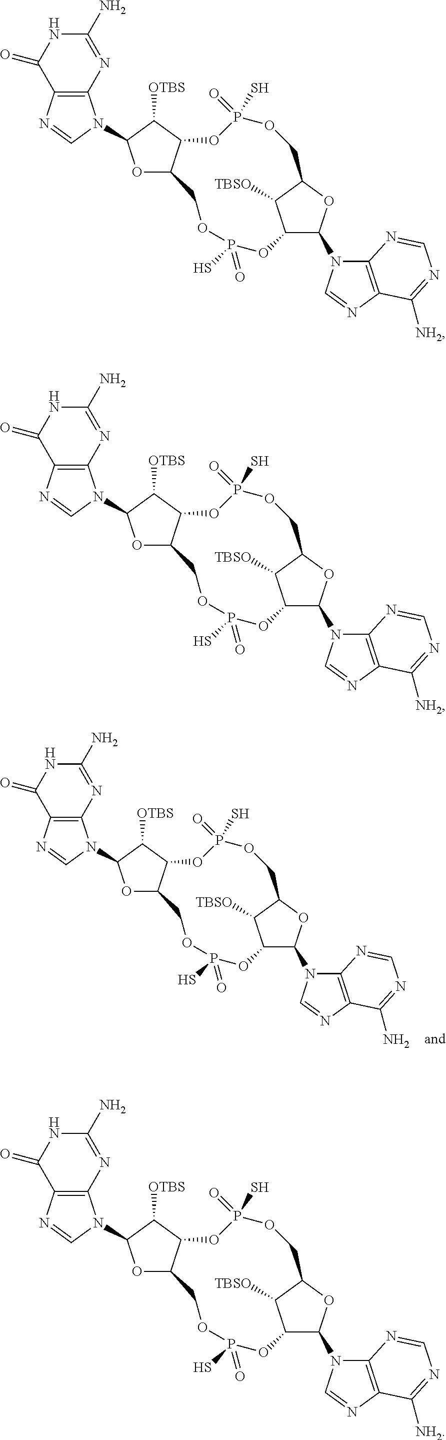

In some embodiments of the first aspect, the scaffold molecule is selected from the group consisting of:

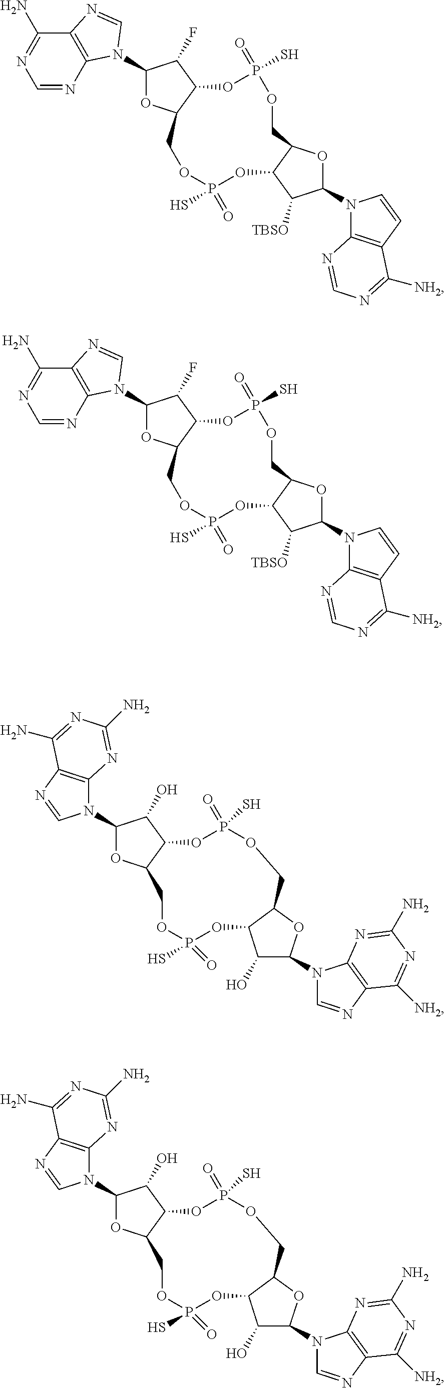

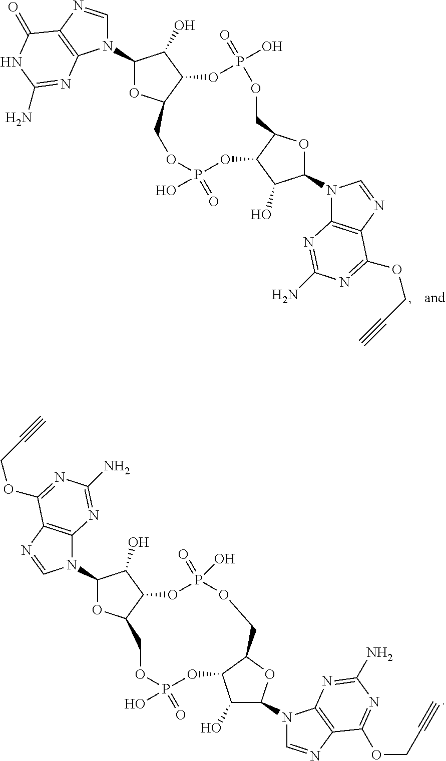 [SMILES: C#CCOc1nc(N)nc2c1ncn2[C@@H]1O[C@@H]2COP(=O)(O)O[C@H]3[C@@H](O)[C@H](n4cnc5c(=O)[nH]c(N)nc54)O[C@@H]3COP(=O)(O)O[C@H]2[C@H]1O ; C#CCOc1nc(N)nc2c1ncn2[C@@H]1O[C@@H]2COP(=O)(O)O[C@H]3[C@@H](O)[C@H](n4cnc5c(OCC#C)nc(N)nc54)O[C@@H]3COP(=O)(O)O[C@H]2[C@H]1O]

In some embodiments of the first aspect, the scaffold molecule is selected from the group consisting of:

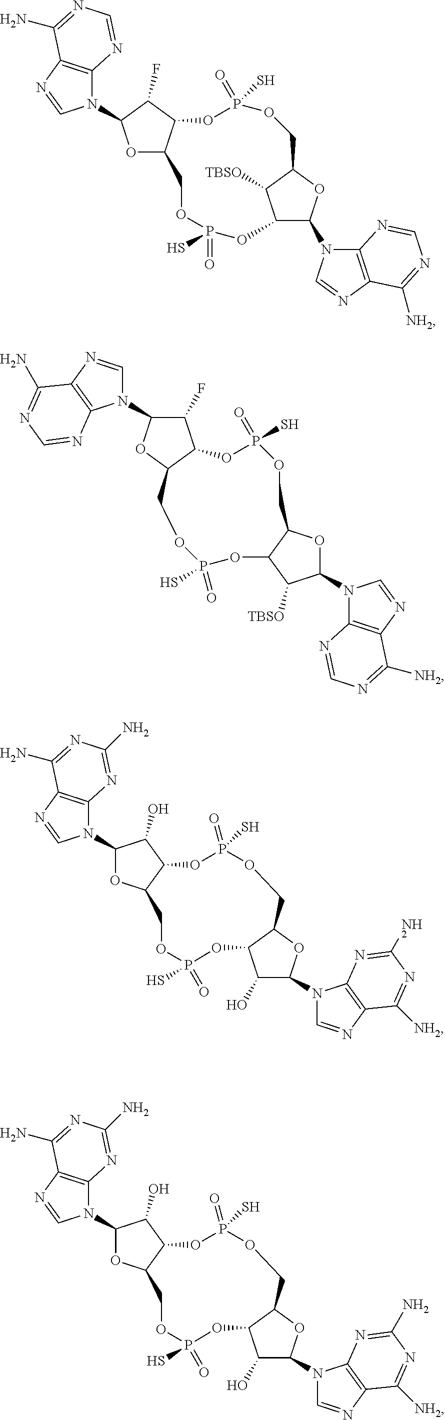

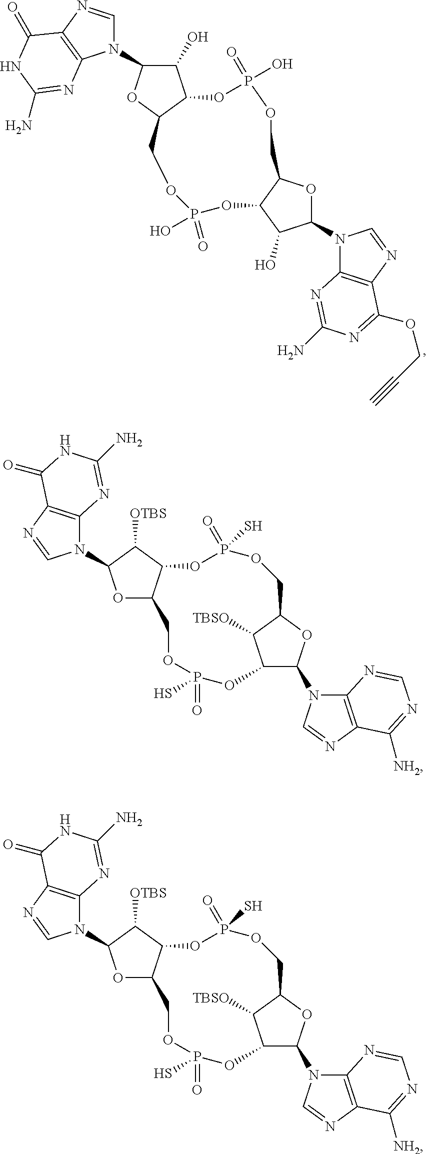 [SMILES: C#CCOc1nc(N)nc2c1ncn2[C@@H]1O[C@@H]2COP(=O)(O)O[C@H]3[C@@H](O)[C@H](n4cnc5c(=O)[nH]c(N)nc54)O[C@@H]3COP(=O)(O)O[C@H]2[C@H]1O ; CC(C)(C)[Si](C)(C)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C]

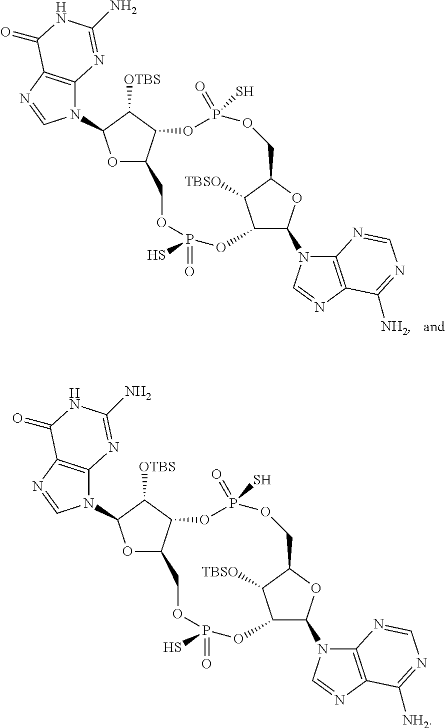 [SMILES: CC(C)(C)[Si](C)(C)O[C@@H]1[C@@H]2O[P@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C]

In some embodiments of the first aspect, the scaffold molecule is selected from the group consisting of:

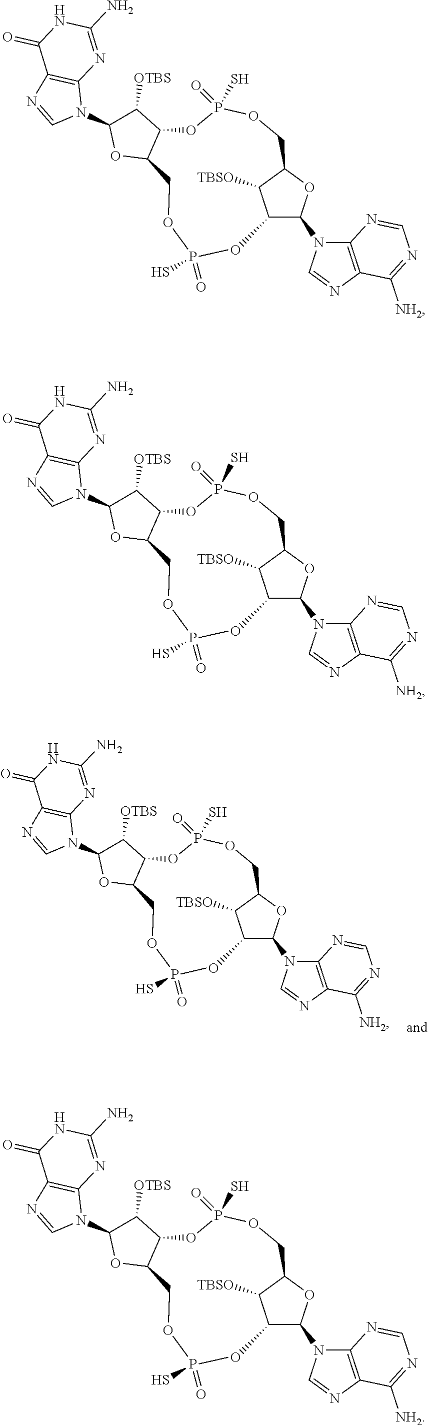

In some embodiments of the first aspect, the scaffold molecule is selected from the group consisting of:

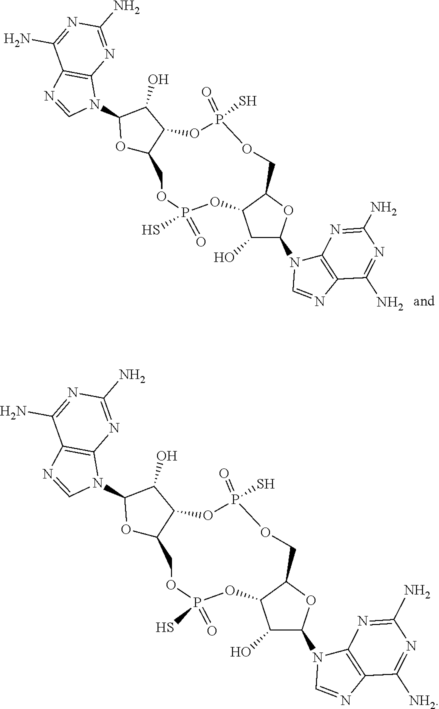 [SMILES: Nc1nc(N)c2ncn([C@@H]3O[C@@H]4CO[P@@](=O)(S)O[C@H]5[C@@H](O)[C@H](n6cnc7c(N)nc(N)nc76)O[C@@H]5CO[P@@](=O)(S)O[C@H]4[C@H]3O)c2n1 ; Nc1nc(N)c2ncn([C@@H]3O[C@@H]4CO[P@@](=O)(S)O[C@H]5[C@@H](O)[C@H](n6cnc7c(N)nc(N)nc76)O[C@@H]5CO[P@](=O)(S)O[C@H]4[C@H]3O)c2n1]

In some embodiments of the first aspect, the scaffold molecule is selected from the group consisting of:

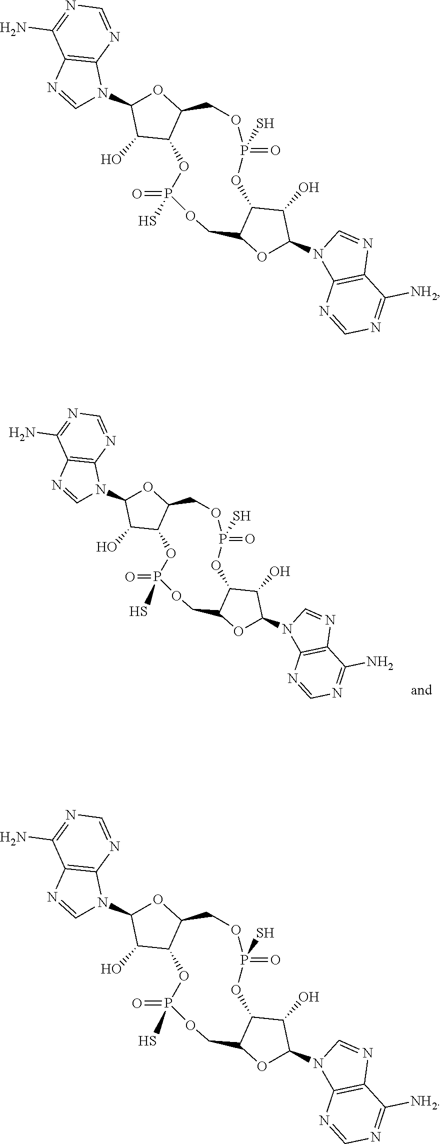

In a second aspect, the present invention provides a method of identifying a STING inhibitor comprising the steps of:
a) providing a scaffold molecule according to the first aspect and all embodiments thereof;
b) synthesizing a derivative of the scaffold molecule;
c) measuring the STING inhibitory activity of the derivative; and
d) identifying the derivative as a STING inhibitor if the derivative has greater STING inhibitory activity than the STING inhibitory activity of the scaffold molecule.

In a first embodiment of the second aspect, the method comprises the step of measuring the binding of the derivative to at least one hSTING allelic protein product (including any one of WT, REF, HAQ, AQ, and Q alleles), preferably one or more of hSTING (WT), hSTING (HAQ), or hSTING (REF). In some embodiments, the binding of the derivative to one or more of hSTING (WT), hSTING (HAQ), or hSTLNG (REF) is greater than the binding of the scaffold molecule to the same hSTING. In some embodiments, the binding of the derivative to two or more of hSTING (WT), hSTING (HAQ), or hSTING (REF) is greater than the binding of the scaffold molecule to the same hSTING. In some embodiments, the binding of the derivative to each of hSTING (WT), hSTING (HAQ), or hSTING (REF) is greater than the binding of the scaffold molecule to the same hSTING. In some embodiments, the binding to hSTING is measured by Tₘ shift in a differential scanning fluorometry assay, for example the assay according to Example 11. In some embodiments, the Tₘ shift measured for the identified hSTING inhibitor is in the range of about 10 to about 30° C. for hSTING (WT) or hSTING (REF) and in the range of about 10 to about 40° C. for hSTING (HAQ). In some embodiments, the hSTING binding of the derivative is measured prior to step (c), wherein compounds that do not demonstrate sufficient binding to any hSTING are eliminated as potential hSTING inhibitors, i.e. they are not measured according to step (c), and are not identified as hSTING inhibitors.

In a second embodiment of the second aspect and first embodiment thereof, the method comprises the step of measuring the hSTING agonist activity of the derivative, e.g. in an assay that measures the induction of STING dependent IFN-β production by the derivative. In some embodiments, the hSTING agonist activity of the derivative is measured in a cellular assay that measures the induction of STING dependent IFN-β production. In some embodiments, the agonist activity is measured as described in US Patent Publication No. 2015056224, the disclosure of which is hereby incorporated by reference as it relates to measurement of hSTING agonist activity, or in the assay according to Example 12 herein below. In some embodiments, the hSTING agonist activity is measured with one or more of hSTING (WT), hSTING-(HAQ), or hSTING (REF). In some embodiments, the hSTING agonist activity of the derivative is measured prior to step (c), wherein compounds having hSTING agonist activity with one or more of hSTING (WT), hSTING (HAQ), or hSTING (REF) are eliminated as potential hSTING inhibitors, i.e. they are not measured according to step (c), and are not identified as hSTING inhibitors.

In some embodiments of the second aspect and first or second embodiments thereof, the STING inhibitor is an hSTING inhibitor, and the hSTING inhibitory activity of the derivative is measured in a competition assay with an hSTLNG agonist. In a preferred embodiment, the competition assay is a cellular assay that measures the induction of STING dependent IFN-production, wherein the assay is performed without the addition of digitonin or other agent that increases permeabilization of the cell to the compounds being assayed. In some embodiments, the hSTING inhibitory activity is measured in the competition assay with one or more of hSTING (WT), hSTING (HAQ), or hSTING (REF). In some embodiments, the hSTING agonist is 2′3′-RR-(A)(A). In some embodiments, the competition assay is the assay according to Example 13. In some embodiments, the identified hSTING inhibitor has an IC50 of less than 100 μM, less than 50 μM, less than 10 μM, less than 5 μM, or less than 1 μM or is in the range of about 1 μM to about 100 μM, 1 μM to about 50 μM, about 1 μM to about 10 μM, about 1 μM to about 5 μM for one or more of hSTING (WT), hSTING (HAQ), or hSTING (REF) in the competition assay with the STING agonist 2′3′-RR-(A)(A). In some embodiments, the identified hSTING inhibitor has an IC50 of less than 100 μM, less than 50 μM, less than 10 μM, less than 5 μM, or less than 1 μM or is in the range of about 1 μM to about 100 μM, 1 μM to about 50 μM, about 1 μM to about 10 μM, about 1 μM to about 5 μM for two or more of hSTING (WT), hSTING (HAQ), or hSTING (REF) in the competition assay with the STING agonist 2′3′-RR-(A)(A). In some embodiments, the identified hSTING inhibitor has an IC50 of less than 100 μM, less than 50 μM, less than 10 μM, less than 5 μM, or less than 1 μM or is in the range of about 1 μM to about 100 μM, 1 μM to about 50 μM, about 1 μM to about 10 μM, about 1 μM to about 5 μM for each of hSTING (WT), hSTING (HAQ), or hSTING (REF) in the competition assay with the STING agonist 2′3′-RR-(A)(A).

In a third embodiment of the second aspect and all embodiments thereof, where the scaffold molecule is of Formula I or Formula II, synthesizing the derivative comprises the modification of one or more of R1, R2, R3 or R4, or the modification of one or more of R5, R6, R7 or R8. In some embodiments, synthesizing the derivative comprises one or more modifications selected from the group consisting of modification of R1 at the 6-position of the adenine or adenine-6-benzamide, or by replacing the adenine or adenine-6-benzamide group with a purine or pyrimidine base or derivative thereof, modification of R2 at the 2-position and/or the 6-position of the guanine or guanine-2-isobutyramide, or by replacing the guanine or guanine-2-isobutyramide with a purine or pyrimidine base or derivative thereof; modification of R3 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R4 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; or one or more modifications selected from the group consisting of modification of R5 at the 6-position of the adenine, at the 2-position and/or 6-position of guanine-2-isobutyramide, or at the 2-position and/or 4-position of cytosine, or by replacing the adenine, guanine-2-isobutyramide, or cytosine group with a purine or pyrimidine base or derivative thereof; modification of R6 at the 6-position of the adenine, at the 2-position and/or 6 position of guanine-2-isobutyramide, or at the 2-position and/or 4-position of cytosine, or by replacing the adenine, guanine-2-isobutyramide, or cytosine group with a purine or pyrimidine base or derivative thereof; modification of R7 by replacing the —F with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R8 by replacing the —F with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula I or Formula II, the synthesizing of the derivative comprises one or more modifications selected from the group consisting of modification of R1 at the 6-position of the adenine or adenine-6-benzamide, or by replacing the adenine or adenine-6-benzamide group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine or adenine-6-benzamide group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R2 at the 2-position and/or the 6-position of the guanine or guanine-2-isobutyramide, or by replacing the guanine or guanine-2-isobutyramide group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the guanine or guanine-2-isobutyramide group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R3 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R4 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; or one or more modifications selected from the group consisting of modification of R5 at the 6-position of the adenine, at the 2-position and/or 6 position of guanine-2-isobutyramide, or at the 2-position and/or 4-position of cytosine, or by replacing the adenine, guanine-2-isobutyramide, or cytosine group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine, guanine-2-isobutyramide, or cytosine group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl, modification of R6 at the 6-position of the adenine, at the 2-position and/or 6 position of guanine-2-isobutyramide, or at the 2-position and/or 4-position of cytosine, or by replacing the adenine, guanine-2-isobutyramide, or cytosine group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine, guanine-2-isobutyramide, or cytosine group with a a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R7 by replacing the —F with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R8 by replacing the —F with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula I or Formula II, synthesizing the derivative comprises one or more modifications selected from the group consisting of modification of R1 at the 6-position of the adenine or adenine-6-benzamide; modification of R2 at the 2-position and/or the 6-position of the guanine or guanine-2-isobutyramide; modification of R3 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R4 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; or one or more modifications selected from the group consisting of modification of R5 at the 6-position of the adenine, at the 2-position and/or 6 position of guanine-2-isobutyramide, or at the 2-position and/or 4-position of cytosine; modification of R6 at the 6-position of the adenine, at the 2-position and/or 6 position of guanine-2-isobutyramide, or at the 2-position and/or 4-position of cytosine; modification of R7 by replacing the —F with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R8 by replacing the —F with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula I or Formula II, the modification of the 6-position of adenine or adenine-6-benzamide, the 2-position of guanine or guanine-2-butyramide, or the 4-position of cytosine in any of R1, R2, R5 and R6 comprises replacing the amine or protected amine group with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and the modification of the 6-position of guanine or guanine-2-butyramide or the 2-position of cytosine in any of R2, R5 and R6 comprises replacing the oxo group with —OR^{x}, where R^{x} is alkyl, alkenyl or alkynyl. In some embodiments, modification of guanine or guanine-2-butyramide is only at the 2-position. In some embodiments, modification of the guanine or guanine-2-butyramide is only at the 6-position. In some embodiments, modification of the guanine or guanine-2-butyramide is independently at both the 2-position and 6-position. In some embodiments, modification of cytosine is only at the 2-position. In some embodiments, modification of the cytosine is only at the 4-position. In some embodiments, modification of the cytosine is independently at both the 2-position and 4-position.

In some embodiments of the second aspect and any of the above embodiments thereof, where the scaffold molecule is of Formula I or Formula II, the synthesizing of a derivative consists of the modification of one of R1, R2, R3 or R4, or the modification of one of R5, R6, R7 or R8.

In some embodiments of the second aspect and any of the above embodiments thereof, where the scaffold molecule is of Formula I or Formula II, the synthesizing of a derivative consists of the modification of one or two of R1, R2, R3 or R4, or the modification of one or two of R5, R6, R7 or R8. In some embodiments the synthesizing of a derivative consists of the modification selected from the group consisting of modification of R1 and R2, modification of R1 and R3, modification of R1 and R4, modification of R2 and R3, modification of R2 and R4, and modification of R3 and R4, or the modification selected from the group consisting of modification of R5 and R6, modification of R5 and R7, modification of R5 and R8, modification of R6 and R7, modification of R6 and R8, and modification of R7 and R8.

In some embodiments of the second aspect and any of the above embodiments thereof, where the scaffold molecule is of Formula I or Formula II, the synthesizing of a derivative consists of the modification of one of R1 or R2 and one of R3 or R4, or the modification of one of R5 or R6 and the modification of one of R7 or R8. In some embodiments the synthesizing of a derivative consists of the modification selected from the group consisting of modification of R1 and R3, modification of R1 and R4, modification of R2 and R3, and modification of R2 and R4, or the modification selected from the group consisting of modification of R5 and R7, modification of R5 and R8, modification of R6 and R7, and modification of R6 and R8.

In some embodiments of the second aspect and any of the above embodiments thereof, where the scaffold molecule is of Formula I or Formula II, the synthesizing of a derivative consists of the modification of one, two or three of R1, R2, R3 or R4, or the modification of one, two or three of R5, R6, R7 or R8. In some embodiments the synthesizing of a derivative consists of the modification selected from the group consisting of modification of R1, R2 and R3, modification of R1, R2 and R4, modification of R1, R3 and R4, and modification of R2, R3 and R4, or the modification selected from the group consisting of modification of R5, R6 and R7, modification of R5, R6 and R8, modification of R5, R7 and R8, and modification of R6, R7 and R8.

In some embodiments of the second aspect and any of the above embodiments thereof, where the scaffold molecule is of Formula I or Formula II, the synthesizing of a derivative consists of the modification of each of R1, R2, R3 and R4, or the modification of each of R5, R6, R7, and R8.

In some embodiments of the second aspect and any of the above embodiments thereof, where the scaffold molecule is of Formula I or Formula II, in describing the replacement of R3, R4, R7 or R8, R¹⁰⁰ is preferably C₁₋₆alkenyl or C₁₋₆alkynyl. In some embodiments, in describing the replacement of R3, R4, R7 or R8, —SiR¹⁰¹R¹⁰²R¹⁰³ is preferably selected from the group consisting of trimethylsilyl (TMS), triethylsilyl (TES), triisopropylsilyl (TIPS), isopropyldimethylsilyl (DEIPS), tert-butyldimethylsilyl (TBS) and tert-butyldiphenylsilyl (TBDPS). In some embodiments, in describing the replacement of R3, R4, R7 or R8, R¹⁰⁰ is preferably C₁₋₆alkenyl or C₁₋₆alkynyl and —SiR¹⁰¹R¹⁰²R¹⁰³ is preferably selected from the group consisting of trimethylsilyl (TMS), triethylsilyl (TES), triisopropylsilyl (TIPS), isopropyldimethylsilyl (DEIPS), tert-butyldimethylsilyl (TBS) and tert-butyldiphenylsilyl (TBDPS).

In some embodiments of the second aspect and any of the above embodiments thereof, where the scaffold molecule is of Formula I or Formula II, in describing the replacement of R3, R4, R7 or R8, the amine of e.g. adenine, guanine, etc. is suitably protected during synthesis, and the modification to the scaffold molecule includes where such nitrogen protecting group remains on the molecule. A suitable nitrogen protected amine includes dimethyl formamidine, benzoyl, or isobutyryl.

In a fourth embodiment of the second aspect and all embodiments thereof, where the scaffold molecule is of Formula III or Formula IV, synthesizing the derivative comprises the modification of one or more of R9, R10, R11 or R12, or the modification of one or more of R13, R14, R15 or R16. In some embodiments, synthesizing the derivative comprises one or more modifications selected from the group consisting of modification of R9 at the 6-position of the adenine, or by replacing the adenine group with a purine or pyrimidine base or derivative thereof; modification of R10 at the 6-position of the adenine, or at the 2-position and/or the 6-position of the guanine or 2,6-diamino-purine, or by replacing the adenine, guanine or 2,6-diamino-purine with a purine or pyrimidine base or derivative thereof; modification of R11 by replacing the —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R12 by replacing the —F, —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; or one or more modifications selected from the group consisting of modification of R13 at the 6-position of the adenine, or at the 2-position and/or 6 position of the guanine, guanine-6-propargyl ether, or 2,6-diamino-purine, or by replacing the adenine, guanine, guanine-6-propargyl ether, or 2,6-diamino-purine group with a purine or pyrimidine base or derivative thereof; modification of R14 at the 6-position of the adenine, or at the 2-position and/or 6 position of the guanine, guanine-6-propargyl ether, or 2,6-diamino-purine, or by replacing the adenine, guanine, guanine-6-propargyl ether, or 2,6-diamino-purine group with a purine or pyrimidine base or derivative thereof; modification of R15 by replacing the —F, —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R16 by replacing the —F, —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula II or Formula IV, the synthesizing of the derivative comprises one or more modifications selected from the group consisting of modification of R9 at the 6-position of the adenine, or by replacing the adenine group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R10 at the 6-position of the adenine, or at the 2-position and/or the 6-position of the guanine or 2,6-diamino-purine, or by replacing the adenine, guanine or 2,6-diamino-purine group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine, guanine or 2,6-diamino-purine group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R11 by replacing the —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R12 by replacing the —F, —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; or one or more modifications selected from the group consisting of modification of R13 at the 6-position of the adenine, or at the 2-position and/or 6 position of the guanine, guanine-6-propargyl ether, or 2,6-diamino-purine, or by replacing the adenine, guanine, guanine-6-propargyl ether, or 2,6-diamino-purine group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine, guanine, guanine-6-propargyl ether, or 2,6-diamino-purine group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R14 at the 6-position of the adenine, or at the 2-position and/or 6 position of the guanine, guanine-6-propargyl ether, or 2,6-diamino-purine, or by replacing the adenine, guanine, guanine-6-propargyl ether, or 2,6-diamino-purine group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine, guanine, guanine-6-propargyl ether, or 2,6-diamino-purine group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R15 by replacing the —F, —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R16 by replacing the —F, —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula II or Formula IV, synthesizing the derivative comprises one or more modifications selected from the group consisting of modification of R9 at the 6-position of the adenine; modification of R10 at the 6-position of the adenine, or at the 2-position and/or the 6-position of the guanine or 2,6-diamino-purine; modification of R^{II} by replacing the —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R12 by replacing the —F, —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; or one or more modifications selected from the group consisting of modification of R13 at the 6-position of the adenine, or at the 2-position and/or 6 position of the guanine, guanine-6-propargyl ether, or 2,6-diamino-purine; modification of R14 at the 6-position of the adenine, or at the 2-position and/or 6 position of the guanine, guanine-6-propargyl ether, or 2,6-diamino-purine; modification of R15 by replacing the —F, —OH or —OTBS with a substituent selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R16 by replacing the —F, —OH or —OTBS with a substituent selected from the group consisting of —H, —CN, halogen, —OH, alkoxy, —OCH₂R¹⁰⁰, wherein R¹⁰³ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula III or Formula IV, the modification of the 6-position of adenine, the 2-position or 6-position amine of 2,6-diamino-purine, or the 2-position of guanine or guanine-6-propargyl ether in any of R9, R10, R13 and R14 comprises replacing the amine or protected amine group with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and the modification of the 6-position of guanine or guanine-6-propargyl ether in any of R10, R13 and R14 comprises replacing the oxo group with —OR^{x}, where R^{x} is alkyl, alkenyl or alkynyl. In some embodiments, modification of guanine or guanine-6-propargyl ether is only at the 2-position. In some embodiments, modification of the guanine or guanine-6-propargyl ether is only at the 6-position. In some embodiments, modification of the guanine or guanine-6-propargyl ether is independently at both the 2-position and 6-position. In some embodiments, modification of 2,6-diamino-purine is only at the 2-position. In some embodiments, modification of the 2,6-diamino-purine is only at the 6-position. In some embodiments, modification of the 2,6-diamino-purine is independently at both the 2-position and 6-position.

In some embodiments of any of the above embodiments where in the scaffold molecule is of Formula IV and X₁ and/or X₂ is —OH, modification can also include, independently of or in addition to any of the above modifications, replacing the —OH with —SH. Preferably where a scaffold molecule of Formula IV has X₁ and/or X₂ as —OH, each —OH is replaced with —SH in the derivative of the scaffold molecule.

In some embodiments of the second aspect and any of the above embodiments thereof, where the scaffold molecule is of Formula III or Formula IV, the synthesizing of a derivative consists of the modification of one of R9, R10, R11 or R12, or the modification of one of R13, R14, R15 or R16.

In some embodiments of the second aspect and any of the above embodiments thereof, where the scaffold molecule is of Formula III or Formula IV, the synthesizing of a derivative consists of the modification of one or two of R9, R10, R11 or R12, or the modification of one or two of R13, R14, R15 or R16. In some embodiments the synthesizing of a derivative consists of the modification selected from the group consisting of modification of R9 and R10, modification of R9 and R11, modification of R9 and R12, modification of R10 and R11, modification of R10 and R12, and modification of R11 and R12, or the modification selected from the group consisting of modification of R13 and R14, modification of R13 and R15, modification of R13 and R16, modification of R14 and R15, modification of R14 and R16, and modification of R15 and R16.

In some embodiments of the second aspect and any of the above embodiments thereof, where the scaffold molecule is of Formula III or Formula IV, the synthesizing of a derivative consists of the modification of one of R9 or R10 and one of R11 or R12, or the modification of one of R13 or R14 and the modification of one of R15 or R16. In some embodiments the synthesizing of a derivative consists of the modification selected from the group consisting of modification of R9 and R11, modification of R9 and R12, modification of R10 and R11, and modification of R10 and R12, or the modification selected from the group consisting of modification of R13 and R15, modification of R13 and R16, modification of R14 and R15, and modification of R14 and R16.

In some embodiments of the second aspect and any of the above embodiments thereof, where the scaffold molecule is of Formula III or Formula IV, the synthesizing of a derivative consists of the modification of one, two or three of R9, R10, R11 or R12, or the modification of one, two or three of R13, R14, R15 or R16. In some embodiments the synthesizing of a derivative consists of the modification selected from the group consisting of modification of R9, R10 and R11, modification of R9, R10 and R12, modification of R9, R11 and R12, and modification of R10, R11 and R12, or the modification selected from the group consisting of modification of R13, R14 and R15, modification of R13, R14 and R16, modification of R13, R15 and R16, and modification of R14, R15 and R16.

In some embodiments of the second aspect and any of the above embodiments thereof, where the scaffold molecule is of Formula III or Formula IV, the synthesizing of a derivative consists of the modification of each of R9, R10, R11 and R12, or the modification of each of R13, R14, R15, and R16.

In some embodiments of the second aspect and any of the above embodiments thereof, where the scaffold molecule is of Formula III or Formula IV, in describing the replacement of R11, R12, R15 or R16, R¹⁰⁰ is preferably C₁₋₆alkenyl or C₁₋₆alkynyl. In some embodiments, in describing the replacement of R11, R12, R15 or R16, —SiR¹⁰¹R¹⁰²R¹⁰³ is preferably selected from the group consisting of trimethylsilyl (TMS), triethylsilyl (TES), triisopropylsilyl (TIPS), isopropyldimethylsilyl (DEIPS), tert-butyldimethylsilyl (TBS) and tert-butyldiphenylsilyl (TBDPS). In some embodiments, in describing the replacement of R11, R12, R15 or R16, R¹⁰⁰ is preferably C₁₋₆alkenyl or C₁₋₆alkynyl and —SiR¹⁰¹R¹⁰²R¹⁰³ is preferably selected from the group consisting of trimethylsilyl (TMS), triethylsilyl (TES), triisopropylsilyl (TIPS), isopropyldimethylsilyl (DEIPS), tert-butyldimethylsilyl (TBS) and tert-butyldiphenylsilyl (TBDPS).

In some embodiments of the second aspect and any of the above embodiments thereof, where the scaffold molecule is of Formula III or Formula IV, in describing the replacement of R11, R12, R15 or R16, the amine of e.g. adenine, guanine, etc. is suitably protected during synthesis, and the modification to the scaffold molecule includes where such nitrogen protecting group remains on the molecule. A suitable nitrogen protected amine includes dimethyl formamidine, benzoyl, or isobutyryl.

In a fifth embodiment of the second aspect and all embodiments thereof, where the scaffold molecule is of Formula V, synthesizing the derivative comprises the modification of one or more of R17, R18, R19 or R20. In some embodiments, synthesizing the derivative comprises one or more modifications selected from the group consisting of modification of R17 at the 6-position of the adenine, or by replacing the adenine group with a purine or pyrimidine base or derivative thereof; modification of R18 at the 6-position of the adenine, or by replacing the adenine group with a purine or pyrimidine base or derivative thereof; modification of R19 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R20 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula V, the synthesizing of the derivative comprises one or more modifications selected from the group consisting of modification of R17 at the 6-position of the adenine, or by replacing the adenine group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine is modified with a protecting group, or the amino is replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-amino of isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R18 at the 6-position of the adenine, or by replacing the adenine group with a suitable purine or derivative thereof, including, but not limited to, adenin-9-yl, guanin-9-yl, hypoxanthin-9-yl, xanthin-9-yl, isoguanin-9-yl, or 2,6-diamino-purin-9-yl, wherein the 6-amino of adenine is modified with a protecting group, or the amino is replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, or by replacing the adenine group with a suitable pyrimidine or derivative thereof, including, but not limited to, cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl; modification of R19 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R20 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula V, synthesizing the derivative comprises one or more modifications selected from the group consisting of modification of R17 at the 6-position of the adenine; modification of R18 at the 6-position of the adenine; modification of R19 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and modification of R20 by replacing the —OH with a substituent selected from the group consisting of —H, —CN, halogen, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl.

In some embodiments of the second aspect and all embodiments thereof, where the scaffold molecule is of Formula V, the modification of the 6-position of adenine in R17 or R18 comprises replacing the amine group with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy.

In some embodiments of the second aspect and any of the above embodiments thereof, where the scaffold molecule is of Formula V, the synthesizing of a derivative consists of the modification of one of R17, R18, R19 or R20.

In some embodiments of the second aspect and any of the above embodiments thereof, where the scaffold molecule is of Formula V, the synthesizing of a derivative consists of the modification of one or two of R17, R18, R19 or R20. In some embodiments the synthesizing of a derivative consists of the modification selected from the group consisting of modification of R17 and R18, modification of R17 and R19, modification of R17 and R20, modification of R18 and R19, modification of R18 and R20, and modification of R19 and R20.

In some embodiments of the second aspect and any of the above embodiments thereof, where the scaffold molecule is of Formula V, the synthesizing of a derivative consists of the modification of one of R17 or R18 and one of R19 or R20. In some embodiments the synthesizing of a derivative consists of the modification selected from the group consisting of modification of R17 and R19, modification of R17 and R20, modification of R18 and R19, and modification of R18 and R20.

In some embodiments of the second aspect and any of the above embodiments thereof, where the scaffold molecule is of Formula V, the synthesizing of a derivative consists of the modification of one, two or three of R17, R18, R19 or R20. In some embodiments the synthesizing of a derivative consists of the modification selected from the group consisting of modification of R17, R18 and R19, modification of R17, R18 and R20, modification of R17, R19 and R20, and modification of R18, R19 and R20.

In some embodiments of the second aspect and any of the above embodiments thereof, where the scaffold molecule is of Formula V, the synthesizing of a derivative consists of the modification of each of R17, R18, R19 and R20.

In some embodiments of the second aspect and any of the above embodiments thereof, where the scaffold molecule is of Formula V, in describing the replacement of R19 or R20, R¹⁰⁰ is preferably C₁₋₆alkenyl or C₁₋₆alkynyl. In some embodiments, in describing the replacement of R19 or R20, —SiR¹⁰¹R¹⁰²R¹⁰³ is preferably selected from the group consisting of trimethylsilyl (TMS), triethylsilyl (TES), triisopropylsilyl (TIPS), isopropyldimethylsilyl (DEIPS), tert-butyldimethylsilyl (TBS) and tert-butyldiphenylsilyl (TBDPS). In some embodiments, in describing the replacement of R19 or R20, R¹⁰⁰ is preferably C₁₋₆alkenyl or C₁₋₆alkynyl and —SiR¹⁰¹R¹⁰²R¹⁰³ is preferably selected from the group consisting of trimethylsilyl (TMS), triethylsilyl (TES), triisopropylsilyl (TIPS), isopropyldimethylsilyl (DEIPS), tert-butyldimethylsilyl (TBS) and tert-butyldiphenylsilyl (TBDPS).

In some embodiments of the second aspect and any of the above embodiments thereof, where the scaffold molecule is of Formula V, in describing the replacement of R17 or R18, the amine of e.g. adenine, guanine, etc. is suitably protected during synthesis, and the modification to the scaffold molecule includes where such nitrogen protecting group remains on the molecule. A suitable nitrogen protected amine includes dimethyl formamidine, benzoyl, or isobutyryl.

In a third aspect, the present invention provides a compound having the structure of Formula VI or Formula VII:

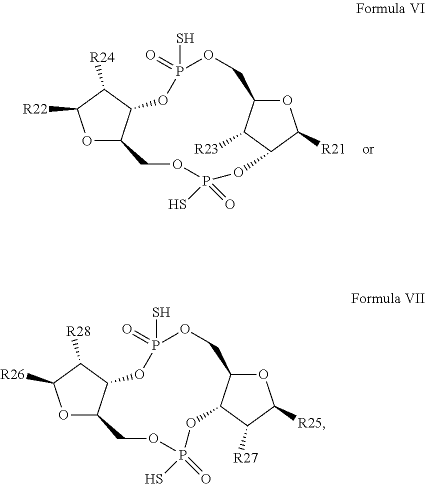

or a prodrug, tautomer, pharmaceutically acceptable salt, pharmaceutically acceptable solvate or pharmaceutically acceptable hydrate thereof,
wherein:
R21 is adenine or adenine-6-benzamide linked to the structure via the N9 position, wherein the 6-position of adenine or adenine-6-benzamide is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy;
R22 is adenine, 2,6-diamino-purine, guanine or guanine-2-isobutyramide linked to the structure via the N9 position, wherein the 2-position of guanine or guanine-2-isobutyramide, the 6-position of adenine, and the 2-position and/or the 6-position of 2,6-diamino-purine are independently optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine or guanine-2-isobutyramide is optionally replaced with —OR^{x}, where R^{x} is alkyl, alkenyl or alkynyl;
R23 is selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl;
R24 is selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl;
R25 is adenine, 2,6-diamino-purine, guanine, guanine-2-isobutyramide or guanine-6-propargyl ether linked to the structure via the N9 position, or cytosine linked to the structure via the N1 position, wherein the 6-position of adenine, 2-position and/or 6-position of 2,6-diamino-purine, 2-position of guanine, guanine-2-isobutyramide or guanine-6-propargyl ether, or 4-position of cytosine are independently optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, guanine-2-isobutyramide or guanine-6-propargyl ether or the 2-position of cytosine is optionally replaced with —OR^{x}, where R^{x} is alkyl, alkenyl or alkynyl;
R26 is adenine, 2,6-diamino-purine, guanine, guanine-2-isobutyramide or guanine-6-propargyl ether linked to the structure via the N9 position, or cytosine linked to the structure via the N1 position, wherein the 6-position of adenine, 2-position and/or 6-position of 2,6-diamino-purine, 2-position of guanine guanine-2-isobutyramide or guanine-6-propargyl ether, or 4-position of cytosine are independently optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, guanine-2-isobutyramide or guanine-6-propargyl ether, or the 2-position of cytosine is optionally replaced with —OR^{x}, where R^{x} is alkyl, alkenyl or alkynyl;
R27 is selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and
R28 is selected from the group consisting of —H, —CN, halogen, —OH, alkoxy, —OCH₂R¹⁰⁰, wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl,
   wherein the compound has STING inhibitory activity.

In a first embodiment of the third aspect, the compound has a structure selected from the group consisting of Formula Via, Formula Villa and Formula VIIb:

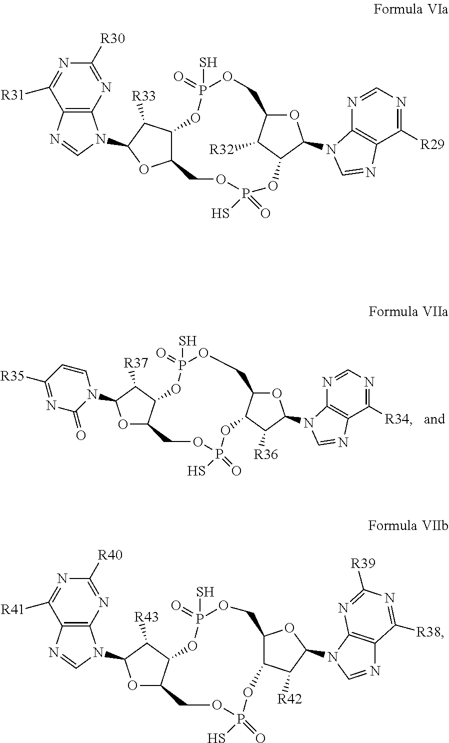

or a prodrug, tautomer, pharmaceutically acceptable salt, pharmaceutically acceptable solvate or pharmaceutically acceptable hydrate thereof,
wherein:
R29, R30, R34, R35, R39 and R40 are independently selected from the group consisting of —NH₂, —NHR^{y}, —NR^{y}R^{z}, —NHCH₂R¹⁰⁴ and —NHC(O)R¹⁰⁴, wherein R^{y} and R^{z} are independently alkyl, and R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy;
R31, R38 and R41 are independently —OR^{w}, where R^{w} is —H, alkyl, alkenyl or alkynyl; and
R32, R33, R36, R37, R42 and R43 are independently selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl.

In some embodiments of the third aspect and first embodiment thereof, the compound has a structure selected from the group consisting of Formula VIa-1, Formula VIa-2 and Formula VIa-3:

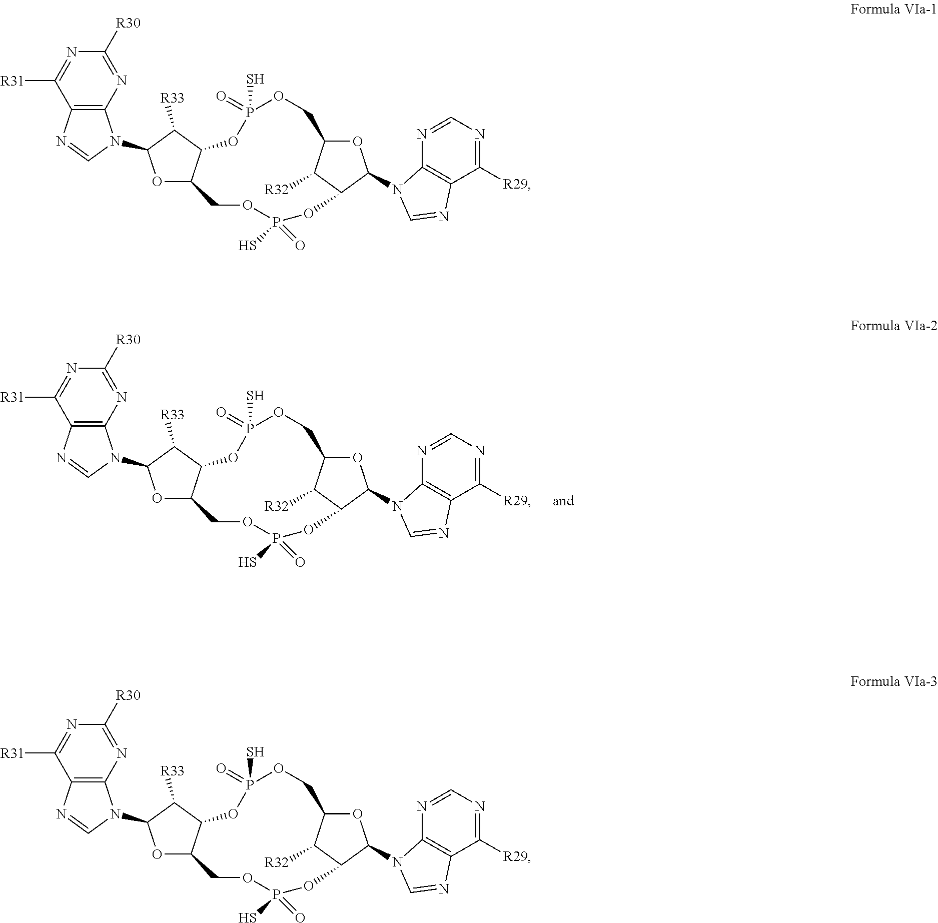

or a prodrug, tautomer, pharmaceutically acceptable salt, pharmaceutically acceptable solvate or pharmaceutically acceptable hydrate thereof, wherein R29, R30, R31, R32 and R33 are as defined for the first embodiment of the third aspect.

In some embodiments of the third aspect and first embodiment thereof, the compound has a structure selected from the group consisting of Formula VIIa-1, Formula VIIa-2, Formula VIIb-1 and Formula VIIb-2:

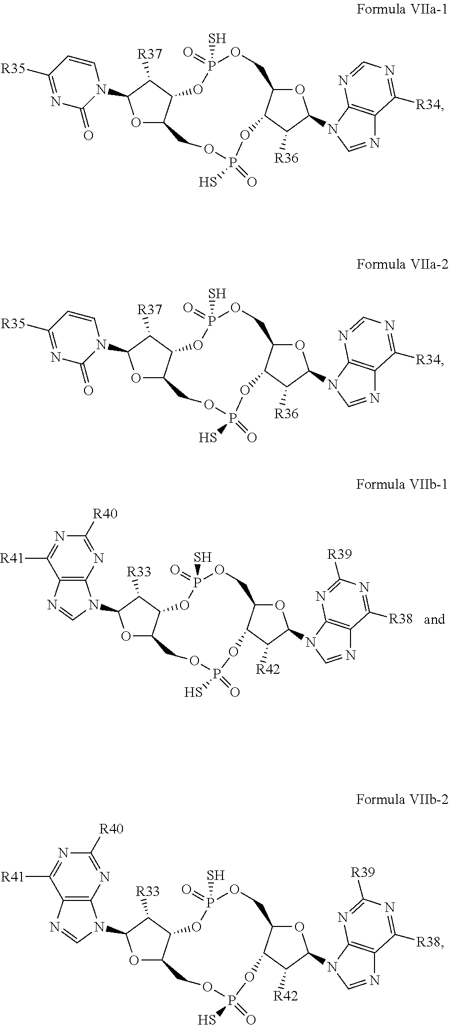

or a prodrug, tautomer, pharmaceutically acceptable salt, pharmaceutically acceptable solvate or pharmaceutically acceptable hydrate thereof, wherein R34, R35, R36, R37, R38, R39, R40, R41, R42 and R43 are as defined for the first embodiment of the third aspect.

In a second embodiment of the third aspect, the compound has a structure selected from the group consisting of Formula VIb, Formula VIc, Formula VId, Formula VIIc, Formula VIId and Formula VIIe:

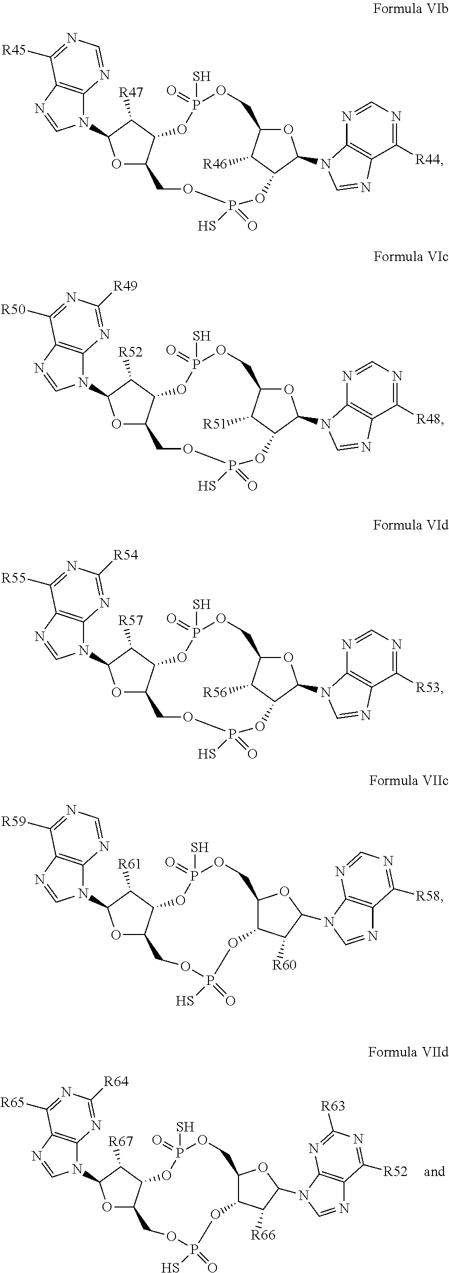

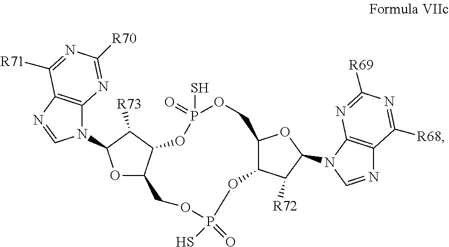

or a prodrug, tautomer, pharmaceutically acceptable salt, pharmaceutically acceptable solvate or pharmaceutically acceptable hydrate thereof,
wherein:
R44, R45, R48, R49, R50, R53, R54, R58, R59, R62, R63, R64, R65, R69 and R70 are independently selected from the group consisting of —NH₂, —NHR^{y}, —NR^{y}R^{z}, —NHCH₂R¹⁰⁴ and —NHC(O)R¹⁰⁴, wherein R^{y} and R^{z} are independently alkyl, and R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy;
R55, R68 and R71 are independently —OR^{w}, where R^{w} is —H, alkyl, alkenyl or alkynyl; and
R46, R47, R51, R52, R56, R57, R60, R61, R66, R67, R72 and R73 are independently selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl.

In some embodiments of the third aspect and second embodiment thereof, the compound has a structure selected from the group consisting of Formula VIb-1, Formula VIb-2, Formula VIc-1, Formula VIc-2, Formula VId-1, Formula VId-2, Formula VId-3, and Formula VId-4:

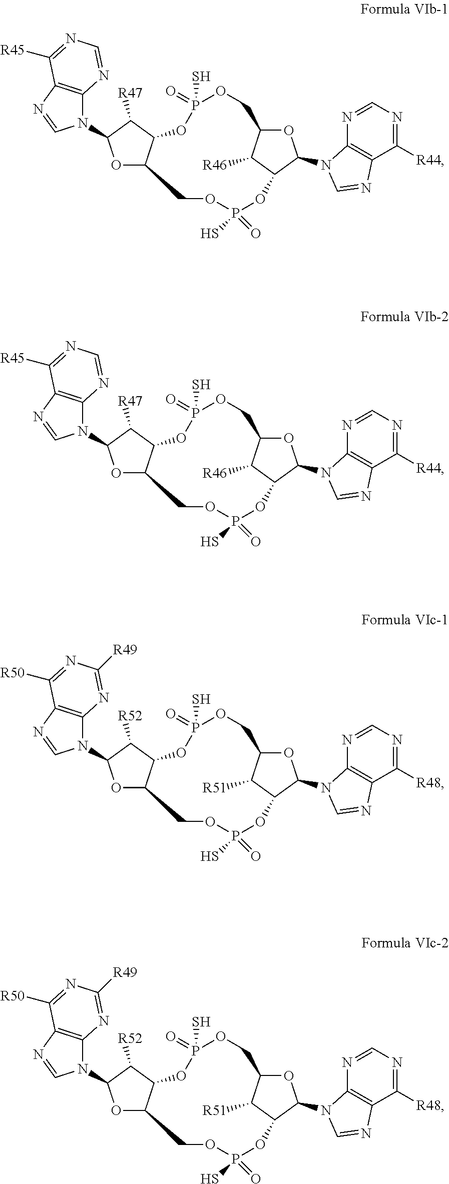

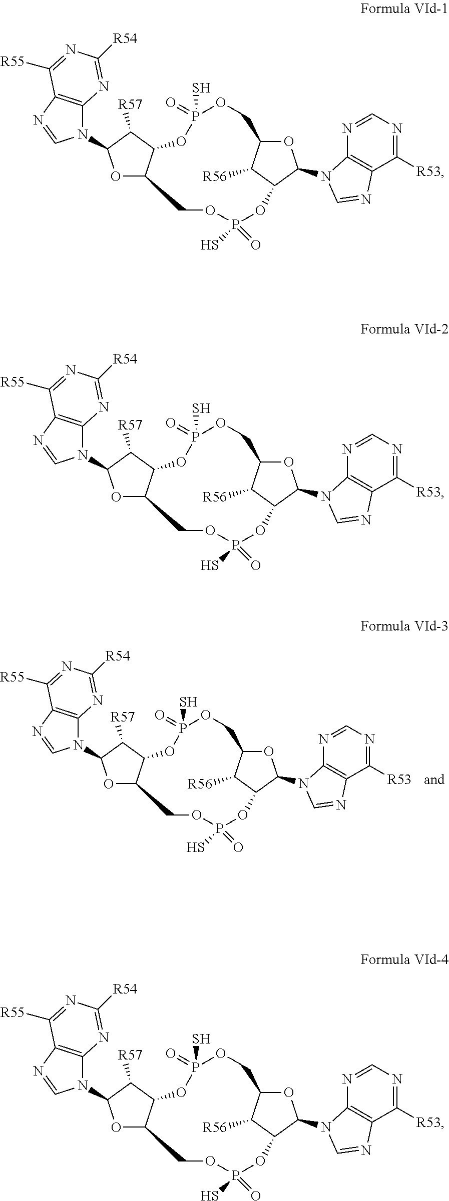

or a prodrug, tautomer, pharmaceutically acceptable salt, pharmaceutically acceptable solvate or pharmaceutically acceptable hydrate thereof, wherein R44, R45, R46, R47, R48, R49, R50, R51, R52, R53, R54, R55, R56 and R57 are as defined for the second embodiment of the third aspect.

In some embodiments of the third aspect and second embodiment thereof, the compound has a structure selected from the group consisting of Formula VIIc-1, Formula VIIc-2, Formula VIId-1 and Formula VIId-2:

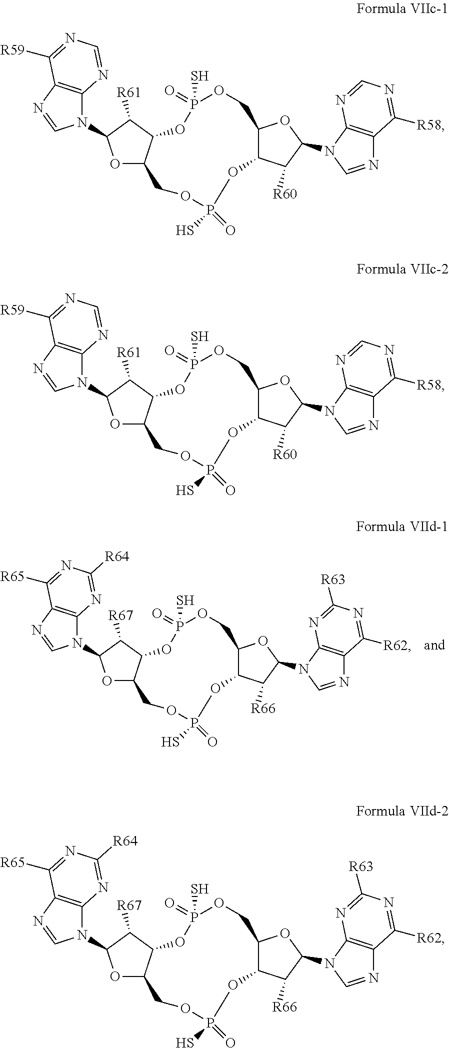

or a prodrug, tautomer, pharmaceutically acceptable salt, pharmaceutically acceptable solvate or pharmaceutically acceptable hydrate thereof, wherein R58, R59, R60, R61, R62, R63, R64, R65, R66 and R67 are as defined for the second embodiment of the third aspect.

In a fourth aspect, the present invention provides a compound having the structure of Formula VIII:

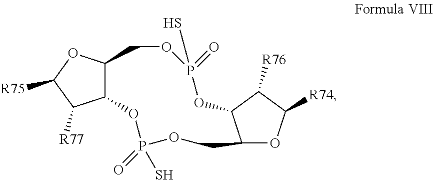

or a prodrug, tautomer, pharmaceutically acceptable salt, pharmaceutically acceptable solvate or pharmaceutically acceptable hydrate thereof,
wherein:
R74 is a purine or modified purine linked to the structure via the N9 position, or a pyrimidine or modified pyrimidine linked to the structure via the N1 position, preferably wherein the purine or modified purine is adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, and wherein the pyrimidine or modified pyrimidine is cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl;
R75 is a purine or modified purine linked to the structure via the N9 position, or a pyrimidine or modified pyrimidine linked to the structure via the N1 position, preferably wherein the purine or modified purine is adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, and wherein the pyrimidine or modified pyrimidine is cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl;
R76 is selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl;
R77 is selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹³⁰, wherein R¹⁰¹, R¹⁰² and R¹⁰³ are independently C₁₋₆alkyl or phenyl,
   wherein the compound has STING inhibitory activity.

In a first embodiment of the fourth aspect, the compound has a structure of Formula VIIIa:

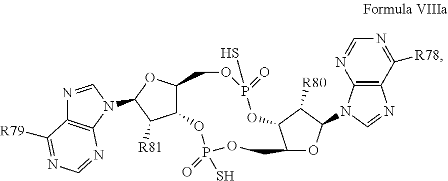

or a prodrug, tautomer, pharmaceutically acceptable salt, pharmaceutically acceptable solvate or pharmaceutically acceptable hydrate thereof,

wherein:
R78 and R79 are independently selected from the group consisting of —NH₂, —NHR^{y}, —NR^{y}R^{z}, —NHCH₂R¹⁰⁴ and —NHC(O)R¹⁰⁴, wherein R^{y} and R^{z} are independently alkyl, and R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy; and
R80 and R81 are independently selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl.

In some embodiments of the fourth aspect and first embodiment thereof, the compound has a structure selected from the group consisting of Formula VIIIa-1, Formula VIIIa-2 and Formula VIIIa-3:

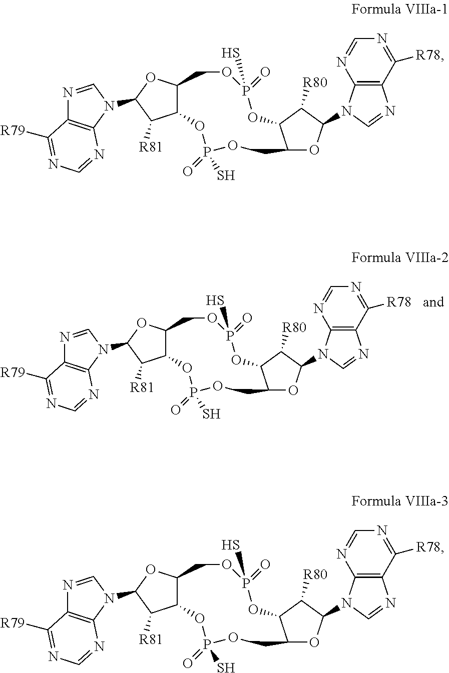

or a prodrug, tautomer, pharmaceutically acceptable salt, pharmaceutically acceptable solvate or pharmaceutically acceptable hydrate thereof, wherein R78, R79, R80 and R81 are as defined for the first embodiment of the fourth aspect.

In a fifth aspect, the present invention provides a pharmaceutical composition comprising one or more compounds of Formula VI, Formula VII or Formula VIII, as described in the third and fourth aspects and any embodiments thereof, including any prodrugs, tautomers, pharmaceutically acceptable salts, pharmaceutically acceptable solvates, or pharmaceutically acceptable hydrates thereof, and a pharmaceutically acceptable excipient. In some embodiments, the pharmaceutical compositions are formulated as aqueous, liposomal, or oil-in-water emulsions.

In a sixth aspect, the present invention provides provides a method for treating an individual suffering from an autoimmune disease, wherein the method comprises administering to the individual in need thereof an effective amount of one or more compounds of Formula VI, Formula VII or Formula VIII, as described in the third and fourth aspects and any embodiments thereof, including any prodrugs, tautomers, pharmaceutically acceptable salts, pharmaceutically acceptable solvates, or pharmaceutically acceptable hydrates thereof, or a pharmaceutical composition thereof as described in the fifth aspect. In some embodiments, the one or more compounds or composition thereof is administered non-parenterally or parenterally. In some embodiments, the administration is subcutaneous, intravenous, intramuscular, intraarterial, intradermal, intrathecal or epidural administrations.

In a first embodiment of the sixth aspect, the individual receiving such treatment may be suffering from an autoimmune disease selected from the group consisting of alopecia areata, autoimmune hemolytic anemia, autoimmune hepatitis, dermatomyositis, type 1 diabetes, autoimmune juvenile idiopathic arthritis, glomerulonephritis, Graves' disease, Guillain-Barré syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, lupus, STING-associated vasculopathy with onset in infancy, Aicardi-Goutières syndrome, some forms of thyroiditis, some forms of uveitis, vitiligo, and granulomatosis with polyangiitis.

In a seventh aspect, the invention provides a method for the treatment of disorders in which shifting of Th1 to Th2 immunity confers clinical benefit, wherein the method comprises administering to the individual in need thereof an effective amount of one or more compounds of Formula VI, Formula VII or Formula VIII, as described in the third and fourth aspects and any embodiments thereof, including any prodrugs, tautomers, pharmaceutically acceptable salts, pharmaceutically acceptable solvates, or pharmaceutically acceptable hydrates thereof, or a pharmaceutical composition thereof as described in the fifth aspect. Cell-mediated immunity (CMI) is associated with TH1 CD4+ T lymphocytes producing cytokines IL-2, interferon (IFN)-γ and tumor necrosis factor (TNF)-α. In contrast, humoral immunity is associated with TH2 CD4+ T lymphocytes producing IL-4, IL-6 and IL-10. Immune deviation towards TH1 responses typically produces activation of cytotoxic T-cell lymphocytes (CTL), natural killer (NK) cells, macrophages and monocytes. Generally, Th1 responses are more effective against intracellular pathogens (viruses and bacteria that are inside host cells) and tumors, while Th2 responses are more effective against extracellular bacteria, parasites including helminths and toxins. In addition, the activation of innate immunity is expected to normalize the T-helper type 1 and 2 (Th1/Th2) immune system balance and to suppress the excessive reaction of Th2 type responses that cause immunoglobulin (Ig) E-dependent allergies and allergic asthma.

In an eighth aspect, the present invention provides a method for treating an individual in need thereof comprising non-parenterally or parenterally administering to the individual an effective amount of a scaffold molecule as described herein in the first aspect and any embodiments thereof, or one or more compounds of Formula VI, Formula VII or Formula VIII, as described in the third and fourth aspects and any embodiments thereof, including any prodrugs, tautomers, pharmaceutically acceptable salts, pharmaceutically acceptable solvates, or pharmaceutically acceptable hydrates thereof, or a pharmaceutical composition thereof as described in the fifth aspect, under conditions where STING activity within the individual is reduced.

In a ninth aspect, the present invention provides a method of inhibiting STING administering to the individual an effective amount of a scaffold molecule as described herein in the first aspect and any embodiments thereof, or one or more compounds of Formula VI, Formula VII or Formula VIII, as described in the third and fourth aspects and any embodiments thereof, including any prodrugs, tautomers, pharmaceutically acceptable salts, pharmaceutically acceptable solvates, or pharmaceutically acceptable hydrates thereof, or a pharmaceutical composition thereof as described in the fifth aspect, under conditions where STING activity within the individual is reduced.

In a tenth aspect, the invention provides one or more compounds of Formula VI, Formula VII or Formula VIII, as described in the third and fourth aspects and any embodiments thereof, including any prodrugs, tautomers, pharmaceutically acceptable salts, pharmaceutically acceptable solvates, or pharmaceutically acceptable hydrates thereof, or a pharmaceutical composition thereof as described in the fifth aspect, for use in treating an autoimmune disease. In some embodiments, the autoimmune disease is selected from the group consisting of alopecia areata, autoimmune hemolytic anemia, autoimmune hepatitis, dermatomyositis, type 1 diabetes, autoimmune juvenile idiopathic arthritis, glomerulonephritis, Graves' disease, Guillain-Barré syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, lupus, STING-associated vasculopathy with onset in infancy, Aicardi-Goutières syndrome, some forms of thyroiditis, some forms of uveitis, vitiligo, and granulomatosis with polyangiitis.

In an eleventh aspect, the invention provides one or more compounds of Formula VI, Formula VII or Formula VIII, as described in the third and fourth aspects and any embodiments thereof, including any prodrugs, tautomers, pharmaceutically acceptable salts, pharmaceutically acceptable solvates, or pharmaceutically acceptable hydrates thereof, or a pharmaceutical composition thereof as described in the fifth aspect, for use the preparation of a medicament for the treatment of an autoimmune disease. In some embodiments, the autoimmune disease is selected from the group consisting of alopecia areata, autoimmune hemolytic anemia, autoimmune hepatitis, dermatomyositis, type I diabetes, autoimmune juvenile idiopathic arthritis, glomerulonephritis, Graves' disease, Guillain-Barré syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, lupus, STING-associated vasculopathy with onset in infancy, Aicardi-Goutières syndrome, some forms of thyroiditis, some forms of uveitis, vitiligo, and granulomatosis with polyangiitisa.

In a twelfth aspect, the invention provides a kit that includes one or more compounds of Formula VI, Formula VII or Formula VIII, as described in the third and fourth aspects and any embodiments thereof, including any prodrugs, tautomers, pharmaceutically acceptable salts, pharmaceutically acceptable solvates, or pharmaceutically acceptable hydrates thereof, or a pharmaceutical composition thereof as described in the fifth aspect. In some embodiments, one or more compounds or compositions thereof is packaged, e.g., in a vial, bottle or similar container, which may be further packaged, e.g., within a box, envelope, or similar container. In some embodiments, one or more compounds or compositions thereof is approved by the U.S. Food and Drug Administration or similar regulatory agency for administration to a mammal, e.g., a human. In one embodiment, such a kit includes written instructions for use and/or other indication that the one or more compounds or compositions thereof is suitable or approved for administration to a mammal, e.g., a human, for a suitable disease or condition. In some embodiments, the compound or composition is packaged in unit dose or single dose form, e.g., single dose pills, capsules, or the like.

### BRIEF DESCRIPTION OF THE FIGURES

FIG. 1A-1B depicts the IRF-3 fold induction in THP-1 cells without digitonin showing an exemplary pattern for antagonist scaffold molecule RR-(2′F-ibG)(2′F-ibG) (FIG. 1A) or antagonist scaffold molecules RR-(2,6-DAP)(2,6-DAP) and RS-(2,6-DAP)(2,6-DAP) (FIG. 1B) in this assay as compared to agonist activity of 2′3′-RR-(A)(A).

FIG. 2A-2B depicts the IRF-3 fold induction in THP-1 cells without digitonin in a competition assay with 2′3′-RR-(A)(A) for control agonists 2′3′-RR-(G)(A) (FIG. 2A) and RR-(A)(A) (FIG. 2B).

FIG. 3A-3O depicts the IRF-3 fold induction in THP-1 cells without digitonin in a competition assay with 2′3′-RR-(A)(A) for scaffold molecules 2′3′-SR-(3′OTBS-A)(2′F-A) (FIG. 3A), SR-(2′F-A)(2′OTBS-A) (FIG. 3B), RR-(2,6-DAP)(2,6-DAP) (FIG. 3C), RS-(2,6-DAP)(2,6-DAP) (FIG. 3D), (6-O-propargyl-G)(G) (FIG. 3E), RR-(2′F-ibG)(2′F-ibG) (FIG. 3F), RR-(2′F-C)(2′F-A) (FIG. 3G), RS-(2′F-C)(2′F-A) (FIG. 3H), 3′2′-RR-(ibG)(BzA) (FIG. 3I), 3′2′-SS-(G)(A) (FIG. 3J), 3′2′-(2′OTBS-G)(3′OTBS-A) dithio isomer 1 (FIG. 3K), 3′2′-(2′OTBS-G)(3′OTBS-A) dithio isomer 2 (FIG. 3L), Beta-L-SS-(A)(A) (FIG. 3M), Beta-L-RS-(A)(A) (FIG. 3N), and Beta-L-RR-(A)(A) (FIG. 3O).

### DETAILED DESCRIPTION OF THE INVENTION

The present invention relates to the use cyclic-di-nucleotide and related scaffold molecules that measurably inhibit signaling at the cytoplasmic receptor known as STING (Stimulator of Interferon Genes), for use in identifying more potent inhibitors of STING signaling. In particular, the methods provided can be used to identify potent inhibitors of STING signaling, which are useful in the treatment of autoimmune and inflammatory diseases. Also provided are compounds of Formula VI, VII or VIII, and embodiments thereof, that are potent inhibitors of STING signaling.

The CDNs cyclic-di-AMP (produced by Listeria monocytogenes) and its analog cyclic-di-GMP (produced by Legionella pneumophila) are recognized by the host cell as a PAMP (Pathogen Associated Molecular Pattern), which bind to the PRR (Pathogen Recognition Receptor) known as STING. STING is an adaptor protein in the cytoplasm of host mammalian cells which activates the TANK binding kinase (TBK1)-IRF3 signaling axis, resulting in the induction of IFN-γ and other IRF-3 dependent gene products that strongly activate innate immunity. It is now recognized that STING is a component of the host cytosolic surveillance pathway, which senses infection with intracellular pathogens and in response induces the production of IFN, leading to the development of an adaptive protective pathogen-specific immune response consisting of both antigen-specific CD4 and CD8 T cells as well as pathogen-specific antibodies.

The STING inhibitors identified as described herein, and compositions thereof, can be used in methods of inhibiting or moderating an immune response in an individual, comprising administering a composition comprising the identified STING inhibitor to an individual in need thereof. The STING inhibitors identified as described herein, and compositions thereof, can be used in methods of inhibiting or moderating type I interferon production in an individual, comprising administering a composition comprising the identified STING inhibitor to an individual in need thereof.

In the case of autoimmune diseases, inhibitors of the STING pathway can provide a therapeutic route which has not been previously exploited. The STING inhibitors identified as described herein, and compositions thereof, can be used in methods for treating an autoimmune disease, comprising administering a composition comprising the identified STING inhibitor to an individual in need thereof. Examples of autoimmune diseases which may be treated using the compositions comprising the identified STING inhibitor include, but are not limited to, alopecia areata, autoimmune hemolytic anemia, autoimmune hepatitis, dermatomyositis, diabetes (type 1), autoimmune juvenile idiopathic arthritis, glomerulonephritis, Graves' disease, Guillain-Barré syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, lupus (e.g. systemic lupus erythematosus), STING-associated vasculopathy with onset in infancy (SAVI), Aicardi-Goutières syndrome (AGS), some forms of thyroiditis, some forms of uveitis, vitiligo, and granulomatosis with polyangiitis (Wegener's granulomatosis).

The STING inhibitors identified as described herein, and compositions thereof, can be used in methods for the treatment of disorders in which shifting of Th1 to Th2 immunity confers clinical benefit. Cell-mediated immunity (CMI) is associated with TH1 CD4+ T lymphocytes producing cytokines IL-2, interferon (IFN)-γ and tumor necrosis factor (TNF)-α. In contrast, humoral immunity is associated with TH2 CD4+ T lymphocytes producing IL-4, IL-6 and IL-10. Immune deviation towards TH1 responses typically produces activation of cytotoxic T-cell lymphocytes (CTL), natural killer (NK) cells, macrophages and monocytes. Generally, Th1 responses are more effective against intracellular pathogens (viruses and bacteria that are inside host cells) and tumors, while Th2 responses are more effective against extracellular bacteria, parasites including helminths and toxins. Type I interferons (IFNs-I) are believed to mediate the lethal effects of endotoxemia and sepsis, and so the methods and compositions of the present invention can find use in the treatment of sepsis. In addition, the activation of innate immunity is expected to normalize the T-helper type 1 and 2 (Th1/Th2) immune system balance and to suppress the excessive reaction of Th2 type responses that cause immunoglobulin (Ig) E-dependent allergies and allergic asthma.

The STING inhibitors identified as described herein, and compositions thereof, may be administered to individuals in need thereof by a variety of parenteral and nonparenteral routes in formulations containing pharmaceutically acceptable carriers, adjuvants and vehicles. Preferred routes are parenteral, and include but, are not limited to, one or more of subcutaneous, intravenous, intramuscular, intraarterial, intradermal, intrathecal and epidural administrations. Particularly preferred is administration by subcutaneous administration. Preferred pharmaceutical compositions are formulated as aqueous, liposomal, or oil-in-water emulsions. Exemplary compositions are described hereinafter.

### Definitions

“Administration” as it is used herein with regard to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell. By “administered together” it is not meant to be implied that two or more agents be administered as a single composition. Although administration as a single composition is contemplated by the present invention, such agents may be delivered to a single subject as separate administrations, which may be at the same or different time, and which may be by the same route or different routes of administration.

An “agonist,” as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a mutein or derivative of GM-CSF, a peptide mimetic of GM-CSF, a small molecule that mimics the biological function of GM-CSF, or an antibody that stimulates GM-CSF receptor.

An “antagonist,” as it relates to a ligand and receptor, comprises a molecule, combination of molecules, or a complex, that inhibits, counteracts, downregulates, and/or desensitizes the receptor. “Antagonist” encompasses any reagent that inhibits a constitutive activity of the receptor. A constitutive activity is one that is manifest in the absence of a ligand/receptor interaction. “Antagonist” also encompasses any reagent that inhibits or prevents a stimulated (or regulated) activity of a receptor. By way of example, an antagonist of GM-CSF receptor includes, without implying any limitation, an antibody that binds to the ligand (GM-CSF) and prevents it from binding to the receptor, or an antibody that binds to the receptor and prevents the ligand from binding to the receptor, or where the antibody locks the receptor in an inactive conformation.

“Specifically” or “selectively” binds, when referring to a ligand/receptor, nucleic acid/complementary nucleic acid, antibody/antigen, or other binding pair (e.g., a cytokine to a cytokine receptor) (each generally referred to herein as a “target biomolecule” or a “target”) indicates a binding reaction which is related to the presence of the target in a heterogeneous population of proteins and other biologics. Specific binding can mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with a non-target molecule.

“Ligand” refers to a small molecule, nucleic acid, peptide, polypeptide, saccharide, polysaccharide, glycan, glycoprotein, glycolipid, or combinations thereof that binds to a target biomolecule. While such ligands may be agonists or antagonists of a receptor, a ligand also encompasses a binding agent that is not an agonist or antagonist, and has no agonist or antagonist properties. Specific binding of a ligand for its cognate target is often expressed in terms of an “Affinity.” In preferred embodiments, the ligands of the present invention bind with affinities of between about 10⁴ M⁻¹ and about 10⁸ M⁻¹. Affinity is calculated as K_{d}=k_{off}/kₒₙ (k_{off} is the dissociation rate constant, Kₒₙ is the association rate constant and K_{d} is the equilibrium constant).

A “scaffold molecule” as used herein refers to a molecule that measurably binds human STING protein, including but not limited to one or more of WT, HAQ allele, and REF allele, and has little or no STING agonist activity.

“Measurable STING binding” as used herein is determined in a DSF assay under the conditions described in Example 11, where a positive Tₘ shift can be measured when human STING protein is assayed with and without the scaffold molecule. Preferably, the Tₘ shift is at least 2° C. under the described conditions.

“STING agonist activity” as used herein is determined in a THP-1 cell assay under the conditions described in Example 12 to measure the STING-dependent induction of type I interferon, where the molecule shows STING agonist activity that can be detected by standard techniques, including the measurement of IRF-3 signaling activity.

Molecules are not considered to be STING agonists as that term is used herein when they have little or no STING agonist activity in such a standard assay. Such molecules may induce STING activity at high concentrations, but would still not be considered a STING agonist if such molecule shows e.g. no more than 10%, preferably no more than 5%, and more preferably no more than 1% or less of the IRF-3 reporter activity when tested at a concentration equal to the concentration of the known STING agonist 2′3′-RR-(A)(A) which gives half maximal activity as measured by the THP-1 assay as described in Example 12 (assayed without digitonin). Such molecules may induce STING activity at high concentrations, but would still not be considered a STING agonist if such molecule shows e.g. less than 100 fold, or less than 50 fold, or less than 10 fold maximal IRF-3 reporter activity over a concentration range of e.g. 0.01 μM to 2,000 μM, or 0.01 μM to 1,000 μM as measured by the THP-1 assay as described in Example 12 (assayed without digitonin).

A molecule has “measurable STING antagonist activity” or “measurable STING inhibitory activity” as those terms are used herein where, when the molecule is assayed in a standard THP-1 competition assay under the conditions described in Example 13 with the known STING agonist 2′3′-RR-(A)(A), the molecule demonstrates an inhibition of STING induction by the agonist, as demonstrated in Example 13.

Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K=equilibrium association constant; and n=number of ligand binding sites per receptor molecule. By graphical analysis, r/c is plotted on the Y-axis versus r on the X-axis, thus producing a Scatchard plot. Affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988. In an alternative, affinity can be measured by isothermal titration calorimetry (ITC). In a typical ITC experiment, a solution of ligand is titrated into a solution of its cognate target. The heat released upon their interaction (ΔH) is monitored over time. As successive amounts of the ligand are titrated into the ITC cell, the quantity of heat absorbed or released is in direct proportion to the amount of binding. As the system reaches saturation, the heat signal diminishes until only heats of dilution are observed. A binding curve is then obtained from a plot of the heats from each injection against the ratio of ligand and binding partner in the cell. The binding curve is analyzed with the appropriate binding model to determine K_{B}, n and ΔH. Note that K_{B}=1/K_{d}.

The term “subject” as used herein refers to a human or non-human organism. Thus, the methods and compositions described herein are applicable to both human and veterinary disease. In certain embodiments, subjects are “patients,” i.e., living humans that are receiving medical care for a disease or condition. This includes persons with no defined illness who are being investigated for signs of pathology.

“Therapeutically effective amount” is defined as an amount of a reagent or pharmaceutical composition that is sufficient to show a patient benefit, i.e., to cause a decrease, prevention, or amelioration of the symptoms of the condition being treated. When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual. “Effective amount” encompasses, without limitation, an amount that can ameliorate, reverse, mitigate, prevent, or diagnose a symptom or sign of a medical condition or disorder or a causative process thereof. Unless dictated otherwise, explicitly or by context, an “effective amount” is not limited to a minimal amount sufficient to ameliorate a condition.

“Treatment” or “treating” (with respect to a condition or a disease) is an approach for obtaining beneficial or desired results including and preferably clinical results. For purposes of this invention, beneficial or desired results with respect to a disease include, but are not limited to, one or more of the following: preventing a disease, improving a condition associated with a disease, curing a disease, lessening severity of a disease, delaying progression of a disease, alleviating one or more symptoms associated with a disease, increasing the quality of life of one suffering from a disease, and/or prolonging survival. Likewise, for purposes of this invention, beneficial or desired results with respect to a condition include, but are not limited to, one or more of the following: preventing a condition, improving a condition, curing a condition, lessening severity of a condition, delaying progression of a condition, alleviating one or more symptoms associated with a condition, increasing the quality of life of one suffering from a condition, and/or prolonging survival. Depending on the context, “treatment” of a subject can imply that the subject is in need of treatment, e.g., in the situation where the subject comprises a disorder expected to be ameliorated by administration of a reagent.

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

### Cyclic Purine Dinucleotides

Prokaryotic as well as eukaryotic cells use various small molecules for cell signaling and intra- and intercellular communication. Cyclic nucleotides like cGMP, cAMP, etc. are known to have regulatory and initiating activity in pro- and eukaryotic cells. Unlike eukaryotic cells, prokaryotic cells also use cyclic purine dinucleotides as regulatory molecules. In prokaryotes, the condensation of two GTP molecules is catalyst by the enzyme diguanylate cyclase (DGC) to give c-diGMP, which represents an important regulator in bacteria.

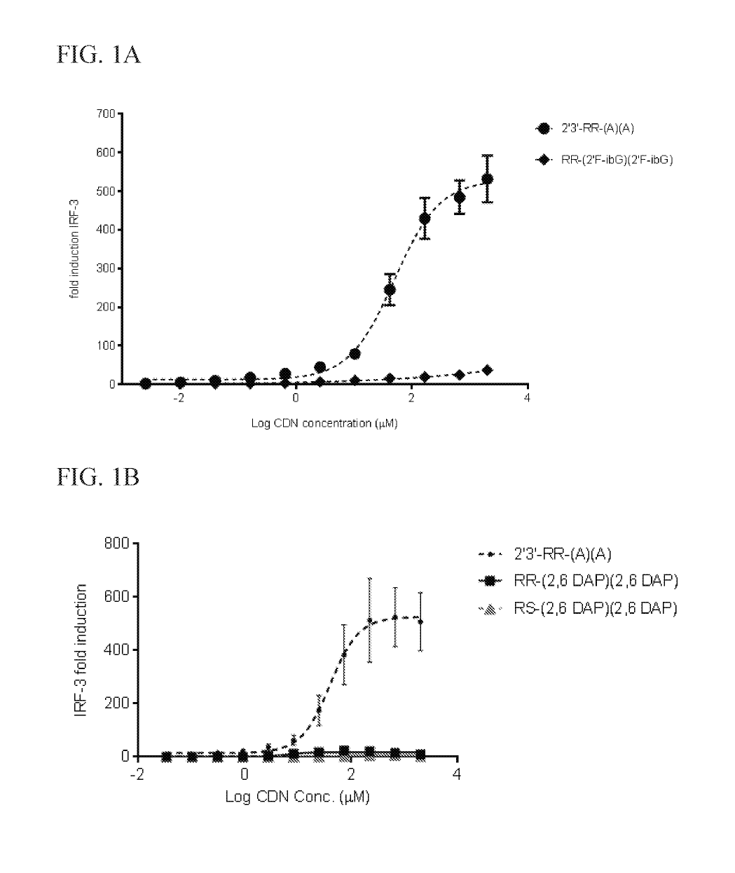

Recent work suggests that cyclic diGMP or analogs thereof can also stimulate or enhance immune or inflammatory response in a patient or can enhance the immune response to a vaccine by serving as an adjuvant in mammals. Cytosolic detection of pathogen-derived DNA requires signaling through TANK binding kinase 1 (TBK1) and its downstream transcription factor, IFN-regulatory factor 3 (IRF3). A transmembrane protein called STING (stimulator of IFN genes; also known as MITA, ERIS, MPYS and TMEM173) functions as the signaling receptor for these cyclic purine dinucleotides, causing stimulation of the TBK1-IRF3 signalling axis and a STING-dependent type I interferon response. See, e.g., FIG. 1. Burdette et al., Nature 478: 515-18, 2011 demonstrated that STING binds directly to cyclic diguanylate monophosphate, but not to other unrelated nucleotides or nucleic acids.

The term “alkyl,” as used herein, refers to a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms with from 1 to about 6 carbon atoms being more preferred. The term “lower alkyl” as used herein includes from 1 to about 6 carbon atoms.

The term “alkenyl,” as used herein, refers to a straight or branched hydrocarbon chain radical containing up to twenty four carbon atoms and having at least one carbon-carbon double bond. Examples of alkenyl groups include without limitation, ethenyl, propenyl, butenyl, l-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms (“lower alkenyl”) being more preferred.

The term “alkynyl,” as used herein, refers to a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms (“lower alkynyl”) being more preferred.

The term “alkoxy,” as used herein, refers to a radical formed between an alkyl group, preferably a lower alkyl group, and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like.

The term “mono-alkylamino” and “di-alkylamino” as used herein, refers to a primary amino group substituted with one (mono-alkyl) or two (di-alkyl)groups, wherein the alkyl groups are preferably lower alkyl. Examples of mono-alkylamine groups include methylamino, ethylamino, propylamino, isopropylamino, n-butylamino, tert-butylamino, and the like. Examples of di-alkylamino can be di-substituted with the same alkyl groups, or two different alkyl groups, such as dimethylamino, diethylamino, ethyl(methyl)amino, methyl(propyl)amino, ethyl(isopropyl)amino, and the like.

The terms “halo” and “halogen,” as used herein, refer to an atom selected from fluorine, chlorine, bromine and iodine.

The term “5 or 6 membered heteroaryl” as used herein, refers to a radical comprising a mono-cyclic aromatic ring wherein the ring includes one or more heteroatoms, preferably one or more nitrogen in a 6-membered heteroaryl (e.g. 1, 2, or 3 nitrogens) and one or more nitrogens and/or sulfur or oxygen in a 5-membered heteroaryl (e.g. 1, 2, 3 or 4 nitrogens; 1, 2 or 3 nitrogens and one sulfur; 1, 2, or 3 nitrogens and one oxygen; 1 sulfur; or 1 oxygen). Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, and the like. Heteroaryl radicals can be attached to a parent molecule directly via a carbon atom or, as appropriate, a nitrogen atom of the ring. Where it is indicated that heteroaryl is optionally substituted with one or more substituents, one or more is typically 1, 2, 3, 4 or 5, also 1, 2, 3, or 4, also 1, 2, or 3, also 1 or 2, or one, where multiple substituents are independently selected unless indicated otherwise. It is understood that any substitutions of heteroaryl, or heteroaryl substituted on another moiety, are attached at any available atom to provide a stable compound.

The term “substantially pure” as used herein with regard to scaffold molecules and identified STING inhibitors as described herein refers to an Rp,Rp or Rp,Sp form which is at least 75% pure relative to other possible stereochemistries at the chiral centers indicated in the figure above. By way of example, a “substantially pure Rp,Rp c-di-GMP thiophosphate” would be at least 75% pure with regard to the Rp,Sp and Sp,Sp forms of c-di-GMP thiophosphate. In preferred embodiments, a substantially pure cyclic purine dinucleotide is at least 85% pure, at least 90% pure, at least 95% pure, at least 97% pure, and at least 99% pure. While a substantially pure cyclic purine dinucleotide preparation of the invention is “stereochemically pure,” this is not meant to indicate that all CDNs within the preparation having a particular stereochemistry at these chiral centers are otherwise identical. For example, a substantially pure cyclic purine dinucleotide preparation may contain a combination of Rp,Rp c-di-GMP thiophosphate and Rp,Rp c-di-AMP thiophosphate and still be a substantially pure cyclic purine dinucleotide preparation. Such a preparation may also include other components as described hereinafter that are advantageous for patient treatment, provided that all CDNs within the preparation having a particular stereochemistry at these chiral centers.

The STING inhibitors identified as described herein, or a compound of Formula VI, VII or VIII, and all embodiments thereof, can be administered to a host, either alone or in combination with a pharmaceutically acceptable excipient, in an amount sufficient to modify an appropriate immune response. The immune response can comprise, without limitation, specific immune response, non-specific immune response, both specific and non-specific response, innate response, primary immune response, adaptive immunity, secondary immune response, memory immune response, immune cell activation, immune cell proliferation, immune cell differentiation, and cytokine expression. In certain embodiments, the STING inhibitors identified as described herein, or a compound of Formula VI, VI or VIII, and all embodiments thereof, are administered in conjunction with one or more additional compositions. The STING inhibitors identified as described herein, or a compound of Formula VI, VI or VIII, and all embodiments thereof, may be administered before, after, and/or together with an additional therapeutic or prophylactic composition. Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). In certain embodiments the one or more therapeutics is selected from anti-TNF agents (e.g., etanercept, infliximab), steroids, azathioprine, cyclosporine, methotrexate, abatacept, PDE4 inhibitors (e.g., roflumilast), etc.

### Delivery Agents

Delivery agents can be used in formulations comprising one or more compounds having STING inhibitory activity as described herein, and compositions thereof described herein in accordance with the present invention. Such additives or delivery vehicles include, without limitation, lipid or lipid-like adjuvants, liposomes, interbilayer crosslinked multilamellar vesicles, nanocarriers, nanoparticles and the like, such as nanoparticles comprising Poly(lactic acid) (PLA), Poly(glycolic acid) (PGA), and/or their copolymers such as biodegradable poly(D,L-lactic-co-glycolic acid) [PLGA]-based or poly anhydride-based nanoparticles or microparticles.

Liposomes are vesicles formed from one (“unilamellar”) or more (“multilamellar”) layers of phospholipid. Because of the amphipathic character of the phospholipid building blocks, liposomes typically comprise a hydrophilic layer presenting a hydrophilic external face and enclosing a hydrophilic core. The versatility of liposomes in the incorporation of hydrophilic/hydrophobic components, their non-toxic nature, biodegradability, biocompatibility, adjuvanticity, induction of cellular immunity, property of sustained release and prompt uptake by macrophages, makes them attractive candidates for the delivery of antigens.

WO2010/104833 describes suitable liposomal preparations. Such liposomal formulations, referred to herein as VesiVax® (Molecular Express, Inc.), with our without the “immunogenic polypeptide(s) or carbohydrate(s)” referred to above, can contain one or more additional components such as peptidoglycan, lipopeptide, lipopolysaccharide, monophosphoryl lipid A, lipoteichoic acid, resiquimod, imiquimod, flagellin, oligonucleotides containing unmethylated CpG motifs, beta-galactosylceramide, muramyl dipeptide, all-trans retinoic acid, double-stranded viral RNA, heat shock proteins, dioctadecyldimethylammonium bromide, cationic surfactants, toll-like receptor agonists, dimyristoyltrimethylammoniumpropane, and nod-like receptor agonists. Advantageously, these liposomal formulations can be used to deliver one or more compounds having STING inhibitory activity as described herein, and compositions thereof described herein in accordance with the present invention.

Moreover, while the liposomal formulations discussed above employ a “steroid derivative” as an anchor for attaching an immunogenic polypeptide or carbohydrate to a liposome, the steroid may simply be provided as an unconjugated steroid such as cholesterol.

Suitable methods for preparing liposomes from lipid mixtures are well known in the art. See, e.g., Basu & Basu, Liposome Methods and Protocols (Methods in Molecular Bilogy), Humana Press, 2002; Gregoriadis, Liposome Technology, 3^{rd} Edition, Informa HealthCare, 2006. Preferred methods include extrusion, homogenization, and sonication methods described therein. An exemplary method for preparing liposomes for use in the present invention, which comprises drying a lipid mixture, followed by hydration in an aqueous vehicle and sonication to form liposomes, is described in WO2010/104833.

In certain embodiments, the liposomnes are provided within a particular average size range. Liposome size can be selected, for example, by extrusion of an aqueous vehicle comprising liposomes through membranes having a preselected pore size and collecting the material flowing through the membrane. In preferred embodiments, the liposomes are selected to be substantially between 50 and 500 nm in diameter, more preferably substantially between 50 and 200 nm in diameter, and most preferably substantially between 50 and 150 nm in diameter. The term “substantially” as used herein in this context means that at least 75%, more preferably 80%, and most preferably at least 90% of the liposomes are within the designated range.

Other lipid and lipid-like adjuvants which may find use in the present invention include oil-in-water (o/w) emulsions (see, e.g., Muderhwa et al., J. Pharmaceut. Sci. 88: 1332-9, 1999)), VesiVax® TLR (Molecular Express, Inc.), digitonin (see, e.g., U.S. Pat. No. 5,698,432), and glucopyranosyl lipids (see, e.g., United States Patent Application 20100310602).

Nanoparticles also represent drug delivery systems suitable for most administration routes. Over the years, a variety of natural and synthetic polymers have been explored for the preparation of nanoparticles, of which Poly(lactic acid) (PLA), Poly(glycolic acid) (PGA), and their copolymers (PLGA) have been extensively investigated because of their biocompatibility and biodegradability. Nanoparticles and other nanocarriers act as potential carries for several classes of drugs such as anticancer agents, antihypertensive agents, immunomodulators, and hormones; and macromolecules such as nucleic acids, proteins, peptides, and antibodies. See, e.g., Crit. Rev. Ther. Drug Carrier Syst. 21:387-422, 2004; Nanomedicine: Nanotechnology, Biology and Medicine 1:22-30, 2005.

### Pharmaceutical Compositions

The term “pharmaceutical” as used herein refers to a chemical substance intended for use in the cure, treatment, or prevention of disease and which is subject to an approval process by the U.S. Food and Drug Administration (or a non-U.S. equivalent thereof) as a prescription or over-the-counter drug product. Details on techniques for formulation and administration of such compositions may be found in Remington, The Science and Practice of Pharmacy 21^{st} Edition (Mack Publishing Co., Easton, Pa.) and Nielloud and Marti-Mestres, Pharmaceutical Emulsions and Suspensions: 2^{nd} Edition (Marcel Dekker, Inc, New York).

For the purposes of this disclosure, the pharmaceutical compositions may be administered by a variety of means including non-parenterally, parenterally, by inhalation spray, topically, or rectally in formulations containing pharmaceutically acceptable carriers, adjuvants and vehicles. “Non-parenteral administration” encompasses oral, buccal, sublingual, topical, transdermal, ophthalmic, otic, nasal, rectal, cervical, pulmonary, mucosal, and vaginal routes. The term parenteral as used here includes but is not limited to subcutaneous, intravenous, intramuscular, intraarterial, intradermal, intrathecal and epidural injections with a variety of infusion techniques. Intraarterial and intravenous injection as used herein includes administration through catheters. Administration via intracoronary stents and intracoronary reservoirs is also contemplated. Intra-tumoral (directly into the tumor mass) or peri-tumoral (around the tumor mass) administration of the compounds of the present invention may directly activate locally infiltrating DC, directly promote tumor cell apoptosis or sensitize tumor cells to cytotoxic agents. The term oral as used herein includes, but is not limited to oral ingestion, or delivery by a sublingual or buccal route. Oral administration includes fluid drinks, energy bars, as well as pill formulations.

Pharmaceutical compositions may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing a drug compound in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents; such as magnesium stearate, stearic acid or talc. Tablets may be uncoated, or may be coated by known techniques including enteric coating, colonic coating, or microencapsulation to delay disintegration and adsorption in the gastrointestinal tract and/or provide a sustained action over a longer period. For example, a time delay material such as glyceryl monostearate or glyceryl distearate alone or with a wax may be employed.

Formulations for oral use may be also presented as hard gelatin capsules where the drug compound is mixed with an inert solid diluent, for example calcium phosphate or kaolin, or as soft gelatin capsules wherein the active ingredient is mixed with water or an oil medium, such as peanut oil, liquid paraffin or olive oil.

Pharmaceutical compositions may be formulated as aqueous suspensions in admixture with excipients suitable for the manufacture of aqueous-suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The pharmaceutical compositions of the disclosure may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 20 to 500 mg of active material compounded with an appropriate and convenient amount of carrier material which may vary from about 5 to about 95% of the total compositions. It is preferred that the pharmaceutical composition be prepared which provides easily measurable amounts for administration. Typically, an effective amount to be administered systemically is about 0.1 mg/kg to about 100 mg/kg and depends upon a number of factors including, for example, the age and weight of the subject (e.g., a mammal such as a human), the precise condition requiring treatment and its severity, the route of administration, and will ultimately be at the discretion of the attendant physician or veterinarian. It will be understood, however, that the specific dose level for any particular patient will depend on a variety of factors including the activity of the specific compound employed, the age, body weight, general health, sex and diet of the individual being treated; the time and route of administration; the rate of excretion; other drugs which have previously been administered; and the severity of the particular condition undergoing therapy, as is well understood by those skilled in the art.

As noted above, formulations of the disclosure suitable for oral administration may be presented as discrete units such as capsules, cachets or tablets each containing a predetermined amount of the active ingredient, as a powder or granules; as a solution or a suspension in an aqueous or non-aqueous liquid, or as an oil-in-water liquid emulsion or a water-in-oil liquid emulsion. The pharmaceutical compositions may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropyl ethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made in a suitable machine using a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric or colonic coating to provide release in parts of the gut other than the stomach. This is particularly advantageous with the STING inhibitor compounds as described herein when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

The STING inhibitors identified as described herein and the compounds of Formula VI, Formula VII or Formula VIII can be prepared as pharmaceutically acceptable salts, including, but not limited to: acetate, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydoxymethyl)aminomethane. Additional pharmaceutically acceptable salts are known to those skilled in the art, or as discussed herein. When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs may have different physical properties such as density, shape, hardness, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjust the conditions used during the crystallization or recrystallization of the compound.

For solvates of compounds of this invention, or salts thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, dimethyl sulfoxide, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

Because of their potential use in medicine, the salts of the compounds of this invention are preferably pharmaceutically acceptable. Suitable pharmaceutically acceptable salts include those described by P. Heinrich Stahl and Camille G. Wermuth in Handbook of Pharmaceutical Salts: Properties, Selection, and Use, 2^{nd} ed. (Wiley-VCH: 2011) and also Remington's Pharmaceutical Sciences, 18^{th} ed. (Mack Publishing, Easton Pa.: 1990) and also Remington: The Science and Practice of Pharmacy, 19^{th} ed. (Mack Publishing, Easton Pa.: 1995). Salt encompassed within the term “pharmaceutically acceptable salts” refer to non-toxic salts of the compounds in this invention.

Salts of the compounds of this invention containing a basic amine or other basic functional group may be prepared by any suitable method known in the art, including treatment of the free bases with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, formic acid, alginic acid, pyruvic acid, oxalic acid, glycolic acid, salicylic acid, pyranosildyl acid, such as glucuronic acid or galacturonic acid, alphahydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, glycolate, resinate, lactates, camsylates, tartrates, mandelates, and sulfonates, such as xylenesulfonates, methanesulfonates, propanesulfonates, naphthalene-1-sulfonates and naphthalene-2-sulfonates.

Salts of the compounds of this invention containing a phosphate diester, phosphorothioate diester or other acidic functional group can be prepared by reacting with a suitable base. Pharmaceutically acceptable salts include, but are not limited to: pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydoxymethyl)aminomethane. Additional pharmaceutically acceptable salts are known to those skilled in the art.

Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, zinc, as well as salts made from physiologically acceptable organic bases such as diethylamine, isopropylamine, olamine, benzathine, benethamine, tromethamine (2-amino-2-(hydroxymethyl)propane-1,3-diol), morpholine, epolamine, piperidine, piperazine, picoline, dicyclohexylamine, N,N′-dibenzylethylenediamine, 2-hydroxyethylamine, tri-(2-hydroxyethyl)amine, chloroprocaine, choline, deanol, imidazole, diethanolamine, ethylenediamine, meglumine (N-methylglucamine), procaine, dibenzylpiperidine, dehydroabietylamine, glucamine, collidine, quinine, quinolone, erbumine and basic amino acids such as lysine and arginine.

The compounds as described herein that include salts thereof can be described by structures wherein the —SH in the thiophosphate bond (or —OH for compounds without thiophosphate) are represented as exemplified below for compounds of Formula I as —S⁻ with a corresponding cation to form salts of the compounds as described herein. For example, salts of compounds of the first aspect as described herein can be represented by the following structures:

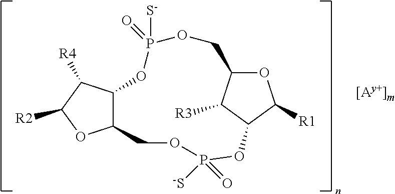

wherein A^{y+} represents a mono or polyvalent salt cation, and n and in are the lowest possible whole number for a given y. For example when A^{y+} is monovalent, i.e. when y is 1, such as Na⁺, K⁺, NH₄⁺, TEAH⁺ or the like, n is 1 and m is 2; when y is 2, such as Ca²⁺, Mg²⁺ and the like, n is 1 and m is 1; when y is 3, e.g. Al³⁺ or the like, n is 3 and m is 2. For example, salts of a monovalent or divalent salt cation can be represented as

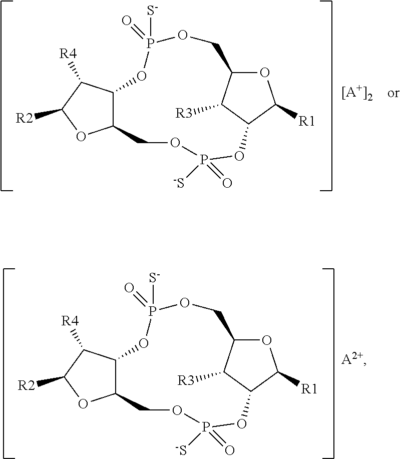

respectively, or in cases where n=1, these can be represented without brackets, e.g. as

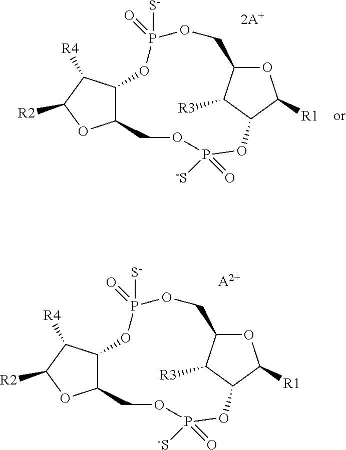

Alternatively, monovalent salts can be depicted with A⁺ adjacent each of the —S⁻ as follows.

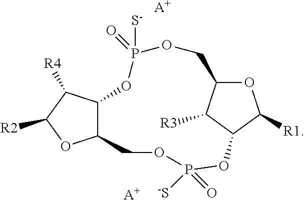

Other non-pharmaceutically acceptable salts, e.g. trifluoroacetate or triethylammonium may be used, for example in the isolation of compounds of the invention, and are included within the scope of this invention.

The invention includes within its scope all possible stoichiometric and non-stoichiometric forms of the salts of the compounds of this invention.

If a compound of this invention containing a basic amine or other basic functional group is isolated as a salt, the corresponding free base form of that compound may be prepared by any suitable method known to the art, including treatment of the salt with an inorganic or organic base, suitably an inorganic or organic base having a higher pKₐ than the free base form of the compound. Similarly, if a compound of this invention containing a phosphate diester, phosphorothioate diester or other acidic functional group is isolated as a salt, the corresponding free acid form of that compound may be prepared by any suitable method known to the art, including treatment of the salt with an inorganic or organic acid, suitably an inorganic or organic acid having a lower pKₐ than the free acid form of the compound.

An effective amount of a compound of the invention, or a pharmaceutically acceptable salt, pharmaceutically acceptable solvate or pharmaceutically acceptable hydrate thereof as described herein, for a particular patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

An effective amount may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of a pharmaceutical composition comprising a compound of the invention, or a pharmaceutically acceptable salt, pharmaceutically acceptable solvate or pharmaceutically acceptable hydrate thereof as described herein. Where there is more than one administration of a pharmaceutical composition in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals.

A dosing schedule of for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). Generally, co-administration or administration together indicates treating a subject with two or more agents, where the agents can be administered simultaneously or at different times. For example, such agents may be delivered to a single subject as separate administrations, which may be at essentially the same time or different times, and which may be by the same route or different routes of administration. Such agents may be delivered to a single subject in the same administration (e.g. same formulation) such that they are administered at the same time by the same route of administration.

As noted, the compositions of the present invention are preferably formulated as pharmaceutical compositions for parenteral or enteral delivery. A typical pharmaceutical composition for administration to an animal subject comprises a pharmaceutically acceptable vehicle such as aqueous solutions, non-toxic excipients, including salts, preservatives, buffers and the like. See, e.g., Remington's Pharmaceutical Sciences, 15th Ed., Easton ed., Mack Publishing Co., pp 1405-1412 and 1461-1487 (1975); The National Formulary XIV, 14th Ed., American Pharmaceutical Association, Washington, D.C. (1975). Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to routine skills in the art.

### Modification of Scaffold Molecules

The scaffold molecules can be readily modified to provide derivative compounds, which can be readily assayed to identify those derivatives that are more potent STING inhibitors. Modifications can be made at suitable steps throughout the synthesis of the scaffold molecules as described in the specific Examples 1-9 below, for example by substituting starting materials and intermediates with a suitable compound, or by modifications of the starting materials, intermediates, and scaffold molecules using suitable organic synthesis methods to provide desired derivatives of the scaffold molecules as described herein. Such methods are readily available to those of skill in the chemical arts, for example, without limitation, methods for modification of the various substituents as described in the following prophetic schemes I-VII. These prophetic schemes are exemplified as modifications of compounds of Formulae I or II in the invention summary, but can also be similarly applied to modification of compounds of Formulae III or IV using suitable starting materials or altering reaction conditions using methods readily available to those skilled in the chemical arts. The Compounds of Formula V can be similarly modified using suitable starting materials readily available to those skilled in the chemical arts (e.g. L-isomers of the nucleotide starting materials as in specific Example 9).

Derivative compounds can be prepared using synthetic methods according to the following reaction schemes, where a skilled chemist can modify conditions as needed by routine methods, such as modification of reaction times, temperature, reagent and reactant concentrations, work-up and purification conditions, and the like, or substitution of suitable solvents and reagents as appropriate. Derivative compounds having modifications in one or more of R1, R2, R3, R4, R5, R6, R7 and R8 (per compounds of Formula I and II above) can be synthesized as described in the following schemes I for modification of R1, R2, R3 or R4 (modified variables as R1′, R2′, R3′ or R4′ defined below) and II for modification of R5, R6, R7 or R8 (modified variables as R5′, R6′, R7′ or R8′ defined below):

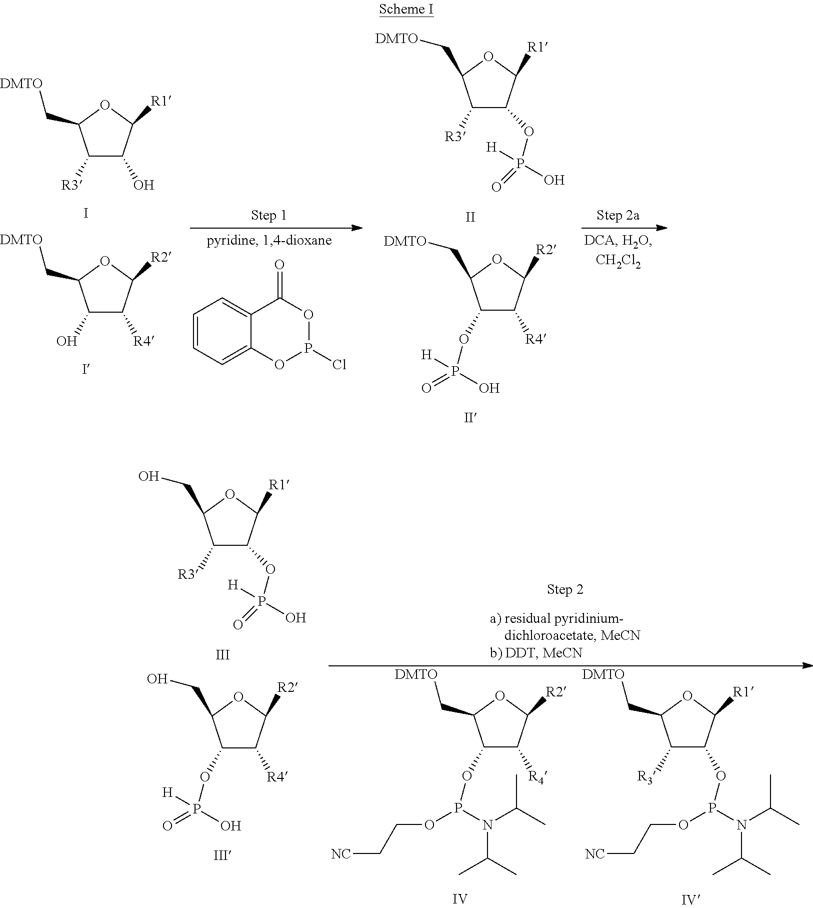

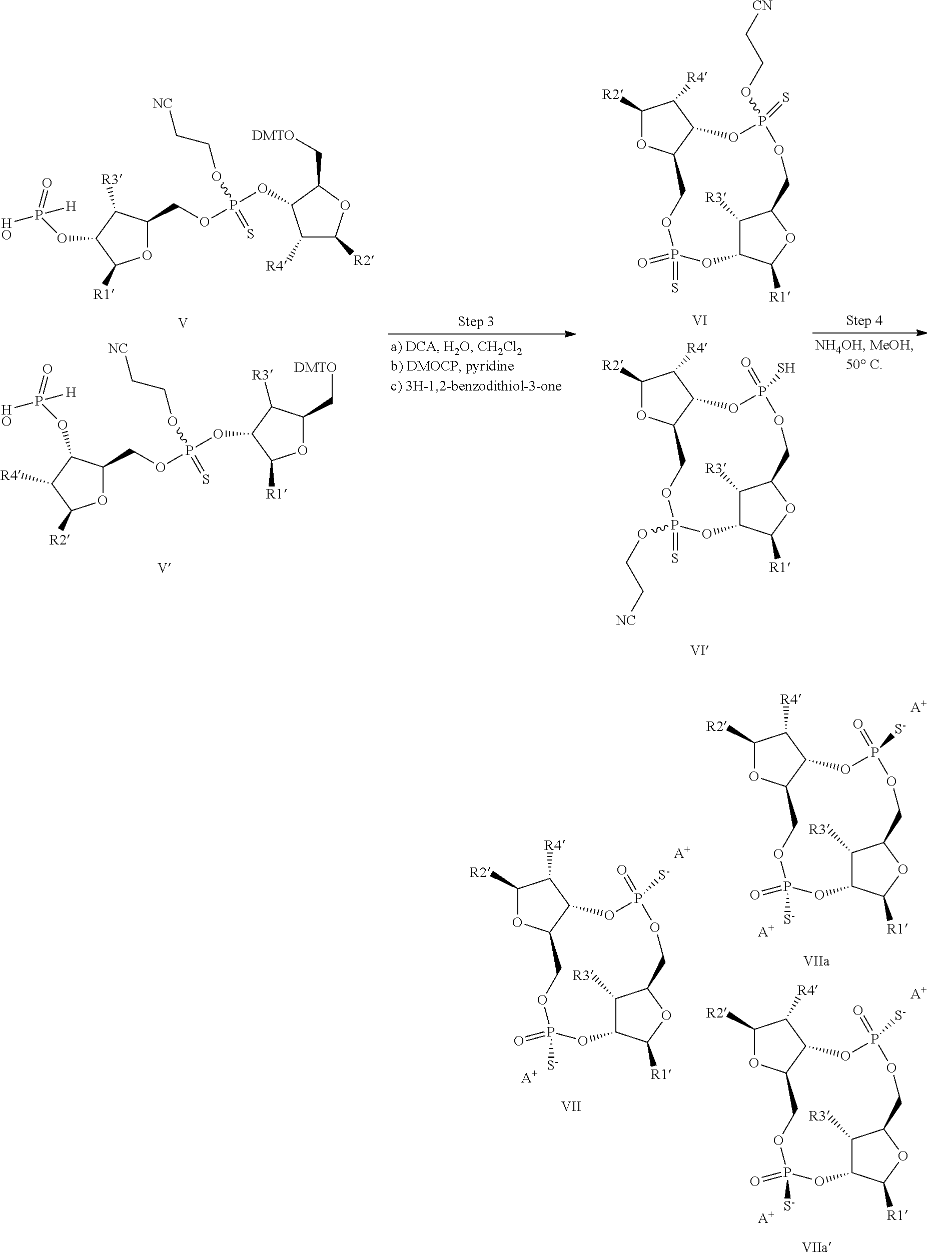

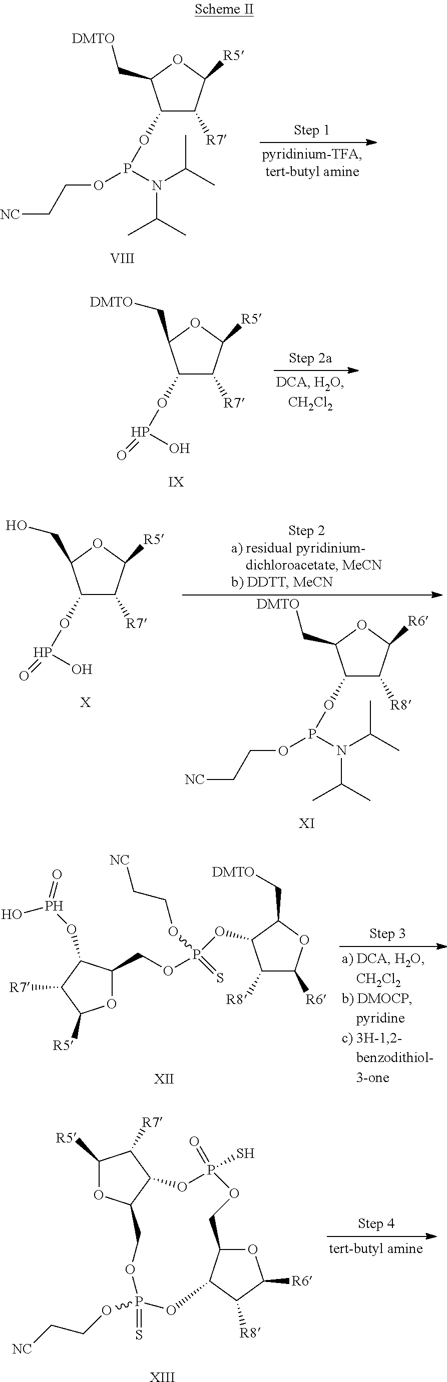

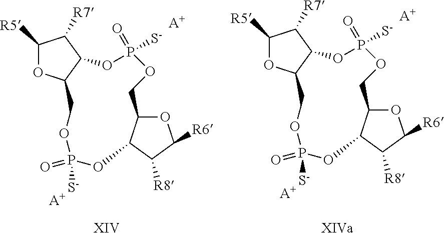

Scheme I, Step 1: Compound II: To a solution of compound I or I′ (1 equiv) in at 0.13 M in dioxane is added pyridine (26 equiv) followed by 2-chloro-4H-1,3,2-benzodioxaphoshorin-4-one (1.2 equiv). The reaction mixture is stirred for 30 min, then quenched with 1 volume of water followed by 8 volumes of 0.4 M aqueous NaHCO₃. This is extracted with EtOAc (3×1 Vol) and DCM (1×1 Vol), and the combined organic layers are dried with Na₂SO₄, filtered and concentrated in vacuo to give compound II or II′, which can be further purified and isolated by standard methods, such as chromatography.

Scheme I, Steps 2a-4: Compounds II, II′, III, III′ and V, V′ can be reacted similarly to the methods of Example 6, Steps 2a, 2 and 3, respectively. The deprotection of compound VI, VI′ can be done according to e.g. Example 7, Step 4, or alternative methods of deprotection, such as used in Scheme 11 (e.g. per Example 6 Step 4). Deprotection of Step 4 can be modified by the skilled chemist to selectively remove some protecting groups and not others to provide derivative compounds of the invention.

Scheme II, Steps 1-4: Compounds VIII, IX, XII, and XIII can be reacted similarly to the methods of Example 6, Steps 1, 2a, 2, 3 and 4, respectively, to provide compounds XIV and XIVa. The deprotection of compound XIII can alternatively be done according to e.g. Example 7, Step 4, or can be modified by the skilled chemist to selectively remove some protecting groups and not others to provide derivative compounds of the invention.

For the compounds described in Scheme I, R1′ is a suitable purine or modified purine, including, but not limited to, a purine selected from the group consisting of adenin-9-yl, guanin-9-yl, hypoxanthin-9-yl, xanthin-9-yl, isoguanin-9-yl, and 2,6-diamino-purin-9-yl, wherein the 6-amino of adenine or isoguanine, 2-amino of guanine, or either or both of the 2- and 6-amino of 2,6-diamino-purine are suitably protected, or these amino groups are optionally substituted with e.g. mono- or di-alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position oxo of guanine or hypoxanthine, the 2-position oxo of isoguanine, or either or both of the 2- and 6-position oxo of xanthine is optionally replaced by —OR^{x}, where R^{x} is alkyl, alkenyl or alkynyl; or R1′ is a suitable pyrimidine or modified pyrimidine, including, but not limited to, a pyrimidine selected from the group consisting of cytosin-1-yl, thymin-1-yl, and uracil-1-yl, wherein the 4-amino of cytosine can be suitably protected, or the 4-amino is optionally substituted with e.g. mono- or di-alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position oxo of cytosine and either or both of the 2- or 4-position oxo of thymine or uracil is optionally replaced by —OR^{x}, where R^{x} is alkyl, alkenyl or alkynyl; R2′ is a suitable purine or modified purine, including, but not limited to, a purine selected from the group consisting of adenin-9-yl, guanin-9-yl, hypoxanthin-9-yl, xanthin-9-yl, isoguanin-9-yl, and 2,6-diamino-purin-9-yl, wherein the 6-amino of adenine or isoguanine, 2-amino of guanine, or either or both of the 2- and 6-amino of 2,6-diamino-purine are suitably protected, or these amino groups are optionally substituted with e.g. mono- or di-alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position oxo of guanine or hypoxanthine, the 2-position oxo of isoguanine, or either or both of the 2- and 6-position oxo of xanthine is optionally replaced by —OR^{x}, where R^{x} is alkyl, alkenyl or alkynyl; or R2′ is a suitable pyrimidine or modified pyrimidine, including, but not limited to, a pyrimidine selected from the group consisting of cytosin-1-yl, thymin-1-yl, and uracil-1-yl, wherein the 4-amino of cytosine can be suitably protected, or the 4-amino is optionally substituted with e.g. mono- or di-alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position oxo of cytosine and either or both of the 2- or 4-position oxo of thymine or uracil is optionally replaced by —OR^{x}, where R^{x} is alkyl, alkenyl or alkynyl; R3′ is selected from the group consisting of H, —OH, —OTBS, halogen, —CN, and —OR wherein R is alkyl, alkenyl, alkynyl; and R4′ is selected from the group consisting of H, —OH, —OTBS, halogen, —CN, and —OR wherein R is alkyl, alkenyl, alkynyl, such that compounds VII, VIIa and VIIa′ are derivatives of the scaffold molecule of Formula I.

For the compounds described in Scheme II, R5′ is a suitable purine or modified purine, including, but not limited to, a purine selected from the group consisting of adenin-9-yl, guanin-9-yl, hypoxanthin-9-yl, xanthin-9-yl, isoguanin-9-yl, and 2,6-diamino-purin-9-yl, wherein the 6-amino of adenine or isoguanine, 2-amino of guanine, or either or both of the 2- and 6-amino of 2,6-diamino-purine are suitably protected, or these amino groups are optionally substituted with e.g. mono- or di-alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position oxo of guanine or hypoxanthine, the 2-position oxo of isoguanine, or either or both of the 2- and 6-position oxo of xanthine is optionally replaced by —OR^{x}, where R^{x} is alkyl, alkenyl or alkynyl; or R5′ is a suitable pyrimidine or modified pyrimidine, including, but not limited to, a pyrimidine selected from the group consisting of cytosin-1-yl, thymin-1-yl, and uracil-1-yl, wherein the 4-amino of cytosine can be suitably protected, or the 4-amino is optionally substituted with e.g. mono- or di-alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position oxo of cytosine and either or both of the 2- or 4-position oxo of thymine or uracil is optionally replaced by —OR^{x}, where R^{x} is alkyl, alkenyl or alkynyl; R6′ is a suitable purine or modified purine, including, but not limited to, a purine selected from the group consisting of adenin-9-yl, guanin-9-yl, hypoxanthin-9-yl, xanthin-9-yl, isoguanin-9-yl, and 2,6-diamino-purin-9-yl, wherein the 6-amino of adenine or isoguanine, 2-amino of guanine, or either or both of the 2- and 6-amino of 2,6-diamino-purine are suitably protected, or these amino groups are optionally substituted with e.g. mono- or di-alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position oxo of guanine or hypoxanthine, the 2-position oxo of isoguanine, or either or both of the 2- and 6-position oxo of xanthine is optionally replaced by —OR^{x}, where R^{x} is alkyl, alkenyl or alkynyl; or R6′ is a suitable pyrimidine or modified pyrimidine, including, but not limited to, a pyrimidine selected from the group consisting of cytosin-1-yl, thymin-1-yl, and uracil-1-yl, wherein the 4-amino of cytosine can be suitably protected, or the 4-amino is optionally substituted with e.g. mono- or di-alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position oxo of cytosine and either or both of the 2- or 4-position oxo of thymine or uracil is optionally replaced by —OR^{x}, where R^{x} is alkyl, alkenyl or alkynyl; R7′ is selected from the group consisting of H, —OH, —OTBS, halogen, —CN, and —OR wherein R is alkyl, alkenyl, alkynyl; and R8′ is selected from the group consisting of H, —OH, —OTBS, halogen, —CN, and —OR wherein R is alkyl, alkenyl, alkynyl, such that compounds XIV and XIVa are derivatives of the scaffold molecule of Formula I.

For the reactions of Schemes I and II, the suitable purine base or modified purine base, or the suitable pyrimidine base or modified pyrimidine base as described above for R1′, R2′, R5′ and R6′ can be readily incorporated into starting materials for these reactions. As an example, without limitation, these modified base starting materials can be readily prepared (see, e.g. Ugarkar et al., J. Med. Chem. 2003, 46:4750-4760; Yu, W. et al., Nature Communications 2012, 3:1288; DOI: 10. 1038/ncomms2304) according to the following Scheme III:

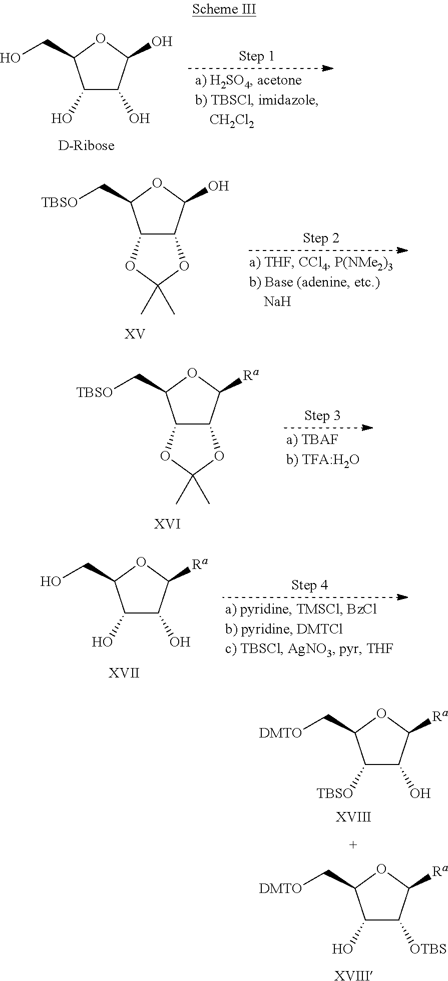

Step 1: (3aR,4R,6R,6aR)-6-(((tert-butyldimethylsilyl)oxy)methyl)-2,2-dimethyltetrahydrofuro[3,4-d][1,3]dioxol-4-ol (XV): D-Ribose (1 equiv) is dissolved to 0.5 M in acetone with addition of sulfuric acid (0.1 equiv) with stirring at room temperature for 3 h to protect the 2′ and 3′ —OH groups. The acid is neutralized with addition of solid NaHCO₃, filtered and the solvent removed in vacuo. The resulting residue is reacted at 0.5 M in CH₂Cl₂ with imidazole (2.4 equiv) and tert-butylchlorodimethylsilane (1.2 equiv). The reaction is allowed to stir at room temperature for 14 h. After quenching with water, the mixture is purified by silica gel chromatography using hexanes:EtOAc (9:1) to provide compound XV.

Step 2: Compound (XVI): Compound XV dissolved to 0.25M in THF and CCl₄ (1.5 equiv) at −70° C. Hexamethylphosphorous triamide (P(NMe₂)₃, 1.1 eq) was added dropwise slowly with stirring at −40° C. for 45 min, then 0° C. for 5 min, and kept as a 0.25M theoretical solution of the intermediate chloride, followed by addition of a suitably protected or modified base (1.6 eq) (R³H, wherein R^{a} is R1′, R2′, R5′ or R6′) and sodium hydride (1.6 equiv) in DMF (0.5 M R^{a}H), with stirring for 22 h at room temperature to provide compound XVI, which can be further purified and/or isolated by standard methods, for example by chromatography.

Step 3: Compound (XVII): Compound XVI is reacted with tetrabutylammonium fluoride (TBAF, 1.2 equiv) and after 30 min at room temperature, is quenched with water. After extraction, the residue is purified by silica gel chromatography. The purified residue is mixed with TFA:water (9:1) (0.5 equiv of TFA) with stirring for 1 h at room temperature. The desired compound XVII is purified and/or isolated by standard methods, such as extraction and chromatography.

Step 4: Compounds (XVIII) and (XVIII′): Compound XVII is reacted at 0.2 M with pyridine, chlorotrimethylsilane (5 equiv) and benzoyl chloride (5 equiv) with stirring for 3 h at room temperature. The solvent is quenched with water at 0° C. and ammonium hydroxide. After extracting with EtOAc and drying, pyridine is added to the resulting residue to 0.5 M along with dimethoxytritylchloride (1.05 equiv) and stirred for 16 h at room temperature. The solvent is removed in vacuo and THE added to 0.1 M, along with tert-butylchlorodimethylsilane (1.3 equiv), pyridine (3.7 equiv) and silver nitrate (1.2 equiv), and is reacted with stirring for 3 h at room temperature. See Hakimelahi, et al., Tetrahedron Letters, 1981, 22(48): 4775-4778. The desired compounds XVIII and XVIII′ are isolated and purified by standard methods, such as extraction and chromatography.

Compounds XVIII and XVIII′ can be used in the reaction of Scheme I Step 1, or further modified to provide materials for use in Scheme I or II above. For example, Compound XVIII can be Compound I of Scheme I, where R^{a} is R1′ and R3′ is —OTBS or —OH (where Step 4 can include TEA.3HF treatment to remove the TBS protecting group). Similarly, Compound XVIII′ can be reacted according to the method of Scheme I, Step 1 to provide a Compound IX, wherein R^{a} is R5′ and R7′ is —OTBS or —OH (where Step 4 can include TEA.3HF treatment to remove the TBS protecting group, see e.g. specific Examples 2 and 4).

Compounds XVIII′ can also be reacted to form the phosphoramidite XIX that can be used in Schemes I and II, for example by the method of Scheme IV:

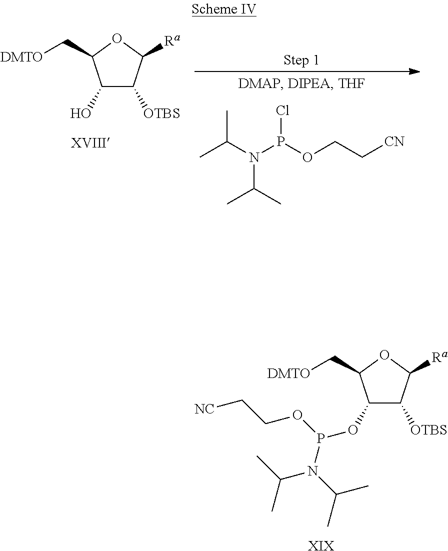

Step 1: Compound XIX: Compound XVIII′ (1 equiv, 0.28M) in THF is reacted with 4-dimethylaminopyridine (DMAP, 0.1 equiv) and diisopropylethylamine (DIPEA, 4 equiv) and 2-Cyanoethyl N,N-diisopropylchlorophosphoramidite (1.1 equiv) is slowly added and the reaction is run for 16 h. The reaction is extracted with EtOAc, washed with brine, and the combined organic layers are combined and dried, and concentrated in vacuo to provide Compound XIX, which is isolated and purified by standard methods, such as chromatography. Compound XIX can be Compound IV of Scheme I, wherein R^{a} is R2′ and R4′ is —OTBS or —OH; or it can be Compound XI of Scheme II, wherein R^{a} is R6′ and R8′ is —OTBS or —OH (where Step 4 can include TEA.3HF treatment to remove the TBS protecting group, see specific Examples 2 and 4).

Additional derivative compounds can be prepared with additional modifications of R3, R4, R7 and R8 positions (as R3′, R4′, R7′ and R8′) using the compounds of Scheme III, or commercially available compounds. Compounds XX and XX′, where R^{b}=R1′, R2′, R5′, R6′, e.g. R^{a} in Scheme III, or as in Examples 1-6, or as commercially available starting materials, can be modified according to Scheme V to provide compounds XXII and XXII′.

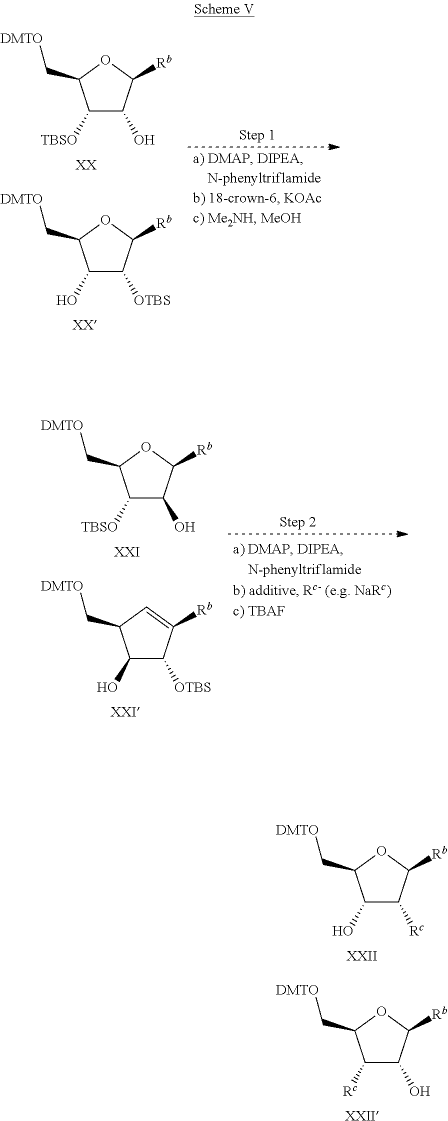

Step 1: Compound XXI or XXI′: Compound XX or XX′ is mixed with DMAP (0.2 equiv) and DIPEA (5 equiv) in THF to provide 0.2 M compound and N-phenyltriflamide (2 equiv) is added. After 4 h at room temperature, the mixture is washed with brine and extracted with EtOAc. After drying, the crude residue is purified on silica gel chromatography (with heptane and EtOAc). The desired intermediate is combined with potassium acetate (5 equiv) and 18-crown-6 (1,4,7,10,13,16-hexaoxacyclooctadecane, 0.5 equiv) in toluene to provide 0.2 M intermediate and the resulting mixture is heated to 110° C. for 4 h. After cooling to room temperature, the mixture is concentrated with silica gel and purified on silica gel chromatography (with heptane and EtOAc). The resulting purified intermediate is dissolved to 0.1 M in methanol and a 2.0 M dimethylamine solution in methanol (5 equiv) is introduced. After 17 h at room temperature, the mixture is combined with silica gel and concentrated in vacuo. The desired compound XXI or XXI′ is isolated and purified by standard methods, such as extraction and chromatography.

Step 2: Compound XXII or XXII′: Compound XXI or XXI′ is mixed with DMAP (0.2 equiv) and DIPEA (5 equiv) in TI-IF to provide 0.2 M compound and N-phenyltriflamide (2 equiv) is added. After 4 h at room temperature, the mixture is washed with brine and extracted with EtOAc. After drying, the crude residue is purified on silica gel chromatography (with heptane and EtOAc). The desired intermediate is combined with the corresponding nucleophile. The nucleophile is selected to provide R^{c} as e.g. —CN, halogen (—F, —Cl, —Br, —I), —OR wherein R is alkyl or alkenyl. The nucleophile (5 equiv) and additive (e.g. 18-crown-6 or 1,4,7,10,13,16-hexaoxacyclooctadecane, DMAP, AgNO₃, etc.) are combined with the intermediate in DMF or appropriate solvent to provide 0.2 M intermediate. After the reaction is complete, the mixture is neutralized with water, extracted with organic solvent, concentrated in vacuo, and purified (e.g. by chromatography). The resulting intermediate is mixed with TBAF (1.2 equiv) in THF to provide 0.1 M intermediate and after 30 min at room temperature, it is quenched with water. After extraction, the desired compound XXII or XXII′ is isolated and purified by standard methods, such as chromatography. Reaction of nucleophiles in Step 2 include, without limitation, reaction with KCN in THF with 18-crown-6 to provide compounds where R^{c} is —CN (see PCT Publication No. WO 2011/144353 page 15, Example 1, the disclosure of which is incorporated herein as it relates to the KCN reaction); compounds where R^{c} is —OR wherein R is alkyl or alkenyl, e.g. by reaction of X-I, where X is a suitable alkyl or —CH₂-alkenyl group (see Hodge et al., Tetrahedron Letters 1995, 36(17):2933-6, the disclosure of which is incorporated herein as it relates to the reaction with X-I) or for R^{c} as —OR, wherein R is alkenyl, reaction with e.g. allyl bromide and NaH in DMF (Prakash et al., J. Org. Chem. 2002, 67:357-369, the disclosure of which is incorporated herein as it relates to the allyl bromide reaction) or with allyl ethyl carbonate, Pd₂(dpa)₂ and dppb in THF (Odadzic et al., Bioorganic and Medicinal Chemistry 2008, 16:518-529); reaction to provide R^{c} as halogen, e.g. Cl, as described in Anderson et al., Organic Process Research & Development 2008, 12:1229-1237 the disclosure of which is incorporated herein as it relates to the chlorine substitution. Compound XXI or XXI′ could also be reacted, e.g. 0.14 M in CH₂Cl₂ with 1 M DAST at −5° C. for 17 h, and purified by extraction and chromatography, to provide the fluoro substituent. Fluoro substitution using DAST is also exemplified in e.g. Wilson et al., Journal of Fluorine Chemistry, 1991, 55:283-289 the disclosure of which is incorporated herein as it relates to the fluorine substitution.

The compounds XXII or XXII′ can be used directly in Scheme I, i.e. can be the compound I or I′ where R^{b} is R1′ and R^{c} is R3′. Alternatively, compounds XXII or XXII′ can be further modified according to the method of Scheme IV to convert the 2′ or 3′ OH to a phosphoramidite XXIII or XXIII′ for use in the methods of Scheme I and II, as shown in the following Scheme VI.

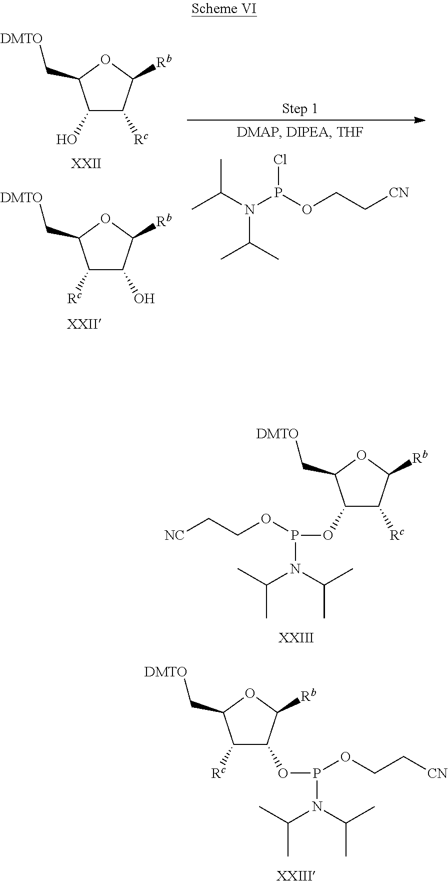

Thus, compound XXIII′ can be compound IV′ of Scheme I where R^{b} is R2′ and R^{c} is R4′. Similarly, compound XXIII can be compound IV of Scheme I where R^{b} is R2′ and R^{c} is R4′ compound VIII of Scheme II where R^{b} is R5′ and R^{c} is R7′, or compound XI of Scheme II where R^{b} is R6′ and R^{c} is R8′. Compounds of Formula XXIII or XXIII′ wherein R^{c} is —OCH₂C≡CH and R^{b} is benzoyl protected adenine or cytosine, isobutyryl protected guanine, or uracil and of Formula XXIII wherein Rc is —OCH₂C≡CCH₃ are commercially available, e.g. from ChemGenes.

The compounds XVIII and XVIII′ can also be modified to be used in Schemes I or II to provide compounds where R3′, R4′, R7′ or R8′ is —H, according to the following Scheme VII.

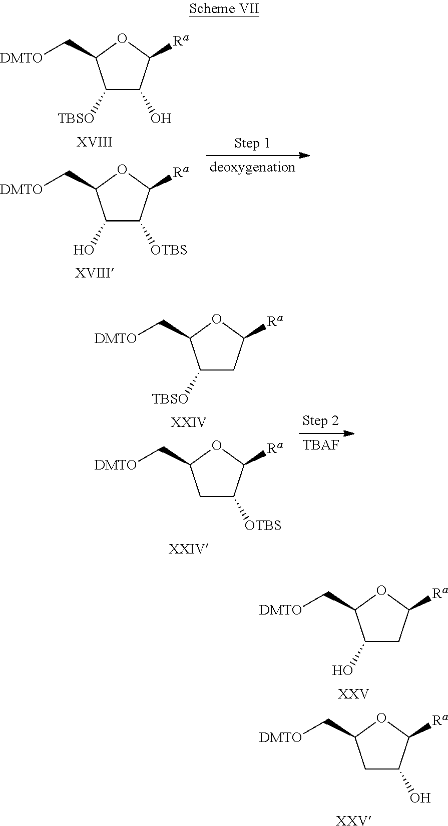

The deoxygenation of XVIII or XVIII′ in Step 1 to provide compounds XXIV or XXIV′ can be done using Barton-McCombie deoxygenation (Barton et al., J Chem Soc. Perkin Trans (1975) 16: 1574-1585, the disclosure of which is incorporated herein as it relates to this reaction), or reacting triphenylphosphine (PPh₃), diethylazodicarboxylate (DEAD) and compound in THF at −30° C. for 20 min, followed by the addition of o-nitrobenzenesulfinic acid (NBSH) in THF at −30° C. for 2 h, then warming to 23° C., as described in Myers et al., J. Am. Chem Soc. 1997, 119:8572-8573 the disclosure of which is incorporated herein as it relates to this reaction. The removal of the TBSproteceting group from XXIV or XXIV′ in step 2 is done by treating under suitable conditions with TBAF, as in step 2c of Scheme V. The resulting compound XXV can be compound I′ or compound XXV′ can be compound I in Scheme I where R^{a} is R1′ and R3′ is H, or XXV or XXV′ can be reacted similarly to Scheme IV to make the phosphoramidite for use in Scheme I where R7 or R8 is H, e.g. as compounds IV, IV′ or XI.

The compound XVII (see Scheme III) can be modified to provide compounds for use in Schemes I or II, wherein R3′, R4′, R7′ or R8′ is an —O-propargyl group, according to the following Scheme VIII.

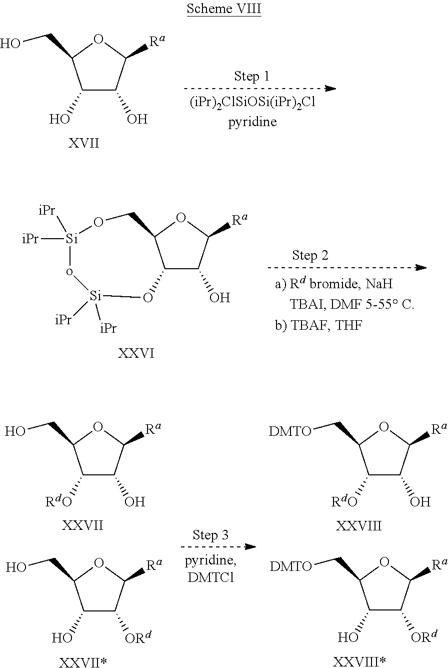

Compound XVII can be reacted in Steps 1 and 2 (e.g. R^{d}=propargyl, Pujari et al., The Journal of Organic Chemistry, the disclosure of which is incorporated herein as it relates to this reaction) to provide the —O-propargyl group in XXVII and XXVII′, followed by protection of the 5′—OH with a dimethoxytrityl group by standard methods as described herein, to provide compounds XXVIII and XXVIII′. Compounds XXVIII or XXVIII′ can be compound I or I′ of Scheme I, or can be further reacted according to Scheme VI to provide compounds that can be used to replace compound IV or IV′ of Scheme I, or to replace compound VIII or compound XI of Scheme II.

Additional compounds to be used in Schemes I or II are commercially available, or can be readily prepared from commercially available compounds. For example, modified bases for use in Scheme III are available, or can be prepared according to the following Schemes.

Modification of Adenine for use in Scheme III is exemplified in the following Schemes IX, X and XI.

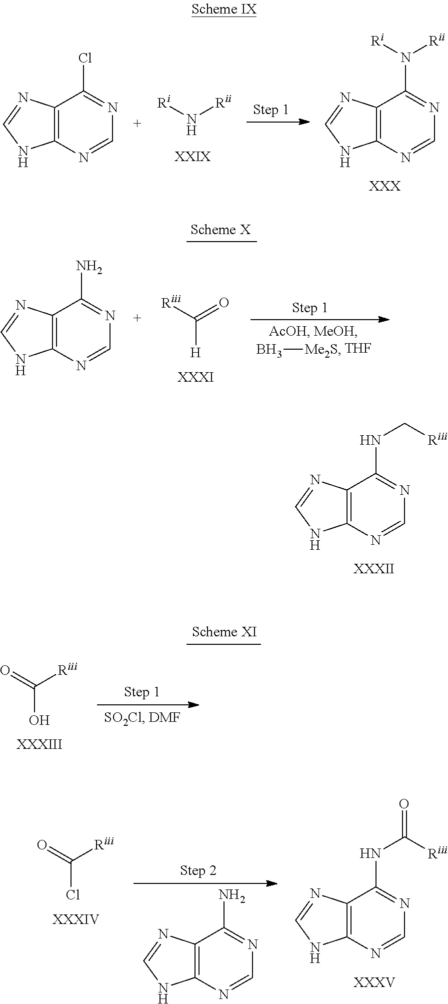

In Scheme IX, commercially available 6-chloro-9H-purine is reacted with a commercially available di-alkylamine (XXIX, where Rⁱ and Rⁱⁱ are independently alkyl) to provide the 6-di-alkylamino-9H-purine XXX In Scheme X, adenine is reacted with a commercially available aldehyde (XXXI, where Rⁱⁱⁱ is independently alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy) in THF with AcOH, MeOH, and BH₃-Me₂S to provide the derivative XXXII (see, e.g., Adamska et al., Nucleosides, Nucleotides & Nucleic Acids, 2012, 31(12):861-871, the disclosure of which is incorporated herein as it relates to this reaction). In Scheme XI, adenine is reacted with XXXIV (prepared from the reaction of commercially available acid XXXIII with SO₂Cl in DMF, each Rⁱⁱⁱ is independently alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy) to provide the 6-acylamino derivative XXXV. The compounds XXX, XXXII and XXXV can be used in Scheme III (as R^{a}H in Step 2). Commercially available adenine derivatives, such as 6-methylamino-, 6-dimethylamino- and 6-ethylamino-9H-purine, can be similarly used in Scheme III. Cytosine can be used in place of adenine in Scheme IX to provide the 4-alkylamino analogs of cytosine for use in Scheme III.

Modification of Guanine for use in Scheme III is exemplified in the following Schemes XII and XIII.

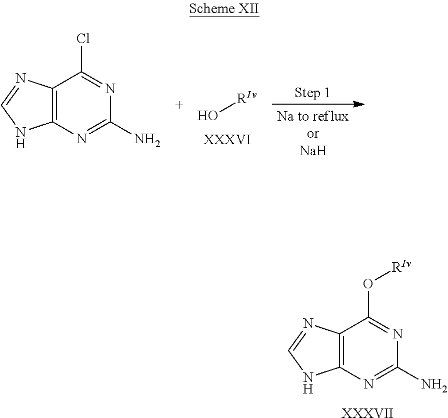

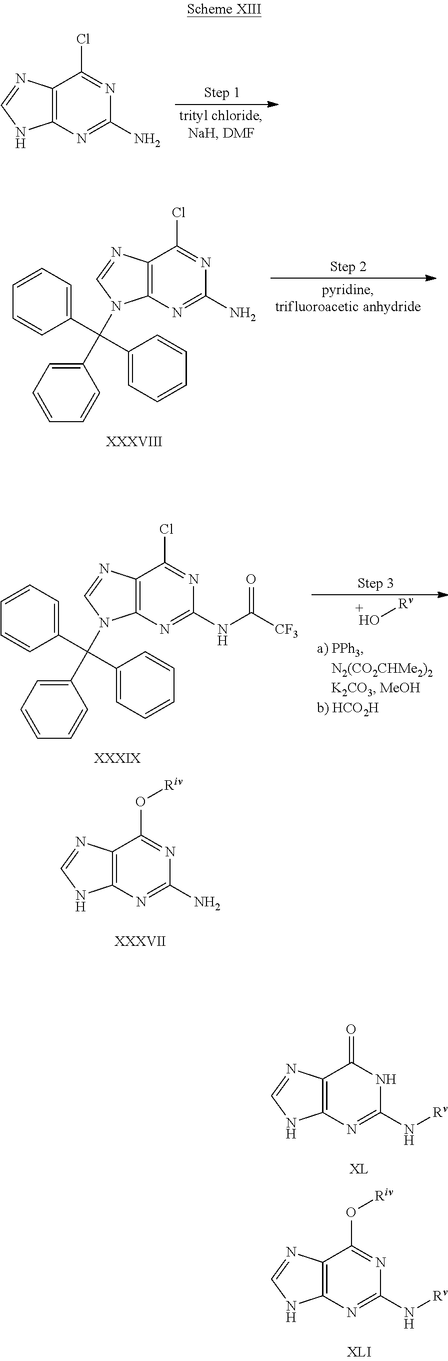

In Scheme XII, commercially available 6-chloro-9H-purin-2-amine is reacted in one step with alcohol XXXVI (R^{iv} is e.g. alkyl, alkenyl or alkynyl) and sodium to reflux, or alternatively with NaH (see Griffin et al., Journal of Medicinal Chemistry, 43(22):4071-4083, the disclosure of which is incorporated herein as it relates to this reaction) to provide compound XXXVII. In Scheme XIII, 6-chloro-9H-purin-2-amine is reacted with trityl chloride and NaH in DMF in Step 1 to provide compound XXXVIII, which is reacted in Step 2 with trifluoroacetic anhydride in pyridine to provide compound XXXIX. In Step 3, compound XXXIX is reacted with commercially available R^{v}OH (R^{v} is e.g. alkyl, alkenyl, alkynyl (e.g. propargyl), or other available alcohol to provide the 2-amine substituted with alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy) in MeOH with PPh₃, N₂(CO₂CHMe₂)₂ and K₂CO₃, followed by formic acid to provide compound XL (see Tetrahedron, 2010, 66(25), 4621-4632, the disclosure of which is incorporated herein as it relates to this reaction). Compound XXXVII from Scheme XII can be similarly reacted to provide compound XLI. Guanine can also be reacted according to the method of Scheme XI, reacting compound XXXIV with guanine in place of adenine to provide the 2-acylamino analogs (i.e. —NHC(O)Rⁱⁱⁱ where each Rⁱⁱⁱ is independently alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy). This guanine derivative or compound XL or XLI can be used in Scheme III (as R^{a}H in Step 2). Commercially available guanine derivatives, such as having the 2-amine substituted as methylamino, dimethylamino, or —N—C(O)t-butyl, can be similarly used in Scheme III.

Modification of 2,6-diamino-purine for use in Scheme III is exemplified in the following Scheme XIV.

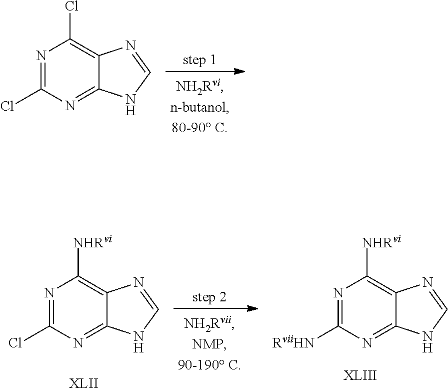

In Scheme XIV, commercially available 2,6-dichloropurine is heated with commercially available amine R^{vi}NH₂ (R^{vi} is alkyl, alkenyl, phenyl, etc.) in n-butanol between 80° C. and 90° C. in Step 1 to provide compound XLII, which is isolated through concentration, filtration, or trituration. Compound XLII is combined with a second commercially available amine R^{vii}NH₂ (R^{vii} is alkyl, alkenyl, phenyl, etc., can be the same or different than R^{vi}) and heated in a higher boiling solvent (NMP, DMF) in Step 2 to give compound XLIII. Compound XLII could also be heated in a microwave reactor at a higher temperature for Step 2 in the appropriate solvent. Multiple examples for these steps are found in the literature (see Busca, et. al. Eur. J. Org. Chem 2006, 2403-2409; Vincetti, et al. J. Med. Chem. 2015, 58, 4964-4975, the disclosures of which are incorporated by reference as they relate to these reactions). 2,6-Dichloropurine could alternatively be combined with the corresponding 2-acylamino analogs (i.e. —NHC(O)Rⁱⁱⁱ as described in Scheme XI) and reacted as in Scheme XI to obtain the 2-acylamino and/or 6-acylamino-purine derivatives. This 2,6-diaminopurine derivative or compound XLIII can be used in Scheme III (as R^{a} H in Step 2). Commercially available 2,6-diaminopurine derivatives, such as having the 2-amine or 6-amine substituted as methylamino, dimethylamino, or —NHC(O)t-butyl, can be similarly used in Scheme III.

### EXAMPLES

The following examples serve to illustrate the present invention. These examples are in no way intended to limit the scope of the invention.

### General Methods

Anhydrous solvents and reagents suitable for solution phase oligonucleotide synthesis were purchased from commercial suppliers (Aldrich, ChemGenes Corporation, Wilmington, Mass., USA) and handled under dry argon or nitrogen using anhydrous technique. Phosphoramidite coupling reactions and H-phosphonate cyclizations were carried out in anhydrous acetonitrile or pyridine under dry argon or nitrogen. The starting materials for all reactions in dry pyridine were dried by concentration (three times) from pyridine, unless indicated otherwise. Chromatography conditions were as follows unless indicated otherwise in the example below. Preparative silica gel flash chromatography was carried out under medium pressure chromatography (MPLC) using RediSep Rf silica columns (Teledyne Isco, Lincoln, Nebr.) on a Combiflash Rf+ UV-Vis (Teledyne Isco) using gradients of methanol in dichloromethane. Reverse phase preparative chromatography was executed under MPLC conditions using RediSep Rf C18 Aq columns (Teledyne Isco) on a Combiflash Rf+ UV-Vis using gradients of acetonitrile in aqueous 10 maM TEAA solution. Analytical high pressure liquid chromatography (HPLC) was performed on a Shimadzu Prominence HPLC with a photodiode array detector monitoring at 254 nm using a either a Microsorb 10 micron C18 250×4.6 mm or a Thermo Scientific Acclaim™ 120 5 μm C18 100×4.6 mm column and gradients of 10 mM TEAA and acetonitrile. Preparative HPLC was carried out on a Shimadzu preparative LC20-AP HPLC system, equipped with a SPD-20A UV/Vis detector monitoring at 254 nm on a Varian Microsorb 60-8 C-18 41.6×250 mm column using gradients of 10 mM TEAA and acetonitrile at a flow rate of 50 ml/min. Solid phase extractions using C-18 Sep-Pak (Waters) were carried out at loadings of 3% (wt/wt). For the compounds of Examples 2-8, Analytical LCMS were recorded using a Shimadzu LCMS system featuring a Prominence HPLC coupled to a Shimadzu LCMS-2020 single quadrupole mass spectrometer, using an electrospray ionization source (ESI).

The final scaffold molecules may exist as the TEA salt, with conversion to other salt forms (including but not limited to Na and NH₄) using standard ion exchange techniques or other well known methods is possible.

Assignments of Stereochemistry at the phosphorus were made in analogy to literature methods (Zhao et al. Nucleosides, Nucleotides, and Nucleic Acid 289:352-378, 2009) or as discussed in the examples below.

Compound names were generated using the software program ChemBioDraw Ultra V 14.0 available from CambridgeSoft Corporation, 100 CambridgePark Drive, Cambridge, Mass. 02140 USA (http://www.cambridgesoft.com). Abridged names of compounds, including those for which a name could not be generated by ChemBioDraw, are provided in the following Table 1. Structures in the examples may also be represented as salts, e.g. —S⁻ A⁺, where A⁺ is the salt cation.

TABLE 1
Scaffold molecule names, including abridged names, and structures.

| Example number and Compound names | Structure |
|---|---|
| Example 1 Compound 6 2′3′-RR-(3′OTBS-A)(2′F-A); dithio-[R_{P}, R_{P}]-cyclic- [3′OTBS-A(2′,5′)p-2′F-A(3′,5′)p] | 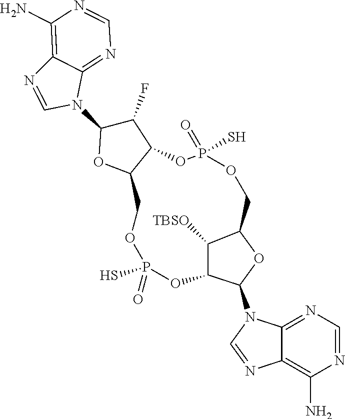 [SMILES: CC(C)(C)[Si](C)(C)O[C@H]1[C@H]2O[P@](=O)(S)OC[C@H]3O[C@@H](n4cnc5c(N)ncnc54)[C@H](F)[C@@H]3O[P@](=O)(S)OC[C@H]1O[C@H]2n1cnc2c(N)ncnc21] |
| Example 1 Compound 6a 2′3′-SR-(3′OTBS-A)(2′F-A); dithio-[S_{P}, R_{P}]-cyclic- [3′OTBS-A(2′,5′)p-2′F-A(3′,5′)p] | 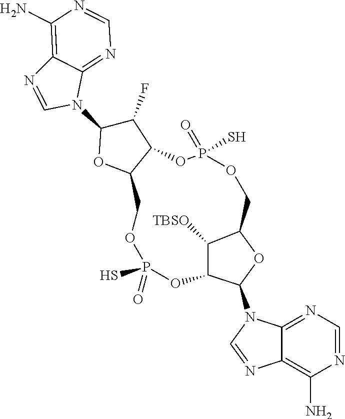 [SMILES: CC(C)(C)[Si](C)(C)O[C@H]1[C@H]2O[P@@](=O)(S)OC[C@H]3O[C@@H](n4cnc5c(N)ncnc54)[C@H](F)[C@@H]3O[P@](=O)(S)OC[C@H]1O[C@H]2n1cnc2c(N)ncnc21] |
| Example 2 Compound 12 2′3′-RR-(A)(2,6-DAP); dithio-[R_{P}, R_{P}]-cyclic- [A(2′,5′)p-2,6-DAP(3′,5′)p] | 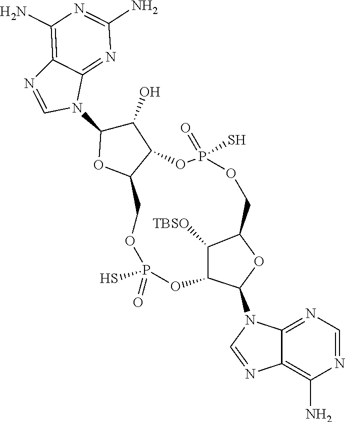 [SMILES: CC(C)(C)[Si](C)(C)O[C@H]1[C@H]2O[P@](=O)(S)OC[C@H]3O[C@@H](n4cnc5c(N)nc(N)nc54)[C@H](O)[C@@H]3O[P@](=O)(S)OC[C@H]1O[C@H]2n1cnc2c(N)ncnc21] |
| Example 2 Compound 12a 2′3′-SR-(A)(2,6-DAP); dithio-[R_{P}, R_{P}]-cyclic- [A(2′,5′)p-2,6-DAP(3′,5′)p] | 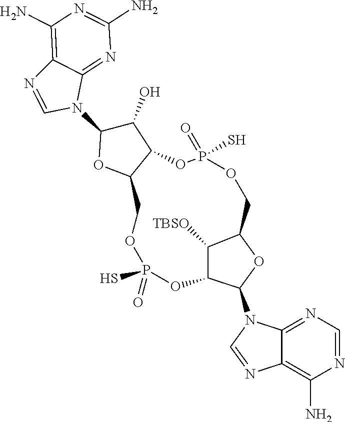 [SMILES: CC(C)(C)[Si](C)(C)O[C@H]1[C@H]2O[P@@](=O)(S)OC[C@H]3O[C@@H](n4cnc5c(N)nc(N)nc54)[C@H](O)[C@@H]3O[P@](=O)(S)OC[C@H]1O[C@H]2n1cnc2c(N)ncnc21] |
| Example 3 Compound 18 RR-(2′F-A)(2′OTBS-A); dithio-[R_{P}, R_{P}]-cyclic- [2′F-A(3′,5′)p-2′OTBS-A(3′,5′)p]; (2R,3R,3aR,5R,7aR,9R,10R,10aR, 12R,14aR)-2,9-bis(6-amino-9H-purin- 9-yl)-3-((tertbutyldimethylsilyl)oxy)- 10-fluoro-5,12-dimercaptooctahydro- 2H,7H-difuro[3,2-d:3′,2′-j][1,3,7,9]tetraoxa [2,8]diphosphacyclododecine 5,12-dioxide | 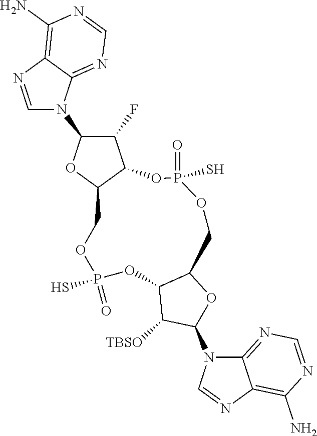 [SMILES: CC(C)(C)[Si](C)(C)O[C@@H]1[C@@H]2O[P@](=O)(S)OC[C@H]3O[C@@H](n4cnc5c(N)ncnc54)[C@H](F)[C@@H]3O[P@](=O)(S)OC[C@H]2O[C@H]1n1cnc2c(N)ncnc21] |
| Example 3 Compound 18a SR-(2′F-A)(2′OTBS-A); dithio-[S_{P}, R_{P}]-cyclic- [2′F-A(3′,5′)p-2′OTBS-A(3′,5′)p]; (2R,3R,3aR,5R,7aR,9R,10R,10aR, 12S,14aR)-2,9-bis(6-amino-9H-purin- 9-yl)-3-((tert-butyldimethylsilyl)oxy)- 10-fluoro-5,12-dimercaptooctahydro- 2H,7H-difuro[3,2-d:3′2′-j][1,3,7,9] tetraoxa[2,8]diphosphacyclododecine 5,12-dioxide | 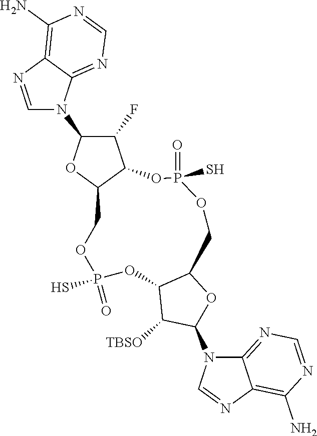 [SMILES: CC(C)(C)[Si](C)(C)O[C@@H]1[C@@H]2O[P@](=O)(S)OC[C@H]3O[C@@H](n4cnc5c(N)ncnc54)[C@H](F)[C@@H]3O[P@@](=O)(S)OC[C@H]2O[C@H]1n1cnc2c(N)ncnc21] |
| Example 4 Compound 23 RR-(2,6-DAP)(2,6-DAP); dithio-[R_{P}, R_{P}]-cyclic- [2,6-DAP(3′,5′)p-2,6-DAP(3′,5′)p]; (2R,3R,3aS,5R,7aR,9R,10R,10aS,12R, 14aR)-2,9-bis(2,6-diamino-9H-purin-9-yl)- 3,10-dihydroxy-5,12-dimercaptooctahydro- 2H,7H-difuro[3,2-d:3′,2′-j][1,3,7,9] tetraoxa[2,8]diphosphacyclododecine 5,12- dioxide | 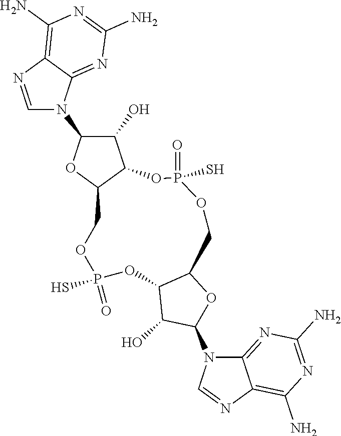 [SMILES: Nc1nc(N)c2ncn([C@@H]3O[C@@H]4CO[P@@](=O)(S)O[C@H]5[C@@H](O)[C@H](n6cnc7c(N)nc(N)nc76)O[C@@H]5CO[P@@](=O)(S)O[C@H]4[C@H]3O)c2n1] |
| Example 4 Compound 23a RS-(2,6-DAP)(2,6-DAP); dithio-[R_{P}, S_{P}]-cyclic-[2,6- DAP(3′5′)p-2,6-DAP(3′,5′)p];(2R,3R, 3aS,5R,7aR,9R,10R,10aS,12S,14aR)- 2,9-bis(2,6-diamino-9H-purin-9-yl)-3,10- dihydroxy-5,12-dimercaptoocytahydro- 2H,7H-difuro[3,2-d:3′2′-j][1,3,7,9] tetraoxa[2,8]diphosphacyclododecine 5,12- dioxide | 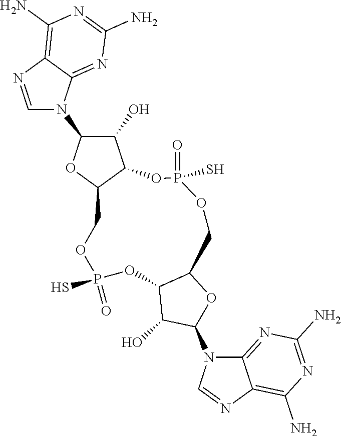 [SMILES: Nc1nc(N)c2ncn([C@@H]3O[C@@H]4CO[P@@](=O)(S)O[C@H]5[C@@H](O)[C@H](n6cnc7c(N)nc(N)nc76)O[C@@H]5CO[P@](=O)(S)O[C@H]4[C@H]3O)c2n1] |
| Example 5 Compound 25 (6-O-propargyl-G)(G); cyclic-[6-O-propargyl-G(3′,5′)p-G(3′,5′)p]; 2-amino-9-((2R,3R,3aS,7aR,9R,10R, 10aS,14aR)-9-(2-amino-6-(prop-2-yn- 1-yloxy)-9H-purin-9-yl)-3,5,10,12- tetrahydroxy-5,12-dioxidooctahydro- 2H,7H-difuro[3,2-d:3′,2′-j][1,3,7,9] tetraoxa[2,8]diphosphacyclododecin- 2-yl)-1,9-dihydro-6H-purin-6-one | 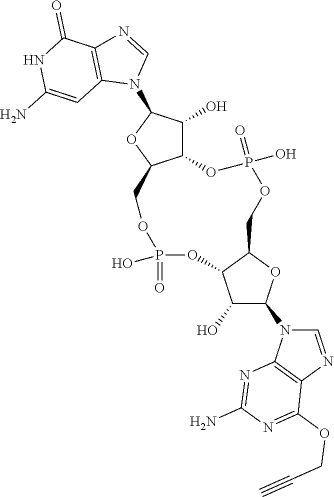 [SMILES: C#CCOc1nc(N)nc2c1ncn2[C@@H]1O[C@@H]2COP(=O)(O)O[C@H]3[C@@H](O)[C@H](n4cnc5c(=O)[nH]c(N)cc54)O[C@@H]3COP(=O)(O)O[C@H]2[C@H]1O] |
| Example 5 Compound 26 (6-O-propargyl-G)(6-O-propargyl-G); cyclic-[6-O-propargyl-G(3′,5′)p-6-O- propargyl-G(3′,5′)p];(2R,3R, 3aS,7aR,9R,10R,10aS,14aR)-2,9-bis(2- amino-6-(prop-2-yn-1-yloxy)-9H-purin-9- yl)-3,5,10,12-tetrahydroxyoctahydro-2H,7H- difuro[3,2-d:3′,2′-j][1,3,7,9]tetraoxa[2,8] diphosphacyclododecine 5,12-dioxide | 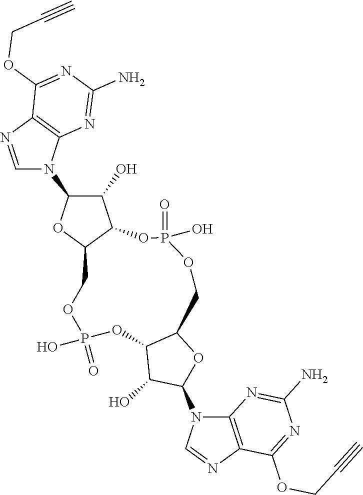 [SMILES: C#CCOc1nc(N)nc2c1ncn2[C@@H]1O[C@@H]2COP(=O)(O)O[C@H]3[C@@H](O)[C@H](n4cnc5c(OCC#C)nc(N)nc54)O[C@@H]3COP(=O)(O)O[C@H]2[C@H]1O] |
| Example 6 Compound 32 RR-(2′F-ibG)(2′F-ibG); dithio-[R_{P}, R_{P}]-cyclic- [2′F-ibG(3′,5′)p-2′F-ibG(3′,5′)p]; N,N′-(((2R,3R,3aR,5R,7aR,9R,10R,10aR, 12R,14aR)-3,10-difluoro-5,12-dimercapto- 5,12-dioxidooctahydro-2H,7H-difuro[3,2-d: 3′2′-j][1,3,7,9]tetraoxa[2,8] diphosphacyclododecine-2,9-diyl)bis(6-oxo- 6,9-dihydro-1H-purine-9,2-diyl))bis(2- methylpropanamide) | 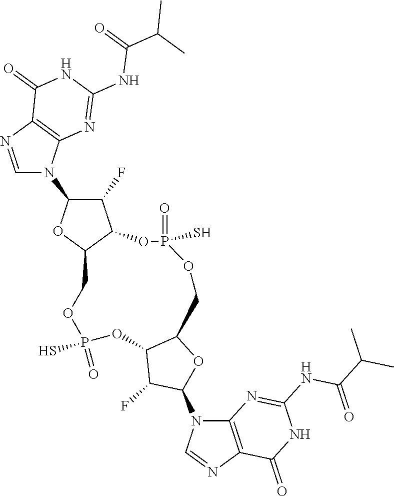 [SMILES: CC(C)C(=O)Nc1nc2c(ncn2[C@@H]2O[C@@H]3CO[P@@](=O)(S)O[C@H]4[C@@H](F)[C@H](n5cnc6c(=O)[nH]c(NC(=O)C(C)C)nc65)O[C@@H]4CO[P@@](=O)(S)O[C@H]3[C@H]2F)c(=O)[nH]1] |
| Example 6 Compound 32a RS-(2′F-ibG)(2′-F-ibG); dithio-[R_{P}, S_{P}]-cyclic- [2′F-ibG(3′,5′)p-2′F-ibG(3′,5′)p]; N,N′-(((2R,3R,3aR,5R,7aR,9R,10R,10aR, 12S,14aR)-3,10-difluoro-5,12-dimercapto- 5,12-dioxidooctahydro-2H,7H-difuro[3,2-d: 3′,2′-j][1,3,7,9]tetraoxa[2,8] diphosphacyclododecine-2,9-diyl)bis(6-oxo- 6,9-dihydro-1H-purine-9,2-diyl))bis(2- methylpropanamide) | 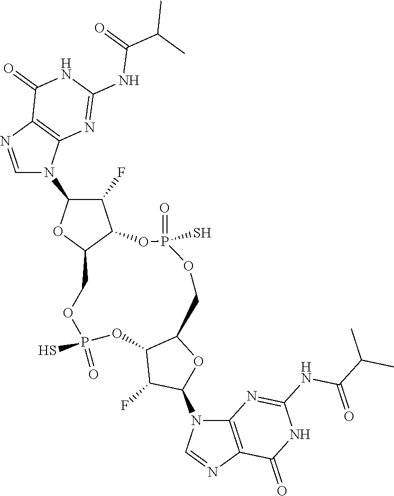 [SMILES: CC(C)C(=O)Nc1nc2c(ncn2[C@@H]2O[C@@H]3CO[P@@](=O)(S)O[C@H]4[C@@H](F)[C@H](n5cnc6c(=O)[nH]c(NC(=O)C(C)C)nc65)O[C@@H]4CO[P@](=O)(S)O[C@H]3[C@H]2F)c(=O)[nH]1] |
| Example 7 Compound 37 RR-(2′F-C)(2′F-A); dithio-[R_{P}, R_{P}]-cyclic- [2′F-C(3′,5′)p-2′F-A(2′,5′)p]; 4-amino-1-((2R,3R,3aR,5R,7aR,9R,10R, 10aR,12R,14aR)-9-(6-amino-9H-purin-9-yl)- 3,10-difluoro-5,12-dimercapto-5,12- dioxidooctahydro-2H,7H-difuro[3,2-d:3′,2′-j] [1,3,7,9]tetraoxa[2,8]diphosphacyclododecin- 2-yl)pyrimidin-2(1H)-one | 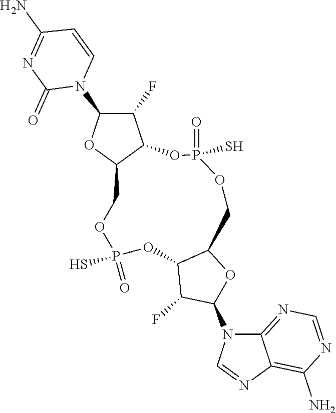 [SMILES: Nc1ccn([C@@H]2O[C@@H]3CO[P@@](=O)(S)O[C@H]4[C@@H](F)[C@H](n5cnc6c(N)ncnc65)O[C@@H]4CO[P@@](=O)(S)O[C@H]3[C@H]2F)c(=O)n1] |
| Example 7 Compound 37a RS-(2′F-C)(2′F-A); dithio-[R_{P}, S_{P}]-cyclic- [2′F-C(3′,5′)p-2′F-A(2′,5′)p]; 4-amino-1-((2R,3R,3aR,5R,7aR,9R,10R, 10aR,12S,14aR)-9-(6-amino-9H-purin-9-yl)- 3,10-difluoro-5,12-dimercapto-5,12- dioxidooctahydro-2H,7H-difuro[3,2-d: 3′,2′-j][1,3,7,9]tetraoxa[2,8] diphosphacyclododecin- 2-yl)pyrimidin-2(1H)-one | 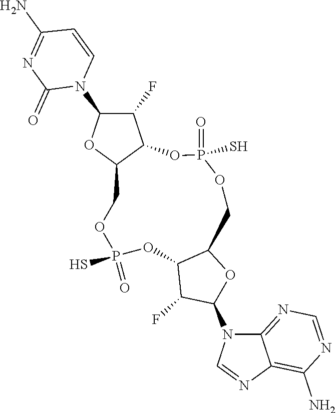 [SMILES: Nc1ccn([C@@H]2O[C@@H]3CO[P@](=O)(S)O[C@H]4[C@@H](F)[C@H](n5cnc6c(N)ncnc65)O[C@@H]4CO[P@@](=O)(S)O[C@H]3[C@H]2F)c(=O)n1] |
| Example 8 Compound 42 3′2′-RR-(ibG)(BzA); dithio-[R_{P}, S_{P}]-cyclic- [ibG(3′,5′)p-BzA(2′,5′)p] | 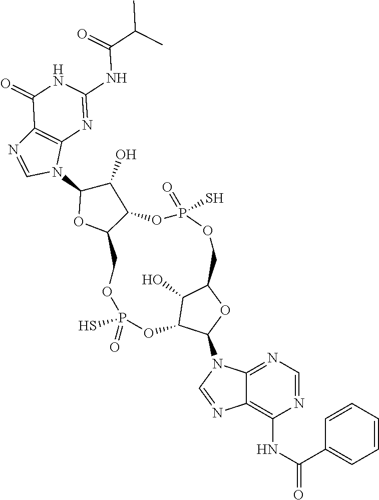 [SMILES: CC(C)C(=O)Nc1nc2c(ncn2[C@@H]2O[C@@H]3CO[P@@](=O)(S)O[C@@H]4[C@H](O)[C@@H](CO[P@@](=O)(S)O[C@H]3[C@H]2O)O[C@H]4n2cnc3c(NC(=O)c4ccccc4)ncnc32)c(=O)[nH]1] |
| Example 8 Compound 42a 3′2′-RS-(ibG)(BzA); dithio-[R_{P}, S_{P}]-cyclic- [ibG(3′,5′)p-BzA(2′,5′)p] | 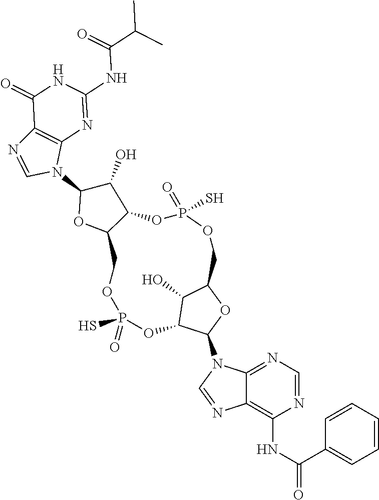 [SMILES: CC(C)C(=O)Nc1nc2c(ncn2[C@@H]2O[C@@H]3CO[P@](=O)(S)O[C@@H]4[C@H](O)[C@@H](CO[P@@](=O)(S)O[C@H]3[C@H]2O)O[C@H]4n2cnc3c(NC(=O)c4ccccc4)ncnc32)c(=O)[nH]1] |
| Example 8 Compound 43 3′2′-SS-(G)(A); cyclic-[G(3′,5′)p-A(2′,5′)p] | 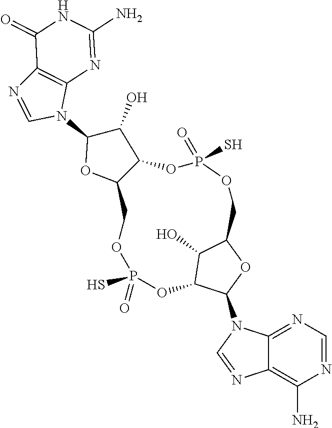 [SMILES: Nc1nc2c(ncn2[C@@H]2O[C@@H]3CO[P@](=O)(S)O[C@@H]4[C@H](O)[C@@H](CO[P@](=O)(S)O[C@H]3[C@H]2O)O[C@H]4n2cnc3c(N)ncnc32)c(=O)[nH]1] |
| Example 9 Compound 48 Beta-L-SS-(A)(A) dithio-[S_{P}, S_{P}]-cyclic- L-A(3′,5′)p-L-A(3′,5′)p] (2S,3S,3aR,5S,7aS,9S,10S,10aR,12S, 14aS)-2,9-bis(6-amino-9H-purin-9-yl)-3,10- dihydroxy-5,12-dimercaptooctahydro-2H,7H- difuro[3,2-d:3′,2′-j][1,3,7,9]tetraoxa[2,8] diphosphacyclododecine 5,12-dioxide | 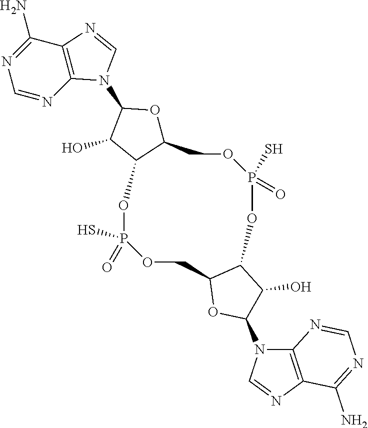 [SMILES: Nc1ncnc2c1ncn2[C@H]1O[C@H]2CO[P@](=O)(S)O[C@@H]3[C@H](O)[C@@H](n4cnc5c(N)ncnc54)O[C@H]3CO[P@](=O)(S)O[C@@H]2[C@@H]1O] |
| Example 9 Compound 48a Beta-L-RS-(A)(A) dithio-[R_{P}, S_{P}]-cyclic- L-A(3′,5′)p-L-A(3′,5′)p] (2S,3S,3aR,5R,7aS,9S,10S,10aR,12S, 14aS)-2,9-bis(6-amino-9H-purin-9-yl)-3,10- dihydroxy-5,12-dimercaptooctahydro-2H,7H- difuro[3,2-d:3′2′-j][1,3,7,9]tetraoxa[2,8] diphosphacyclododecine 5,12-dioxide | 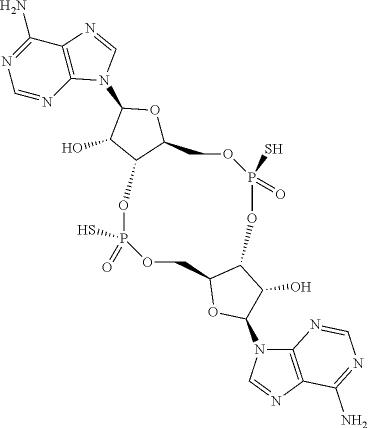 [SMILES: Nc1ncnc2c1ncn2[C@H]1O[C@H]2CO[P@@](=O)(S)O[C@@H]3[C@H](O)[C@@H](n4cnc5c(N)ncnc54)O[C@H]3CO[P@](=O)(S)O[C@@H]2[C@@H]1O] |
| Example 9 Compound 48b Beta-L-RR-(A)(A) dithio-[R_{P}, R_{P}]-cyclic- L-A(3′,5′)p-L-A(3′,5′)p] (2S,3S,3aR,5R,7aS,9S,10S,10aR,12R, 14aS)-2,9-bis(6-amino-9H-purin-9-yl)-3,10- dihydroxy-5,12-dimercaptooctahydro-2H,7H- difuro[3,2-d:3′2′-j][1,3,7,9]tetraoxa[2,8] diphosphacyclododecine 5,12-dioxide | 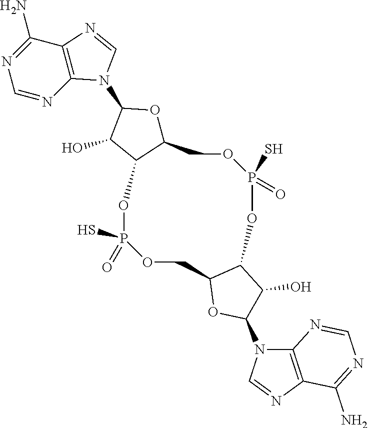 [SMILES: Nc1ncnc2c1ncn2[C@H]1O[C@H]2CO[P@@](=O)(S)O[C@@H]3[C@H](O)[C@@H](n4cnc5c(N)ncnc54)O[C@H]3CO[P@@](=O)(S)O[C@@H]2[C@@H]1O] |
| Example 10 compound 49 3′2′-dithio-(2′OTBS-G)(3′OTBS-A); dithio-cyclic-[2′OTBS-G(3′,5′)p-3′OTBS- A(2′,5′)p] | 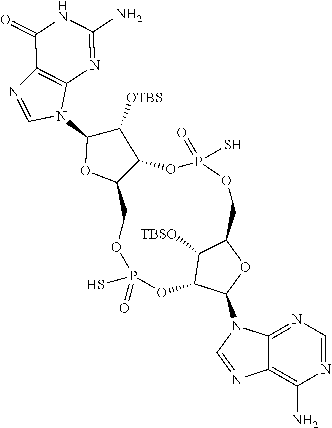 [SMILES: CC(C)(C)[Si](C)(C)O[C@@H]1[C@@H]2OP(=O)(S)OC[C@H]3O[C@@H](n4cnc5c(N)ncnc54)[C@H](OP(=O)(S)OC[C@H]2O[C@H]1n1cnc2c(=O)[nH]c(N)nc21)[C@@H]3O[Si](C)(C)C(C)(C)C] |
| Example 10 compound 49a 3′2′-RR-(2′OTBS-G)(3′OTBS-A); dithio-[R_{P}, R_{P}]-cyclic-[2′OTBS-G(3′,5′)p- 3′OTBS-A(2′,5′)p] | 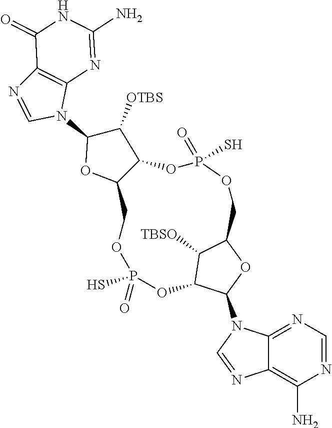 [SMILES: CC(C)(C)[Si](C)(C)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C] |
| Example 10 compound 49b 3′2′-SR-(2′OTBS-G)(3′OTBS-A); dithio-[S_{P}, R_{P}]-cylic-[2′OTBS-G(3′,5′)p- 3′OTBS-A(2′,5′)p] | 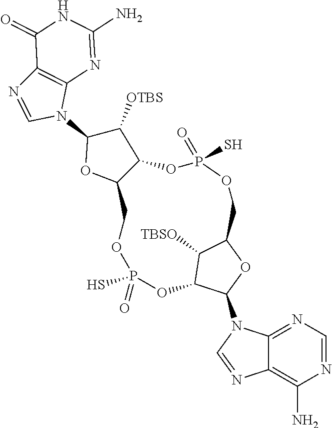 [SMILES: CC(C)(C)[Si](C)(C)O[C@@H]1[C@@H]2O[P@@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C] |
| Example 10 compound 49c 3′2′-RS-(2′OTBS-G)(3′OTBS-A); dithio-[R_{P}, S_{P}]-cyclic-[2′OTBS-G(3′,5′)p- 3′OTBS-A(2′,5′)p] | 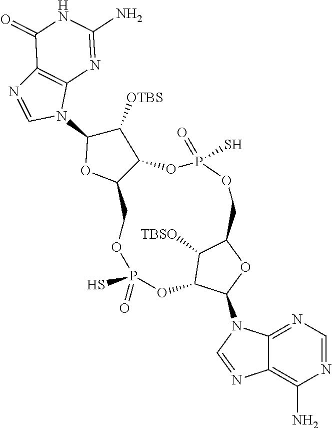 [SMILES: CC(C)(C)[Si](C)(C)O[C@@H]1[C@@H]2O[P@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C] |
| Example 10 compound 49d 3′2′-SS-(2′OTBS-G)(3′OTBS-A); dithio-[S_{P}, S_{P}]-cyclic-[2′OTBS-G(3′,5′)p- 3′OTBS-A(2′,5′)p] | 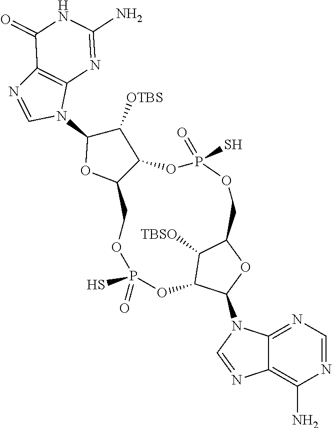 [SMILES: CC(C)(C)[Si](C)(C)O[C@@H]1[C@@H]2O[P@@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C] |

### Abbreviations and Acronyms

SalPCl=Salicyl chlorophosphite. DCA=dichloroacetic acid. DDTT=3-((N,N-dimethyl-aminomethylidene)amino)-3H-1,2,4-dithiazole-5-thione. DAST=diethylaminosulfur trifluoride. NaHCO₃=sodium bicarbonate. DCM=CH₂Cl₂=dichloromethane. EtOH=ethanol. EtOAc=ethyl acetate. KOAc=potassium acetate. MeCN=acetonitrile. MeOH=methanol. DMAP=N,N-dimethylpyridin-4-amine. DIPEA=diisopropylethylamine. DMOCP=2-chloro-5,5-dimethyl-1,3,2-dioxaphosphorinane-2-oxide. DMTCI=4,4′-dimethoxytrityl chloride. DMT=4,4-dimethoxytrityl. N-phenyltriflamide=1,1,1-trifluoro-N-phenyl-N-((trifluoromethyl)sulfonyl)methanesulfonamide. TBAF=tetrabutylammonium fluoride. TBS=tert-butyldimethylsilyl. TEAA=Triethyl ammonium acetate. TEA=trimethylamine. TEAH=triethyl ammonium. TEAB=treithylammonium bicarbonate. TFA=trifluoroacetic acid. TMSCI=trimethylsilyl chloride. HF=hydrofluoric acid. THF=tetrahydrofuran. G=Guanine. G^{ib}=isobutyryl guanine. 6-O-propargyl-G=guanine-6-propargyl ether (also 6-(prop-2-yn-1-yloxy)-9H-purin-2-amine). A=adenine. A^{B}^{_{Z}}=benzoyl adenine. 2,6-DAP=2,6-diamino purine. DAP^{dPAC}=diphenoxyacetyl 2,6-diamino purine. C=Cytosine. AMA=ammonium hydroxide/40% methylamine solution in water.

### Example 1: Synthesis of 2′3′-RR-(3′OTBS-A)(2′F-A) (6) and 2′3′-SR-(3′OTBS-A)(2′F-A) (6a)

2′3′-(3′OTBS-A)(2′F-A) (6), also referred to as dithio-[R_{P}, R_{P}]-cyclic-[3′OTBS-A(2′,5′)p-2′F-A(3′,5′)p], and 2′3′-SR-(3′OTBS-A)(2′F-A) (6a), also referred to as dithio-[S_{P}, R_{P}]-cyclic-[3′OTBS-A(2′,5′)p-2′F-A(3′,5′)p] were prepared as the triethylammonium salts according to the following Scheme 1:

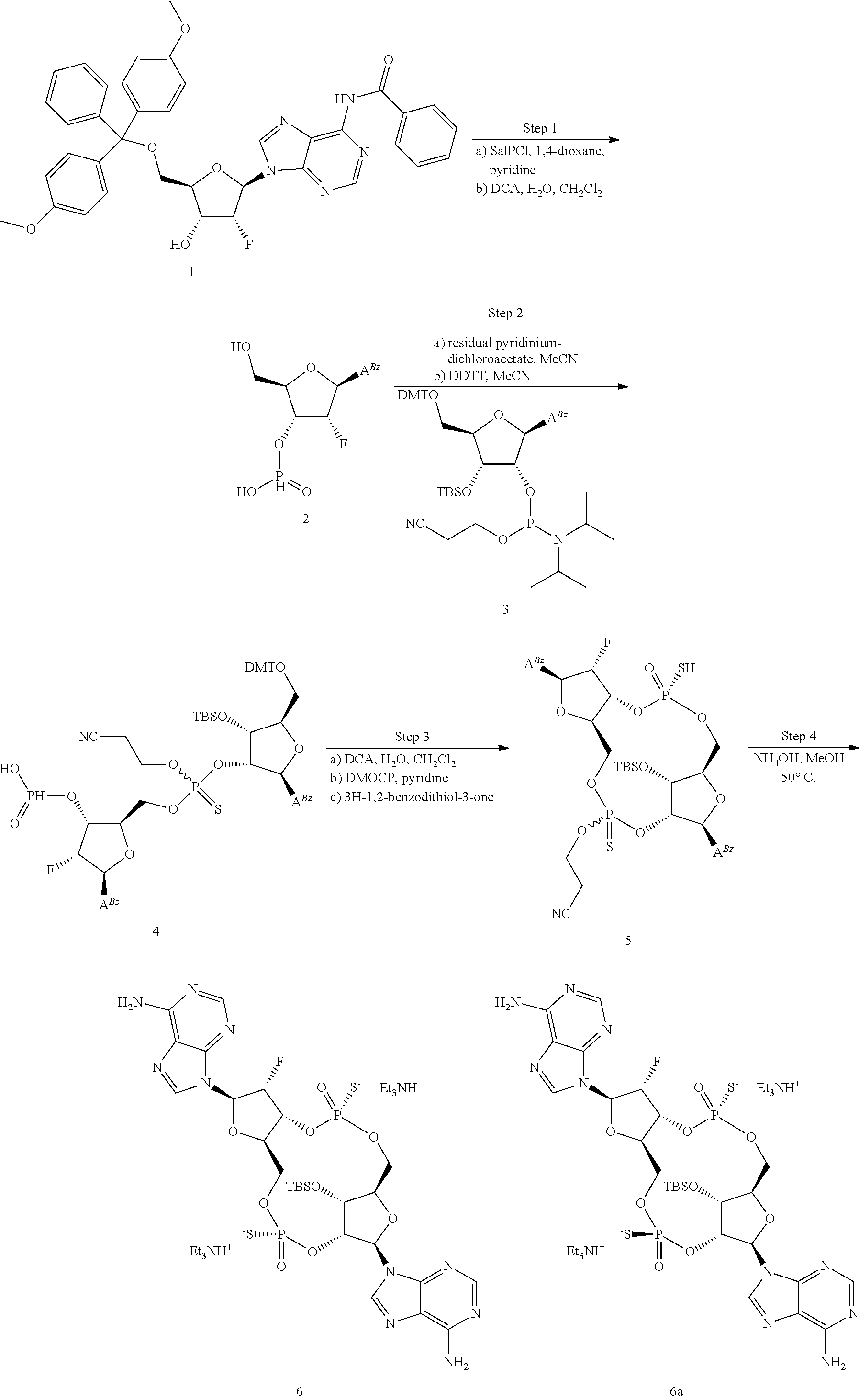

Step 1: (2R,3R,4R,5R)-5-(6-benzamido-9H-purin-9-yl)-4-fluoro-2-(hydroxymethyl)tetrahydrofuran-3-yl hydrogen phosphonate (2): To a stirring solution of N-(9-((2R,3R,4R,5R)-5-((bis(4-methoxyphenyl)(phenyl)methoxy)methyl)-3-fluoro-4-hydroxytetrahydrofuran-2-yl)-9H-purin-6-yl)benzamide (1, 1.5 g, 2.3 mmol, ChemGenes) in 1,4-dioxane (20 mL) and pyridine (6.7 mL) was added a solution of SalPCl (0.45 g, 2.3 mmol) in 1,4-dioxane (10 mL). After 30 min, to the stirring reaction mixture at room temperature was introduced water (3 mL), and the mixture was poured into a 1N aqueous NaHCO₃ solution (60 mL). This aqueous mixture was extracted with EtOAc (2×120 mL) and the layers were separated. The EtOAc extracts were combined and concentrated to dryness as a colorless foam. The colorless foam was dissolved in CH₂Cl₂ (25 mL) to give a colorless solution. To this solution was added water (0.4 mL) and a 6% (v/v) solution of DCA in CH₂Cl₂ (23 mL). After 10 min, to the red solution was charged pyridine (3.0 mL), which turned the red solution into a white mixture. This white mixture was concentrated in vacuo and water was removed as an azeotrope after concentration with MeCN (33 mL). This azeotrope process was repeated two more times with MeCN (33 mL). On the last evaporation, the white slurry of compound 2 was left in MeCN (10 mL).

Step 2: (2R,3R4R,5R)-5-(6-benzamido-9H-purin-9-yl)-2-((((((2R,3R,4R,5R)-2-(6-benzamido-9H-purin-9-yl)-5-((bis(4-methoxyphenyl)(phenyl)methoxy)methyl)-4-((tert-butyldimethyl silyl)oxy)tetrahydrofuran-3-yl)oxy)(2-cyanoethoxy)phosphorothioyl)oxy)methyl)-4-fluorotetrahydrofuran-3-yl hydrogen phosphonate (4): A solution of (2R,3R,4R,5R)-2-(6-benzamido-9H-purin-9-yl)-5-((bis(4-methoxyphenyl)(phenyl)methoxy)methyl)-4-((tert-butyldimethylsilyl)oxy)tetrahydrofuran-3-yl (2-cyanoethyl) diisopropylphosphoramidite (3, 1.0 g, 2.3 mmol, ChemGenes) in MeCN (15 mL) was dried through concentration in vacuo. This process was repeated three more times to remove water as an azeotrope. On the last azeotrope, to the solution of compound 3 in MeCN (8 mL) was introduced ten 3 Å molecular sieves and this solution was stored under an atmosphere of nitrogen. To a stirring mixture of compound 2 with residual pyridinium dichloroacetate in MeCN (10 mL) was added the solution of compound 3 in MeCN (8 mL). After 5 min, to the stirring mixture was added DDTT (520 mg, 2.6 mmol), which resulted in a yellow mixture. After 30 min, the yellow mixture was concentrated in vacuo to give compound 4 as a yellow oil.

Step 3: Protected 2′3′-RR/SR-(3′OTBS-A)(2′F-A) (5): To a solution of compound 4 in CH₂Cl₂ (30 mL) was added water (0.2 mL) and a 6% (v/v) solution of DCA in CH₂Cl₂ (30 mL). After 10 min, to the red solution was introduced pyridine (10 mL), which turned the solution into a yellow mixture. The yellow mixture was concentrated in vacuo until approximately 10 mL of the yellow mixture remained. To the yellow mixture was introduced pyridine (40 mL) and the mixture was evaporated until approximately 20 mL of the yellow mixture remained, and addition of pyridine and evaporation to 20 mL was repeated. To the stirring yellow mixture in pyridine (20 mL) was added DMOCP (0.9 g, 4.8 mmol). After 7 min, to the dark orange solution was added water (0.8 mL), followed immediately by the introduction of 3H-1,2-benzodithiol-3-one (0.4 g, 2.4 mmol). After 5 min, the dark orange solution was poured into a 1N aqueous NaHCO₃ solution (200 mL), which resulted in a biphasic mixture. After stirring for 10 min, the biphasic mixture was extracted with EtOAc (100 mL) and diethyl ether (100 mL). After separating the layers, the aqueous layer was back extracted twice with 200 ml of EtOAc/diethyl ether (1:1). The organic extracts were combined and concentrated in vacuo. To the concentrated yellow oil was added toluene (75 mL) and the mixture was evaporated to remove residual pyridine. This procedure was repeated twice with toluene (75 mL). The resulting oil was purified by silica gel chromatography (0% to 10% MeOH in CH₂CH₂) to obtain compound 5 (180 mg, 20%) as an orange oil.

Step 4: 2′3′-RR-(3′OTBS-A)(2′F-A) (6) and 2′3′-SR-(3′OTBS-A)(2′F-A) (6a): To a stirring solution of compound 5 (180 mg, 0.17 mmol) in methanol (1.5 mL) was added aqueous ammonium hydroxide (1.5 mL) and the orange slurry was heated to 50° C. After 2 h, the orange solution was allowed to cool and concentrated in vacuo. To the residual solid was introduced 10 mM aqueous TEAA (2 mL) and acetonitrile (0.5 mL) to form a solution, which was purified by reverse phase silica gel chromatography (0% to 20% MeCN in 10 mM aqueous TEAA) to separate compound 6 (2.3 mg, 3%, purity 98%) from the S,R diastereomer 6a as a white bis-triethylammonium salt after lyophilization. Compound 6: LCMS-ESI: 804 [M−H]⁻ (calculated for C₂₀H₂₃FN₁₀O₉P₂S₂: 804.30); Rₜ: 8.826 min. The S,R diastereomer 6a was isolated as the bis-triethylammonium salt (1.3 mg 2%, purity 72%). LCMS-ESI: 804 [M−H]⁻ (calculated for C₇₆H₃₇FN₁₀O₉P₂S₂Si: 804.30); Rₜ: 7.662 min.

### Example 2: Synthesis of 2′3′ RR-(A)(2,6-DAP) (12) and 2′3′-SR-(A)(2,6-DAP) (12a)

2′3′-RR-(A)(2,6-DAP) 12, also referred to as dithio-[R_{P}, R_{P}]-cyclic-[A(2′,5′)p-2,6-DAP(3′,5′)p] and 2′3′-SR-(A)(2,6-DAP) 12a, also referred to as dithio-[S_{P}, R_{P}]-cyclic-[A(2′,5′)p-2,6-DAP(3′,5′)p], were prepared as the triethylammonium salts according to the following Scheme 2:

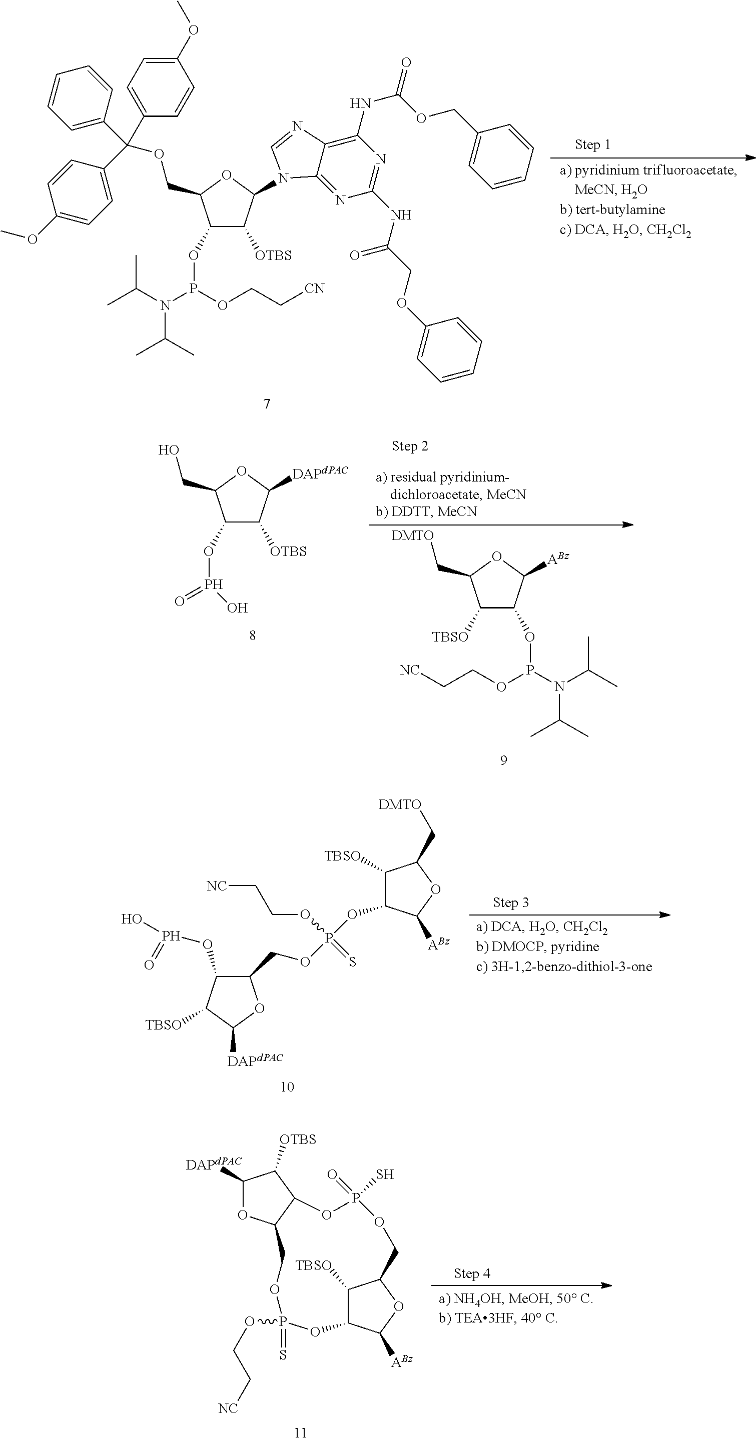

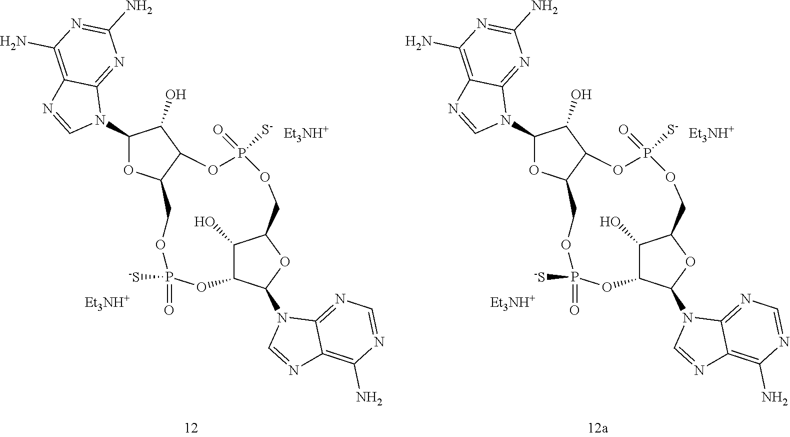

Step 1: (2R,3R,4R,5R)-5-(2,6-bis(2-phenoxyacetamido)-9H-purin-9-yl)-4-((tert-butyldimethylsilyl)oxy)-2-(hydroxymethyl)tetrahydrofuran-3-yl hydrogen phosphonate (8): To a solution of (21R,3R,4R,5R)-5-(2,6-bis(2-phenoxyacetamido)-9H-purin-9-yl)-2-((bis(4-ethoxyphenyl)(phenyl)methoxy)methyl)-4-((tert-buty)dimethylsilyl)oxy)tetrahydrofuran-3-yl (2-cyanoethyl) diisopropylphosphoramidite (7, 0.5 g, 0.43 mmol, ChemGenes) in THF (5.0 mL) and water (20 μL) was added pyridinium trifluoroacetate (0.1 g, 0.52 mmol, 1.2 equiv). After 10 min, to the stirring reaction mixture at room temperature was added tert-butylamine (2.0 mL, 20 mmol). After 10 min, the reaction solution was concentrated in vacuo and water was removed as an azeotrope after concentration with MeCN (3×30 mL). After the last evaporation, the resulting colorless foam was dissolved in CH₂Cl₂ (9 mL) to give a colorless solution. To this solution was added water (0.055 mL) and a 6% (v/v) solution of DCA in CH₂Cl₂ (9 mL), which turned the colorless solution into a bright orange solution. After 10 min of stirring at room temperature, to the orange solution was charged with pyridine (3 mL), which reverted the orange solution back into a colorless solution. After 10 min of stirring, the colorless solution was concentrated in vacuo and water was removed as an azeotrope after concentration with MeCN (20 mL). This process was repeated two more times with MeCN (20 mL) to remove the residual water as an azeotrope. On the last evaporation, the resulting white paste of compound 8 was dissolved in MeCN (5 mL).

Step 2: (2R,3R,4R,5R)-2-((((((2R,3R,4R,5R)-2-(6-benzamido-9H-purin-9-yl)-5-((bis(4-methoxyphenyl)(phenyl)methoxy)methyl)-4-((tert-butyldimethylsilyl)oxy)tetrahydrofuran-3-yl)oxy)(2-cyanoethoxy)phosphorothioyl)oxy)methyl)-5-(2,6-bis(2-phenoxyacetamido)-9H-purin-9-yl)-4-((tert-butyldimethylsilyl)oxy)tetrahydrofuran-3-yl hydrogen phosphonate (10): A solution of (2R,3R,4R,5R)-2-(6-benzamido-9H-purin-9-yl)-5-((bis(4-methoxyphenyl)(phenyl)methoxy)methyl)-4-((tert-butyldimethylsilyl)oxy)tetrahydrofuran-3-yl (2-cyanoethyl) diisopropylphosphoramidite (9, 0.43 g, 0.43 mmol, ChemGenes) in MeCN (20 mL) was dried through concentration in vacuo. This process was repeated two more times with MeCN (2×20 mL) and on the final concentration, 2 mL of MeCN remained. To this colorless solution in MeCN (2 mL) was introduced ten pieces of 3 Å molecular sieves. This solution was stored under an atmosphere of nitrogen. To a stirring mixture of compound 8 with residual pyridinium dichloroacetate in MeCN (5 mL) was added the solution of compound 9 in MeCN (2 mL). After 5 min, to the stirring mixture was added DDTT (100 mg, 0.48 mmol), which resulted in a yellow mixture. After 30 min, the yellow mixture was concentrated in vacuo to give compound 10 as a yellow paste.

Step 3: Protected 2′3′-RR/SR-(A)(2,6-dap) (11): To a solution of compound 10 in CH₂Cl₂ (10 mL) was added water (0.06 mL) and a 6% (v/v) solution of DCA in CH₂Cl₂ (10 mL). After 10 min of stirring at room temperature, to the orange solution was introduced pyridine (3 mL), which changed the color of the solution from orange to yellow. The yellow solution was concentrated in vacuo until approximately 5 mL of the yellow mixture remained. To the yellow solution was introduced pyridine (10 mL) and the solution was evaporated until approximately 5 mL of the yellow mixture remained. To the stirring yellow solution in pyridine (5 mL) was added DMOCP (240 mg, 1.3 mmol). After 5 min, to the dark orange solution was added water (0.250 mL), followed immediately by the introduction of 3H-1,2-benzodithiol-3-one (110 mg, 0.65 mmol). After 10 min, the yellow solution was poured into a 1N aqueous NaHCO₃ solution (50 mL). After 10 min, the stirred biphasic mixture was extracted with EtOAc (75 mL) and diethyl ether (50 mL). After separating the layers, the aqueous layer was back extracted twice with a mixture of EtOAc (75 mL) and diethyl ether (50 mL). The organic extracts were combined and concentrated. To the concentrated yellow oil was added toluene (40 mL) and the mixture was evaporated to remove residual pyridine. This procedure was repeated with toluene (40 mL). The resulting oil was purified by silica gel chromatography (0% to 10% MeOH in CH₂Cl₂) to obtain a mixture of protected 2′3′-RR/RS-(A)(2,6-dap) (11) stereoisomers (210 mg, 36%), as a yellow solid.

Step 4: 2′3′-RR-(A)(2,6-dap) (12) and 2′3′-SR-(A)(2,6-dap) (12a): To a stirring solution of a mixture of compound 11 (200 mg, 0.15 mmol) in methanol (2.0 mL) at 50° C. was added 30% v/v aqueous ammonium hydroxide (2 mL) and the orange slurry was heated to 50° C. After 18 h, the yellow mixture was allowed to cool and concentrated in vacuo. To the residual solid (104 mg, 0.11 mmol) was introduced triethylamine trihydrofluoride (1.0 mL) and the yellow solution was heated to 40° C. After 2 h, the yellow solution was allowed to cool to room temperature. This yellow solution was slowly added to an ice-water cooled solution of 1M TEAB (5 mL) and triethylamine (0.8 mL). The yellow mixture was allowed to stir for 1 h. The yellow mixture was purified by reverse phase silica gel chromatography (0% to 15% MeCN in 10 mM aqueous TEAA) to obtain compound 12 (6.0 mg, 15%) as a white bis-triethylammonium salt after lyophilization and compound 12a (11 mg, 28%) as a white bis-triethylammonium salt after lyophilization. Characterization for compound 12: LCMS-ESI: 704.25 [M−H]⁻ (calculated for C₂₀H₂₅N₁₁O₁₀P₂S₂: 705.07); Rₜ: 14.739 min. ¹H NMR (400 MHz, 45° C., D₂O) δ 8.69 (s, 1H), 8.30 (s, 1H), 7.99 (s, 1H), 6.41 (d, J=8.0 Hz, 1H), 6.12 (d, J=2.8 Hz, 1H), 5.54 (td, J=9.0, 4.0 Hz, 1H), 5.31 (q, J=4.4 Hz, 1H), 5.03 (d, J=4.0 Hz, 2H), 4.70-4.66 (m, 1H), 4.58-4.53 (m, 1H), 4.47-4.43 (m, 2H), 4.29 (dd, J=12.0, 4.0 Hz, 1H), 3.21 (q, J=7.2 Hz, 18H), 1.37 (t, J=7.2 Hz, 28H). Characterization for compound 12a: LCMS-ESI: 704.25 [M−H]⁻ (calculated for C₂₀H₂₅N₁₁O₁₀P₂S₂: 705.07); Rₜ: 12.472 min. ¹H NMR (400 MHz, 45° C., D₂O) δ 8.81 (s, 1H), 8.38 (s, 1H), 8.13 (s, 1H), 6.45 (d, J=8.4 Hz, 1H), 6.13 (d, J=5.6 Hz, 1H), 5.52-5.47 (m, 2H), 5.24 (t, J=4.8 Hz, 1H), 4.75-4.63 (m, 1H), 4.62-4.54 (m, 3H), 4.34-4.30 (m, 1H), 4.19-4.16 (m, 1H), 3.31 (q, J=7.2 Hz, 18H), 1.42 (t, J=7.2 Hz, 28H).

### Example 3: Synthesis of RR-(2′F-A)(2′OTBS-A) (18) and SR-(2′F-A)(2′OTBS-A) (8a)

RR-(2′F-A)(2′OTBS-A) (18), also referred to as dithio-[R_{P}, R_{P}]-cyclic-[2′F-A(3′,5′)p-2′OTBS-A(3′,5′)p] or (2R,3R,3aR,5R,7aR,9R,10R,10aR,12R,14aR)-2,9-bis(6-amino-9H-purin-9-yl)-3-((tert-butyldimethylsilyl)oxy)-10-fluoro-5,12-dimercaptooctahydro-2H,7H-difuro[3,2-d:3′,2′-j][1,3,7,9]tetraoxa[2,8]diphosphacycldodecine 5,12-dioxide, and SR-(2′F-A)(2′OTBS-A) (18a), also referred to as dithio-[S_{P}, R_{P}]-cyclic-[2′F-A(3′,5′)p-2′ OTBS-A(3′,5′)p] or (2R,3R,3aR,5R,7aR,9R,10R,10aR,12S,14aR)-2,9-bis(6-amino-9H-purin-9-yl)-3-((tert-butyldimethylsilyl)oxy)-10-fluoro-5,12-dimercaptooctahydro-2H,7H-difuro[3,2-d:3′,2′-j][1,3,7,9]tetraoxa[2,8]diphosphacyclododecine 5,12-dioxide were prepared as the ammonium salts according to the following Scheme 3:

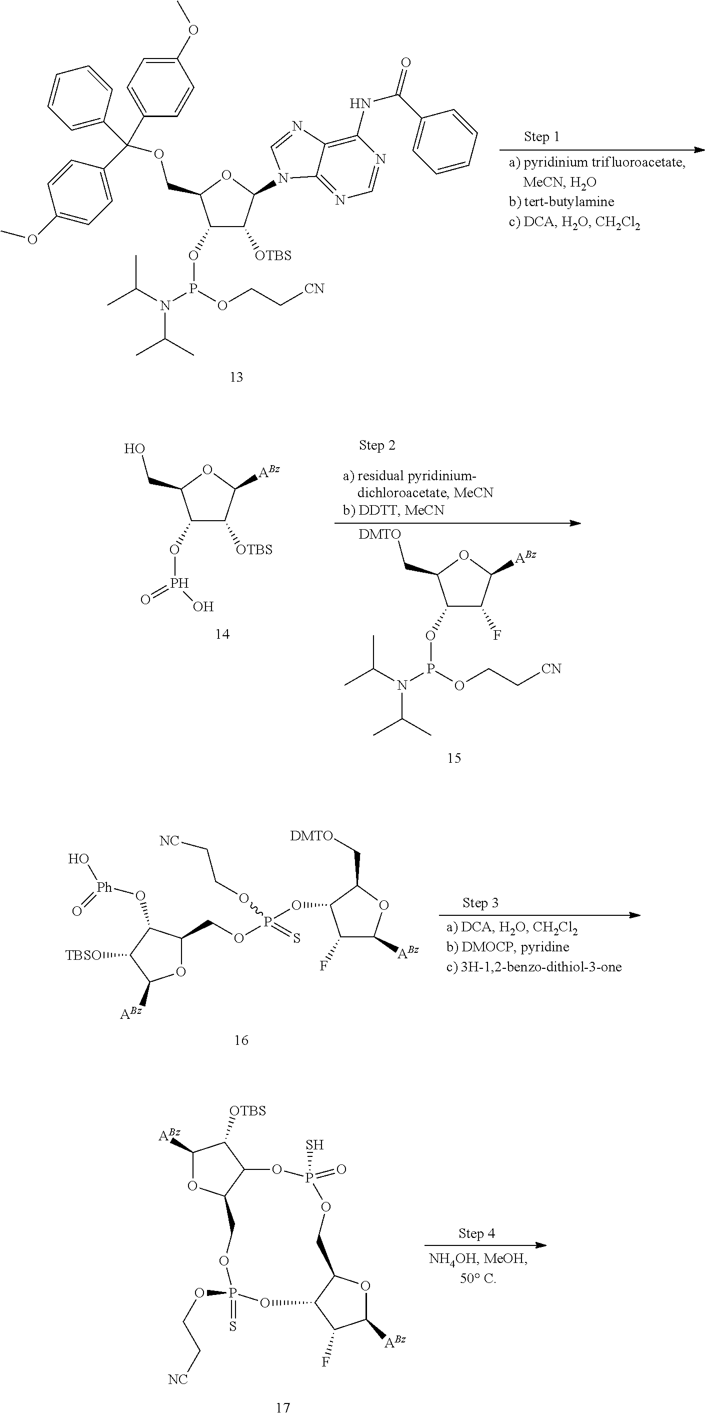

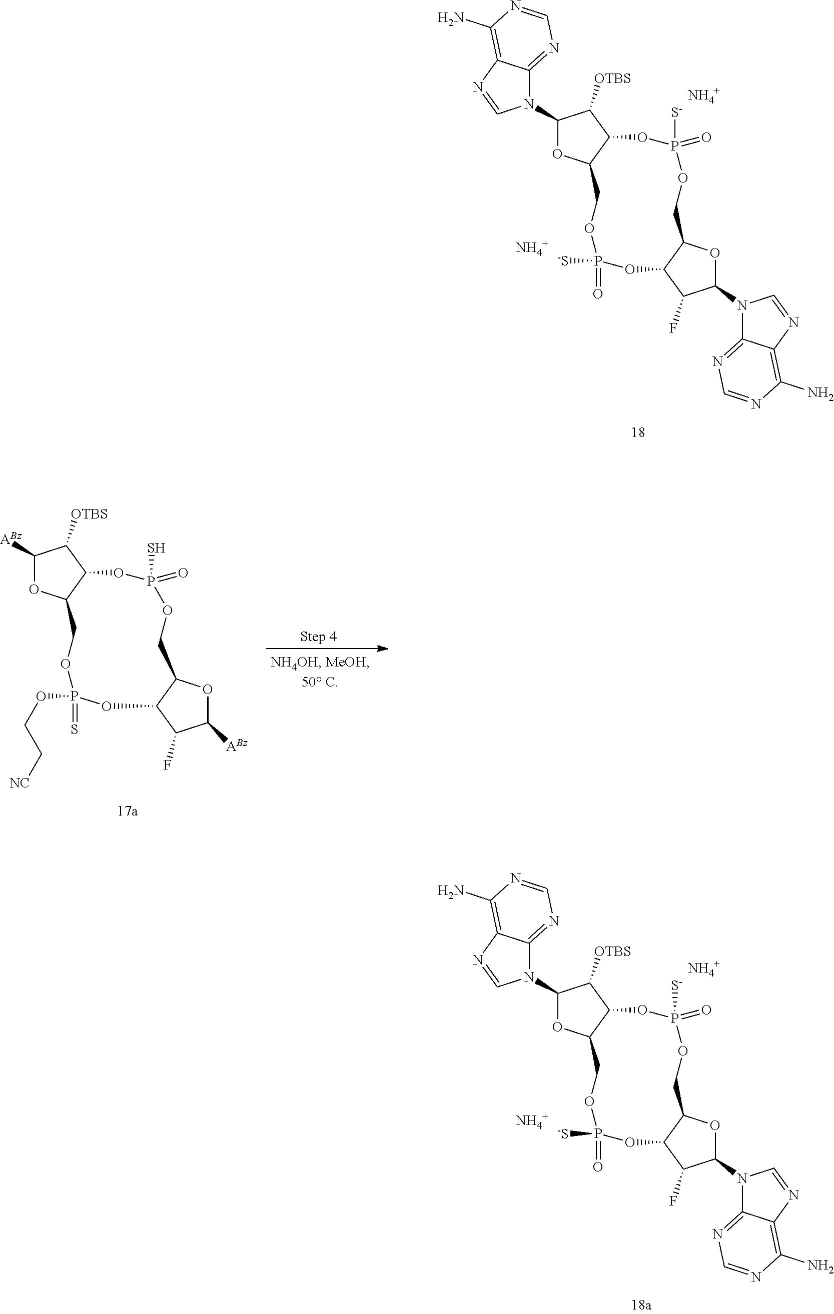

Step 1: (2R,3R,4R,5R)-5-(6-benzamido-9H-purin-9-yl)-4-((tert-butyldimethylsilyl)oxy)-2-(hydroxymethyl)tetrahydrofuran-3-yl hydrogen phosphonate (14): To a solution of (21R,3R,4R,5R)-5-(6-benzamido-9H-purin-9-yl)-2-((bis(4-methoxyphenyl)(phenyl)methoxy)methyl)-4-((tert-butyldimethylsilyl)oxy)tetrahydrofuran-3-yl (2-cyanoethyl) diisopropylphosphoramidite (13, 2.0 g, 2.0 mmol, ChemGenes) in MeCN (10 mL) and water (70 μL) was added pyridinium trifluoroacetate (0.47 g, 2.4 mmol, 1.2 equiv). After 8 min, to the stirring reaction mixture at room temperature was added tert-butylamine (10 mL, 95 mmol). After 10 min, the reaction solution was concentrated in vacuo and water was removed as an azeotrope after concentration with MeCN (3×30 mL). After the last evaporation, the resulting colorless foam was dissolved in CH₂Cl₂ (25 mL) to give a colorless solution. To this solution was added water (0.36 mL) and a 6% (v/v) solution of DCA in CH₂Cl₂ (25 mL), which turned the colorless solution into a bright orange solution. After 10 min of stirring at room temperature, to the orange solution was charged pyridine (3 mL), which reverted the orange solution back into a colorless solution. After 10 min of stirring, the colorless solution was concentrated in vacuo and water was removed as an azeotrope after concentration with MeCN (30 mL). This process was repeated three more times with MeCN (30 mL) to remove the residual water as an azeotrope. On the last evaporation, the resulting white paste of compound 14 was left in MeCN (15 mL) as a milky white mixture.

Step 2: (2R,3R,4R,5R)-5-(6-benzamido-9H-purin-9-yl)-2-((((((2R,3R,4R,5R)-5-(6-benzamido-9H-purin-9-yl)-2-((bis(4-methoxyphenyl)(phenyl)methoxy)methyl)-4-fluorotetrahydrofuran-3-yl)oxy)(2-cyanoethoxy)phosphorothioyl)oxy)methyl)-4-((tert-butyldimethylsilyl)oxy)tetrahydrofuran-3-yl hydrogen phosphonate (16): A solution of (2R,3R,4R,5R)-5-(6-benzamido-9H-purin-9-yl)-2-((bis(4-methoxyphenyl)(phenyl)methoxy)methyl)-4-fluorotetrahydrofuran-3-yl (2-cyanoethyl) diisopropylphosphoramidite (15, 1.77 g, 2.02 mmol) in MeCN (20 mL) was dried through concentration in vacuo. This process was repeated two more times with MeCN (20 mL) and on the final concentration, 6 mL of MeCN remained. To this colorless solution in MeCN (2 mL) was introduced six pieces of 3 Å molecular sieves. This solution was stored under an atmosphere of nitrogen. To a stirring mixture of compound 14 with residual pyridinium dichloroacetate in MeCN (15 mL) was added the solution of compound 15 in MeCN (6 mL). After 10 min, to the stirring mixture was added DDTT (460 mg, 2.2 mmol), which resulted in a yellow mixture. After 30 min, the yellow mixture was concentrated in vacuo to give compound 16 as a yellow oil.

Step 3: N′,N′-(((2R,3R,3aR,5R,7aR,9R,10R,10aR,14aR)-3-((tert-butyldimethylsilyl)oxy)-12-(2-cyanoethoxy)-10-fluoro-5-mercapto-5-oxido-12-sulfidooctahydro-2H, 7H-difluro[3,2-d:3′,2′-j][1,3,7,9]tetraoxa[2,8]diphosphacyclododecine-2,9-diyl)bis(9H-purine-9,6-diyl))dibenzamide (17) and N,N′-(((2R,3R,3aR,5R,7aR,9R,10R,10aR,12S,14aR)-3-((tert-butyldimethylsilyl)oxy)-12-(2-cyanoethoxy)-10-fluoro-5-mercapto-5-oxido-12-sulfidooctahydro-2H,7H-difuro[3,2-d:3′,2′-j][1,3,7,9]tetraoxa[2,8]diphosphacyclododecine-2,9-diyl)bis(9H-purine-9,6-diyl))dibenzamide (17a): To a solution of compound 16 in CH₂Cl₂ (50 mL) was added water (0.24 mL) and a 6% (v/v) solution of DCA in CH₂Cl₂ (50 mL), After 10 min of stirring at room temperature, to the orange solution was introduced pyridine (15 mL), which changed the color of the solution from orange to yellow. The yellow solution was concentrated in vacuo until approximately 10 mL of the yellow mixture remained. To the yellow solution was introduced pyridine (50 mL) and the solution was evaporated until approximately 25 mL of the yellow mixture remained. On two successive occasions, to the yellow mixture was added pyridine (50 mL) and the solution was concentrated until approximately 30 mL of the yellow solution remained. To the stirring yellow solution in pyridine (30 mL) was added DMOCP (1.1 g, 6 mmol). After 5 min, to the dark orange solution was added water (1 mL), followed immediately by the introduction of 3H-1,2-benzodithiol-3-one (0.5 g, 3.0 mmol). After 30 min, the yellow solution was poured into a 1N aqueous sodium bicarbonate solution (400 mL). After stirring for 10 min, the biphasic mixture was extracted with EtOAc (200 mL) and CH₂Cl₂ (200 mL). After separating the layers, the aqueous layer was back extracted twice with CH₂Cl₂ (400 mL). The organic extracts were combined and concentrated. To the concentrated yellow oil was added toluene (100 mL) and the mixture was evaporated to remove residual pyridine. This procedure was repeated with toluene (100 mL). The resulting oil was purified by silica gel chromatography (0% to 10% MeOH in CH₂Cl₂) to obtain two diastereomers, compound 17 (150 mg, 7%) as a yellow solid and compound 17a (120 mg, 6%) as a yellow solid.

Step 4: Ammonium (2R,3R,3aR,5R,7aR,9R,10R,10aR,12R,14aR)-2,9-bis(6-amino-9H-purin-9-yl)-3-((tert-butyldimethylsilyl)oxy)-10-fluorooctahydro-2H, 7H-difuro[3,2-d:3′,2′-j][1,3,7,9]tetraoxa[2,8]diphosphacyclododecine-5,12-bis(thiolate) 5,12-dioxide (18): To a stirring solution of compound 17 (150 mg, 0.14 mmol) in methanol (2.0 mL) was added 30% v/v aqueous ammonium hydroxide (2 mL) and the yellow solution was heated to 50° C. After 3 h, the yellow solution was allowed to cool and concentrated in vacuo. After drying under high vacuum overnight, compound 18 (120 mg, 99%, purity 57%) was obtained as a white bis-ammonium salt after lyophilization. LCMS-ESI: 806 [M−H]⁻ (calculated for C₂₀H₂₃FN₁₀O₉P₂S₂: 805.35); Rₜ: 8.345 min.

Ammonium (2R,3R,3aR,5R,7aR,9R,10R,10aR,12S,14aR)-2,9-bis(6-amino-9H-purin-9-yl)-3-((tert-butyldimethylsilyl)oxy)-10-fluorooctahydro-2H,7H-difuro[3,2-d:3′,2′-j][1,3,7,9]tetraoxa[2,8]diphosphacyclododecine-5,12-bis(thiolate) 5,12-dioxide (18a): To a stirring solution of compound 17a (120 mg, 0.11 mmol) in methanol (1.5 mL) was added 30% v/v aqueous ammonium hydroxide (1.5 mL) and the yellow solution was heated to 50° C. After 3 h, the yellow solution was allowed to cool and concentrated in vacuo. After drying under high vacuum overnight, compound 18a (110 mg, 99%, purity 61%) was obtained as a white bis-ammonium salt after lyophilization. LCMS-ESI: 806 [M−H]⁻ (calculated for C₂₀H₂₃FN₁₀O₉P₂S₂: 805.35); Rₜ: 7.383 min.

### Example 4: Synthesis of RR-(2,6-DAP)(2,6-DAP) (23) and SR-(2,6-DAP)(2,6-DAP) (23a)

(2R,3R,3aS,5R,7aR,9R,10R,10aS,12R,14aR)-2,9-bis(2,6-diamino-9H-purin-9-yl)-3,10-dihydroxy-5,12-dimercaptooctahydro-2H,7H-difuro[3,2-d:3′,2′-j][1,3,7,9]tetraoxa[2,8]diphosphacyclododecine 5,12-dioxide 23 and (2R,3R,3aS,5R,7aR,9R,10R,10aS,12S,14aR)-2,9-bis(2,6-diamino-9H-purin-9-yl)-3,10-dihydroxy-5,12-dimercaptooctahydro-2H,7H-difuro[3,2-d:3′,2′-j][1,3,7,9]tetraoxa[2,8]diphosphacyclododecine 5,12-dioxide 23a were prepared as the triethylammonium salts according to the following Scheme 4:

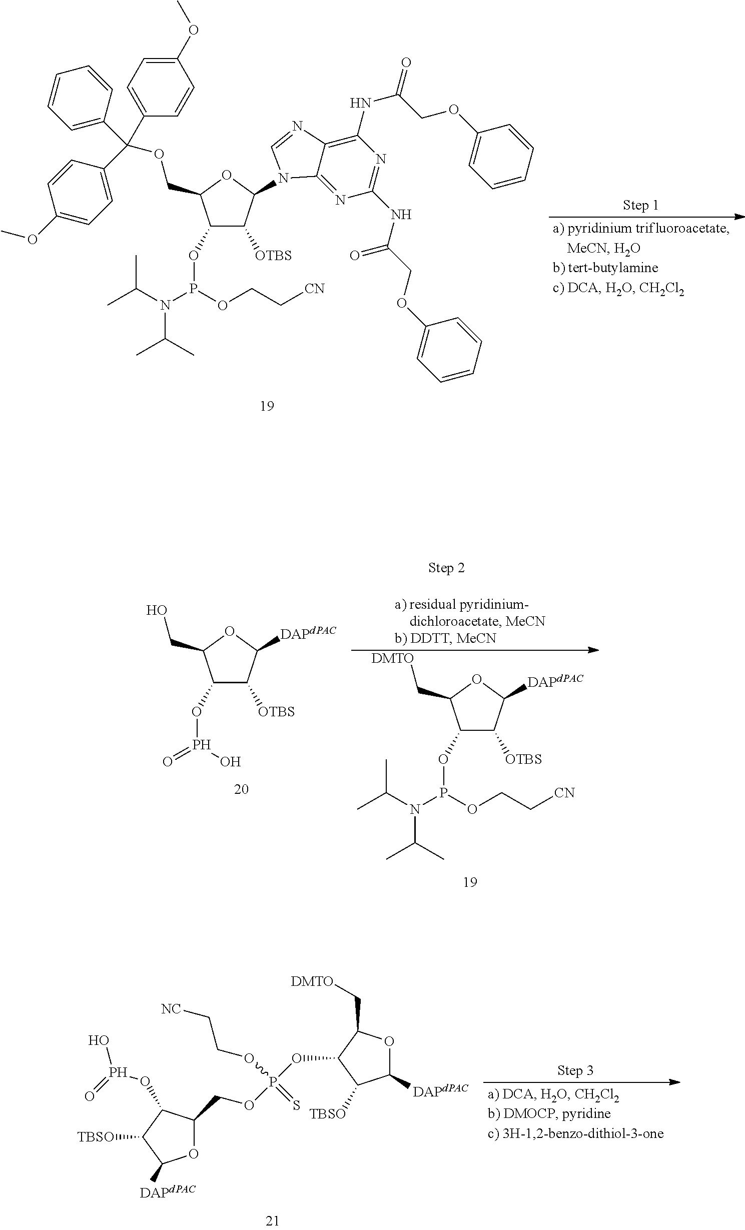

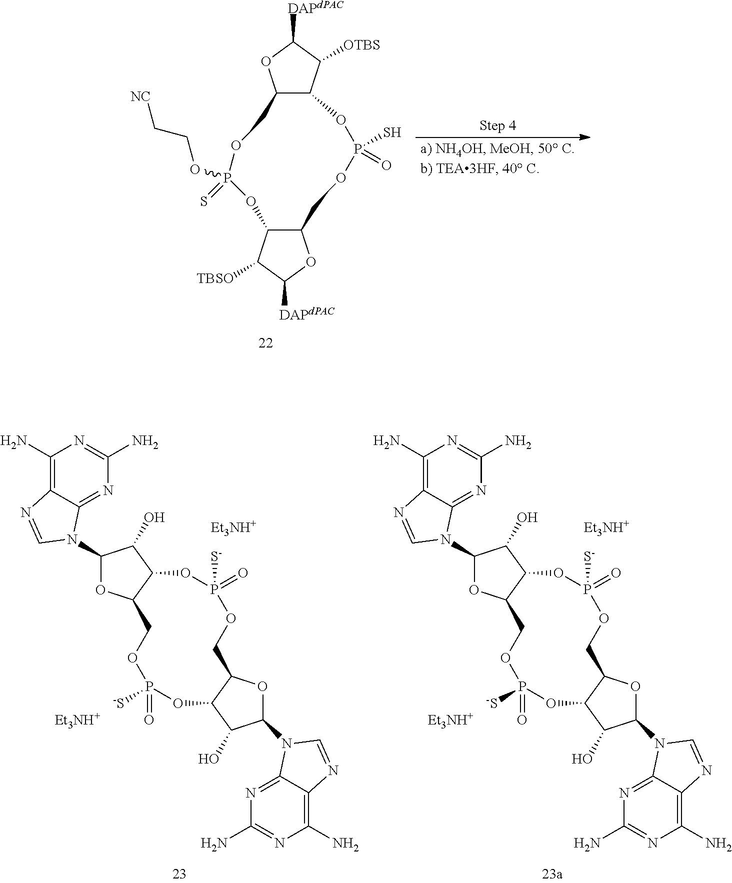

Step 1: (2R,3R,4R,5R)-5-(2,6-bis(2-phenoxyacetamido)-9H-purin-9-yl)-4-((tert-butyldimethylsilyl)oxy)-2-(hydroxymethyl)tetrahydrofuran-3-yl hydrogen phosphonate (20): To a solution of (2R,3R,4R,5R)-5-(2,6-bis(2-phenoxyacetamido)-9H-purin-9-yl)-2-((bis(4-methoxyphenyl)(phenyl)methoxy)methyl)-4-((tert-butyldimethylsilyl)oxy)tetrahydrofuran-3-yl (2-cyanoethyl) diisopropylphosphoramidite (19, 1.0 g, 0.9 mmol, ChemGenes) in MeCN (7.0 mL) and water (45 μL) was added pyridinium trifluoroacetate (0.23 g, 1.2 mmol). After 10 min, to the stirring reaction mixture at room temperature was added tert-butylamine (5.0 mL, 47.6 mmol). After 15 min, the reaction solution was concentrated in vacuo and water was removed as an azeotrope after concentration with MeCN (3×20 mL). After the last evaporation, the resulting colorless foam was dissolved in CH₂Cl₂ (13 mL) to give a colorless solution. To this solution was added water (0.16 ml) and a 5% (v/v) solution of DCA in CH₂Cl₂ (13 mL). After 10 min of stirring at room temperature, to the orange solution was charged pyridine (1.5 mL), which turned the orange solution into a colorless solution. After 10 min of stirring, the colorless solution was concentrated in vacuo and water was removed as an azeotrope after concentration with MeCN (15 mL.). This azeotrope process was repeated two more times with MeCN (15 mL). On the last evaporation, the resulting white paste of compound 20 was dissolved in MeCN (25 mL).

Step 2: (2R,3R,4R,5R)-5-(2,6-bis(2-phenoxyacetamido)-9H-purin-9-yl)-2-((((((2R,3R,4R,5R)-5-(2,6-bis(2-phenoxyacetamido)-9H-purin-9-yl)-2-((bis(4-methoxyphenyl)(phenyl)methoxy)methyl)-4-((tert-butyldimethylsilyl)oxy)tetrahydrofran-3-yl)oxy)(2-cyanoethoxy)phosphorothioyl)oxy)methyl)-4-((tert-butyldimethylsilyl)oxy)tetrahydrofuran-3-yl hydrogen phosphonate (21): A solution of compound 19 (1.0 g, 0.9 mmol, ChemGenes) in MeCN (15 mL) was dried through concentration in vacuo. To this solid was added anhydrous THF (15 mL). To this solution of compound 19 in THF (15 mL) was introduced a couple dozen of 3 Å molecular sieves. This solution was stored under an atmosphere of nitrogen. To a stirring mixture of compound 20 with residual pyridinium dichloroacetate in MeCN (10 mL) was added the solution of compound 19 in THF (15 mL). After 10 min, to the stirring mixture was added DDTT (283 mg, 1.4 mmol), which resulted in a yellow mixture. After 30 min, the yellow mixture was concentrated in vacuo to give compound 21 as a yellow paste.

Step 3: N,N′-(((2R,3R,3aR,7aR,9R,10R,10aR,12R,14aR)-3,10-bis((tert-butyldimethsiylsilyl)oxy)-5-(2-cyanoethoxy)-12-mercapto-12-oxido-5-sulfidooctahydro-2H, 7H-difuro[3,2-d:3′,2′-j][1,3,7,9]tetraoxa[2,8]diphosphacyclododecine-2,9-diyl)bis(2-(2-phenoxyacetamido)-9H-purine-9,6-diyl))bis(3-phenylpropanamide) (22): To a solution of compound 21 in CH₂Cl₂ (35 mL) was added water (0.16 mL) and a 4% (v/v) solution of DCA in CH₂Cl₂ (30 mL). After 10 min of stirring at room temperature, to the orange solution was introduced pyridine (15 mL), which changed the color of the solution from orange to yellow. The yellow solution was concentrated in vacuo until approximately 10 mL of the yellow mixture remained. To the yellow solution was introduced pyridine (20 mL) and the solution was evaporated until approximately 6 mL of the yellow mixture remained. To the yellow mixture was added pyridine (20 mL) and the solution was concentrated until approximately 15 mL of the yellow solution remained. To the stirring yellow solution in pyridine (15 mL) was added DMOCP (472 mg, 2.6 mmol). After 5 min, to the dark orange solution was added water (0.8 mL), followed immediately by the introduction of 3H-1,2-benzodithiol-3-one (217 mg, 1.5 mmol). After 30 min, the yellow solution was poured into a 1N aqueous NaHCO₃ solution (200 mL). After stirring for 30 min, the biphasic mixture was extracted with EtOAc (100 mL) After separating the layers, the aqueous layer was back extracted twice with EtOAc (2×100 mL). The organic extracts were combined and concentrated. To the concentrated yellow oil was added toluene (30 mL) and the mixture was evaporated to remove residual pyridine. This procedure was repeated with toluene (70 mL). The resulting oil was purified by silica, gel chromatography (0% to 10% MeOH in CH₂Cl₂) to obtain a mixture of compound 22 along with an impurity (696 mg, 53%), as a yellow solid.

Step 4: Triethylammonium (2R,3R,3aS,5R,7aR,9R,10R,10aS,12R,14aR)-2,9-bis(2,6-diamino-9H-purin-9-yl)-3,10-dihydroxyoctahydro-2H,7H-difuro[3,2-d:3′,2′-j][1,3,7,9]tetraoxa[2,8]diphosphacyclododecine-5,12-bis(thiolate) 5,12-dioxide (23) and triethylammonium (2R,3R,3aS,5R,7aR,9R,10R,10aS,12S,14aR)-2,9-bis(2,6-diamino-9H-purin-9-yl)-3,10-dihydroxyoctahydro-2H,7H-difuro[3,2-d:3′,2′-j][1,3,7,9]tetraoxa[2,8]diphosphacyclododecine-5,12-bis(thiolate) 5,12-dioxide (23a): To a stirring solution of a mixture of compound 22 (696 mg, 0.08 mmol) in methanol (5.0 mL) at 50° C. was added 30% v/v aqueous ammonium hydroxide (5.4 mL) and the orange slurry was heated to 50° C. After 19 h, the yellow mixture was allowed to cool and concentrated in vacuo. To the residual solid (357 mg, 0.38 mmol) was introduced triethylamine trihydrofluoride (5.0 mL) and the yellow solution was heated to 40° C. After 3 h, the yellow solution was allowed to cool to room temperature. This yellow solution was slowly added to a cooled solution of 1M TEAB (21 mL) and triethylamine (3.5 mL). The yellow mixture was allowed to stir for 1 h. The yellow mixture was purified by reverse phase silica gel chromatography (0% to 15% MeCN in 10 mM aqueous TEAA) to obtain compound 23 (14.5 mg, 2%) as a white bis-triethylammonium salt after lyophilization and compound 23a (7.22 mg, 1%) as a white bis-triethylammonium salt after lyophilization. Characterization for compound 23: LCMS-ESI: 719.75 [M−H]⁻ (calculated for C₂₀H₂₆N₁₂O₁₀P₂S₂: 720.57); Rₜ: 6.860 min. ¹H NMR (400 MHz, 45° C., D₂O) δ 8.05 (s, 2H), 5.95 (s, 2H), 4.89 (br s, 2H), 4.63-4.61 (d, 2H), 4.42-4.36 (m, 4H), 3.97 (d, J=11.2 Hz, 2H), 3.10 (q, J=7.2 Hz, 12H), 1.17 (t, J=7.2 Hz, 18H). ³¹P NMR (45° C., D₂O) δ 54.6. Characterization for compound 23a: LCMS-ESI: 719.80 [M−H]⁻ (calculated for C₂₀H₂₆N₁₂O₁₀P₂S₂: 720.57); Rₜ: 8.028 min. ¹H NMR (400 MHz, 45° C., D₂O) δ 8.38 (s, 1H), 8.24 (s, 1H), 6.16 (s, 2H), 5.19-5.16 (m, 2H), 5.12 (s, 11H), 4.88 (s, 11H), 4.55-4.51 (m, 4H), 4.23-4.20 (m, 2H), 3.34 (q, J=6.8 Hz, 12H), 1.42 (t, J=6.8 Hz, 18H). ³¹P NMR (45° C., D₂O) δ 55.83, 54.72.

### Example 5: Synthesis of (6-O-propargyl-G)(G) (25) and 6-O-propargyl-G) (6-O-propargyl-G)(26)

(6-O-propargyl-G)(G) 25, also referred to as cyclic-[6-O-propargyl-G(3′,5′)p-G(3′,5′)p] or 2-amino-9-((2R,3R,3aS,7aR,9R,10R,10aS,14aR)-9-(2-amino-6-(prop-2-yn-1-yloxy)-9H-purin-9-yl)-3,5,10,12-tetrahydroxy-5,12-dioxidooctahydro-2H,7H-difuro[3,2-d:3′,2′-j][1,3,7,9]tetraoxa[2,8]diphosphacyclododecin-2-yl)-1,9-dihydro-6H-purin-6-one and (6-O-propargyl-G)(6-O-propargyl-G) 26, also referred to as (2R,3R,3aS,7aR,9R,10R,10aS,14aR)-2,9-bis(2-amino-6-(prop-2-yn-1-yloxy)-9H-purin-9-yl)-3,5,10,12-tetrahydroxyoctahydro-2H,7H-difuro[3,2-d: 3′,2′-j][1,3,7,9]tetraoxa[2,8]diphosphacyclododecine 5,12-dioxide were prepared as the triethylammonium salts according to the following Scheme 5:

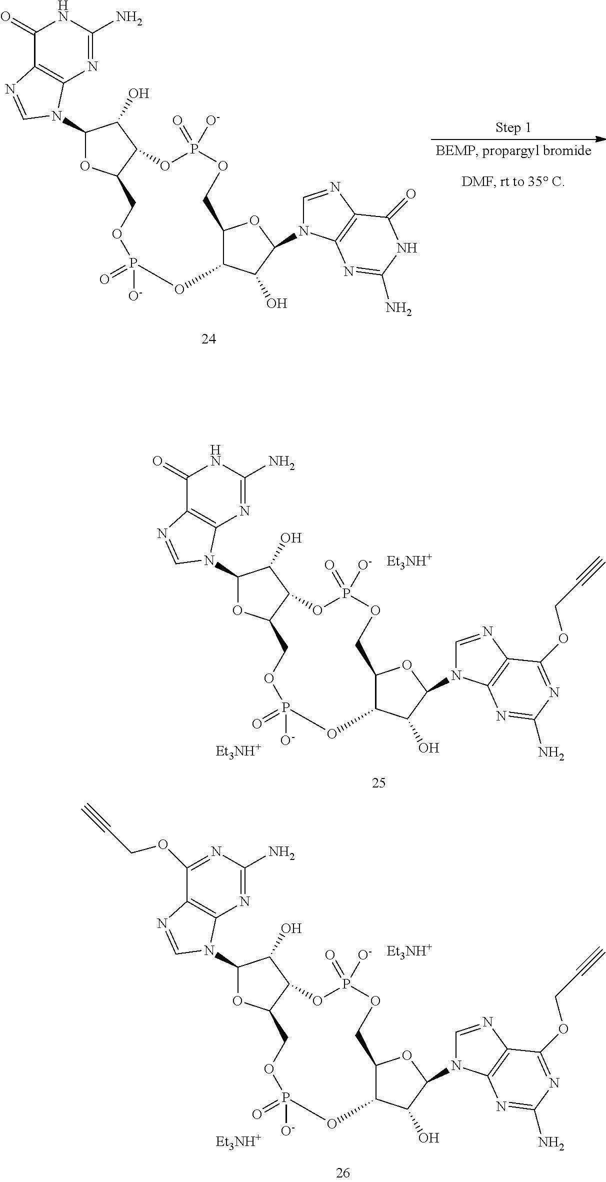

Step 1: To a solution of cyclic-[G(3′,5′)p-G(3′,5′)p] (CDG, 120 mg, 0.13 mmol, 1 equiv) in anhydrous acetonitrile (4 mL) and anhydrous pyridine (4 mL) and anhydrous DMF (2.6 mL), was added an 80% solution of propargyl bromide in toluene (25 μL, 0.26 mmol, 2 equiv) followed by BEMP (2-tert-Butylimino-2-diethylamino-1,3-dimethylperhydro-1,3,2-diazaphosphosphorine) (150 μL, 0.52 mmol, 4 equiv). The reaction mixture was stirred between 25 and 35° C. for 2 h. It was quenched with trimethylamine bicarbonate (0.75 mL) and concentrated. The mono and bis propargyl compounds 25 and 26 were separated by preparative reverse phase HPLC to give 20.1 mg of compound 25 and 20 mg of compound 26. Compound 25: LCMS-ESI: 727.15 [M−H]⁻ (Calculated for C₂₃H₂₆N₁₀O₁₄P₂: 728.11); 1H NMR (45° C., D₂O) δ 8.26 (s, 1H), δ 8.16 (s, 1H), 6.12 (s, 1H), 6.09 (s, 1H), 5.15-5.12 (m, 2H), 5.00-4.92 (m, 4H), 4.59-4.49 (m, 4H), 4.26-4.23 (m, 2H), 3.36 (q, J=76, 26H), 2.94 (s, 1H), 2.15 (s, 14H), 1.43 (t, J=7.2 H, 40H). Compound 26: LCMS-ESI: 765.25 [M−H]⁻ (Calculated for C₂₆H₂₈N₁₀O₁₄P₂: 766.13); 1H NMR 1H NMR (45° C., D₂O) δ 8.20 (s, 2H), 6.14 (s, 2H), 5.10-4.93 (m, 8H), 4.56-4.49 (m, 4H), 4.24 (d, J=12.4, 2H), 3.35 (q, J=7.2, 17H), 2.90 (s, 2H), 2.12 (s, 4H), 1.43 (t, J=72 H, 26H) The CDG starting material was prepared as described in Giaffney et al., Organic Letters 2010, 12(14):3269-3271, the disclosure of which is incorporated by reference as it relates to the synthesis of CDG.

### Example 6: Synthesis of RR-(2′F-ibG)(2′F-ibG) (32) and RS-(2′F-ibG)(2′F-ibG) (32a)

RR-(2′F-ibG)(2′F-ibG) 6, also referred to as dithio-[R_{P}, R_{P}]-cyclic-[2′F-ibG (3′,5′)p-2′F-ibG(3′,5′)p] or N,N′-(((2R,3R,3aR,5R,7aR,9R,10R,10aR,12R,14aR)-3,10-difluoro-5,12-dimercapto-5,12-dioxidooctahydro-2H, 7H-difuro[3,2-d:3′,2′-j][1,3,7,9]tetraoxa[2,8]diphosphacyclododecine-2,9-diyl)bis(6-oxo-6,9-dihydro-1H-purine-9,2-diyl))bis(2-methylpropanamide) and RS-(2′F-ibG)(2′F-ibG) 6a, also referred to as dithio-[R_{P}, S_{P}]-cyclic-[2′F-ibG(3′,5′)p-2′F-ibG(3′,5′)p] or N,N′-(((2R,3R,3aR,5R,7aR,9R,10R,10aR,12S,14aR)-3,10-difluoro-5,12-dimercapto-5,12-dioxidooctahydro-2H,7H-difuro[3,2-d:3′,2′-j][1,3,7,9]tetraoxa[2,8]diphosphacyclododecine-2,9-diyl)bis(6-oxo-6,9-dihydro-1H-purine-9,2-diyl))bis(2-methylpropanamide) were prepared as the triethylammonium salts according to the following Scheme 6:

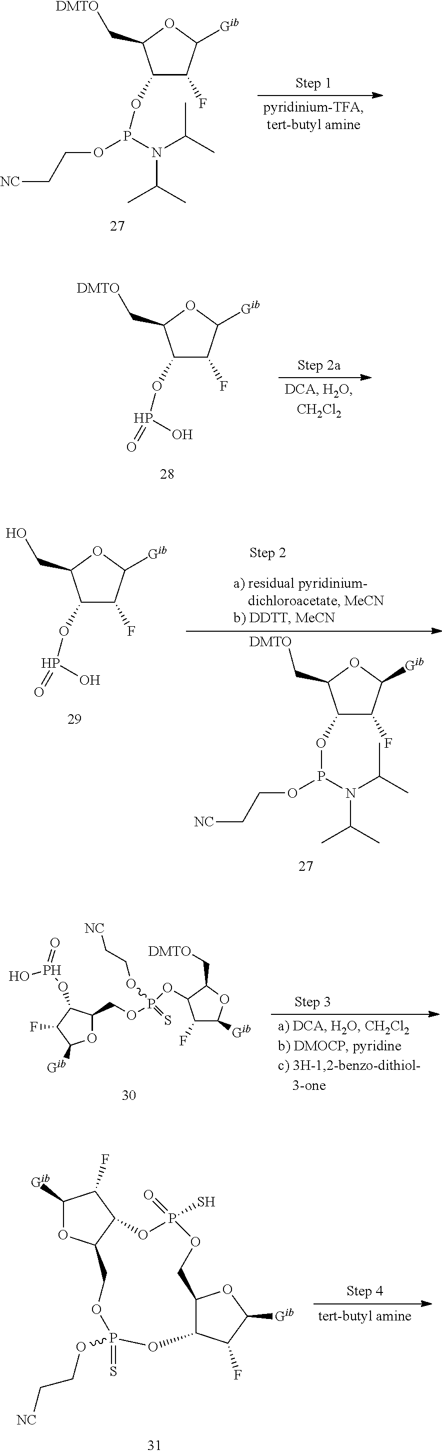

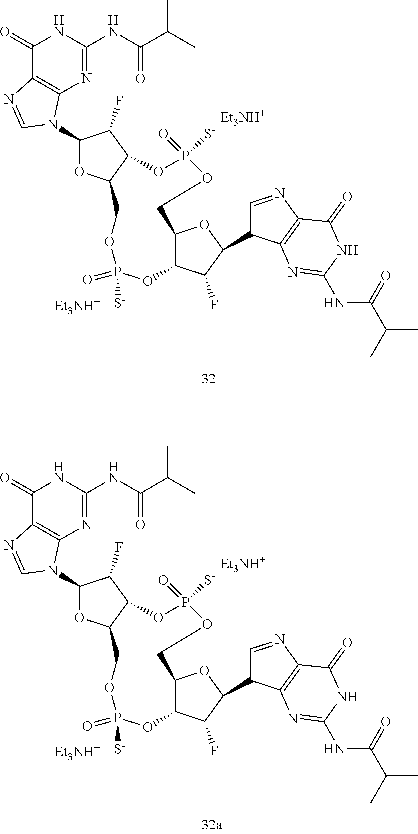 [SMILES: "Et3NH+" ; "Et3NH+" ; "Et3NH+" ; CC(C)C(=O)Nc1nc2c(c(=O)[nH]1)N=CC2[C@@H]1O[C@@H]2CO[P@@](=O)([S-])O[C@H]3[C@@H](F)[C@H](n4cnc5c(=O)[nH]c(NC(=O)C(C)C)nc54)O[C@@H]3CO[P@@](=O)([S-])O[C@H]2[C@H]1F ; "Et3NH+" ; CC(C)C(=O)Nc1nc2c(c(=O)[nH]1)N=CC2[C@@H]1O[C@@H]2CO[P@@](=O)([S-])O[C@H]3[C@@H](F)[C@H](n4cnc5c(=O)[nH]c(NC(=O)C(C)C)nc54)O[C@@H]3CO[P@](=O)([S-])O[C@H]2[C@H]1F]

Step 1: (2R,3R,4R,5R)-2-((bis(4-methoxyphenyl)(phenyl)methoxy)methyl)-4-fluoro-5-(2-isobutyramido-6-oxo-1,6-dihydro-9H-purin-9-yl)tetrahydrofuran-3-yl hydrogen phosphonate (28): To a solution of (2R,3R,4R,5R)-2-((bis(4-methoxyphenyl)(phenyl)methoxy)methyl)-4-fluoro-5-(2-isobutyramido-6-oxo-1,6-dihydro-9H-purin-9-yl)tetrahydrofuran-3-yl (2-cyanoethyl) diisopropylphosphoramidite (27, 2.0 g, 2.4 mmole, 1 equiv, ChemGenes) in acetonitrile (10 mL) was added water (0.072 mL) followed by pyridinium trifluoroacetate (0.57 g, 2.4 mmole, 1 equiv). The reaction was stirred for 1 min and tert-butyl amine (10 mL, 0.10 mole, 42 equiv) was added. After 10 min of stirring the reaction was concentrated in vacuo to give crude compound 28.

Step 2: (2R,3R,4R,5R)-2-((((((2R3R,4R,5R)-2-((bis(4-methoxyphenyl)(phenyl)methoxy)methyl)-4-fluoro-5-(2-isobutyramido-6-oxo-1,6-dihydro-9H-purin-9-yl)tetrahydrofuran-3-yl)oxy)(2-cyanoethoxy)phosphorothioyl)oxy)methyl)-4-fluoro-5-(2-isobutyramido-6-oxo-1,6-dihydro-9H-purin-9-yl)tetrahydrofuran-3-yl hydrogen phosphonate (30): Compound 27 (2.5 g, 3.0 mmole, 1.2 equiv, ChemGenes) was coevaporated with anhydrous acetonitrile (3×15 mL) leaving 8 mL of acetonitrile. (Step 2a) To a solution of compound 28 (2.4 mmole, 1 equiv) in DCM (24 mL) was added water (0.36 mL) followed by 24 mL of 6% DCA in DCM solution. The reaction mixture was stirred for 10 min, then quenched with pyridine (2.8 mL) and concentrated in vacuo to give (2R,3R,4R,5R)-4-fluoro-2-(hydroxymethyl)-5-(2-isobutyramido-6-oxo-1,6-dihydro-9H-purin-9-yl)tetrahydrofuran-3-yl hydrogen phosphonate compound 29. The crude mixture of 29 was coevaporated with anhydrous acetonitrile (3×15 mL) leaving 2.8 mL of acetonitrile. The solution of compound 27 (2.5 g) in anhydrous acetonitrile (8 mL) was added to the solution of compound 29 in acetonitrile and stirred for 2 min. After the addition of DDTT (0.45 g), the reaction mixture was stirred for 30 min then concentrated in vacuo to give 8.1 g of crude compound 30.

Step 3: N,N′-(((2R,3R,3aR,7aR,9R,10R,10aR,12R,14aR)-5-(2-cyanoethoxy)-3,10-difluoro-12-mercapto-12-oxido-5-sulfidooctahydro-2H,7H-difuro[3,2-d: 3′,2′-j][1,3,7,9]tetraoxa[2,8]diphosphacyclododecine-2,9-diyl)bis(6-oxo-6,9-dihydro-1H-purine-9,2-diyl))bis(2-methylpropanamide) (31): To a solution of crude compound 30 (8.1 g, 2.5 mmole, 1 equiv) in DCM (48 mL) was added water (0.24 mL) followed by 48 mL of a 6% DCA in DCM solution. The reaction mixture was stirred for 10 min then quenched with pyridine (20 mL). The mixture was condensed in vacuo to remove the DCM, then coevaporated with anhydrous pyridine (60 mL) leaving 40 mL. DMOCP (1.2 g, 6.3 mmole) was added and stirred for 3 min followed by water (1 mL, 55 mmole) and immediately after with 3H-1,2-benzodithiol-3-one (0.52 g, 3.1 mmole). The reaction was allowed to proceed for 5 min and was diluted with a 1M solution of NaHCO₃ (300 mL) and extracted with EtOAc:Ether (1:1, 150 mL) then DCM (150 mL). The combined organic layers were concentrated in vacuo to give crude compound 31. Prep MPLC-SiO₂ (100% DCM to 50% DCM/MeOH) gave 150 mg of compound 31 as the RR diastereomer and 260 mg of 31 as a mixture of diastereomers.

Step 4: Triethylammonium (2R,3R,3aR,5R,7aR,9R,10R,10aR,12R,14aR)-3,10-difluoro-2,9-bis(2-isobutyramido-6-oxo-1,6-dihydro-9H-purin-9-yl)octahydro-2H,7H-difuro[3,2-d:3′,2′-j][1,3,7,9]tetraoxa[2,8]diphosphacyclododecine-5,2-bis(thiolate) 5,12-dioxide (32): To a solution of compound 31 (RR diastereomer, 75 mg, 0.082 mmole, 1 equiv) in acetonitrile (0.5 mL) was added tert-butyl amine (0.43 mL, 50 equiv). The mixture was capped and stirred for 45 min. The mixture was concentrated in vacuo to give 93 mg of crude 32. The reaction was purified using a prep-MPLC-C18 (100% 10 mM TEAA to 50% acetonitrile/10 mM TEAA) to give 22 mg of 32 (>95% pure) as the bis-triethylammonium salt. LCMS-ESI: 867.0 [M−H]⁻ (Calculated for C₂₆H₃₄F₂N₁₀O₁₂P₂S₂: 866.12); ¹H NMR (400 MHz, 45° C., MeOD) δ 8.25 (s, 2H), 6.25 (s, 1H), 6.20 (s, 1H), 5.16 (t, J=11.2, 2H), 4.54 (d, J=11.2, 2H), 4.34 (d, J=7.6, 2H), 4.03 (d, J=11.2, 2H), 2.76-2.73 (m, 2H), 1.21-1.18 (m, 12H). ¹⁹F NMR (400 MHz, 45° C., MeOD) δ −200.7, ³¹P NMR (45° C. MeOD) δ 55.7. Triethylammonium (2R,3R,3aR,5R,7aR,9R,10R,10aR,12S,14aR)-3,10-difluoro-2,9-bis(2-isobutyramido-6-oxo-1,6-dihydro-9H-purin-9-yl)octahydro-2H,7H-difuro[3,2-d:3′,2′-j][1,3,7,9]tetraoxa[2,8]diphosphacyclododecine-5,12-bis(thiolate) 5,12-dioxide (32a): To a solution of compound 31 (mixture of diastereomers, 260 mg, 0.283 mmole, 1 equiv) in acetonitrile (1.75 mL) was added tert-butyl amine (1.49 mL, 70 equiv). The mixture was capped and stirred for 45 min. The mixture was concentrated in vacuo to give a crude mixture of 32 and 32a. The reaction was purified using a prep-MPLC-C18 (100% 10 mM TEAA to 30% acetonitrile/10 mM TEAA) to give 36 mg of 32a RS diastereomer (>90% pure) as the bis-triethylammonium salt. LCMS-ESI: 867.0 [M−H]⁻ (Calculated for C₂₈H₃₄F₂N₁₀O₁₂P₂S₂: 866.12); 31P NMR (45° C., MeOD) δ 56.9; 55.8.

### Example 7: Synthesis of RR-(2′F-C)(2′F-A) (37) and RS-(2′F-C)(2′F-A) (37a)

RR-(2′F-C)(2′F-A) 37, also referred to as dithio-[R_{P}, R_{P}]-cyclic-[2′F-C(3′,5′)p-2′ F-A(2′,5′)p] or 4-amino-1-((2R,3R,3aR,5R,7aR,9R,10R,10aR,12R,14aR)-9-(6-amino-9H-purin-9-yl) 3,10-difluoro-5,12-dimercapto 5,12-dioxidooctahydro-2H,7H-difuro[3,2-d:3′,2′-j][1,3,7,9]tetraoxa[2,8]diphosphacyclododecin-2-yl)pyrimidin-2(1H)-one and RS-(2′F-C)(2′-A) 37a, also referred to as dithio-[R_{P}, R_{P}]-cyclic-[2′F-C(3′,5′)p-2′F-(2′,5′)p] or 4-amino-1-((2R,3R,3aR,5R,7aR,9R,10R,10aR,12R,14aR)-9-(6-amino-9H-purin-9-yl)-3,10-difluoro-5,12-dimercapto-5,12-dioxidooctahydro-2H,7H-difuro[3,2-d:3′,2′-j][1,3,7,9]tetraoxa[2,8]diphosphacyclododecin-2-yl)pyrimidin-2(1H)-one were prepared as the triethylammonium salts according to the following Scheme 7:

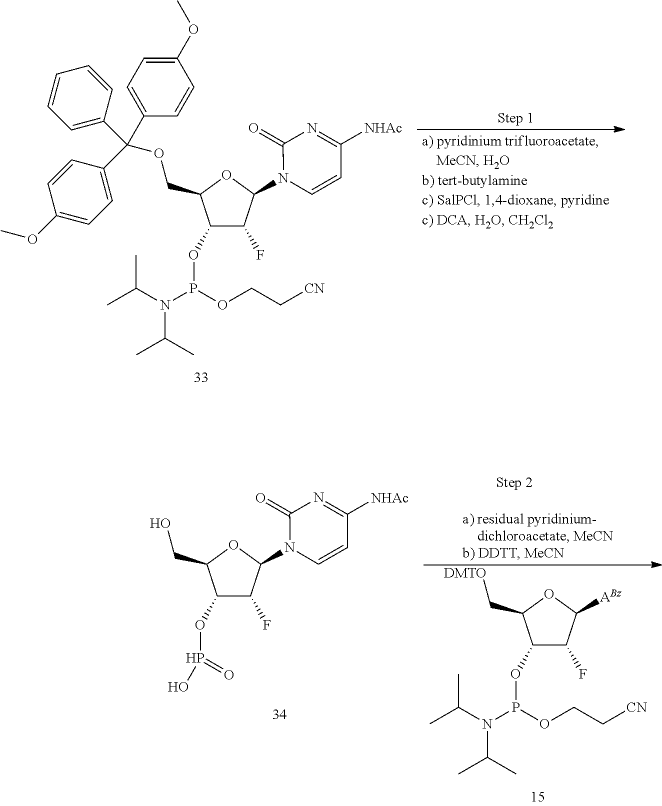

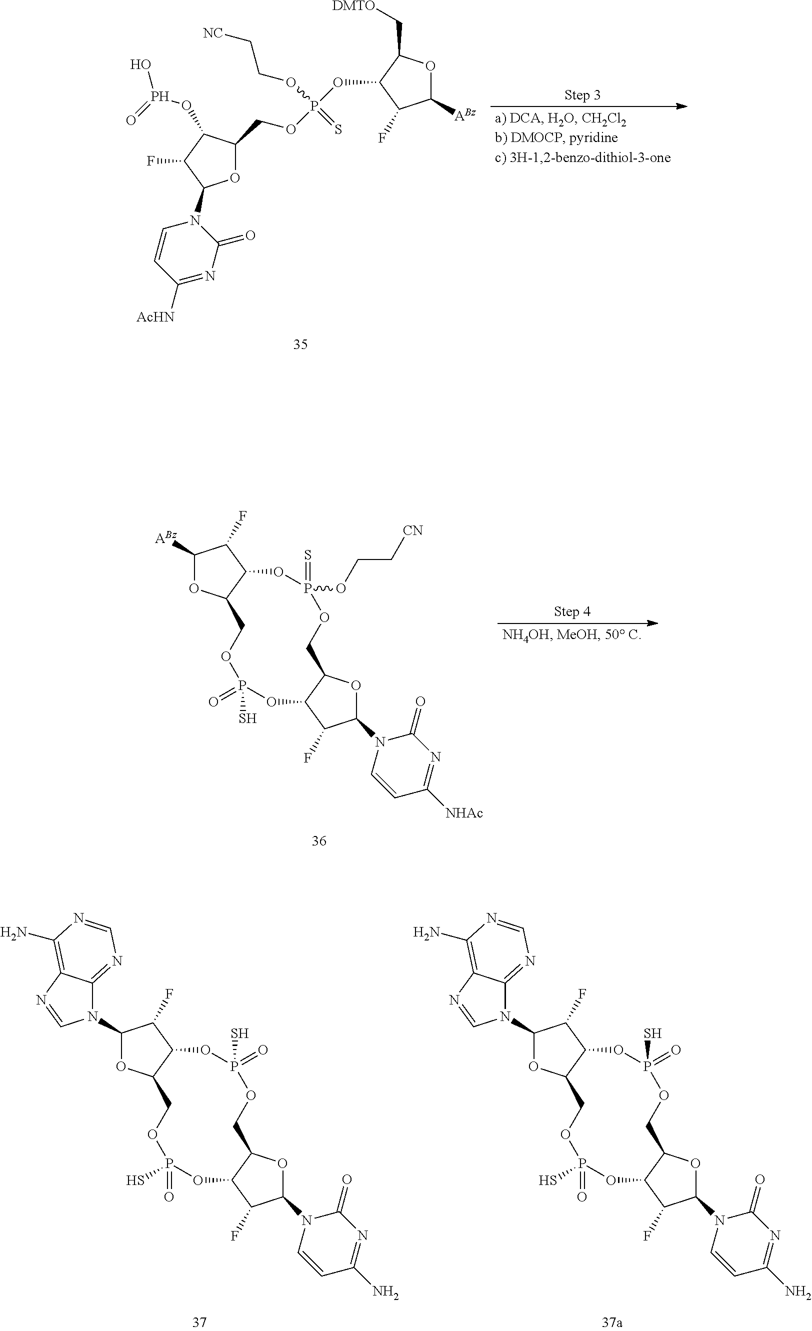

Step 1: (2R,3R,4R,5R)-5-(4-acetamido-2-oxopyrimidin-1(2H)-yl)-4-fluoro-2-(hydroxymethyl)tetrahydrofuran-3-yl hydrogen phosphonate (34): To a solution of (2R,3R,4R,5R)-5-(4-acetamido-2-oxopyrimidin-1(2H)-yl)-2-((bis(4-methoxyphenyl)(phenyl)methoxy)methyl)-4-fluorotetrahydrofuran-3-yl (2-cyanoethyl) diisopropylphosphoramidite (33, 0.9 g, 1.14 mmol, ChemGenes) in MeCN (5.5 mL) and water (40 μL) was added pyridinium trifluoroacetate (264 mg, 1.4 mmol, 1.2 equiv). After 10 min, to the stirring reaction mixture at room temperature was added tert-butylamine (5.5 mL, 54 mmol). After 7 min, the reaction solution was concentrated in vacuo and water was removed as an azeotrope after concentration with MeCN (3×15 mL). After the last evaporation, the resulting colorless foam was dissolved in 1,4-dioxane (8 mL) and pyridine (3 mL). To this colorless solution was added a solution of SalPCl (0.28 g, 1.4 mmol) in 1,4-dioxane (4 mL). After 1 h, to the stirring reaction mixture at room temperature was introduced a 1N aqueous NaHCO₃ solution (60 mL). This aqueous mixture was extracted three times with EtOAc (80 mL) and the layers were separated. The EtOAc extracts were combined and concentrated to dryness as a colorless foam. The colorless foam was purified by MPLC-SiO₂ (99% DCM:1% MeOH, with 0.5% pyridine to 50% DCM:50% MeOH, with 0.5% pyridine) to provide 275 mg (37%) of the desired intermediate, which was dissolved in CH₂Cl₂ (6 mL) to give a colorless solution. To this solution was added water (0.08 mL) and a 6% (v/v) solution of DCA in CH₂Cl₂ (6 mL), which turned the colorless solution into a bright orange solution. After 10 min of stirring at room temperature, to the orange solution was charged pyridine (0.6 mL), which reverted the orange solution back into a colorless solution. After 10 min of stirring, the colorless solution was concentrated in vacuo and water was removed as an azeotrope after concentration with MeCN (20 mL). This process was repeated two more times with MeCN (20 mL) to remove the residual water as an azeotrope. On the last evaporation, the resulting white paste of compound 34 was left in MeCN (5 mL) as a milky white mixture.

Step 2: (2R,3R,4R,5R)-5-(4-acetamido-2-oxopyrimidin-1(2H)-yl)-2-((((((2R,3R,4R,5R)-5-(6-benzamido-9H-purin-9-yl)-2-((bis(4-methoxyphenyl)(phenyl)methoxy)methyl)-4-fluorotetrahydrofuran-3-yl)oxy)(2-cyanoethoxy)phosphorothioyl)oxy)methyl)-4-fluorotetrahydrofuran-3-yl hydrogen phosphonate (35): A solution of compound 15 (0.56 g, 0.64 mmol, ChemGenes) in MeCN (15 mL) was dried through concentration in vacuo. This process was repeated three more times with MeCN (15 mL) and on the final concentration, 3 mL of MeCN remained. To this colorless solution in MeCN (3 mL) was introduced eight pieces of 3 Å A molecular sieves. This solution was stored under an atmosphere of nitrogen. To a stirring mixture of compound 34 with residual pyridinium dichloroacetate in MeCN (5 mL) was added the solution of compound 15 in MeCN (3 mL). After 10 min, to the stirring mixture was added DDTT (100 mg, 0.48 mmol), which resulted in a yellow slurry. After 30 min, the yellow slurry was concentrated in vacuo to provide compound 35 as a yellow oil.

Step 3: N-(9-((2R,3R,3aR,7aR,9R,10R,10aR,12R,14aR)-9-(4-acetamido-2-oxopyrimidin-1(2H)-yl)-5-(2-cyanoethoxy)-3,10-difluoro-2-mercapto-12-oxido-5-sulfidooctahydro-2H,7H-difuro[3,2-d:3′,2′-j][1,3,7,9]tetraoxa[2,8]diphosphacyclododecine-2-yl)-9H-purin-6-yl))dibenzamide (36): To a solution of compound 35 in CH₂Cl₂ (10 mL) was added water (0.08 mL) and a 6% (v/v) solution of DCA in CH₂Cl₂ (10 mL). After 10 min of stirring at room temperature, to the bright orange solution was introduced pyridine (2 mL), which changed the color of the solution from bright orange to yellow. The yellow solution was concentrated in vacuo until approximately 10 mL of the yellow mixture remained. To the yellow solution was introduced pyridine (12 mL) and the solution was evaporated until approximately 10 mL of the yellow mixture remained. On two successive occasions, to the yellow mixture was added pyridine (12 mL) and the solution was concentrated until approximately 8 mL of the yellow solution remained. To the stirring yellow solution in pyridine (8 mL) was added DMOCP (0.354 g, 2 mmol). After 5 min, to the dark orange solution was added water (0.3 mL), followed immediately by the introduction of 3H-1,2-benzodithiol-3-one (161 mg, 1.0 mmol). After 15 min, the yellow solution was poured into a 1N aqueous sodium bicarbonate solution (100 mL). After stirring for 30 min, the biphasic mixture was extracted with EtOAc (100 mL). After separating the layers, the aqueous layer was back extracted twice with EtOAc (100 mL). The organic extracts were combined and concentrated. To the concentrated yellow oil was added toluene (50 mL) and the mixture was evaporated to remove residual pyridine. This procedure was repeated with toluene (50 mL). The resulting oil was purified by silica gel chromatography (0% to 10% MeOH in CH₂Cl₂) to obtain of compound 36 (71 mg, 13%, a mixture of diastereomers) as a yellow solid.

Step 4: Triethylammonium (2R,3R,3aR,3R,7aR,9R,10R,10aR,12R,14aR)-2-(4-amino-2-oxopyrimidin-1-(2H)-yl)-9-(6-amino-9H-purin-9-yl)-3,10-difluorooctahydro-2H, 7H-difuro[3,2-d: 3′,2′-j][1,3,7,9]tetraoxa[2,8]diphosphacyclododecine-5,12-bis(thiolate) 5,12-dioxide (37) and and triethylammonium (2R,3R,3aR,5R,7aR,9R,10R,10aR,12S,14aR)-2-(4-amino-2-oxopyrimidin-1-(2H)-yl)-9-(6-amino-9H-purin-9-yl)-3,10-difluorooctahydro-2H, 7H-difuro[3,2-d: 3′,2′-j][1,3,7,9]tetraoxa[2,8]diphosphacyclododecine-5,12-bis(thiolate) 5,12-dioxide (37a): To a stirring solution of compound 36 (71 mg, 0.08 mmol) in methanol (1.0 mL) was added 30% v/v aqueous ammonium hydroxide (1 mL) and the orange slurry was heated to 50° C. After 4 h, the orange solution was allowed to cool and concentrated in vacuo to give a beige solid. The beige solid was dissolved in water (1 mL) and purified by reverse phase silica gel chromatography (0% to 20% MeCN in 10 mM aqueous TEAA) to obtain compound 37 (16.4 mg, 30%) as a white bis-triethylammonium salt after lyophilization and compound 37a (14.9 mg, 27%) as a white solid after lyophilization. Characterization for compound 37: LCMS-ESI: 668.90 [M−H]⁻ (calculated for C₁₉H₂₂F₂N₈O₉P₂S₂: 670.04); Rₜ: 7.129 min. ¹H NMR (400 MHz, 45° C., D₂O) δ 8.54 (s, 1H), 8.39 (s, 1H), 8.27 (d, J=7.6 Hz, 1H), 6.62 (d, J=17.2 Hz, 1H), 6.25 (d, J=16.8 Hz, 1H), 6.10 (d, J=7.6 Hz, 1H), 6.02 (d, J=49.6 Hz, 1H), 5.36 (d, J=50.4 Hz, 1H), 5.16-5.10 (m, 11H), 4.92-4.86 (m, 4H), 4.62-4.59 (m, 2H), 4.27-4.18 (m, 1H), 3.35 (q, J=7.2 Hz, 22H), 1.43 (t, J=7.2 Hz, 33-H). ¹⁹F NMR (400 MHz, 45° C., D₂O) δ −201.37 to −200.21 (m). ³¹P NMR (45° C., D₂O) δ 54.83, 54.34, 54.26. Characterization for compound 37a: LCMS-ESI: 668.90 [M−H]⁻ (calculated for C₁₉H₂₂F₂N₈O₉P₂S₂: 670.04); Rₜ: 6.257 min. ¹H NMR (400 MHz, 45° C., D₂O) δ 8.54 (s, 1H), 8.38 (s, 1H), 8.06 (d, J=8.0 Hz, 1H), 6.61 (d, J=18.0 Hz, 1H), 6.21 (d, J=18.0 Hz, 1H), 6.08 (d, J=6.4 Hz, 1H), 5.66 (d, J=50.4 Hz, 1H), 5.38 (d, J=54.4 Hz, 1H), 5.23-5.19 (m, 1H), 5.00-4.94 (m, 1H), 4.61-4.58 (m, 4H), 4.19-4.17 (m, 2H), 3.33 (q, J=7.2 Hz, 22H), 1.43 (t, J=7.2 Hz, 33H). ¹⁹F NMR (400 MHz, 45° C., D₂O) δ −200.24 to −199.33 (m). ³¹P NMR (45° C., D₂O) δ 54.77.

### Example 8: Synthesis of 3′2′-RR (ibG)(BzA) (42) and 3′2′ RS-(ibG)(BzA) (42)

3′2′-RR-(ibG)(BzA) (42), also referred to as dithio-[R_{P}, R_{P}]-cyclic-[ibG(3′,5′)p-BzA(2′,5′)p] and 3′2′-RS-(ibG)(BzA) (42a), also referred to as dithio-[R_{P}, S_{P}]-cyclic-[ibG(3′,5′)p-BzA(2′,5′)p], were prepared according to the following Scheme 8:

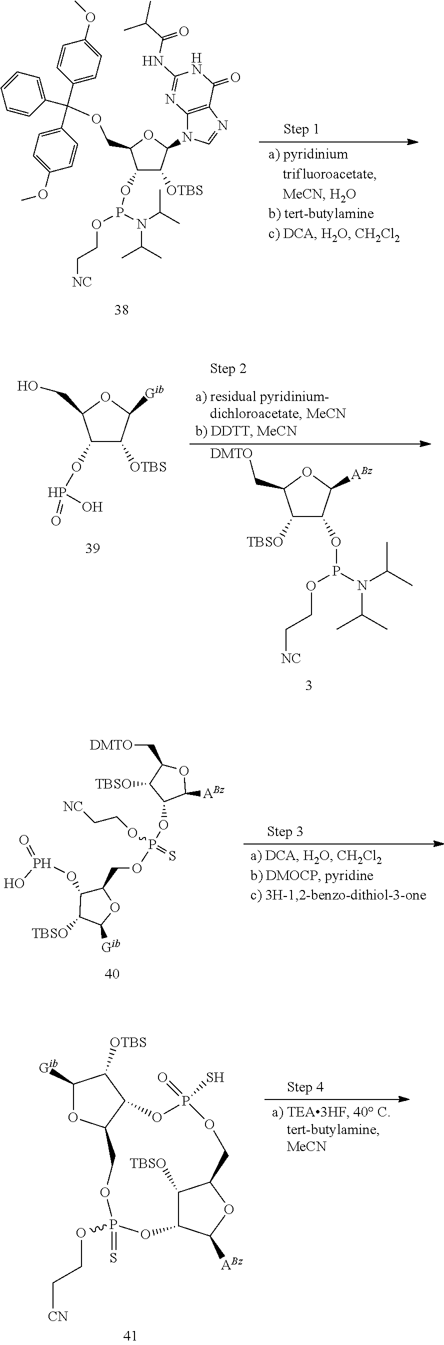

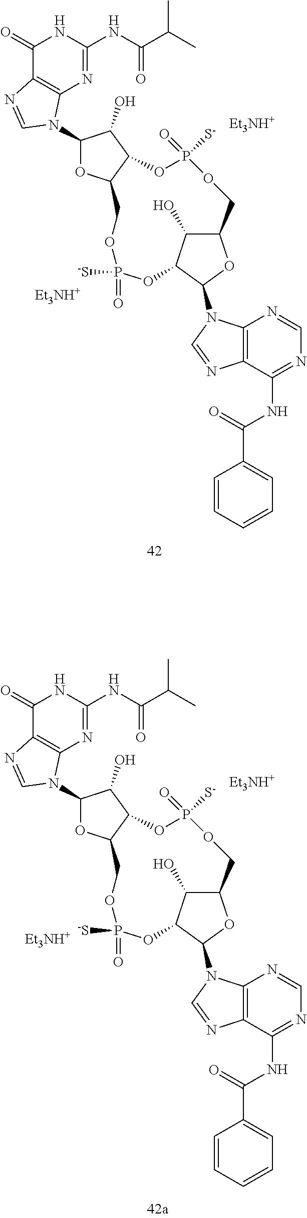

Step 1: (2R,3R,4R,5R)-4-((tert-butyldimethylsilyl)oxy)-2-(hydroxymethyl)-5-(2-isobutyramido-6-oxo-1,6-dihydro-9H-purin-9-yl)tetrahydrofuran-3-yl hydrogen phosphonate (39): To a solution of (2R,3R,4R,5R)-2-((bis(4-methoxyphenyl)(phenyl)methoxy)methyl)-4-((tert-butyldimethylsilyl)oxy)-5-(2-isobutyramido-6-oxo-1,6-dihydro-9H-purin-9-yl)tetrahydrofuran-3-yl (2-cyanoethyl) diisopropylphosphoramidite (38, 2.5 g, 2.6 mmol) in MeCN (18 mL) and H₂O (0.12 mL) was added pyridinium trifluoroacetate (0.65 mg, 3.4 mmol). After 15 min, to the colorless reaction solution was introduced tert-butylamine (12.5 mL). After 10 min, the colorless solution was concentrated under reduced pressure and water was removed as an azeotrope after concentration with MeCN (30 mL) to give a colorless foam of (2R,3R,4R,5R)-2-((bis(4-methoxyphenyl)(phenyl)methoxy)methyl)-4-((tert-butyldimethylsilyl)oxy)-5-(2-isobutyramido-6-oxo-1,6-dihydro-9H-purin-9-yl)tetrahydrofuran-3-yl hydrogen phosphonate. The colorless foam was dissolved in CH₂Cl₂ (30 mL) to give a colorless solution. To this colorless solution was added water (0.33 mL) and a 7.5% (v/v) solution of DCA in CH₂Cl₂ (25 mL). After 10 min of stirring at room temperature, to the orange solution was charged pyridine (3.0 mL), which turned the orange solution into a pale yellow solution. This yellow solution was concentrated under reduced pressure and water was removed as an azeotrope after concentration with MeCN (30 mL). This azeotrope process was repeated one more time with MeCN (30 mL). On the last evaporation, the resulting peach mixture of compound 39 was left in MeCN (15 mL).

Step 2: (2R,3R,4R,5R)-2-((((((2R,3R,4R,5R)-2-(6-benzamido-9H-purin-9-yl)-5-((bis(4-methoxyphenyl)(phenyl)methoxy)methyl)-4-((tert-butyldimethylsilyl)oxy)tetrahydrofuran-3-yl)oxy)(2-cyanoethoxy)phosphorothioyl)oxy)methyl)-4-((tert-butyldimethylsilyl)oxy)-5-(2-isobutyramido-6-oxo-1,6-dihydro-9H-purin-9-yl)tetrahydrofuran-3-yl hydrogen phosphonate (40): A solution of (2R,3R,4R,5R)-2-(6-benzamido-9H-purin-9-yl)-5-((bis(4-methoxyphenyl)(phenyl)methoxy)methyl)-4-((tert-butyldimethylsilyl)oxy)tetrahydrofuran-3-yl (2-cyanoethyl) diisopropylphosphoramidite (3, 3.0 g, 3.1 mmol) in MeCN (20 mL) was dried through concentration under reduced pressure until 7 mL of the solution remained. To this 7 mL solution was added MeCN (15 mL) and the solution was concentrated under reduced pressure until 7 mL of the solution remained. This process was repeated one more times to remove water as an azeotrope. On the last azeotrope, to the remaining solution in MeCN (7 mL) was introduced 3 Å molecular sieves and this solution was stored under an atmosphere of nitrogen. To a stirring mixture of compound 39 with residual pyridin-1-ium dichloroacetate in MeCN (15 mL) was added the solution of compound 3 in MeCN (7 mL). After 15 min, to the stirred mixture was added DDTT (597 mg, 2.9 mmol), which resulted in a yellow solution. After 45 min, the yellow solution was concentrated under reduced pressure to give the desired compound 40 as a yellow paste.

Step 3: Protected 3′2′-RR-(2′F-G)(A) (41): To a solution of compound 40 in CH₂Cl₂ (35 mL) was added water (0.4 mL) and a 12% (v/v) solution of DCA in CH₂Cl₂ (30 mL). After 10 min of stirring at room temperature, to the dark orange solution was introduced pyridine (15 mL), which turned the solution into a yellow solution. The yellow solution was concentrated under reduced pressure until approximately 15 mL of the yellow mixture remained. To the yellow mixture was introduced pyridine (30 mL) and the mixture was evaporated until approximately 15 mL of the yellow mixture remained. This azeotrope process was repeated two more times with pyridine (30 mL). To the remaining solution (15 mL) was added an additional amount of pyridine (30 mL). To the stirring yellow solution in pyridine (45 mL) was added DMOCP (1.5 g, 8.2 mmol). After 5 min, to the brownish-yellow solution was added water (1.5 mL), followed immediately by the introduction of 3H-1,2-benzodithiol-3-one (673 mg, 4.0 mmol). After 10 min, the brownish solution was poured into a 1N aqueous NaHCO₃ solution (400 mL). After 15 min, the biphasic mixture was extracted with EtOAc (250 mL). After the layers were separated, the aqueous layer was back extracted twice with EtOAc (250 mL and then 100 mL). The organic extracts were combined and concentrated. To the concentrated yellow paste was added toluene (30 mL) and the mixture was evaporated to remove residual pyridine. The resulting paste was purified by silica gel chromatography (0% to 15% MeOH in CH₂Cl₂) to obtain compound 41 (2.5 g, 82%) as a beige solid.

Step 4: 3′2′-RR-(ibG)(BzA) (42) and 3′2′-RS-(ibG)(BzA) (42a): To a portion of compound 41 (1.0 mg, 0.89 mmol) was introduced triethylamine trihydrofluoride (5.1 mL) and the yellow solution was heated to 40° C. After 3 h, the yellow solution was allowed to cool to room temperature. This yellow solution was slowly added to a cooled solution of LM TEAB (36 mL) and triethylamine (6 mL). The yellow mixture was allowed to stir for 1.5 h. The yellow mixture was purified by reverse phase silica gel chromatography (0% to 20% MeCN in 10 mM aqueous TEAA) to obtain the des-TBS product of 41 (96 mg, 0.1 mmol). To the des-TBS product of 41 in MeCN (6 mL) was added tert-butylamine (0.65 mL). After 1 h of stirring, the reaction solution was condensed to dryness under reduced pressure. The solid was suspended in 1:1 10 mM TEAA and MeCN; and the solution was purified by preparative-reverse phase silica gel chromatography (2% to 80% MeCN in 10 mM aqueous TEAA) to obtain compound 42 (43 mg, 47%) and compound 42a (14 mg, 15%), 42: LCMS-EST: 879.90 [M−H]⁻ (calculated for C₃₁H₃₄N₁₀O₁₃P₂S₂: 880.74); Rₜ: 6.57 min. ¹H NMR (400 MHz, 45° C., D₂O) δ 8.89 (s, 1H), 8.83 (s, 1H), 8.34 (s, 1H), 8.11-8.09 (d, J=6 Hz, 2H), 7.76-7.74 (m, 1H), 7.66-7.64 (m, 2H), 6.45 (s, 1H), 6.19 (s, 1H), 5.19-5.14 (m, 2H), 5.02 (m, 2H), 4.62-4.60 (m, 2H), 4.26-4.24 (m, 2H), 3.38-3.35 (q, J=12 Hz, 17H), 2.80 (m, 1H), 2.07 (s, 1H), 1.45-1.43 (t, J=4 Hz, 25H), 1.26-1.20 (dd, J=24 Hz, 6H). ³¹P NMR (45° C., D₂O) δ 54.21, 54.06. 42a: LCMS-ESI: 879.90 [M−H]⁻ (calculated for C₃₁H₃₄N₁₀O₁₃P₂S₂: 880.74); Rₜ: 6.46 min.

The compound 3′2′-SS-(G)(A) (43) was prepared from compound 41: To a stirred solution of compound 41 (939 mg, 0.81 mmol) in ethanol (8.0 mL) was added AMA (14.0 mL) and the yellow slurry was heated to 50° C. After 2 h, the yellow solution was allowed to cool and concentrated under reduced pressure. To a portion of the solid, beige product (534 mg, 0.57 mmol) was introduced triethylamine trihydrofluoride (5.1 mL) and the yellow solution was heated to 40° C. After 2 h, the yellow solution was allowed to cool to room temperature. This yellow solution was slowly added to a cooled solution of 1M TEAB (30 mL) and triethylamine (5 mL). The yellow mixture was allowed to stir for 1.5 h. The yellow mixture was purified by reverse phase silica gel chromatography (0% to 20% MeCN in 10 mM aqueous TEAA) to obtain compound 43 (10 mg, 2.5%) as a white solid after lyophilization. LCMS-ESI: 704.95 [M−H]⁻ (calculated for C₂₀H₂₄N₁₀O₁₁P₂S₂: 706.54); Rₜ: 5.17 min. ¹H NMR (400 MHz, 45° C., D₂O) δ 9.16 (s, 1H), 8.41 (s, 1H), 8.09 (s, 1H), 6.46-6.44 (d, J=8 Hz, 1H), 6.10 (s, 1H), 5.59-5.48 (m, 3H), 5.31 (s, 1H), 4.76 (s, 1H), 4.67 (s, 2H), 4.46-4.38 (m, 4H), 4.18-4.15 (m, 1H), 4.00 (m, 1H), 3.37-3.32 (q. J=20 Hz, 43H), 2.06 (s, 6H), 1.45-1.41 (t, J=16 Hz, 66H). ³¹P NMR (45° C., D₂O) δ 59.54, 56.61.

### Example 9: Synthesis of Beta-L-3′3′-SS-(A)(A) (48), Beta-L-3′3′-RS-(A)(A) (48a) and Beta-L-3′3′-RR-(A)(A) (48b)

(2S,3S,3aR,5S,7aS,9S,10S,10aR,12S,14aS)-2,9-bis(6-amino-9-purin-9-yl)-3,10-dihydroxy-5,12-dimercaptooctahydro-2H,7H-difuro[3,2-d:3′,2′-j][1,3,7,9]tetraoxa[2,8]diphosphacyclododecine 5,12-dioxide (48), also referred to as Beta-L-SS-(A)(A) or dithio-[S_{P}, S_{P}]-cyclic-L-A(3′,5′)p-L-A(3′,5′)p] and isomers Beta-L-RS-(A)(A) (48a) and Beta-L-RR-(A)(A) (48b) were prepared according to the following Scheme 9:

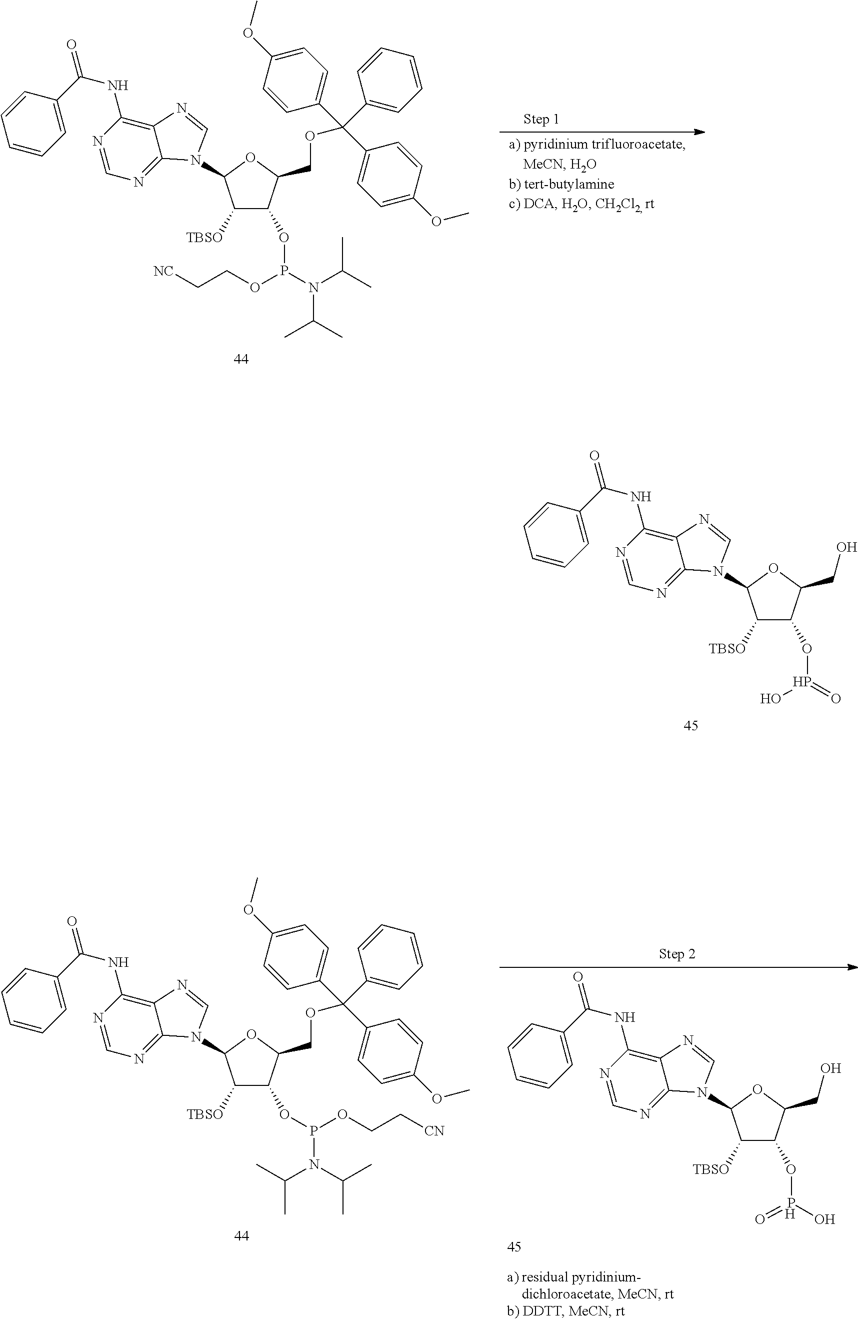

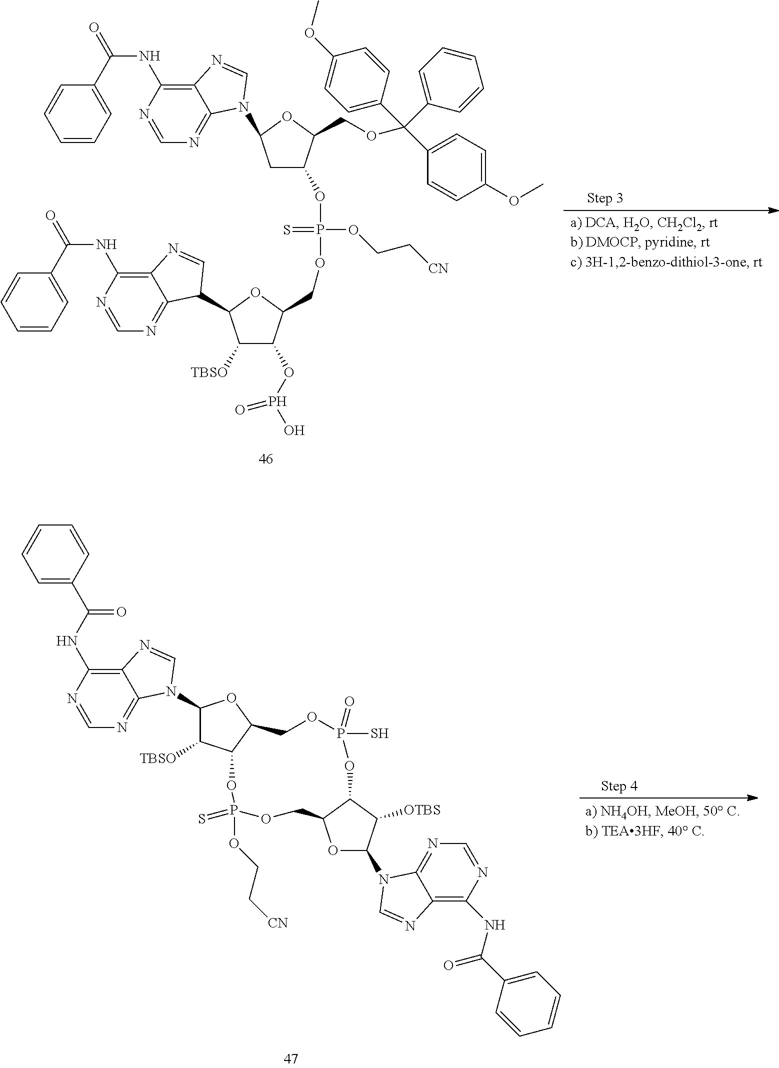

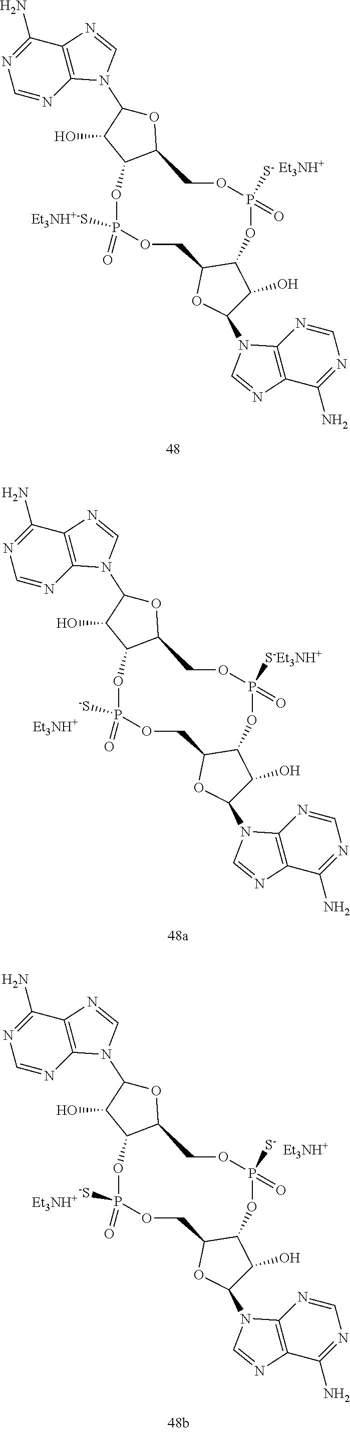

Step 1 (2S,3S,4S,5S)-5-(6-benzamido-9H-purin-9-yl)-4-((tert-butyldimethylsilyl)oxy)-2-(hydroxymethyl)tetrahydrofuran-3-yl hydrogen phosphonate (45) To a solution of (2S,3S,4S,5S)-5-(6-benzamido-9H-purin-9-yl)-2-((bis(4-methoxyphenyl)(phenyl)methoxy)methyl)-4-((tert-butyldimethylsilyl)oxy)tetrahydrofuran-3-yl (2-cyanoethyl) diisopropylphosphoramidite (44)(1.0 g, 1.0 mmol, ChemGenes) in MeCN (5.0 mL) and water (35 μL) was added pyridinium trifluoroacetate (0.23 g, 1.2 mmol). After 10 min, to the stirring reaction mixture at room temperature was added tert-butylamine (5.0 mL, 47.6 mmol). After 45 min, the reaction solution was concentrated in vacuo and water was removed as an azeotrope after concentration with MeCN (3×50 mL). After the last evaporation, the resulting colorless foam was dissolved in CH₂Cl₂ (14 mL) to give a colorless solution. To this solution was added water (0.18 mL) and a 6% (v/v) solution of DCA in CH₂Cl₂ (13 mL). After 11 min of stirring at room temperature, to the orange solution was charged pyridine (1.4 mL), which turned the orange solution into a colorless solution. After 11 min of stirring, the colorless solution was concentrated in vacuo and water was removed as an azeotrope after concentration with MeCN (15 mL). This azeotrope process was repeated three more times with MeCN (15 mL). On the last evaporation, the resulting white paste of compound 45 was dissolved in MeCN (15 mL).

Step 2: (2S,3S,4S,5S)-5-(6-benzamido-9H-purin-9-yl)-2-((((((2S,3S,4S,5S)-5-(6-benzamido-9H-purin-9-yl)-2-((bis(4-methoxyphenyl)(phenyl)methoxy)methyl)-4-((tert-butyldimethylsilyl)oxy)tetrahydrofuran-3-yl)oxy)(2-cyanoethoxy)phosphorothioyl)oxy)methyl)-4-((tert-butyldimethylsilyl)oxy)tetrahydrofuran-3-yl hydrogen phosphonate (46): A solution of compound 44 (1.05 g, 1.0 mmol) in MeCN (20 mL) was dried through concentration in vacuo and this process was repeated two more times with MeCN (2×20 mL). On the final azeotrope, the dried material was left in a solution of MeCN (6 mL) and eight pieces of 3 Å molecular sieves were introduced. This solution was stored under an atmosphere of nitrogen. To a stirring mixture of compound 45 with residual pyridinium dichloroacetate in MeCN (15 mL) from Step 1 was added the solution of compound 44 in MeCN (6 mL). After 7 min, to the stirring mixture was added DDTT (233 mg, 1.13 mmol), which resulted in a yellow mixture. After 25 min, the yellow mixture was concentrated in vacuo to give compound 46 as a yellow paste.

Step 3: N,N′-(((2S,3S,3aS,7aS,9S,10S,10aS,14aS)-3,10-bis((tert-butyldimethylsilyl)oxy)-5-(2-cyanoethoxy)-12-mercapto-12-oxido-5-sulfidooctahydro-2H, 7H-difuro[3,2-d: 3′,2′-j][1,3,7,9]tetraoxa[2,8]diphosphacyclododecine-2,9-diyl)bis(9H-purine-9,6-diyl))dibenzamide (47): To a solution of compound 46 in CH₂Cl₂ (20 mL) was added water (0.12 mL) and a 5% (v/v) solution of DCA in CH₂Cl₂ (19 mL). After 12 min of stirring at room temperature, to the orange solution was introduced pyridine (4 mL), which changed the color of the solution from orange to yellow. The yellow solution was concentrated in vacuo until approximately 20 mL of the yellow mixture remained. To the yellow solution was introduced pyridine (35 mL) and the solution was evaporated until approximately 30 mL of the yellow mixture remained. To the yellow mixture was added pyridine (2×30 mL) and the solution was concentrated in vacuo until approximately 35 mL of the yellow solution remained. To the stirring yellow solution in pyridine (35 mL) was added DMOCP (560 mg, 3.0 mmol). After 5 min, to the dark orange solution was added water (0.55 mL), followed immediately by the introduction of 3H-1,2-benzodithiol-3-one (255 mg, 1.5 mmol). After 15 min, the yellow solution was poured into a 1N aqueous NaHCO₃ solution (100 mL). After stirring for 30 min, the biphasic mixture was extracted with EtOAc (100 mL). After separating the layers, the aqueous layer was back extracted twice with EtOAc (2×100 mL). The organic extracts were combined and concentrated. To the concentrated yellow oil was added toluene (50 mL) and the mixture was evaporated to remove residual pyridine. This procedure was repeated with toluene (50 mL). The resulting oil was purified by silica gel chromatography (0% to 10% MeOH in CH₂Cl₂) to obtain a mixture of compound 47, (456 mg, 38%), as a yellow solid.

Step 4: (2S,3S,3aR,5S,7aS,9S,10S,10aR,12S,14aS)-2,9-bis(6-amino-9H-purin-9-yl)-3,10-dihydroxy-5,12-dimercaptooctahydro-2H,7H-difuro[3,2-d:3′,2′-j][1,3,7,9]tetraoxa[2,8]diphosphacyclododecine 5,12-dioxide (48): To a stirring solution of a mixture of compound 47 (122 mg, 0.1 mmol) in methanol (2.0 mL) at rt was added 30% v/v aqueous ammonium hydroxide (2.0 mL) and the yellow solution was heated to 50° C. After 2 h, the yellow solution was allowed to cool and concentrated in vacuo. To the residual solid (84 mg, 0.09 mmol) was introduced triethylamine trihydrofluoride (1.0 mL) and the yellow solution was heated to 40° C. After 4 h, the yellow solution was allowed to cool to room temperature. This yellow solution was slowly added to a cooled solution (in an ice-water bath 4° C.) of 1M TEAB (6 mL) and triethylamine (1.0 mL). The yellow mixture was allowed to stir for 40 min. The yellow mixture was purified by reverse phase silica gel chromatography (0% to 20% MeCN in 10 mM aqueous TEAA) to obtain the title compound 48 (8.7 mg, 14%) as a white bis-triethylammonium salt after lyophilization. Two other diastereomers were obtained from this sequence. Triethylammonium (2S,3S,3aR,5R,7aS,9S,10S,10aR,12S,14aS)-2,9-bis(6-amino-9H-purin-9-yl)-3,10-dihydroxyoctahydro-2H, 7H-difuro[3,2-d: 3′,2′-j][1,3,7,9]tetraoxa[2,8]diphosphacyclododecine-5,12-bis(thiolate) 5,12-dioxide (48a, Beta-L-RS-(A)(A)): (8.0 mg, 13%) was isolated as a white bis-triethylammonium salt after lyophilization and also recovered was triethylammonium (2S,3S,3R,5R,7aS,9S,10S,10aR,12R,14aS)-2,9-bis(6-amino-9H-purin-9-yl)-3,10-dihydroxyoctahydro-2H,7H-difuro[3,2-d: 3′,2′-j][1,3,7,9]tetraoxa[2,8]diphosphacyclododecine-5,12-bis(thiolate) 5,12-dioxide (48b, Beta-L-RR-(A)(A)): (3.5 mg, 6%). Characterization for (48): LCMS-ESI: 691.10 [M+H]⁺ (calculated for C₂₀H₂₄N₁₀O₁₀P₂S₂: 690.06); Rₜ: 1.938 min by UPLC. ¹H NMR (400 MHz, 45° C., D₂O) δ 8.54 (s, 2H), 8.31 (s, 2H), 6.31 (s, 2H), 5.19 (br s, 2H), 4.92 (s, 2H), 4.70-4.65 (m, 4H), 4.30-4.21 (m, 2H), 3.33 (q, J=7.2 Hz, 12H), 1.42 (t, J=7.2 Hz, 18H). ³¹P NMR (45° C., D₂O) δ 54.5. Characterization for (48a): LCMS-ESI: 691.05 [M+H]⁺ (calculated for C₂₀H₂₄N₁₀O₁₀P₂S₂: 690.06); Rₜ: 1.293 min by UPLC ¹H NMR (400 MHz, 45° C., D₂O) δ 8.69 (s, 1H), 8.55 (s, 1H), 8.36 (s, 1H), 8.33 (s, 1H), 6.33 (s, 2H), 5.25-5.10 (m, 3H), 4.90 (s, 1H), 4.70-4.51 (m, 4H), 4.25-4.20 (m, 2H), 3.34 (q, J=7.2 Hz, 12H), 1.42 (t, J=7.2 Hz, 18H). ³¹P NMR (45° C., D₂O) δ 55.13, 54.55. Characterization for (48b): LCMS-ESI: 691.05 [M+H]⁺ (calculated for C₂₀H₂₄N₁₀O₁₀P₂S₂: 690.06); Rₜ: 1.075 min by UPLC.

### Example 10: Synthesis of Additional Scaffold Molecules

Additional scaffold molecules can be prepared similarly to the above examples and prophetic examples. The compound dithio-3′2′-(2′OTBS-G)(3′OTBS-A), also referred to as dithio-cyclic-[2′ OTBS-G(3′,5′)p-3′OTBS-A(2′,5′)p] was prepared and the four isomers isolated as 3′2′-RR-(2′OTBS-G)(3′OTBS-A), 3′2′-SR-(2′OTBS-G)(3′OTBS-A), 3′2′-RS-(2′OTBS-G)(3′OTBS-A) and 3′2′-SS-(2′OTBS-G)(3′OTBS-A), as compounds 49a, 49b, 49c and 49d, respectively. The compound where stereochemistry is a mixture or unknown will be referenced as compound 49.

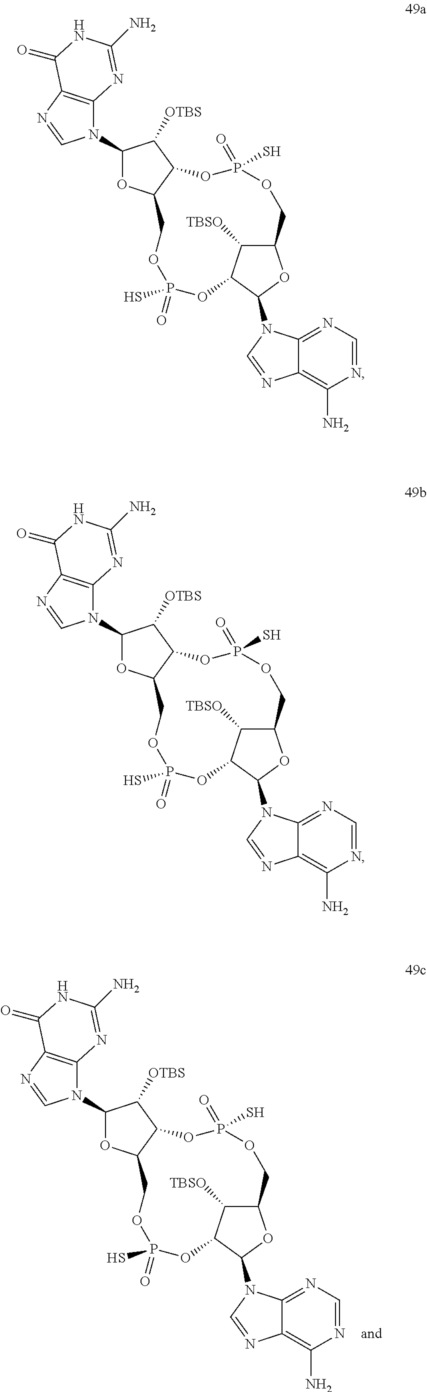 [SMILES: CC(C)(C)[Si](C)(C)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C]

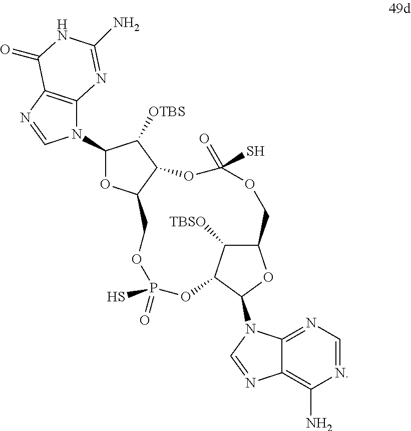

### Example 11: In Vitro Binding Analysis of Scaffold Molecules with Purified STING Protein

DNA encoding amino acids 140-379 (amino acid numbering corresponding to Swiss Prot Q86WV6) was amplified from plasmids containing the full length sequence of human STING alleles via polymerase chain reaction with the following primers: forward TACTTCCAATCCAATGCAGCCCCAGCTGAGATCTCTG (SEQ ID NO: 9) and reverse TTATCCACTTCCAATGTTATTATTATCAAGAGAAATCCGTGCCAG (SEQ ID NO: 10). STING variant alleles were assigned according to Yi, et al, (2013), PLoS One, 8(10), e77846 (DOI: 10.1371/journal.pone.0077846. PCR products were cloned into bacterial expression vector encoding a N-terminal hexa-histidine affinity tag (6×HIS) followed by a small ubiquitin-like modifier (SUMO) solubility sequence (Butt, et al, (2005) Protein expression and purification 43.1, 1-9) and tobacco etch virus protease cleavage site (TEV) using ligation independent cloning (Aslanidis, et al, (1990) Nucleic acids research, 18.20, 6069-6074).

Plasmids encoding 6×HIS-SUMO-TEV-STING amino acids 140-379 were transformed into Rosetta2 (DE3) E. coli cells (EMD Millipore) for protein expression. Cells were grown in lysogeny broth at 37° C. until a 600 nM absorbance of 0.6 was reached. Cells were then transferred to 18° C. and protein expression was induced overnight by the addition of isopropyl β-D-1-thiogalactopyranoside to the media at a concentration of 0.25 mM. Cells were harvested by centrifugation at 6,000 times gravity for 10 minutes. Cell pellets were re-suspended on ice in a buffer containing 50 mM Tris hydrochloride (Tris-HCl) pH 7.5, 500 mM sodium chloride (NaCl), 20 mM imidazole, 10% glycerol, 1 mM tris(2-carboxyethyl)phosphine hydrochloride (TCEP) and protease inhibitor tablet (Pierce) (Buffer A). Cells were lysed using an S-450D sonifier (Emmerson industrial) on ice. Cell lysate was centrifuged at 15,000 times gravity for 30 minutes at 4° C. Soluble material was applied to nickel-nitrilotriacetic acid (Ni-NTA) coupled Sepharose CL-6B (Qiagen) for 1 hour with gentle rocking at 4° C. After transfer to a gravity flow poly-prep column (Bio-Rad), resin was washed extensively in buffer A. Protein was eluted from the column in a buffer containing 20 mM Tris-HCl pH 7.5, 150 mM NaCl, 300 mM imidazole, 10% glycerol and 0.5 mM TCEP. To remove the 6×HIS-SUMO tag eluted protein was mixed with TEV protease (Sigma) at a ratio of 1:250 (w:w) and dialyzed overnight against a buffer containing 20 mM Tris-HCl pH 7.5, 150 mM NaCl, 5 mM imidazole, 10% glycerol and 0.5 mM TCEP. TEV protease and 6×HIS-SUMO tags were depleted by the addition of Ni-NTA resin (Qiagen) to the sample, purified STING amino acids 140-379 was collected by removal of the resin using a poly-prep column STING AA140-379 was concentrated with a 10,000 Dalton molecular weight cutoff centrifuge concentrator (EMD Millipore) to a final concentration of approximately 10 mg/ml. Protein was aliquoted, flash frozen in liquid nitrogen and stored at −80° C. until use.

Differential scanning fluorometry (DSF) is a technique that measures the ability of ligands to bind to and stabilize purified proteins (Niesen, et al, (2007) Nature protocols 2.9, 2212-2221). The protein is heated in the presence of a dye that binds to and fluoresces in hydrophobic environments. The protein is thermally denatured by heating resulting in increased dye binding to the unfolded protein and fluorescence. The temperature midpoint (Tₘ) of a proteins denaturation is established by calculating the half maximal value of the denaturation curve. The temperature midpoint of the protein in the presence of a ligand is directly related to the affinity of the ligand for the protein and therefore its ability to stabilize the protein at higher temperatures.

DSF was performed in a 20 μL reaction comprising 20 mM Tris-HCl pH 7.5, 150 mM NaCl, 1:500 dilution of SYPRO Orange (Life Technologies), 1 mg/ml purified STING AA140-379 protein and ligand at a concentration of 1 mM. Wild type hSTING, HAQ allele hSTING and/or REF allele hSTING were used with scaffold molecules of the invention as listed in Table 2. SR-(2′F-A)(2′OTBS-A) compound 18a resulted in high background fluorescence in this assay and was not able (NA) to determine the Tₘ shift.

TABLE 2
Tₘ shifts in hSTING WT, REF allele and HAQ allele

| | | hSTING Tₘ Shift | | |
|---|---|---|---|---|
| | | (° C.) | | |
| Example/Scaffold | Compound name | WT | REF | HAQ |
| Example 1 | 2′3′-RR-(3′OTBS-A)(2′F-A) | | | 18.1 |
| Compound 6 | | | | |
| Example 1 | 2′3′-SR-(3′OTBS-A)(2′F-A) | 9.0 | 4.8 | 20.2 |
| Compound 6a | | | | |
| Example 2 | 2′3′-RR-(A)(2,6-DAP) | | | 14.3 |
| Compound 12 | | | | |
| Example 2 | 2′3′-SR-(A)(2,6-DAP) | | | 14.5 |
| Compound 12a | | | | |
| Example 3 | SR-(2′F-A)(2′OTBS-A) | NA | NA | NA |
| Compound 18a | | | | |
| Example 4 | RR-(2,6-DAP)(2,6-DAP) | 12.2 | 8.3 | 15.1 |
| Compound 23 | | | | |
| Example 4 | RS-(2,6-DAP)(2,6-DAP) | 10.0 | 3.1 | 6.3 |
| Compound 23a | | | | |
| Example 5 | (6-O-propargyl-G)(G) | | | 7.2 |
| compound 25 | | | | |
| Example 5 | (6-O-propargyl-G)(6-O- | | | 5.4 |
| compound 26 | propargyl-G) | | | |
| Example 6 | RR-(2′F-ibG)(2′F-ibG) | 10.7 | 10.5 | 11.5 |
| Compound 32 | | | | |
| Example 6 | RS-(2′F-ibG)(2′F-ibG) | | | 9.9 |
| Compound 32a | | | | |
| Example 7 | RR-(2′F-C)(2′F-A) | 8.2 | 7.5 | 10.3 |
| Compound 37 | | | | |
| Example 7 | RS-(2′F-C)(2′F-A) | 5.6 | 5.3 | 6.4 |
| Compound 37a | | | | |
| Example 8 | 3′2′-RR-(ibG)(BzA) | | | 7.6 |
| Compound 42 | | | | |
| Example 8 | 3′2′-RS-(ibG)(BzA) | | | 4.2 |
| Compound 42a | | | | |
| Example 8 | 3′2′-SS-(G)(A) | | | 10.5 |
| Compound 43 | | | | |
| Example 9 | Beta-L-SS-(A)(A) | 2.0 | 2.3 | 2.3 |
| Compound 48 | | | | |
| Example 9 | Beta-L-RS-(A)(A) | 2.9 | 6.3 | 6.6 |
| Compound 48a | | | | |
| Example 9 | Beta-L-RR-(A)(A) | 3.0 | 6.4 | 3.5 |
| Compound 48b | | | | |

### Example 12: Scaffold Molecules do not Activate Human STING Signaling in THP1 Cells

The scaffold molecules assessed in Example 11 were also assessed to determine their activity as STING agonists by measuring their ability to induce type I interferon production in human cells. THP1-Dual cells (a human monocyte cell line containing the hSTING HAQ allele transfected with an IRF-3 inducible secreted luciferase reporter gene (Invivogen) which express secreted luciferase under the control of a promoter comprised of five IFN-stimulated response elements) were used in the assay. These cells (100,000) were activated with 30 ng/ml phorbol 12-myristate 13-acetate overnight in a 96-well dish. Cells were washed with fresh media and incubated for 30 min at 37° C. with 5% CO₂ with compounds in 3 fold titration steps from 2,000 to 0.0338 μM in PB buffer (50 mM 4-(2-hydroxyethyl)-1-piperazineethanesulfonic acid, 100 mM KCl, 3 mM MgCl₂, 0.1 mM dithiothreitol, 85 mM sucrose, 1 mM ATP, 0.1 mM GTP and 0.2% bovine serum albumin). To measure type I interferon activation with uniform compound cell penetration, cells were stimulated with compounds in 4 fold titration steps from 12 to 0.00001 μM in PB buffer containing 10 μg/ml digitonin. After 30 minutes, cells were washed and fresh RPMI media containing 10% FBS was added, and cells were incubated at 37° C. with 5% CO₂. Cell culture supernatants from each sample were collected after overnight incubation, and 10 μL of the cell culture supernatants was added to 50 μL QUANTI-Luc reagent (Invivogen). Type I interferon activation was determined by measuring secreted luciferase levels on a SpectraMax M3 spectrophotometer (Molecular Devices). The results of this assay with and without digitonin indicates that these compounds are not agonists of the STING-dependent type I interferon production. The scaffold molecule RR-(2′F-ibG)(2′F-ibG) (Example 6 compound 32) assayed without digitonin is compared to the agonist 2′3′-RR-(A)(A) in this assay in FIG. 1A, and the scaffold molecules RR-(2,6-DAP)(2,6-DAP) and RS-(2,6-DAP)(2,6-DAP) (Example 4, compounds 23 and 23a) assayed without digitonin are compared to the agonist 2′3′-RR-(A)(A) in this assay in FIG. 1B.

### Example 13: Antagonist Activity of Scaffold Molecules in THP-1 Competition Assay with 2′3′-RR-(A)(A)

The scaffold molecules and STING inhibitory compounds as described herein are assayed for their ability to inhibit the induction of STING dependent IFN-β production by an agonist compound in a competition assay. Relative induction of IRF-3 signaling in THP-1 cells (HAQ allele) was measured after stimulating the cells with a mix of a scaffold molecule (or any putative STING antagonist) titrated against a constant concentration of the STING agonist 2′3′-RR-(A)(A) in PB buffer (50 mM HEPES pH 7.5, 100 mM KCl, 3 mM MgCl₂, 0.1 mM DTT, 85 mM sucrose, 0.2% BSA, 1 mM ATP, 0.1 mM GTP).

One day prior to performing the assay, THP-1 dual (Invivogen) reporter cells were plated at 500,000 cells/mL in 200 μL per well in a 96-well plate and differentiated overnight with 3 μg/mL phorbal myristate acetate in RPMI medium 1640 (4.5 g/L D-glucose, 2.383 g/L HEPES, 300 mg/L L-glutamine, 1.5 g/L sodium bicarbonate, 110 mg/L sodium pyruvate, 10% heat-inactivated fetal bovine serum, 1× penicillin/streptomycin, 0.1 mg/mL normocin).

On the day of the assay, the media was removed from the wells and replaced with 150 μL of fresh media. The titrations against 2′ 3′-RR-(A)(A) in a final volume of 100 μL were made as follows: two-fold dilutions (2,000 μM-3.9 μM) of the scaffold molecule into PB buffer containing 100 μM 2′3′-RR-(A)(A) for a final concentration of 50 μM 2′3′-RR-(A)(A). Compounds were tested in biological triplicate.

The cells were then stimulated with 25 μL of each titration for 30 minutes at 37° C. in a CO₂ incubator. After stimulating, the cells were washed twice, first with 100 μL and then 150 μL of fresh media. After washing, 150 μL of media was added to each well and the cells were incubated for 17 hours overnight at 37° C. in a CO₂ incubator.

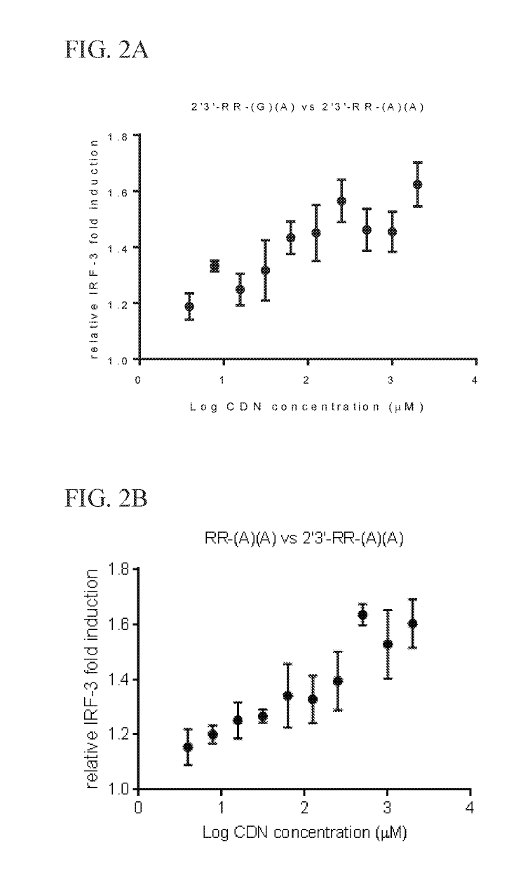

To detect IRF-3 signaling, 50 μL of luciferase substrate (Invivogen QUANTI-Luc coelenterazine-based luminescence reagent reconstituted in 25 mL water) was added to 10 μL of supernatant from each well in a 96-well white opaque plate. Luminescence was measured using a SpectraMax M3 plate reader (Molecular Devices) and relative fold-induction was calculated as the values obtained from wells containing both the scaffold molecule and 2′3′-RR-(A)(A) divided by the values obtained from wells containing 2′3′-RR-(A)(A) alone. The average of the relative fold-induction was plotted against the log concentration of the antagonist. Agonist compounds 2′3′-RR-(G)(A) and RR-(A)(A) were run as controls in this assay (see FIGS. 2A and 2B).

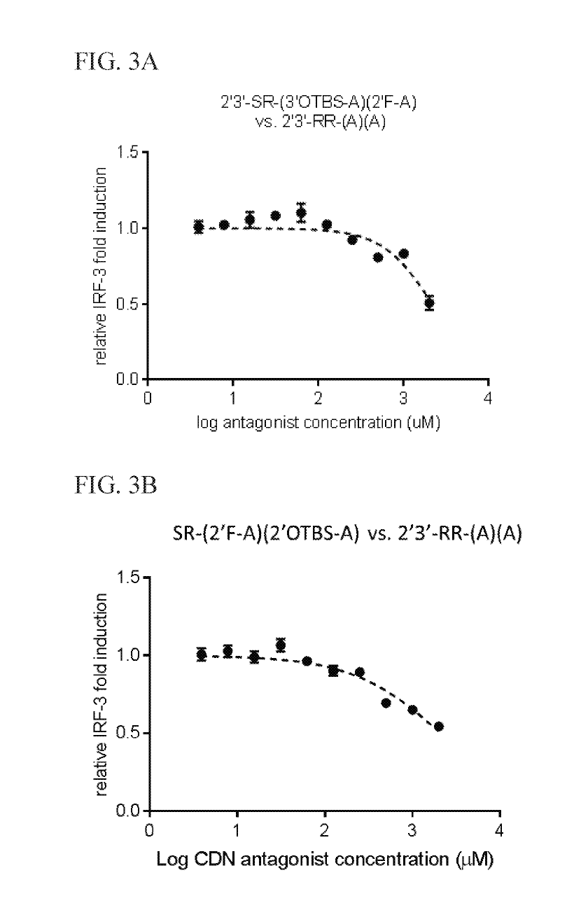

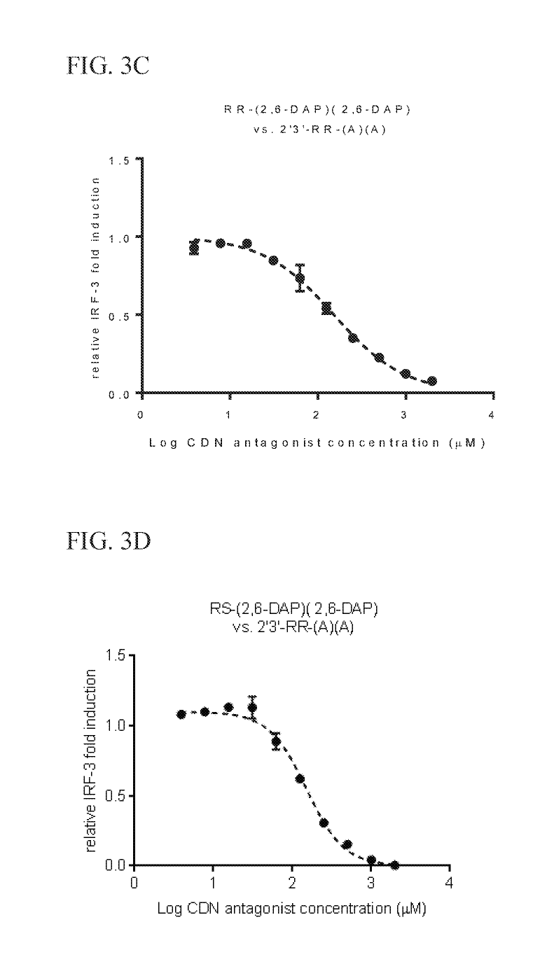

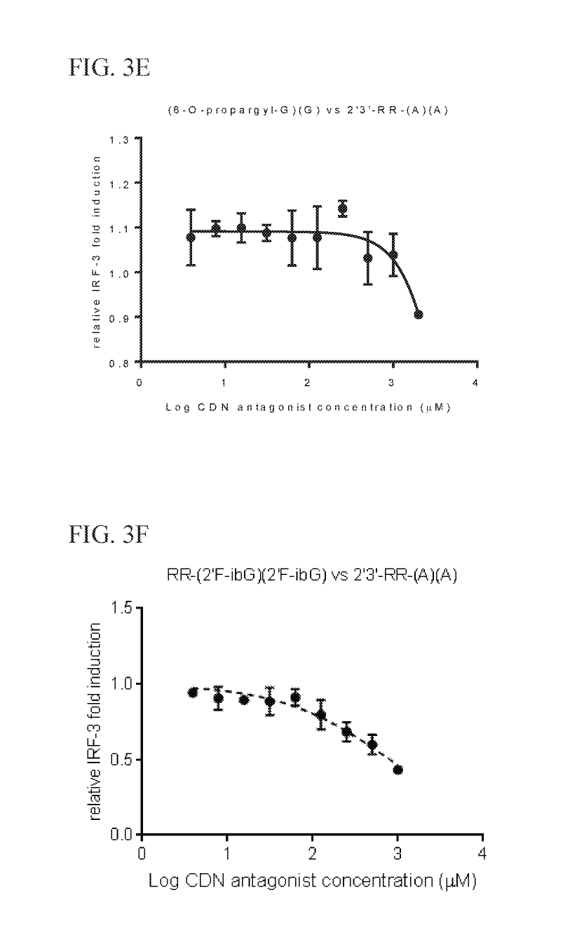

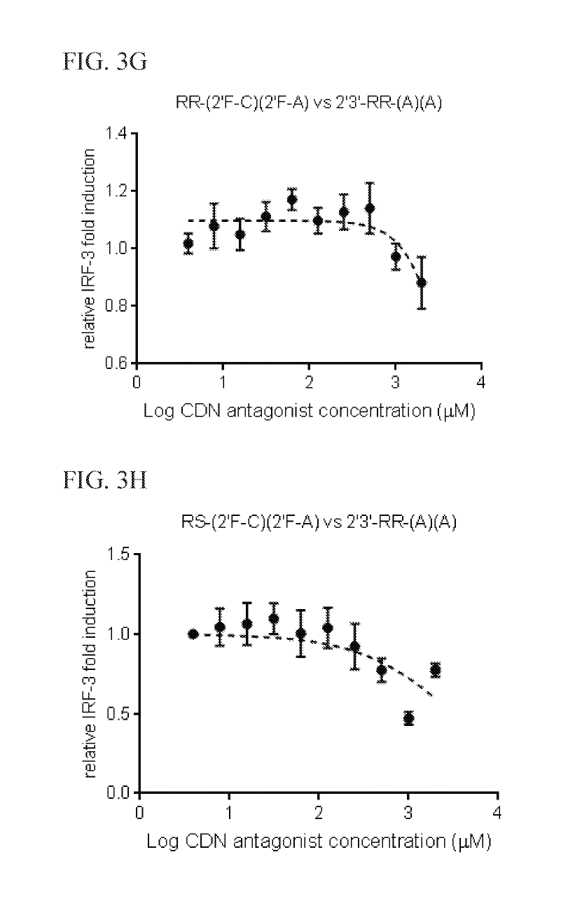

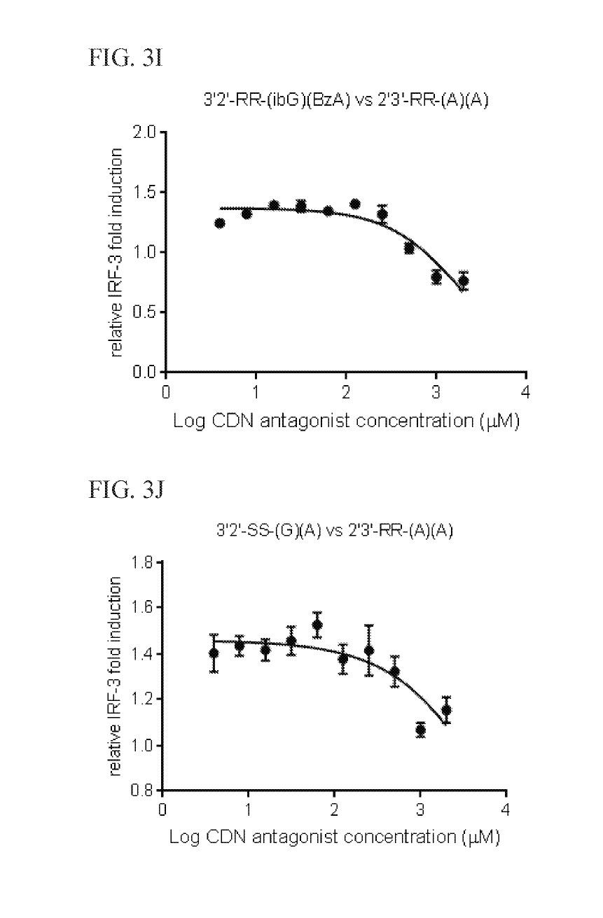

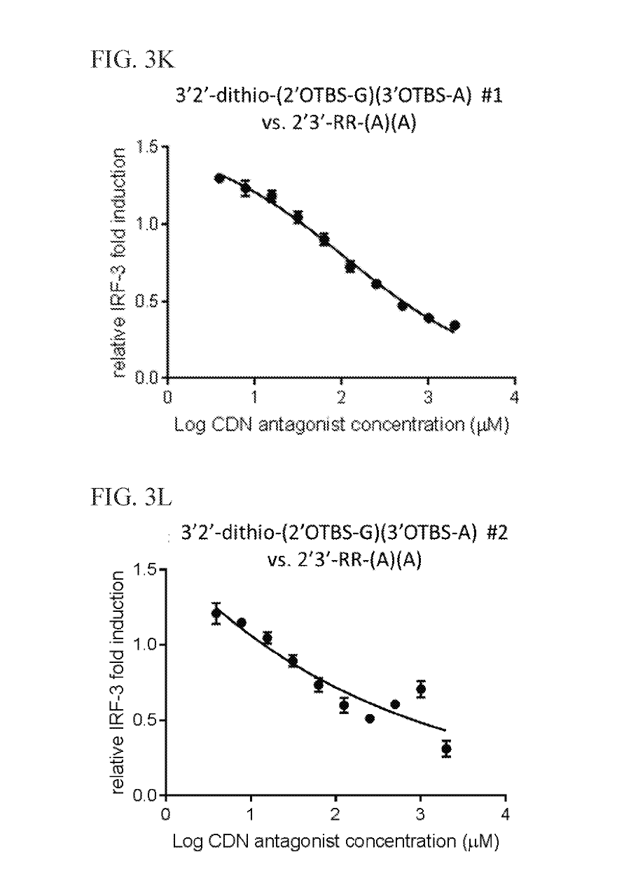

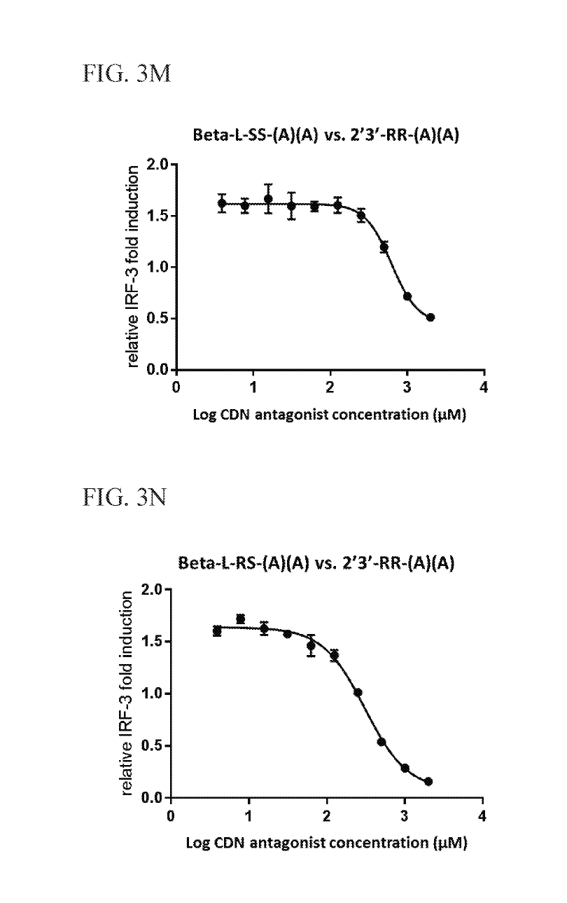

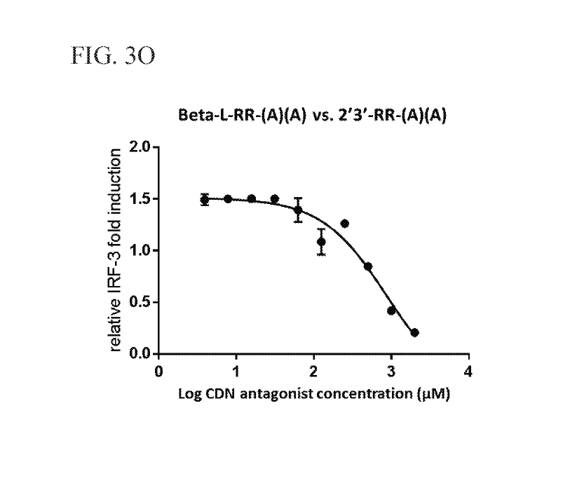

The results are provided for control compounds in FIGS. 2A and 2B and scaffold molecules in FIGS. 3A-3L: 2′3′-SR-(3′OTBS-A)(2′F-A) (FIG. 3A, Comp. 6a), SR-(2′F-A)(2′OTBS-A) (FIG. 3B, Comp. 18a), RR-(2,6-DAP)(2,6-DAP) (FIG. 3C, Comp. 23), RS-(2,6-DAP)(2,6-DAP) (FIG. 3D, Comp. 23a), (6-O-propargyl-G)(G) (FIG. 3E, Comp. 25), RR-(2′F-ibG)(2′F-ibG) (FIG. 3F, Comp. 32), RR-(2′F-C)(2′F-A) (FIG. 3G, Comp. 37), RS-(2′F-C)(2′F-A) (FIG. 3H, Comp. 37a), 3′2′-RR-(ibG)(BzA) (FIG. 3I, Comp. 42), 3′2′-SS-(G)(A) (FIG. 3J, Comp. 43), dithio-3′2′-(2′OTBS-G)(3′OTBS-A) isomer 1 (FIG. 3K, Comp. 49, isomer 1), dithio-3′2′-(2′OTBS-G)(3′OTBS-A) isomer 2 (FIG. 3L, Comp. 49, isomer 2), Beta-L-SS-(A)(A) (FIG. 3M, Comp. 48), Beta-L-RS-(A)(A) (FIG. 3N, Comp. 48a), and Beta-L-RR-(A)(A) (FIG. 3O, Comp. 48b). Isomer 1 and 2 of Compound 49 are different isomers, and are each one of compound 49a, 49b, 49c or 49d. The scaffold molecules demonstrate antagonist activity in the competition assay with 2′3′-RR-(A)(A) without digitonin. The IC50 was determined from these plots, and are provided in the following Table 3.

TABLE 3
IC50 inhibition of 2′3′-RR-(A)(A) agonist activity
without digitonin in THP1 cells (HAQ allele).

| Example/Scaffold | Compound name | IC50 (μM) |
|---|---|---|
| Example 1 | 2′3′-SR-(3′OTBS-A)(2′F-A) | 2174 |
| Compound 6a | | |
| Example 3 | SR-(2′F-A)(2′OTBS-A) | 2110 |
| Compound 18a | | |
| Example 4 | RR-(2,6-DAP)(2,6-DAP) | 152 |
| Compound 23 | | |
| Example 4 | RS-(2,6-DAP)(2,6-DAP) | 150 |
| Compound 23a | | |
| Example 5 | (6-O-propargyl-G)(G) | 5012 |
| compound 25 | | |
| Example 6 | RR-(2′F-ibG)(2′F-ibG) | 803 |
| Compound 32 | | |
| Example 7 | RR-(2′F-C)(2′F-A) | 4120 |
| Compound 37 | | |
| Example 7 | RS-(2′F-C)(2′F-A) | 3196 |
| Compound 37a | | |
| Example 8 | 3′2′-RR-(ibG)(BzA) | 966 |
| Compound 42 | | |
| Example 8 | 3′2′-SS-(G)(A) | 4041 |
| Compound 43 | | |
| Example 9 | Beta-L-SS-(A)(A) | 662 |
| Compound 48 | | |
| Example 9 | Beta-L-RS-(A)(A) | 308 |
| Compound 48a | | |
| Example 9 | Beta-L-RR-(A)(A) | 342 |
| Compound 48b | | |
| Example 10 | Dithio-3′2′-(2′OTBS-G) | 109 |
| compound 49 | (3′OTBS-A) | |
| isomer 1 | | |
| Example 10 | Dithio-3′2′-(2′OTBS-G) | 420 |
| compound 49 | (3′OTBS-A) | |
| isomer 2 | | |

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

1. A scaffold molecule having the structure of Formula I, Formula II, Formula III, Formula IV or Formula V: 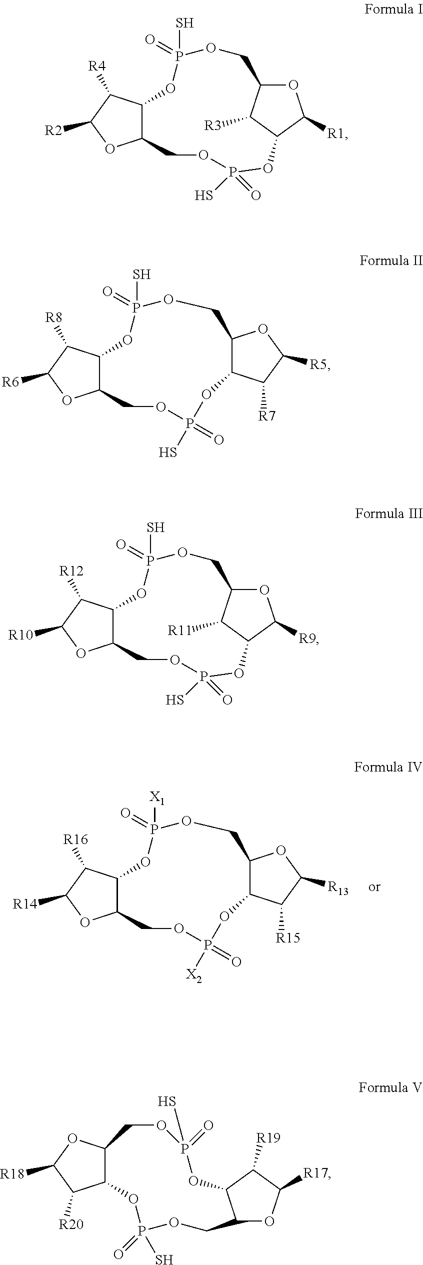
wherein:
R1 is adenine or adenine-6-benzamide linked to the structure via the N9 position;
R2 is guanine or guanine-2-isobutyramide linked to the structure via the N9 position;
R3 is —OH;
R4 is —OH;
R5 and R6 are both guanine-2-isobutyramide linked to the structure via the N9 position; or one of R5 and R6 is adenine linked to the structure via the N9 position, and the other of R5 and R6 is cytosine linked to the structure via the N1 position;
R7 is —F;
R8 is —F;
R9 is adenine linked to the structure via the N9 position;
R10 is adenine, guanine or 2,6-diamino-purine linked to the structure via the N9 position;
R11 is —OH or —OTBS;
R12 is —F, —OH or —OTBS;
R13 is adenine, 2,6-diamino-purine, guanine or guanine-6-propargyl ether linked to the structure via the N9 position;
R14 is adenine, 2,6-diamino-purine, guanine or guanine-6-propargyl ether linked to the structure via the N9 position;
R15 is —F, —OH or —OTBS;
R16 is —F, —OH or —OTBS;
R17 and R18 are both adenine linked to the structure via the N9 position;
R19 is —OH;
R20 is —OH; and
X₁ and X₂ are independently —OH or —SH;
wherein the scaffold molecule (i) exhibits measurable STING inhibitory activity and/or (ii) exhibits measurable STING binding but is not a STING agonist.

2. The scaffold molecule according to claim 1, wherein the scaffold molecule measurably binds to one or more of wild type hSTING, hSTING HAQ allele, or hSTING REF allele.

3. The scaffold molecule according to claim 2, wherein binding to hSTING is measured by Tₘ shift in a differential scanning fluorometry assay.

4. The scaffold molecule according to claim 3, wherein the Tₘ shift is measured according to the assay of Example 11, and the Tₘ shift is in the range of about 2 to about 15° C. for WT hSTING or hSTING REF allele and in the range of about 2 to about 25° C. for hSTING HAQ allele.

5. The scaffold molecule according to claim 1, wherein the measurable STING inhibitory activity is measured in a competition assay with a STING agonist.

6. The scaffold molecule according to claim 5, wherein the STING agonist is 2′3′-RR-(A)(A).

7. The scaffold molecule according to claim 5, wherein the scaffold molecule has an IC50 in the competition assay of less than 10 mM.

8. The scaffold molecule according to claim 5, wherein the scaffold molecule has an IC50 in the competition assay of less than 5 mM.

9. The scaffold molecule according to claim 5, wherein the scaffold molecule has an IC50 in the competition assay in the range of 100 μM to 5 μM.

10. (canceled)

11. The scaffold molecule according to claim 1 wherein X₁ and X₂ are —SH.

12. The scaffold molecule according to claim 1, wherein the scaffold molecule is of Formula Ia, Ib, Ic, Id, IIa, IIb, IIc, IId, IIIa, IIIb, IIIc, IIId, IVa, IVb, IVc, IVd, IVe, Va, Vb, Vc or Vd: 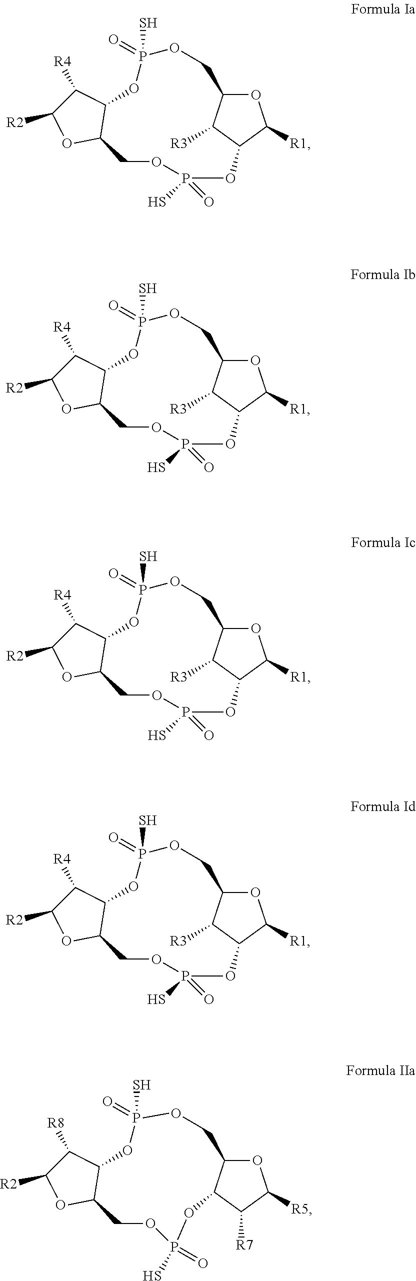 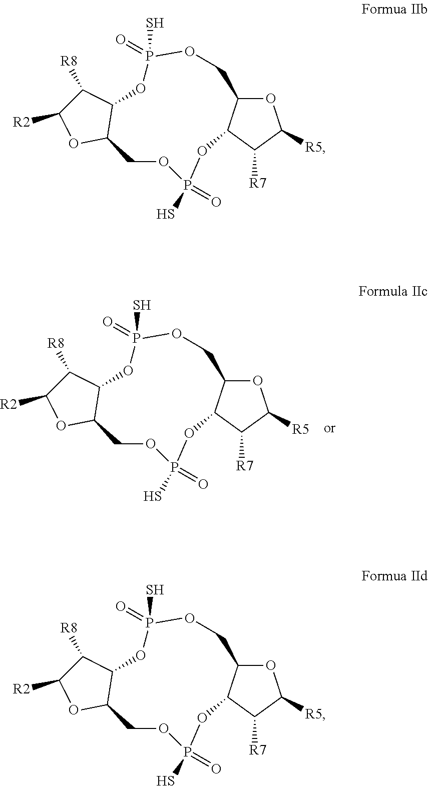
wherein R1, R2, R3, R4, R5, R6, R7, R8, R9, R10, R11, R12, R13, R14, R15, R16, R17, R18, R19, R20, X₁ and X₂ are as defined in claim 1.

13. The scaffold molecule according to claim 1, wherein the scaffold molecule is selected from the group consisting of:

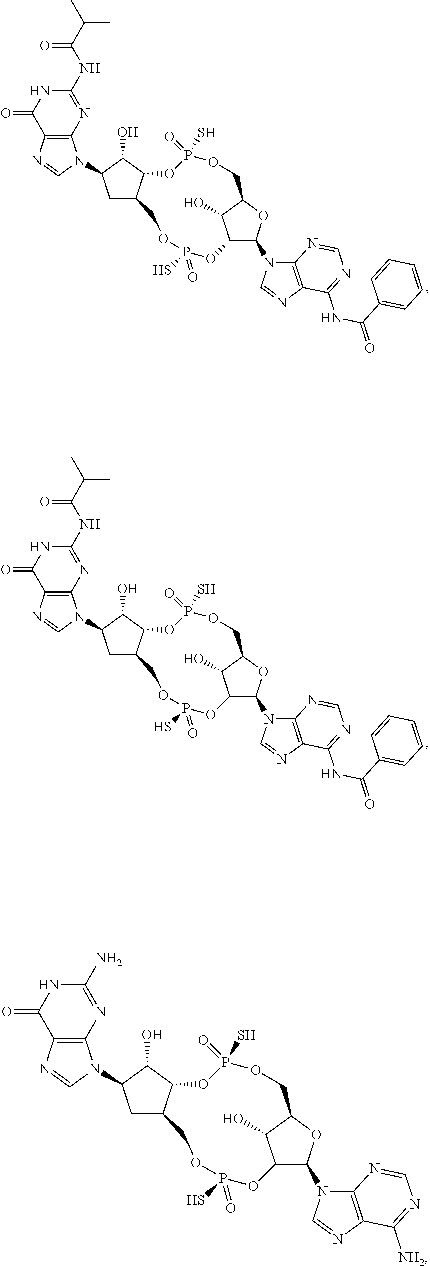 [SMILES: CC(C)C(=O)Nc1nc2c(ncn2[C@@H]2C[C@@H]3CO[P@@](=O)(S)O[C@@H]4[C@H](O)[C@@H](CO[P@@](=O)(S)O[C@H]3[C@H]2O)O[C@H]4n2cnc3c(NC(=O)c4ccccc4)ncnc32)c(=O)[nH]1 ; CC(C)C(=O)Nc1nc2c(ncn2[C@@H]2C[C@@H]3CO[P@](=O)(S)OC4[C@H](n5cnc6c(NC(=O)c7ccccc7)ncnc65)O[C@H](CO[P@@](=O)(S)O[C@H]3[C@H]2O)[C@H]4O)c(=O)[nH]1 ; Nc1nc2c(ncn2[C@@H]2C[C@@H]3CO[P@](=O)(S)OC4[C@H](n5cnc6c(N)ncnc65)O[C@H](CO[P@](=O)(S)O[C@H]3[C@H]2O)[C@H]4O)c(=O)[nH]1]

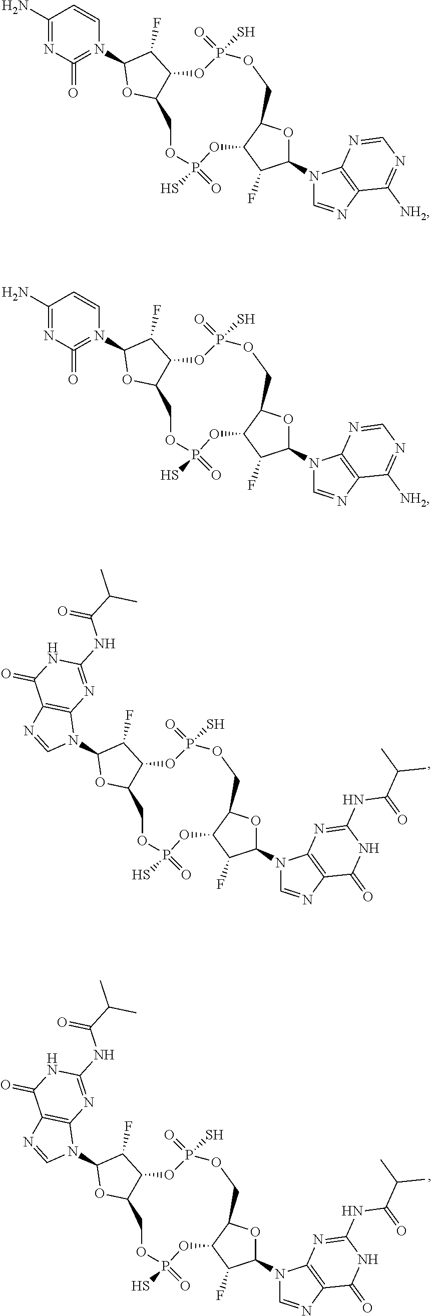

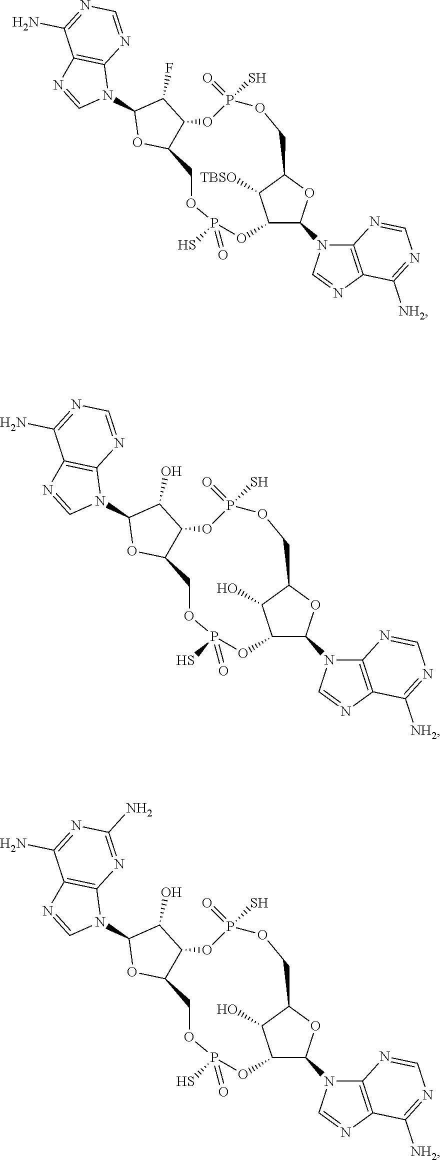 [SMILES: CC(C)(C)[Si](C)(C)O[C@H]1[C@H]2O[P@](=O)(S)OC[C@H]3O[C@@H](n4cnc5c(N)ncnc54)[C@H](F)[C@@H]3O[P@](=O)(S)OC[C@H]1O[C@H]2n1cnc2c(N)ncnc21 ; Nc1ncnc2c1ncn2[C@@H]1O[C@@H]2CO[P@](=O)(S)O[C@@H]3[C@H](O)[C@@H](CO[P@@](=O)(S)O[C@H]2[C@H]1O)O[C@H]3n1cnc2c(N)ncnc21 ; Nc1nc(N)c2ncn([C@@H]3O[C@@H]4CO[P@@](=O)(S)O[C@@H]5[C@H](O)[C@@H](CO[P@@](=O)(S)O[C@H]4[C@H]3O)O[C@H]5n3cnc4c(N)ncnc43)c2n1]

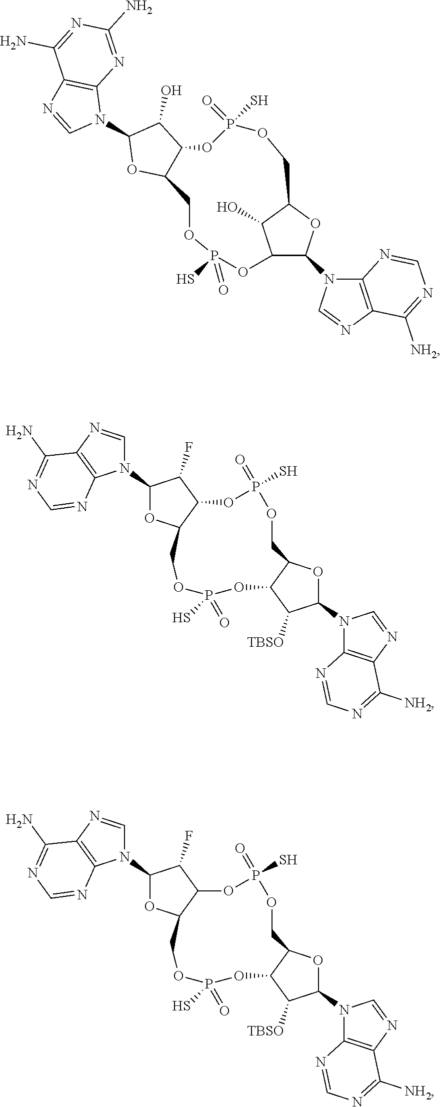 [SMILES: Nc1nc(N)c2ncn([C@@H]3O[C@@H]4CO[P@](=O)(S)OC5[C@H](n6cnc7c(N)ncnc76)O[C@H](CO[P@@](=O)(S)O[C@H]4[C@H]3O)[C@H]5O)c2n1 ; CC(C)(C)[Si](C)(C)O[C@@H]1[C@@H]2O[P@](=O)(S)OC[C@H]3O[C@@H](n4cnc5c(N)ncnc54)[C@H](F)[C@@H]3O[P@](=O)(S)OC[C@H]2O[C@H]1n1cnc2c(N)ncnc21 ; CC(C)(C)[Si](C)(C)O[C@@H]1[C@@H]2O[P@](=O)(S)OC[C@H]3O[C@@H](n4cnc5c(N)ncnc54)[C@H](F)C3O[P@@](=O)(S)OC[C@H]2O[C@H]1n1cnc2c(N)ncnc21]

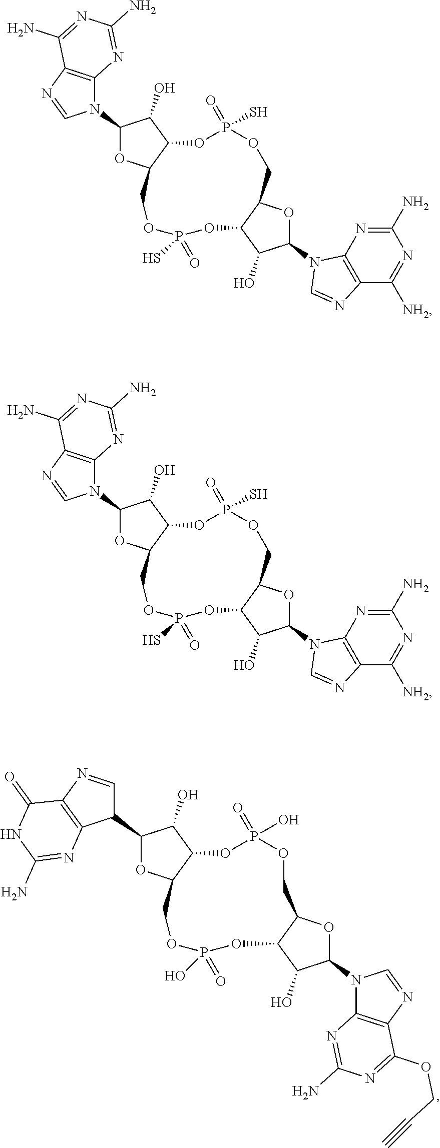 [SMILES: Nc1nc(N)c2ncn([C@@H]3O[C@@H]4CO[P@@](=O)(S)O[C@H]5[C@@H](O)[C@H](n6cnc7c(N)nc(N)nc76)O[C@@H]5CO[P@@](=O)(S)O[C@H]4[C@H]3O)c2n1 ; Nc1nc(N)c2ncn([C@@H]3O[C@@H]4CO[P@@](=O)(S)O[C@H]5[C@@H](O)[C@H](n6cnc7c(N)nc(N)nc76)O[C@@H]5CO[P@](=O)(S)O[C@H]4[C@H]3O)c2n1 ; C#CCOc1nc(N)nc2c1ncn2[C@@H]1O[C@@H]2COP(=O)(O)O[C@H]3[C@@H](O)[C@H](C4C=Nc5c4nc(N)[nH]c5=O)O[C@@H]3COP(=O)(O)O[C@H]2[C@H]1O]

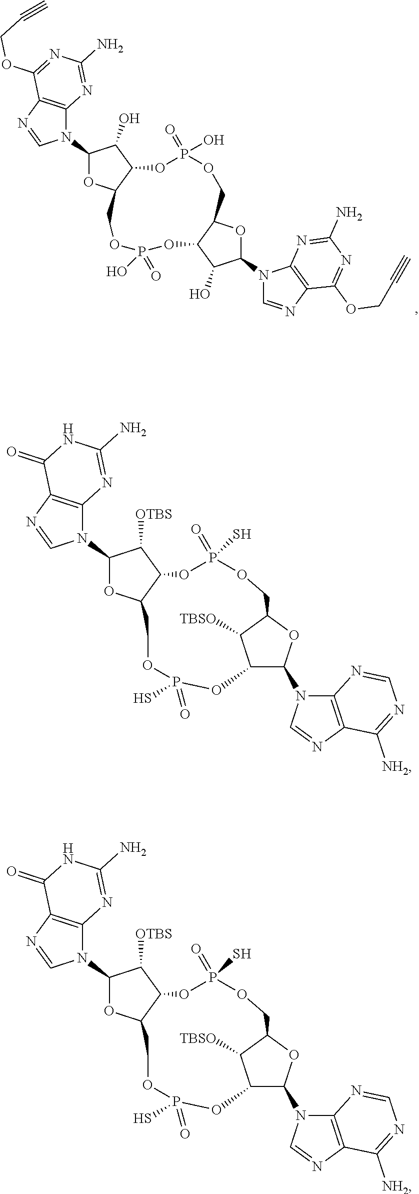 [SMILES: C#CCOc1nc(N)nc2c1ncn2[C@@H]1O[C@@H]2COP(=O)(O)O[C@H]3[C@@H](O)[C@H](n4cnc5c(OCC#C)nc(N)nc54)O[C@@H]3COP(=O)(O)O[C@H]2[C@H]1O ; CC(C)(C)[Si](C)(C)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C]

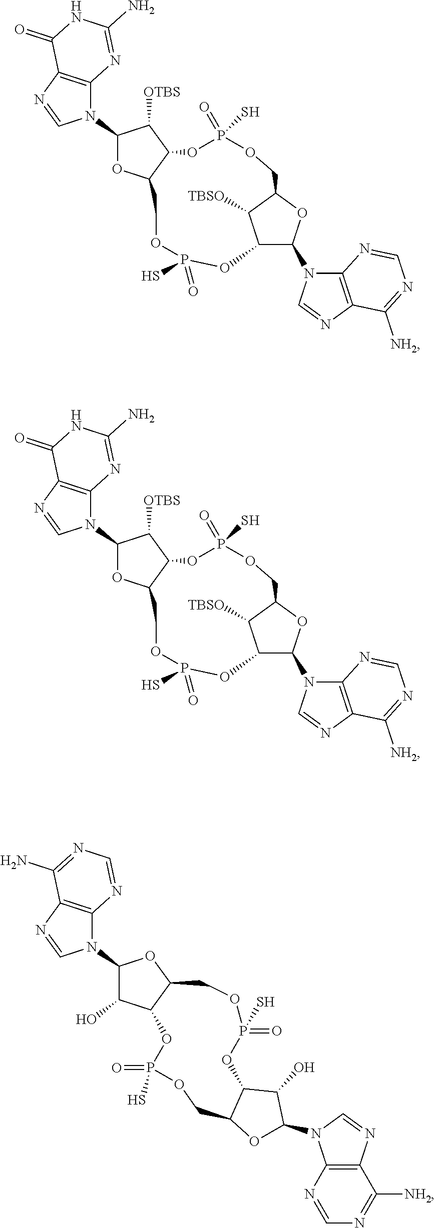 [SMILES: CC(C)(C)[Si](C)(C)O[C@@H]1[C@@H]2O[P@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C ; Nc1ncnc2c1ncn2[C@H]1O[C@H]2CO[P@](=O)(S)O[C@@H]3[C@H](O)[C@@H](n4cnc5c(N)ncnc54)O[C@H]3CO[P@](=O)(S)O[C@@H]2[C@@H]1O]

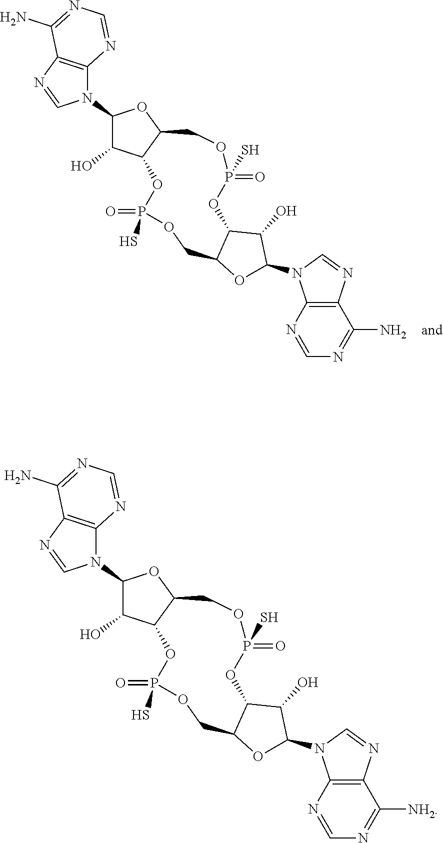 [SMILES: Nc1ncnc2c1ncn2[C@H]1O[C@H]2CO[P@@](=O)(S)O[C@@H]3[C@H](O)[C@@H](n4cnc5c(N)ncnc54)O[C@H]3CO[P@](=O)(S)O[C@@H]2[C@@H]1O ; "and" ; Nc1ncnc2c1ncn2[C@H]1O[C@H]2CO[P@@](=O)(S)O[C@@H]3[C@H](O)[C@@H](n4cnc5c(N)ncnc54)O[C@H]3CO[P@@](=O)(S)O[C@@H]2[C@@H]1O]

14. The scaffold molecule according to claim 1, wherein the scaffold molecule is selected from the group consisting of:

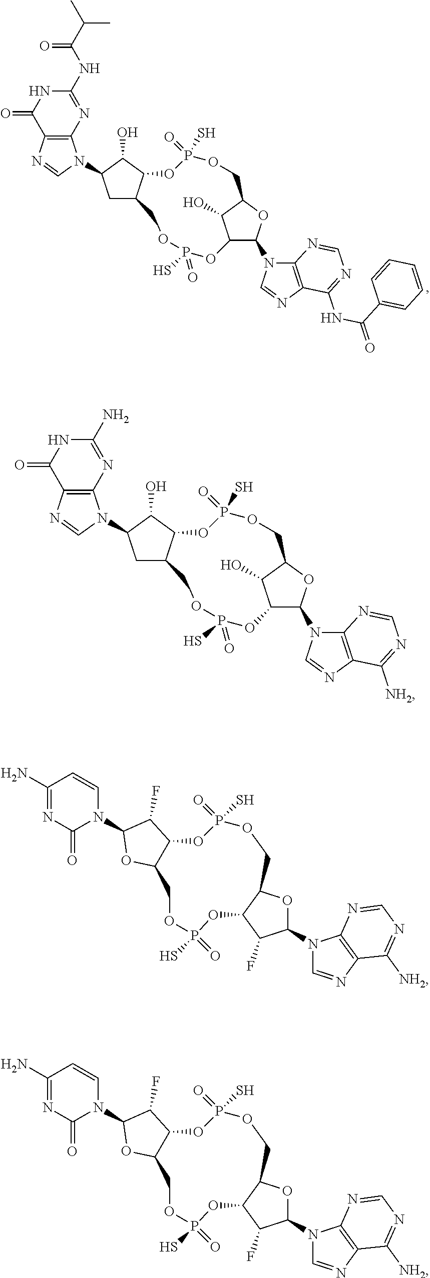

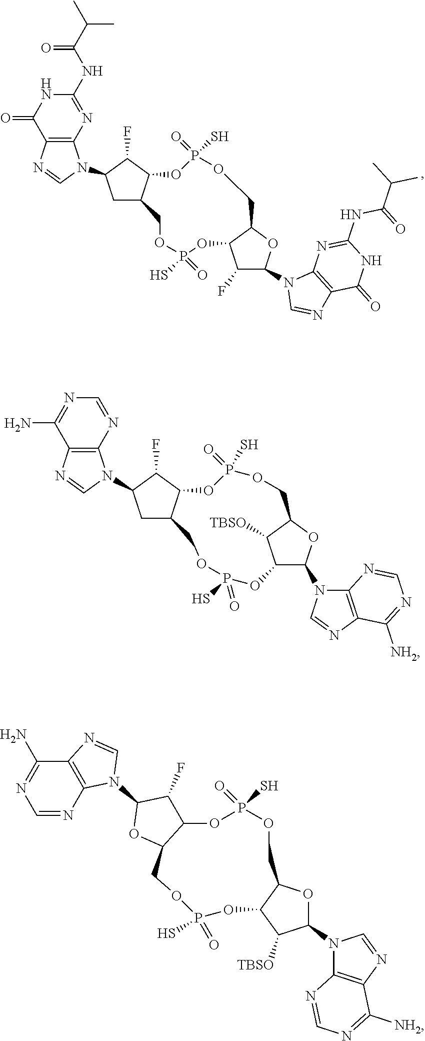 [SMILES: CC(C)C(=O)Nc1nc2c(ncn2[C@@H]2C[C@@H]3CO[P@@](=O)(S)O[C@H]4[C@@H](F)[C@H](n5cnc6c(=O)[nH]c(NC(=O)C(C)C)nc65)O[C@@H]4CO[P@@](=O)(S)O[C@H]3[C@H]2F)c(=O)[nH]1 ; CC(C)(C)[Si](C)(C)O[C@H]1[C@H]2O[P@@](=O)(S)OC[C@H]3C[C@@H](n4cnc5c(N)ncnc54)[C@H](F)[C@@H]3O[P@](=O)(S)OC[C@H]1O[C@H]2n1cnc2c(N)ncnc21 ; CC(C)(C)[Si](C)(C)O[C@@H]1[C@@H]2O[P@](=O)(S)OC[C@H]3O[C@@H](n4cnc5c(N)ncnc54)[C@H](F)C3O[P@@](=O)(S)OC[C@H]2O[C@H]1n1cnc2c(N)ncnc21]

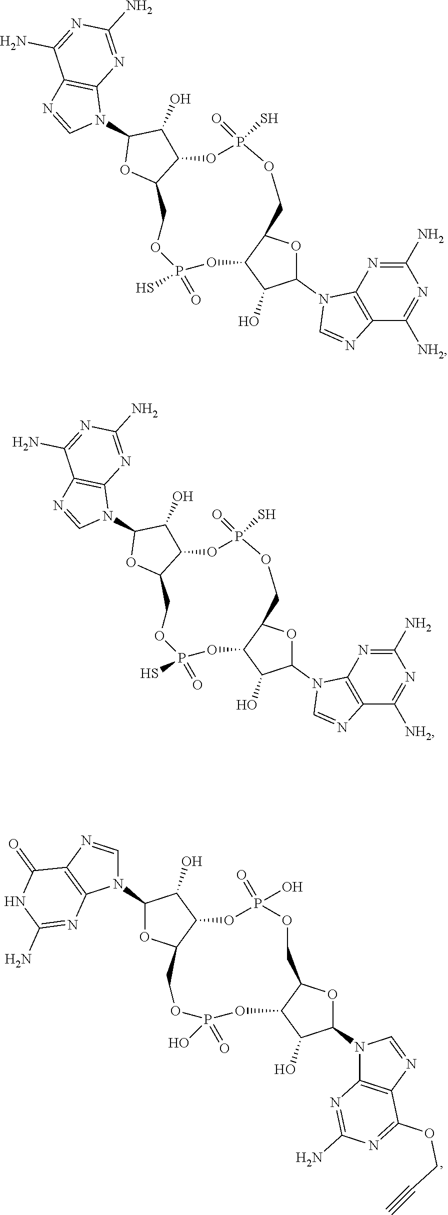 [SMILES: Nc1nc(N)c2ncn(C3O[C@@H]4CO[P@@](=O)(S)O[C@H]5[C@@H](O)[C@H](n6cnc7c(N)nc(N)nc76)O[C@@H]5CO[P@@](=O)(S)O[C@H]4[C@H]3O)c2n1 ; Nc1nc(N)c2ncn(C3O[C@@H]4CO[P@@](=O)(S)O[C@H]5[C@@H](O)[C@H](n6cnc7c(N)nc(N)nc76)O[C@@H]5CO[P@](=O)(S)O[C@H]4[C@H]3O)c2n1 ; C#CCOc1nc(N)nc2c1ncn2[C@@H]1O[C@@H]2COP(=O)(O)O[C@H]3[C@@H](O)[C@H](n4cnc5c(=O)[nH]c(N)nc54)O[C@@H]3COP(=O)(O)O[C@H]2[C@H]1O]

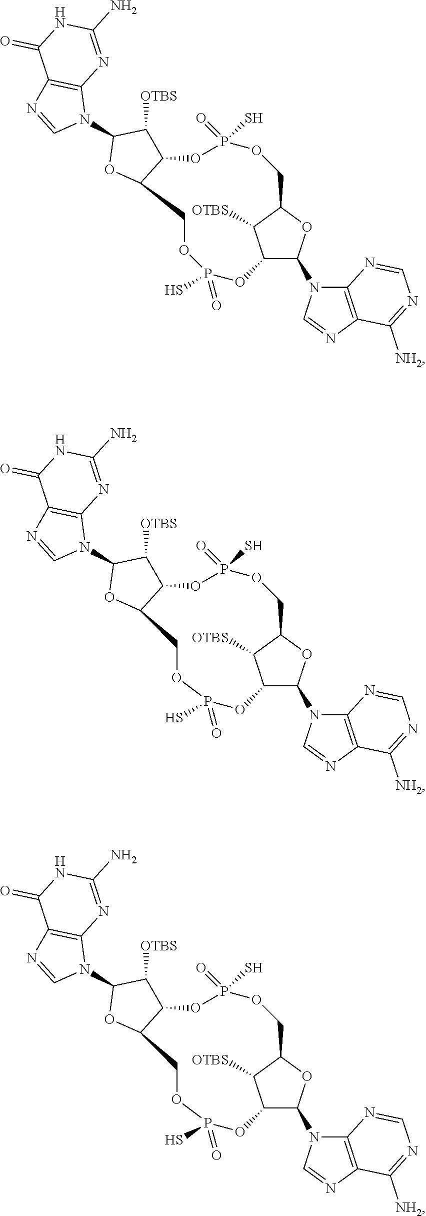 [SMILES: CC(C)(C)[Si](C)(C)O[C@@H]1[C@@H]2O[P@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@@](=O)(S)OC[C@H]3O[C@@H](n4cnc5c(N)ncnc54)[C@H](O[P@](=O)(S)OC[C@H]2O[C@H]1n1cnc2c(=O)[nH]c(N)nc21)[C@@H]3O[Si](C)(C)C(C)(C)C ; CC(C)(C)[Si](C)(C)O[C@@H]1[C@@H]2O[P@](=O)(S)OC[C@H]3O[C@@H](n4cnc5c(N)ncnc54)[C@H](O[P@@](=O)(S)OC[C@H]2O[C@H]1n1cnc2c(=O)[nH]c(N)nc21)[C@@H]3O[Si](C)(C)C(C)(C)C]

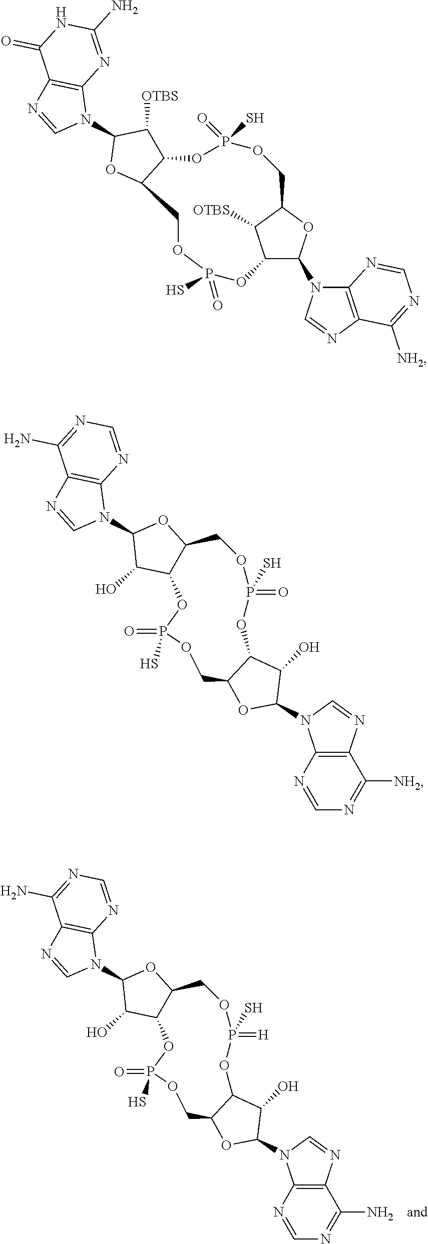

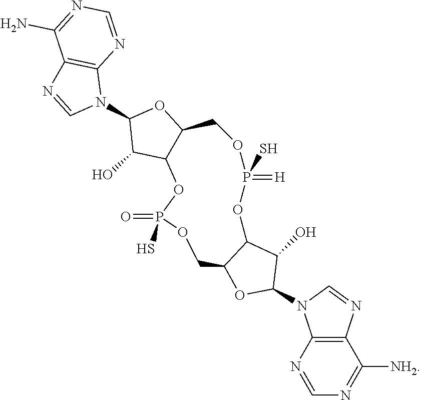

15. A method of identifying a STING inhibitor comprising the steps of:
a) providing a scaffold molecule according to claim 1;
b) synthesizing a derivative of the scaffold molecule;
c) measuring the STING inhibitory activity of the derivative; and
d) identifying the derivative as a STING inhibitor if the derivative exhibits greater STING inhibitory activity than the STING inhibitory activity of the scaffold molecule.

16-25. (canceled)

26. A compound having the structure of Formula VI or VII: 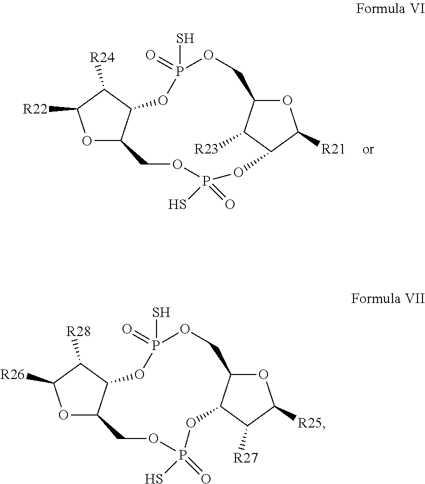
or a prodrug, tautomer, pharmaceutically acceptable salt, pharmaceutically acceptable solvate or pharmaceutically acceptable hydrate thereof,
wherein:
R21 is adenine or adenine-6-benzamide linked to the structure via the N9 position, wherein the 6-position of adenine or adenine-6-benzamide is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy;
R22 is adenine, 2,6-diamino-purine, guanine or guanine-2-isobutyramide linked to the structure via the N9 position, wherein the 2-position of guanine or guanine-2-isobutyramide, the 6-position of adenine, and the 2-position and/or the 6-position of 2,6-diamino-purine are independently optionally replaced with a substituent selected from the group consisting of mono-alkylamino, di-alkylamino, —NHCH₂R¹⁰⁴ and —NHC(O)R¹⁰⁴ wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine or guanine-2-isobutyramide is optionally replaced with —OR^{x}, where R^{x} is alkyl, alkenyl or alkynyl;
R23 is selected from the group consisting of —H, —CN, halogen, —OH, alkoxy, —OCH₂R¹⁰⁰ wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl;
R24 is selected from the group consisting of —H, —CN, halogen, —OH, alkoxy, —OCH₂R¹⁰⁰ wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl;
R25 is adenine, 2,6-diamino-purine, guanine, guanine-2-isobutyramide or guanine-6-propargyl ether linked to the structure via the N9 position, or cytosine linked to the structure via the N1 position, wherein the 6-position of adenine, 2-position and/or 6-position of 2,6-diamino-purine, 2-position of guanine, guanine-2-isobutyramide or guanine-6-propargyl ether, or 4-position of cytosine are independently optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, guanine-2-isobutyramide or guanine-6-propargyl ether or the 2-position of cytosine is optionally replaced with —OR^{x}, where R^{x} is alkyl, alkenyl or alkynyl;
R26 is adenine, 2,6-diamino-purine, guanine, guanine-2-isobutyramide or guanine-6-propargyl ether linked to the structure via the N9 position, or cytosine linked to the structure via the N1 position, wherein the 6-position of adenine, 2-position and/or 6-position of 2,6-diamino-purine, 2-position of guanine guanine-2-isobutyramide or guanine-6-propargyl ether, or 4-position of cytosine are independently optionally replaced with a substituent selected from the group consisting of mono-alkylamino, di-alkylamino, —NHCH₂R¹⁰⁴ and —NHC(O)R¹⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, guanine-2-isobutyramide or guanine-6-propargyl ether, or the 2-position of cytosine is optionally replaced with —OR^{x}, where R^{x} is alkyl, alkenyl or alkynyl;
R27 is selected from the group consisting of —H, —CN, halogen, —OH, alkoxy, —OCH₂R¹⁰⁰ wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl; and
R28 is selected from the group consisting of —H, —CN, halogen, —OH, alkoxy, —OCH₂R¹⁰⁰ wherein R¹⁰⁰ is alkenyl or alkynyl and —SiR¹⁰¹R¹⁰²R¹⁰³, wherein R¹⁰¹, R¹⁰² and R¹⁰³ are independently C₁₋₆alkyl or phenyl;
wherein the compound exhibits measurable STING inhibitory activity.

27. The compound according to claim 26, wherein the compound has a structure selected from the group consisting of Formula VIa, Formula VIIa and Formula VIIb: 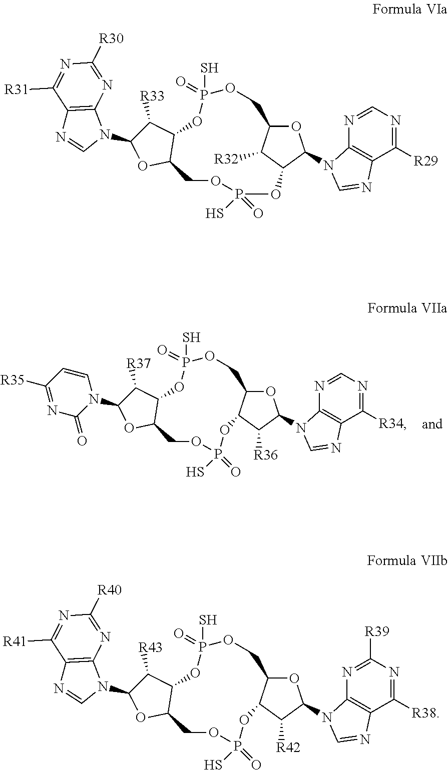
or a prodrug, tautomer, pharmaceutically acceptable salt, pharmaceutically acceptable solvate or pharmaceutically acceptable hydrate thereof,
wherein:
R29, R30, R34, R35, R39 and R40 are independently selected from the group consisting of —NH₂, —NHR^{y}, —NR^{y}R^{z}, —NHCH₂R¹⁰⁴ and —NHC(O)R¹⁰⁴, wherein R^{y} and R^{z} are independently alkyl, and R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy;
R31, R38 and R41 are independently —OR^{w}, where R^{w} is —H, alkyl, alkenyl or alkynyl; and
R32, R33, R36, R37, R42 and R43 are independently selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl.

28. A compound having the structure of Formula VIII: 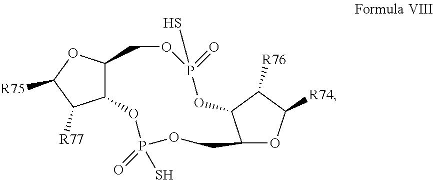
or a prodrug, tautomer, pharmaceutically acceptable salt, pharmaceutically acceptable solvate or pharmaceutically acceptable hydrate thereof,
wherein:
R74 is a purine or modified purine linked to the structure via the N9 position, or a pyrimidine or modified pyrimidine linked to the structure via the N1 position, preferably wherein the purine or modified purine is adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, and wherein the pyrimidine or modified pyrimidine is cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl;
R75 is a purine or modified purine linked to the structure via the N9 position, or a pyrimidine or modified pyrimidine linked to the structure via the N1 position, preferably wherein the purine or modified purine is adenin-9-yl, guanin-9-yl, hypoxanthin-9-yl, xanthin-9-yl, isoguanin-9-yl, or 2,6-diamino-purin-9-yl, wherein the 6-amino of adenine or isoguanine, 2-amino of guanine, or either or both of the 2- and 6-amino of 2,6-diamino-purine are optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 6-position of guanine, 6-position of hypoxanthine, either or both of the 2- or 6-position of xanthine, or 2-position of isoguanine are optionally replaced with —OR^{x}, where R^{x} is alkyl, alkenyl or alkynyl, and wherein the pyrimidine or modified pyrimidine is cytosin-1-yl, thymin-1-yl, or uracil-1-yl, wherein the 4-amino of cytosine is optionally modified with a protecting group, or the amino is optionally replaced with a substituent selected from the group consisting of mono-alkylamino, di-alkylamino, —NHCH₂R¹⁰⁴ and —NHC(O)R¹⁰⁴, wherein R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy, and wherein the 2-position of cytosine or the 2-position and/or 4-position of thymine or uracil are optionally replaced with —OR^{x}, where R^{x} is alkyl, alkenyl or alkynyl;
R76 is selected from the group consisting of —H, —CN, halogen, —OH, alkoxy, —OCH₂R¹⁰⁰ wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl;
R77 is selected from the group consisting of —H, —CN, halogen, —OH, alkoxy, —OCH₂R¹⁰⁰ wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl;
wherein the compound exhibits measurable STING inhibitory activity.

29. The compound according to claim 28, wherein the compound has a structure of Formula VIIIa: 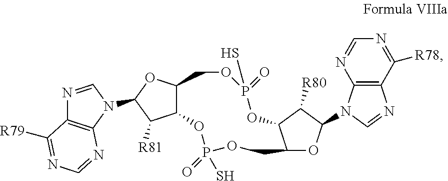
or a prodrug, tautomer, pharmaceutically acceptable salt, pharmaceutically acceptable solvate or pharmaceutically acceptable hydrate thereof,
wherein:
R78 and R79 are independently selected from the group consisting of —NH₂, —NHR^{y}, —NR^{y}R^{z}, —NHCH₂R¹⁰⁴ and —NHC(O)R¹⁰⁴, wherein R^{y} and R^{z} are independently alkyl, and R¹⁰⁴ is alkyl, alkenyl, alkynyl, phenyl or 5 or 6 membered single ring heteroaryl, where phenyl and 5 or 6 membered single ring heteroaryl are optionally substituted with one or more (e.g. 1, 2, 3, 4 or 5, preferably 1, 2, or 3) substituents independently selected from the group consisting of halogen, —OH, —CN, alkyl, and alkoxy; and
R80 and R81 are independently selected from the group consisting of —H, —CN, halogen, —OH, alkoxy, —OCH₂R¹⁰⁰, wherein R¹⁰⁰ is alkenyl or alkynyl, and —SiR¹⁰¹R¹⁰²R¹⁰³, wherein R¹⁰¹, R¹⁰² and R¹⁰³ are independently C₁₋₆alkyl or phenyl.

30-38. (canceled)

39. A pharmaceutical composition comprising a molecule of claim 1 and a pharmaceutically acceptable excipient.

40. A method for treating an individual in need of thereof, comprising: administering to the individual an effective amount of a pharmaceutical composition according to claim 39, wherein the molecule has measurable STING antagonist activity, under conditions where STING activity within the individual is reduced.

41-47. (canceled)

48. A method of inhibiting STING in a cell, comprising: administering a molecule according to claim 1 to the cell, wherein the molecule has measurable STING antagonist activity, under conditions where the molecule binds to STING present within the cell.

49. (canceled)
